(19) United States
(12) Patent Application Publication
(10) Pub. No.: US 2003/0055220 A1
(43) Pub. Date: Mar. 20, 2003

(54) Protein-protein interactions between Shigella flexneri polypeptides and mammalian polypeptides

(76) Inventor: Pierre Legrain, Paris (FR)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/043,487

(22) Filed: Jan. 11, 2002

Related U.S. Application Data
(60) Provisional application No. 60/261,130, filed on Jan. 12, 2001.

(51) Int. Cl. C07K 1/00; C07K 14/00; C07K 17/00

(52) U.S. Cl. 530/350

(57) ABSTRACT

The present invention relates to protein-protein interactions between Shigella polypeptides and mammalian polypeptides. More specifically, the present invention relates to complexes of polypeptides or polynucleotides encoding the polypeptides, fragments of the polypeptides, antibodies to the complexes, Selected Interacting Domains (SID®) which are identified due to the protein-protein interactions, methods for screening drugs for agents which modulate the interaction of proteins and pharmaceutical compositions that are capable of modulating the protein-protein interactions.

### PRIORITY

This application claims priority on the basis of U.S. Provisional Application No. 60/261,130, filed Jan. 12, 2001, the contents of which are hereby incorporated by reference.

### BACKGROUND OF THE INVENTION

Most biological processes involve specific protein-protein interactions. Protein-protein interactions enable two or more proteins to associate. A large number of non-covalent bonds form between the proteins when two protein surfaces are precisely matched. These bonds account for the specificity of recognition. Thus, protein-protein interactions are involved, for example, in the assembly of enzyme subunits, in antibody-antigen recognition, in the formation of biochemical complexes, in the correct folding of proteins, in the metabolism of proteins, in the transport of proteins, in the localization of proteins, in protein turnover, in first translation modifications, in the core structures of viruses and in signal transduction.

General methodologies to identify interacting proteins or to study these interactions have been developed. Among these methods are the two-hybrid system originally developed by Fields and co-workers and described, for example, in U.S. Pat. Nos. 5,283,173, 5,468,614 and 5,667,973, which are hereby incorporated by reference.

The earliest and simplest two-hybrid system, which acted as basis for development of other versions, is an in vivo assay between two specifically constructed proteins. The first protein, known in the art as the “bait protein” is a chimeric protein which binds to a site on DNA upstream of a reporter gene by means of a DNA-binding domain or BD. Commonly, the binding domain is the DNA-binding domain from either Gal4 or native E. coli LexA and the sites placed upstream of the reporter are Gal4 binding sites or LexA operators, respectively.

The second protein is also a chimeric protein known as the “prey” in the art. This second chimeric protein carries an activation domain or AD. This activation domain is typically derived from Gal4, from VP16 or from B42.

Besides the two hybrid systems, other improved systems have been developed to detected protein-protein interactions. For example, a two-hybrid plus one system was developed that allows the use of two proteins as bait to screen available cDNA libraries to detect a third partner. This method permits the detection between proteins that are part of a larger protein complex such as the RNA polymerase II holoenzyme and the TFIIH or TFIID complexes. Therefore, this method, in general, permits the detection of ternary complex formation as well as inhibitors preventing the interaction between the two previously defined fused proteins.

Another advantage of the two-hybrid plus one system is that it allows or prevents the formation of the transcriptional activator since the third partner can be expressed from a conditional promoter such as the methionine-repressed Met25 promoter which is positively regulated in medium lacking methionine. The presence of the methionine-regulated promoter provides an excellent control to evaluate the activation or inhibition properties of the third partner due to its “on” and “off” switch for the formation of the transcriptional activator. The three-hybrid method is described, for example in Tirode et al., The Journal of Biological Chemistry, 272, No. 37 pp. 22995-22999 (1997). incorporated herein by reference.

Besides the two and two-hybrid plus one systems, yet another variant is that described in Vidal et al., Proc. Natl. Sci. 93 pgs. 10315-10320 called the reverse two- and one-hybrid systems where a collection of molecules can be screened that inhibit a specific protein-protein or protein/DNA interactions, respectively.

A summary of the available methodologies for detecting protein-protein interactions is described in Vidal and Legrain, Nucleic Acids Research Vol. 27, No. 4 pgs.919-929 (1999) and Legrain and Selig, FEBS Letters 480 pgs. 32-36 (2000) which references are incorporated herein by reference.

However, the above conventionally used approaches and especially the commonly used two-hybrid methods have their drawbacks. For example, it is known in the art that, more often than not, false positives and false negatives exist in the screening method. In fact, a doctrine has been developed in this field for interpreting the results and in common practice an additional technique such as co-immunoprecipitation or gradient sedimentation of the putative interactors from the appropriate cell or tissue type are generally performed. The methods used for interpreting the results are described by Brent and Finley, Jr. in Ann. Rev. Genet., 31 pgs. 663-704 (1997). Thus, the data interpretation is very questionable using the conventional systems.

One method to overcome the difficulties encountered with the methods in the prior art is described in WO 99/42612, incorporated herein by reference. This method is similar to the two-hybrid system described in the prior art in that it also uses bait and prey polypeptides. However, the difference with this method is that a step of mating at least one first haploid recombinant yeast cell containing the prey polypeptide to be assayed with a second haploid recombinant yeast cell containing the bait polynucleotide is performed. Of course the person skilled in the art would appreciate that either the first recombinant yeast cell or the second recombinant yeast cell also contains at least one detectable reporter gene that is activated by a polypeptide including a transcriptional activation domain.

The method described in WO 99/42612 permits the screening of more prey polynucleotides with a given bait polynucleotide in a single step than in the prior art systems due to the cell to cell mating strategy between haploid yeast cells. Furthermore, this method is more thorough and reproducible, as well as sensitive. Thus, the presence of false negatives and/or false positives is extremely minimal as compared to the conventional prior art methods.

The genus Shigella includes four species (major serogroups): S. dysenteriae (Grp. A), S. flexneri (Grp. B), S. boydii (Grp. C) and S. sonnei (Grp. D) as classified in Bergey's Manual for Systematic Bacteriology (N. R. Krieg, ed., pp. 423-427 (1984)). The genera Shigella and Escherichia are phylogenetically closely related. Brenner and others have suggested that the two are more correctly considered sibling species based on DNA/DNA reassociation studies (D. J. Brenner et al., International J. Systematic Bacteriology, 23:1-7 (1973)). These studies showed that Shigella species are on average 80-89% related to E. coli at the DNA level. Also, the degree of relatedness between Shigella species is on average 80-89%.

The genus Shigella is pathogenic in humans; it causes bacillary dysentery at levels of infection of 10 to 100 organisms.

Shigellosis or bacillary dysentery is a disease that is endemic throughout the world. The disease presents a particularly serious public health problem in tropical regions and developing countries where Shigella dysenteriae and S. flexneri predominate. In industrialized countries, the principal etiologic agent is S. sonnei although sporadic cases of shigellosis are encountered due to S. flexneri, S. boydii and certain entero-invasive Escherichia coli.

The primary step in the pathogenesis of bacillary dysentery is invasion of the human colonic mucosa by Shigella (Labrec, E. H., H. Schneider, T. J. Magnani, and S. B. Formal. 1964. Epithelial cell penetration as an essential step in the pathogenesis of bacillary dysentery. J. Bacteriol. 88:1503). Mucosal invasion encompasses several steps which include penetration of the bacteria into epithelial cells, intracellular multiplication, killing of host cells, and final spreading to adjacent cells and to connective tissue (Formal, S. B., T. L. Hale, and P. J. Sansonetti. 1983. Invasive enteric pathogens. Rev. Infect. Dis. 5:S702, Rout, W. R., S. B. Formal, R. A. Giannella, and G. J. Dammin. 1975. The pathophysiology of Shigella diarrhea in the Rhesus monkey; intestinal transport, morphology and bacteriological studies. Gastroenterology 68:270, Takeuchi, A., H. Spring, E. H. LaBrec, and S. B. Formal. 1965. Experimental acute colitis in the Rhesus monkey following peroral infection with Shigella flexneri. Am. J. Pathol. 52:503, Takeuchi, A. 1967. Electron microscope studies of experimental Salmonella infection. I. Penetration into cells of the intestinal epithelium by Salmonella typhimurium. Am. J. Pathol. 47:1011). The overall process which is usually limited to the mucosal surface leads to a strong inflammatory reaction which is responsible for abscesses and ulcerations (Labrec, E. H., H. Schneider, T. J. Magnani, and S. B. Formal. 1964. Epithelial cell penetration as an essential step in the pathogenesis of bacillary dysentery. J. Bacteriol. 88:1503., Rout, W. R., S. B. Formal, R. A. Giannella, and G. J. Dammin. 1975. The pathophysiology of Shigella diarrhea in the Rhesus monkey; intestinal transport, morphology and bacteriological studies. Gastroenterology 68:270, Takeuchi, A., H. Spring, E. H. LaBrec, and S. B. Formal. 1965. Experimental acute colitis in the Rhesus monkey following peroral infection with Shigella flexneri. Am. J. Pathol. 52:503).

Even though dysentery is characteristic of shigellosis, it may be preceded by watery diarrhea. Diarrhea appears to be the result of disturbances in colonic reabsorption and increased jejunal secretion whereas dysentery is a purely colonic process (Kinsey, M. D., S. B. Formal, G. J. Dammin, and R. A. Giannella. 1976. Fluid and electrolyte transport in Rhesus monkeys challenged intraceacally with Shigella flexneri 2a. Infect. Immun. 14:368). These include toxic megacolon, leukemoid reactions and hemolytic-uremic syndrome (“HUS”). The latter is a major cause of mortality from shigellosis in developing areas (Gianantonio, C., H. Vitacco, F. Mendilaharzu, A. Rutty, and J. Mendilaharzu. 1964. The hemolytic-uremic syndrome. J. Pediatr. 64:478, Koster, F., J. Levin, L. Walker, K. S. K. Tung, R. H. Gilman, M. M. Rajaman, M. A. Majid, S. Islam, and R. C. Williams Jr. 1977. Hemolyticuremic syndrome after shigellosis. Relation to endotoxin and circulating immune complexes. N. Engl. J. Med. 298:927).

The role of Shiga-toxin produced at high level by S. dysenteriae 1 (Conradi, H., 1903. Ueber loshlishe, durch aseptische Autolyse, erhaltene Giftstoffe von Ruhr--un Typhus bazillen. Dtsch. Med. Wochenschr. 29:26) and Shiga-like toxins (“SLT”) produced at low level by S. flexneri and S. sonnei (Keusch, G. T., and M. Jacewicz. 1977. The pathogenesis of Shigella diarrhea. VI. Toxin and antitoxin in Shigella flexneri and Shigella sonnei infections in humans. J. Infect. Dis. 135:552) in the four major stages of shigellosis (i.e., invasion of individual epithelial cells, tissue invasion, diarrhea and systemic symptoms) is not well understood. For review see O'Brien and Holmes (O'Brien, A. D., and R. K. Holmes. 1987. Shiga and Shiga-like toxins. Microbiol. Rev. 51:206). Plasmids of 180-220 kilobases (“kb”) are essential in all Shigella species for invasion of individual epithelial cells (Rout, W. R., S. B. Formal, R. A. Giannella, and G. J. Dammin. 1975. The pathophysiology of Shigella diarrhea in the Rhesus monkey; intestinal transport, morphology and bacteriological studies. Gastroenterology 68:270, Sansonetti, P. J., D. J. Kopecko, and S. B. Formal. 1981. Shigella sonnei plasmids: evidence that a large plasmid is neceessary for virulence. Infect. Immun. 34:75, Sansonetti, P. J., T. L. Hale, G. I. Dammin, C. Kapper, H. H. Collins Jr., and S. B. Formal. 1983. Alterations in the pathogenesis of Escherichia coli K12 after transfer of plasmids and chromosomal genes from Shigella flexneri . Infect. Immun. 39:1392). This includes entry, intracellular multiplication and early killing of host cells (Clerc, P., A. Ryter, J. Mounier, and P. J. Sansonetti. 1987. Plasmid-mediated early killing of eucaryotic cells by Shigella flexneri as studied by infection of J774 macrophages. Infect. Immun. 55:521, Clerc, P., and P. J. Sansonetti. 1987. Entry of Shigella flexneri into HeLa cells: Evidence for directed phagocytosis involving actin polymerization and myosin accumulation. Infect. Immun. 55:2681). The role of Shiga-toxin and SLT at this stage is unclear.

Recent evidence indicates that Shiga-toxin is cytotoxic for primary cultures of human colonic cells (Moyer, M. P., P. S. Dixon, S. W. Rothman, and J. E. Brown. 1987. Cytotoxicity of Shiga toxin for human colonic and ileal epithelial cells. Infect. Immun. 55:1533). Tissue invasion requires additional chromosomally encoded products among which are smooth lipopolysaccharides (“LPS”) (Sansonetti, P. J., T. L. Hale, G. I. Dammin, C. Kapper, H. H. Collins Jr., and S. B. Formal. 1983. Alterations in the pathogenesis of Escherichia coli K12 after transfer of plasmids and chromosomal genes from Shigella flexneri. Infect. Immun. 39:1392), the non-characterized product of the Kcp locus, and aerobactin. A region of the S. flexneri chromosome necessary for fluid production in rabbit ileal loops has been localized to the rha-mt1 regions and near the lysine decarboxylase locus (Sansonetti, P. J., T. L. Hale, G. I. Dammin, C. Kapper, H. H. Collins Jr., and S. B. Formal. 1983. Alterations in the pathogenesis of Escherichia coli K12 after transfer of plasmids and chromosomal genes from Shigella flexneri . Infect. Immun. 39:1392). However, no evidence has been adduced to show that the ability to cause fluid accumulation is due to the SLT of S. flexneri. Thus, the role of Shiga-toxin in causing the systemic complications of shigellosis is still hypothetical. However, Shiga-toxin can mediate vascular damage since capillary lesions observed in HUS resemble those observed in cerebral vessels of animals injected with this toxin (Bridgewater, F. A. I., R. S. Morgan, K. E. K. Rowson, and G. P. Wright. 1955. the neurotoxin of Shigella shigae. Morphological and functional lesions produced in the central nervous system of rabbits. Br. J. Exp. Pathol. 36: 447, Cavanagh, J. B., J. G. Howard, and J. L. Whitby. 1956. The neurotoxin of Shigella shigae. A comparative study of the effects produced in various laboratory animals. Br. J. Exp. Med. 37:272).

As described before, the genera of Shigella and Escherichia are phylogenetically closely related. Furthermore, the pathogenesis of enteroinvasive E. coli is very similar to that of Shigella. In both, dysentery results from invasion of the colonic epithelial cells followed by intracellular multiplication which leads to bloody, mucous discharge with scanty diarrhea.

Pathogenic E. coli serotypes are collectively referred to as Enterovirulent E. coli (EVEC) (J. R. Lupski, et al., J. Infectious Diseases, 157:1120-1123 (1988); M. M. Levine, J. Infectious Diseases, 155:377-389 (1987); M. A. Karmali, Clinical Microbiology Reviews, 2:15-38 (1989)). This group includes at least 5 subclasses of E. coli, each having a characteristic pathogenesis pathway resulting in diarrheal disease. The subclasses include Enterotoxigenic E. coli (ETEC), Verotoxin-Producing E. coli (VTEC), Enteropathogenic E. coli (EPEC), Enteroadherent E. coli (EAEC) and Enteroinvasive E. coli (EIEC). The VTEC include Enterohemorrhagic E. coli (EHEC) since these produce verotoxins.

Thus, detection of Shigella and EIEC is important in various medical contexts. For example, the presence of either Shigella or EIEC in stool samples is indicative of gastroenteritis, and the ability to screen for their presence is useful in treating and controlling that disease. Detection of Shigella or EIEC in any possible transmission vehicle such as food is also important to avoid spread of gastroenteritis.

That is why there is a great need to construct Protein Interaction Map between Shigella polypeptides and human polypeptides in order to understand mechanisms of Shigella pathogenesis and to identify drug target to treat Shigella associated diseases and Shigella detection means.

### SUMMARY OF THE PRESENT INVENTION

Thus, it is an object of the present invention to identify protein-protein interactions between Shigella polypeptides and mammalian, preferably human, polypeptides.

It is another object of the present invention to identify protein-protein interactions between Shigella polypeptides and mammalian, preferably human, polypeptides for the development of more effective and better targeted therapeutic applications.

It is yet another object of the present invention to identify complexes of polypeptides or polynucleotides encoding the polypeptides and fragments of the polypeptides of Shigella genus and polypeptides and fragments of the polypeptides of mammals, preferably human.

It is yet another object of the present invention to identify antibodies to these complexes of polypeptides or polynucleotides encoding the polypeptides and fragments of the polypeptides of Shigella genus and mammals, preferably human, including polyclonal, as well as monoclonal antibodies that are used for detection.

It is still another object of the present invention to identify selected interacting domains of the polypeptides, called SID® polypeptides.

It is still another object of the present invention to identify selected interacting domains of the polynucleotides, called SID® polynucleotides.

It is another object of the present invention to generate protein-protein interactions maps called PIM®s.

It is yet another object of the present invention to provide a method for screening drugs for agents which modulate the interaction of proteins and pharmaceutical compositions that are capable of modulating the protein-protein interactions between Shigella polypeptides and mammalian, preferably human, polypeptides.

It is another object to administer the nucleic acids of the present invention via gene therapy.

It is yet another object of the present invention to provide protein chips or protein microarrays.

It is yet another object of he present invention to provide a report in, for example paper, electronic and/or digital forms, concerning the protein-protein interactions, the modulating compounds and the like as well as a PIM®.

Thus the present invention, in one aspect thereof, relates to a protein complex between a Shigella polypeptide and a mammalian polypeptide. In another embodiment, the Shigella and the mammalian polypeptides are polypeptides set forth on columns 1 and 3 respectively of Table II.

Furthermore, the present invention provides SID® polynucleotides and SID® polypeptides of Table III, as well as a PIM® between Shigella polypeptides and mammalian, preferably human, polypeptides.

The present invention also provides antibodies to the protein-protein complexes between Shigella polypeptides and mammal, preferably human, polypeptides.

In another embodiment the present invention provides a method for screening drugs for agents that modulate the protein-protein interactions and pharmaceutical compositions that are capable of modulating protein-protein interactions.

In another embodiment the present invention provides protein chips or protein microarrays.

In yet another embodiment the present invention provides a report in, for example, paper, electronic and/or digital forms.

### BRIEF DESCRIPTION OF THE DRAWINGS

FIG. 1 is a schematic representation of the pB1 plasmid.

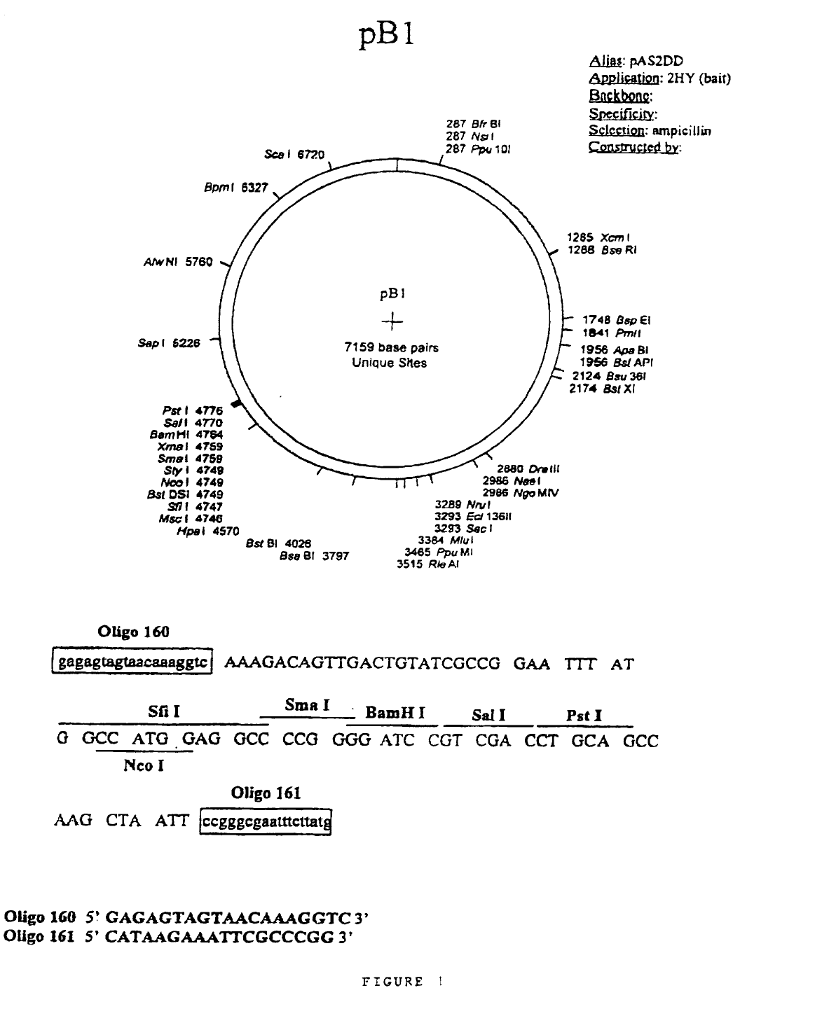

FIG. 2 is a schematic representation of the pB5 plasmid.

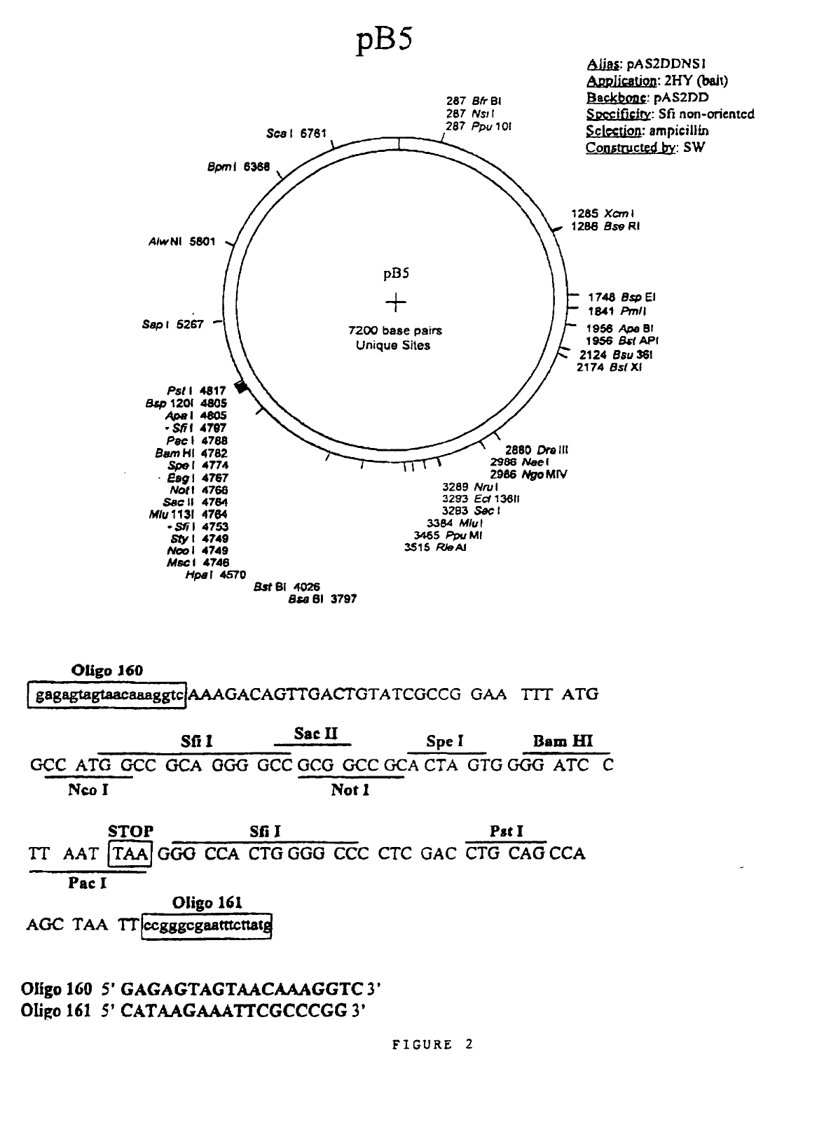

FIG. 3 is a schematic representation of the pB6 plasmid.

FIG. 4 is a schematic representation of the pB13 plasmid.

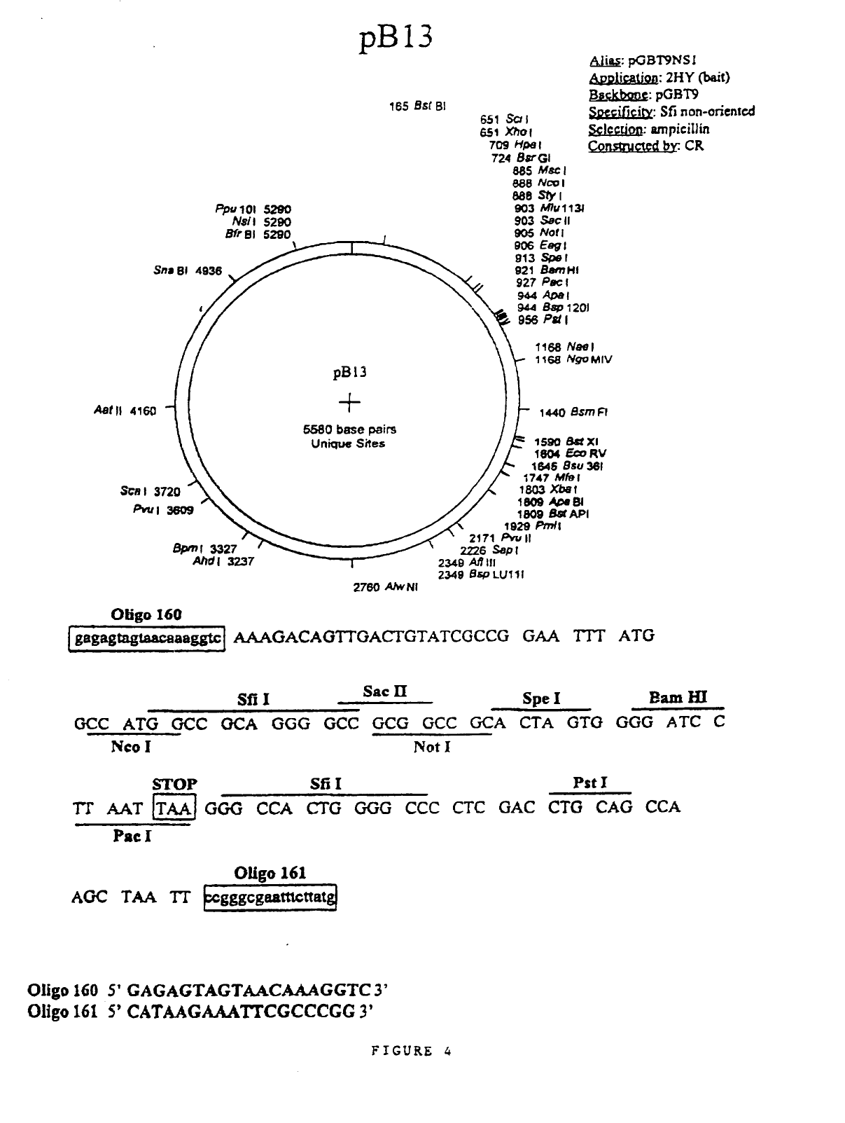

FIG. 5 is a schematic representation of the pB14 plasmid.

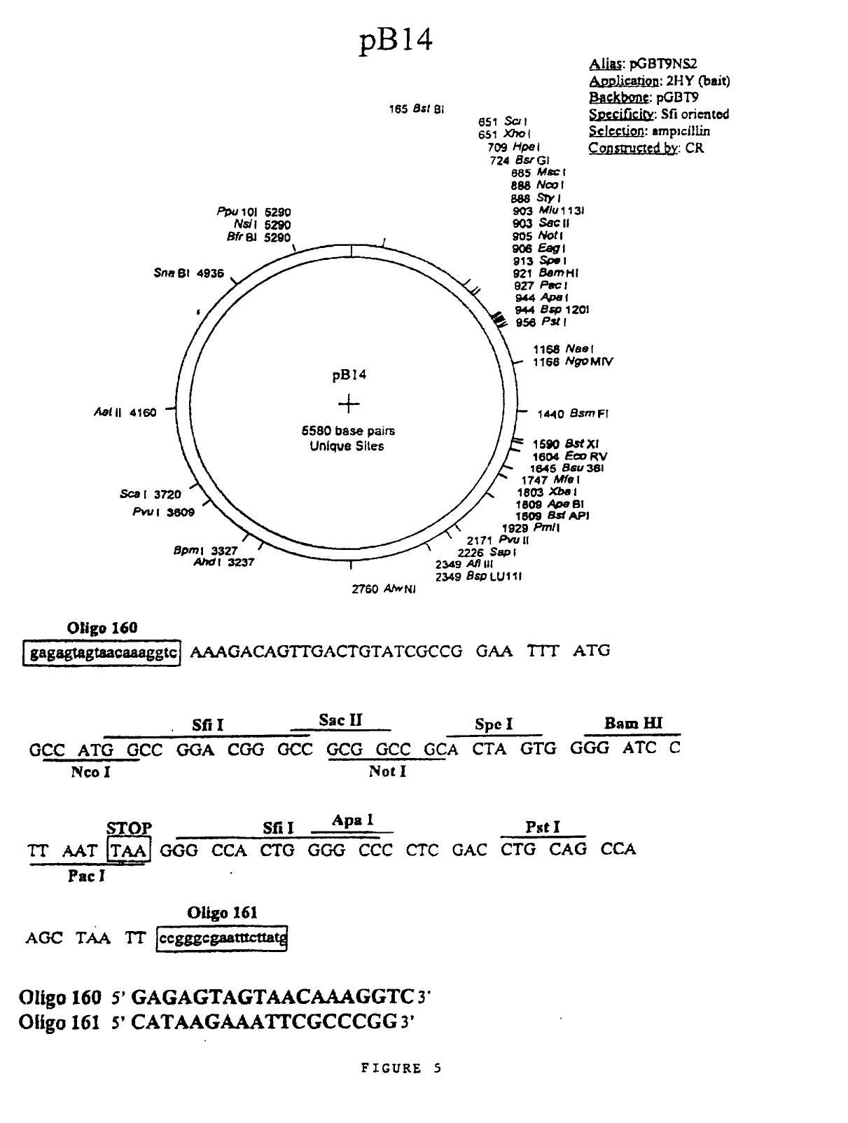

FIG. 6 is a schematic representation of the pB20 plasmid.

FIG. 7 is a schematic representation of the pP1 plasmid.

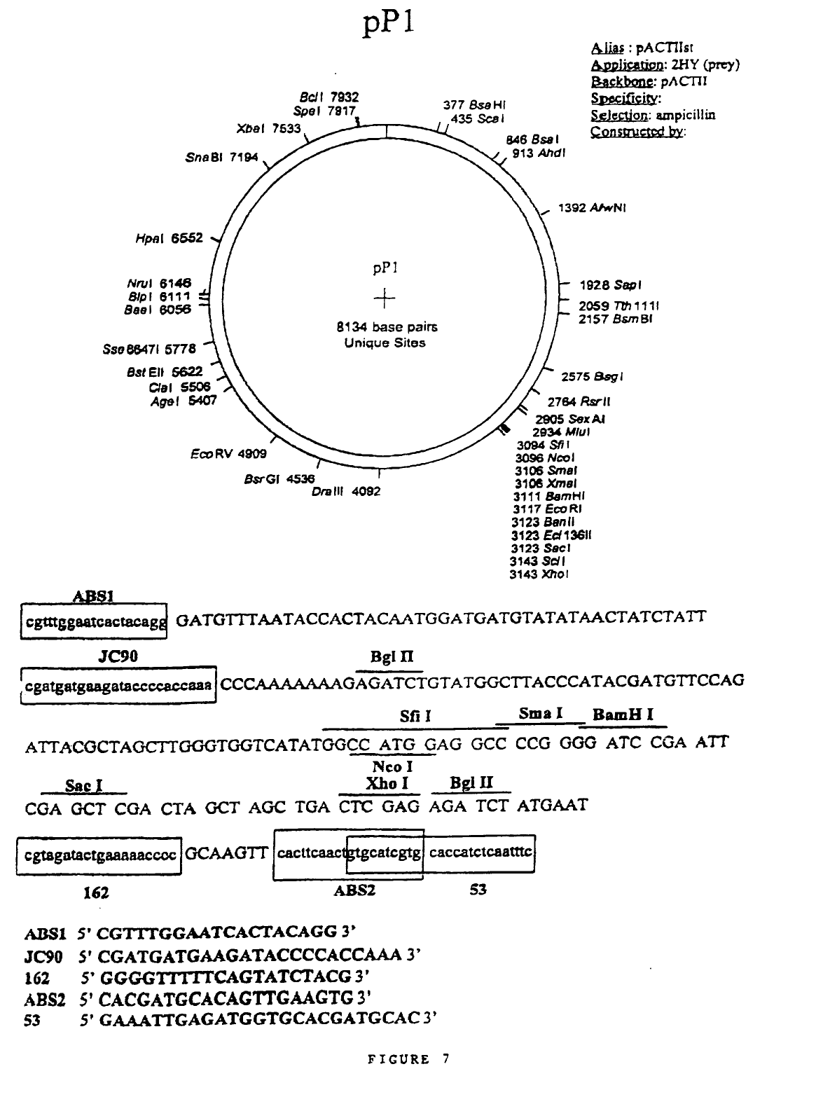

FIG. 8 is a schematic representation of the pP2 plasmid.

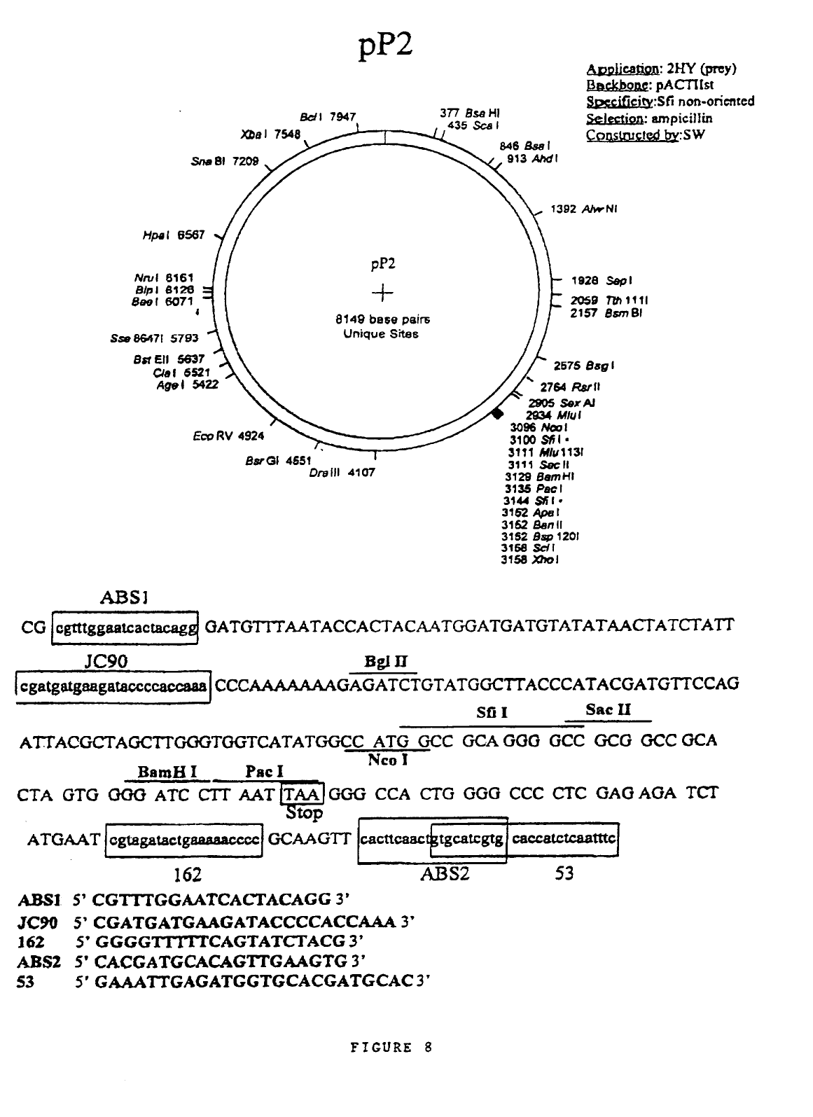

FIG. 9 is a schematic representation of the pP3 plasmid.

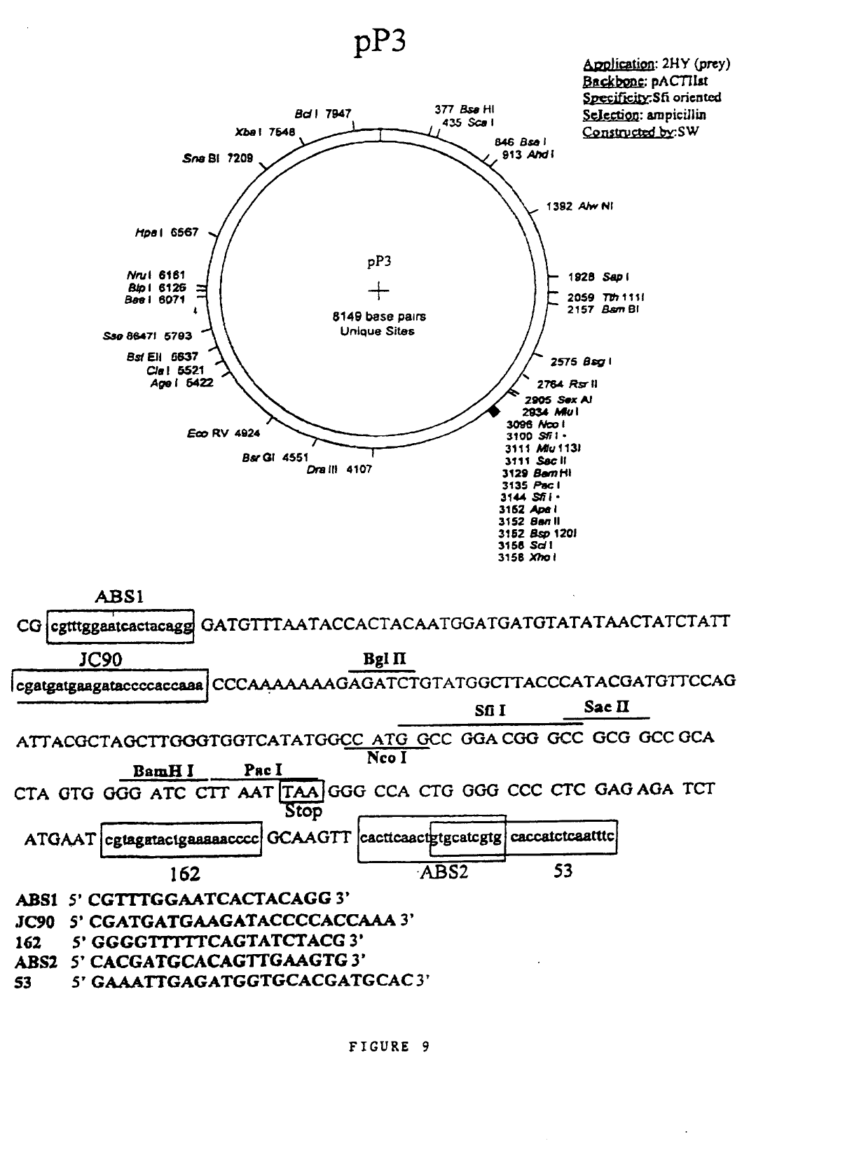

FIG. 10 is a schematic representation of the pP6 plasmid.

FIG. 11 is a schematic representation of the pP7 plasmid.

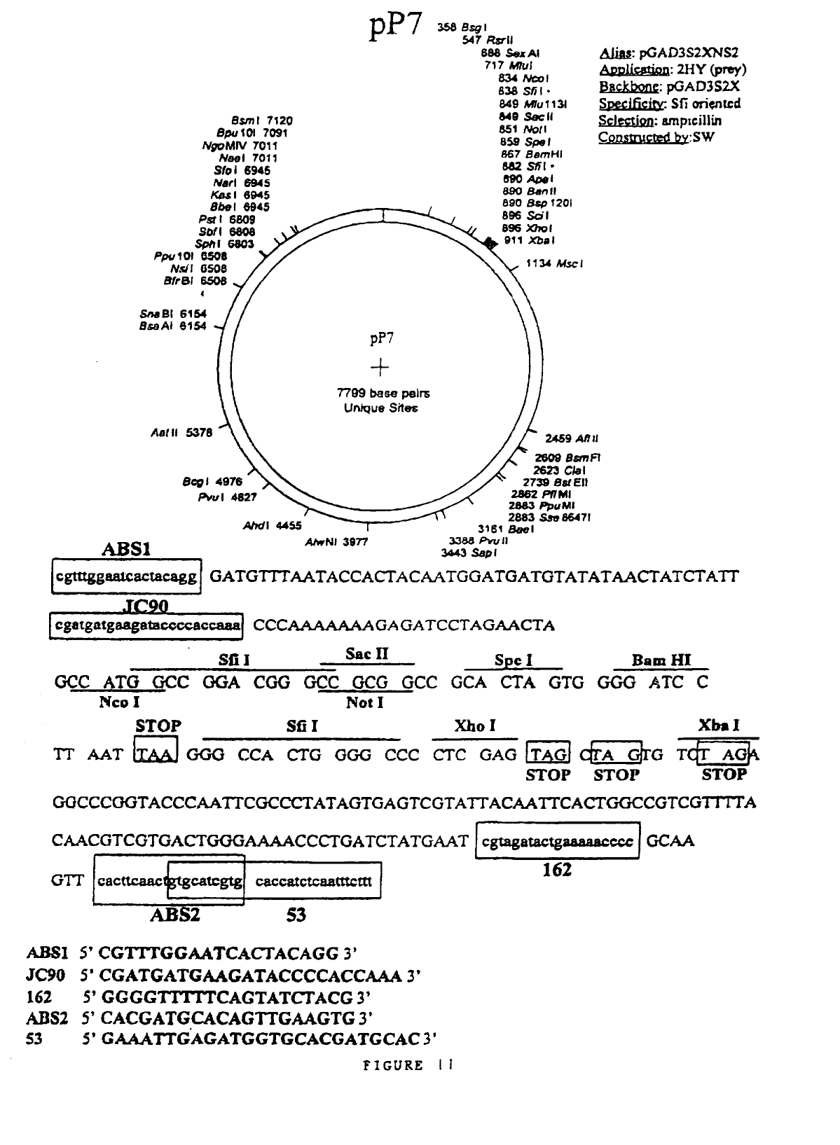

FIG. 12 is a schematic representation of vectors expressing the T25 fragment.

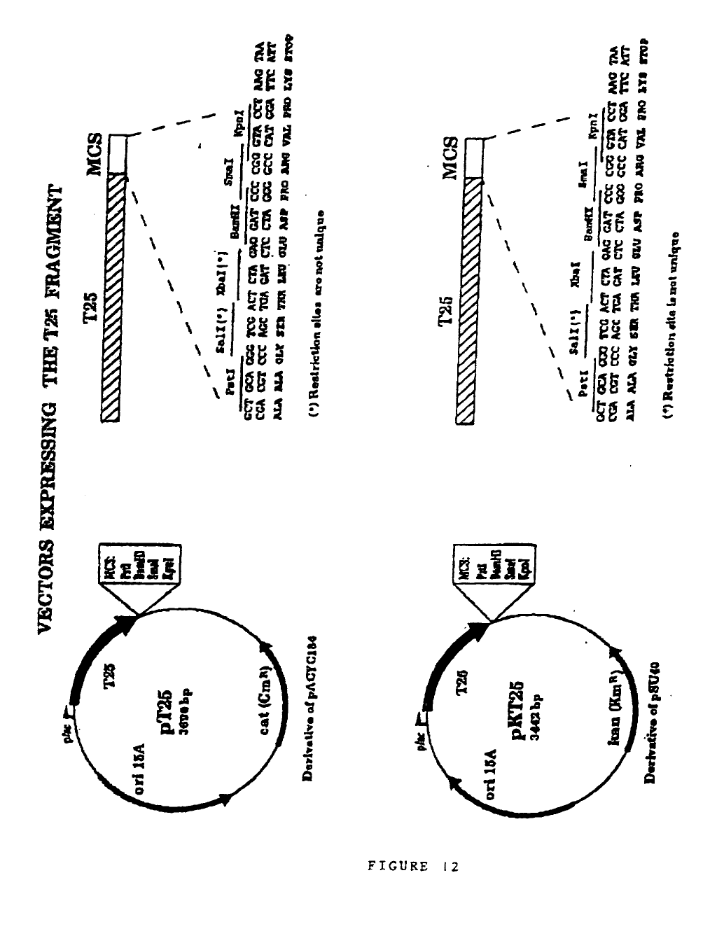

FIG. 13 is a schematic representation of vectors expressing the T18 fragment.

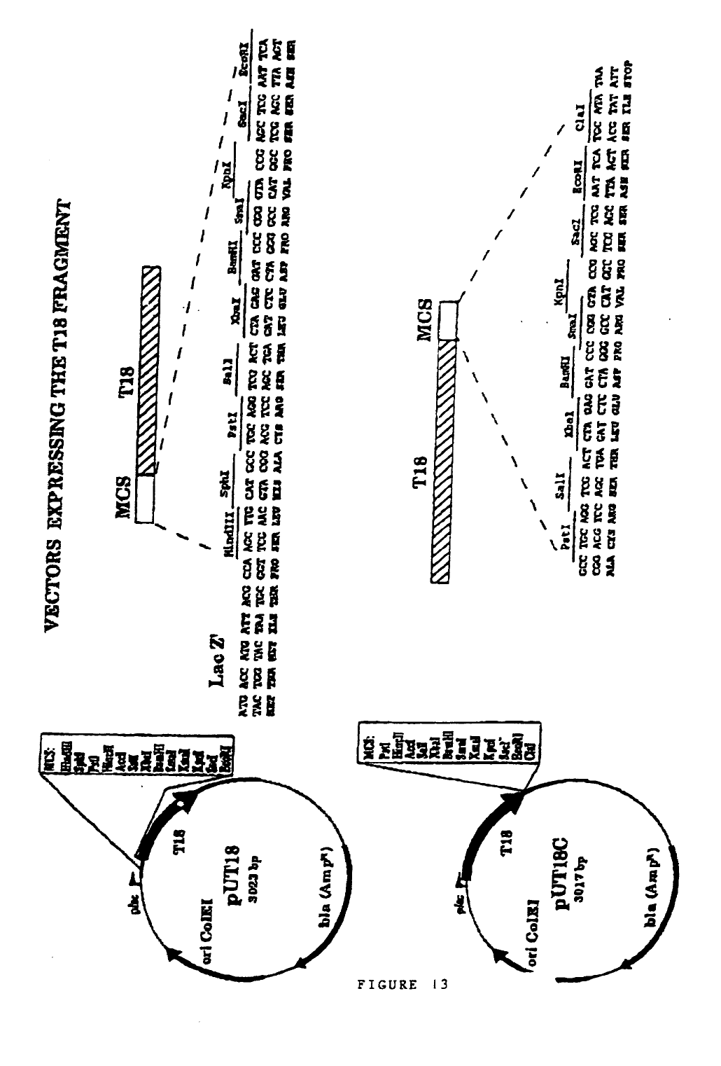

FIG. 14 is a schematic representation of various vectors of pCmAHL1, pT25 and pT18.

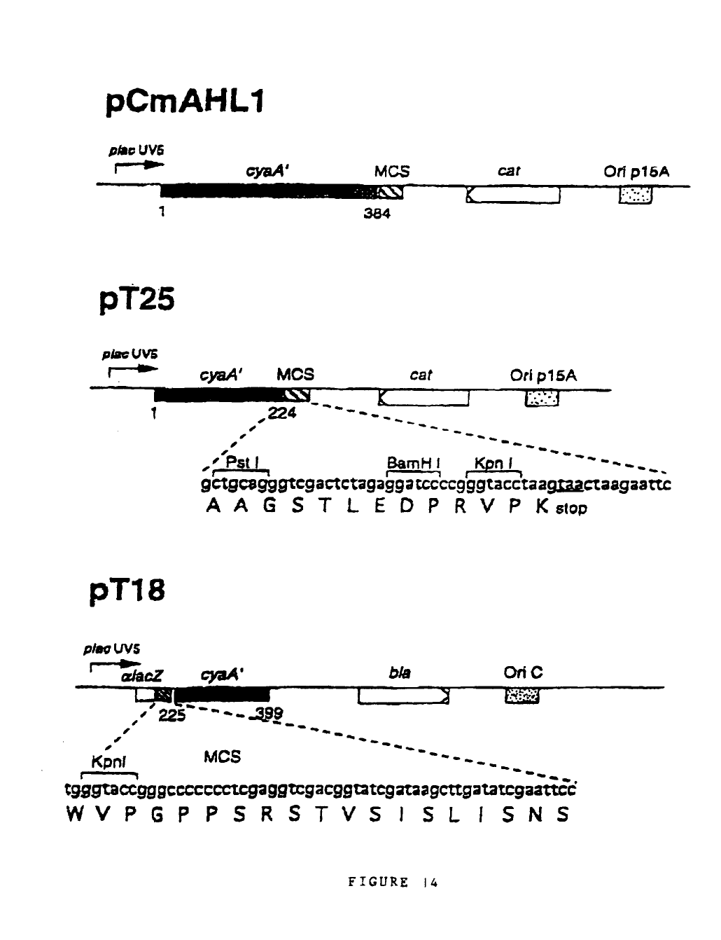

FIG. 15 is a schematic representation of identification of SID®. In this figure the “Full-length prey protein” is the Open Reading Frame (ORF) or coding sequence (CDS) where the identified prey polypeptides are included. The Selected Interaction Domain (SID®) is determined by the commonly shared polypeptide domain of every selected prey fragment.

FIG. 16 is a protein map (PIM®).

### DETAILED DESCRIPTION OF THE INVENTION

As used herein the terms “polynucleotides”, “nucleic acids” and “oligonucleotides” are used interchangeably and include, but are not limited to RNA, DNA, RNA/DNA sequences of more than one nucleotide in either single chain or duplex form. The polynucleotide sequences of the present invention may be prepared from any known method including, but not limited to, any synthetic method, any recombinant method, any ex vivo generation method and the like, as well as combinations thereof.

The term “polypeptide” means herein a polymer of amino acids having no specific length. Thus, peptides, oligopeptides and proteins are included in the definition of “polypeptide” and these terms are used interchangeably throughout the specification, as well as in the claims. The term “polypeptide” does not exclude post-translational modifications such as polypeptides having covalent attachment of glycosyl groups, aceteyl groups, phosphate groups, lipid groups and the like. Also encompassed by this definition of “polypeptide” are homologs thereof.

By the term “homologs” is meant structurally similar genes contained within a given species, orthologs are functionally equivalent genes from a given species or strain, as determined for example, in a standard complementation assay. Thus, a polypeptide of interest can be used not only as a model for identifying similiar genes in given strains, but also to identify homologs and orthologs of the polypeptide of interest in other species. The orthologs, for example, can also be identified in a conventional complementation assay. In addition or alternatively, such orthologs can be expected to exist in bacteria (or other kind of cells) in the same branch of the phylogenic tree, as set forth, for example, at ftp://ftp.cme.msu.edu/pub/rdp/SSU-rRNA/SSU/Prok.phylo.

As used herein the term “prey polynucleotide” means a chimeric polynucleotide encoding a polypeptide comprising (i) a specific domain; and (ii) a polypeptide that is to be tested for interaction with a bait polypeptide. The specific domain is preferably a transcriptional activating domain.

As used herein, a “bait polynucleotide” is a chimeric polynucleotide encoding a chimeric polypeptide comprising (i) a complementary domain; and (ii) a polypeptide that is to be tested for interaction with at least one prey polypeptide. The complementary domain is preferably a DNA-binding domain that recognizes a binding site that is further detected and is contained in the host organism.

As used herein “complementary domain” is meant a functional constitution of the activity when bait and prey are interacting; for example, enzymatic activity.

As used herein “specific domain” is meant a functional interacting activation domain that may work through different mechanisms by interacting directly or indirectly through intermediary proteins with RNA polymerase II or III-associated proteins in the vicinity of the transcription start site.

As used herein the term “complementary” means that, for example, each base of a first polynucleotide is paired with the complementary base of a second polynucleotide whose orientation is reversed. The complementary bases are A and T (or A and U) or C and G.

The term “sequence identity” refers to the identity between two peptides or between two nucleic acids. Identity between sequences can be determined by comparing a position in each of the sequences which may be aligned for the purposes of comparison. When a position in the compared sequences is occupied by the same base or amino acid, then the sequences are identical at that position. A degree of sequence identity between nucleic acid sequences is a function of the number of identical nucleotides at positions shared by these sequences. A degree of identity between amino acid sequences is a function of the number of identical amino acid sequences that are shared between these sequences. Since two polypeptides may each (i) comprise a sequence (i.e., a portion of a complete polynucleotide sequence) that is similar between two polynucleotides, and (ii) may further comprise a sequence that is divergent between two polynucleotides, sequence identity comparisons between two or more polynucleotides over a “comparison window” refers to the conceptual segment of at least 20 contiguous nucleotide positions wherein a polynucleotide sequence may be compared to a reference nucleotide sequence of at least 20 contiguous nucleotides and wherein the portion of the polynucleotide sequence in the comparison window may comprise additions or deletions (i.e., gaps) of 20 percent or less compared to the reference sequence (which does not comprise additions or deletions) for optimal alignment of the two sequences.

To determine the percent identity of two amino acids sequences or two nucleic acid sequences, the sequences are aligned for optimal comparison. For example, gaps can be introduced in the sequence of a first amino acid sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position.

The percent identity between the two sequences is a function of the number of identical positions shared by the sequences. Hence % identity=number of identical positions/total number of overlapping positions X 100.

In this comparison the sequences can be the same length or may be different in length. Optimal alignment of sequences for determining a comparison window may be conducted by the local homology algorithm of Smith and Waterman (J. Theor. Biol., 91 (2) pgs. 370-380 (1981), by the homology alignment algorithm of Needleman and Wunsch, J. Miol. Biol., 48(3) pgs. 443-453 (1972), by the search for similarity via the method of Pearson and Lipman, PNAS, USA, 85(5) pgs. 2444-2448 (1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection.

The best alignment (i.e., resulting in the highest percentage of identity over the comparison window) generated by the various methods is selected.

The term “sequence identity” means that two polynucleotide sequences are identical (i.e., on a nucleotide by nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size) and multiplying the result by 100 to yield the percentage of sequence identity. The same process can be applied to polypeptide sequences.

The percentage of sequence identity of a nucleic acid sequence or an amino acid sequence can also be calculated using BLAST software (Version 2.06 of September 1998) with the default or user defined parameter.

The term “sequence similarity” means that amino acids can be modified while retaining the same function. It is known that amino acids are classified according to the nature of their side groups and some amino acids such as the basic amino acids can be interchanged for one another while their basic function is maintained.

The term “isolated” as used herein means that a biological material such as a nucleic acid or protein has been removed from its original environment in which it is naturally present. For example, a polynucleotide present in a plant, mammal or animal is present in its natural state and is not considered to be isolated. The same polynucleotide separated from the adjacent nucleic acid sequences in which it is naturally inserted in the genome of the plant or animal is considered as being “isolated.”

The term “isolated” is not meant to exclude artificial or synthetic mixtures with other compounds, or the presence of impurities which do not interfere with the biological activity and which may be present, for example, due to incomplete purification, addition of stabilizers or mixtures with pharmaceutically acceptable excipients and the like.

“Isolated polypeptide” or “isolated protein” as used herein means a polypeptide or protein which is substantially free of those compounds that are normally associated with the polypeptide or protein in a naturally state such as other proteins or polypeptides, nucleic acids, carbohydrates, lipids and the like.

The term “purified” as used herein means at least one order of magnitude of purification is achieved, preferably two or three orders of magnitude, most preferably four or five orders of magnitude of purification of the starting material or of the natural material. Thus, the term “purified” as utilized herein does not mean that the material is 100% purified and thus excludes any other material.

The term “variants” when referring to, for example, polynucleotides encoding a polypeptide variant of a given reference polypeptide are polynucleotides that differ from the reference polypeptide but generally maintain their functional characteristics of the reference polypeptide. A variant of a polynucleotide may be a naturally occurring allelic variant or it may be a variant that is known naturally not to occur. Such non-naturally occurring variants of the reference polynucleotide can be made by, for example, mutagenesis techniques, including those mutagenesis techniques that are applied to polynucleotides, cells or organisms.

Generally, differences are limited so that the nucleotide sequences of the reference and variant are closely similar overall and, in many regions identical.

Variants of polynucleotides according to the present invention include, but are not limited to, nucleotide sequences which are at least 95% identical after alignment to the reference polynucleotide encoding the reference polypeptide. These variants can also have 96%, 97%, 98% and 99.999% sequence identity to the reference polynucleotide.

Nucleotide changes present in a variant polynucleotide may be silent, which means that these changes do not alter the amino acid sequences encoded by the reference polynucleotide.

Substitutions, additions and/or deletions can involve one or more nucleic acids. Alterations can produce conservative or non-conservative amino acid substitutions, deletions and/or additions.

Variants of a prey or a SID® polypeptide encoded by a variant polynucleotide can possess a higher affinity of binding and/or a higher specificity of binding to its protein or polypeptide counterpart, against which it has been initially selected. In another context, variants can also loose their ability to bind to their protein or polypeptide counterpart.

By “anabolic pathway” is meant a reaction or series of reactions in a metabolic pathway that synthesize complex molecules from simpler ones, usually requiring the input of energy. An anabolic pathway is the opposite of a catabolic pathway.

As used herein, a “catabolic pathway” is a series of reactions in a metabolic pathway that break down complex compounds into simpler ones, usually releasing energy in the process. A catabolic pathway is the opposite of an anabolic pathway.

As used herein, “drug metabolism” is meant the study of how drugs are processed and broken down by the body. Drug metabolism can involve the study of enzymes that break down drugs, the study of how different drugs interact within the body and how diet and other ingested compounds affect the way the body processes drugs.

As used herein, “metabolism” means the sum of all of the enzyme-catalyzed reactions in living cells that transform organic molecules.

By “secondary metabolism” is meant pathways producing specialized metabolic products that are not found in every cell.

As used herein, “SID®” means a Selected Interacting Domain and is identified as follows: for each bait polypeptide screened, selected prey polypeptides are compared. Overlapping fragments in the same ORF or CDS define the selected interacting domain.

As used herein the term “PIM®” means a protein-protein interaction map. This map is obtained from data acquired from a number of separate screens using different bait polypeptides and is designed to map out all of the interactions between the polypeptides.

The term “affinity of binding”, as used herein, can be defined as the affinity constant Ka when a given SID® polypeptide of the present invention which binds to a polypeptide and is the following mathematical relationship:

Ka=[SID®/polypeptide complex]/[free SID®]/[free polypeptide]

wherein [free SID®], [free polypeptide] and [SID®/polypeptide complex] consist of the concentrations at equilibrium respectively of the free SID® polypeptide, of the free polypeptide onto which the SID® polypeptide binds and of the complex formed between SID® polypeptide and the polypeptide onto which said SID® polypeptide specifically binds.

The affinity of a SID® polypeptide of the present invention or a variant thereof for its polypeptide counterpart can be assessed, for example, on a Biacore™ apparatus marketed by Amersham Pharmacia Biotech Company such as described by Szabo et al Curr Opin Struct Biol 5 pgs. 699-705 (1995) and by Edwards and Leartherbarrow, Anal. Biochem 246 pgs. 1-6 (1997).

As used herein the phrase “at least the same affinity” with respect to the binding affinity between a SID® polypeptide of the present invention to another polypeptide means that the Ka is identical or can be at least two-fold, at least three-fold or at least five fold greater than the Ka value of reference.

As used herein, the term “modulating compound” means a compound that inhibits or stimulates or can act on another protein which can inhibit or stimulate the protein-protein interaction of a complex of two polypeptides or the protein-protein interaction of two polypeptides.

More specifically, the present invention comprises complexes of polypeptides or polynucleotides encoding the polypeptides composed of a bait polypeptide, or a bait polynucleotide encoding a bait polypeptide and a prey polypeptide or a prey polynucleotide encoding a prey polypeptide. The prey polypeptide or prey polynucleotide encoding the prey polypeptide is capable of interacting with a bait polypeptide of interest in various hybrid systems.

As described in the Background of the present invention there are various methods known in the art to identify prey polypeptides that interact with bait polypeptides of interest. These methods, include, but are not limited to, generic two-hybrid systems as described by Fields et al in Nature, 340:245-246 (1989) and more specifically in U.S. Pat. Nos. 5,283,173, 5,468,614 and 5,667,973, which are hereby incorporated by reference; the reverse two-hybrid system described by Vidal et al., supra; the two plus one hybrid method described, for example, in Tirode et al., supra; the yeast forward and reverse ‘n’-hybrid systems as described in Vidal and Legrain, supra; the method described in WO 99/42612; those methods described in Legrain et al FEBS Letters 480 pgs. 32-36 (2000) and the like.

The present invention is not limited to the type of method utilized to detect protein-protein interactions and therefore any method known in the art and variants thereof can be used. It is however better to use the method described in WO 99/42612 or WO 00/66722, both references incorporated herein by reference due to the methods' sensitivity, reproducibility and reliability.

Protein-protein interactions can also be detected using complementation assays such as those described by Pelletier et al. at http://www.abrf.org/JBT/Articles/JBT0012/jbt0012.html, WO 00/07038 and WO98/34120.

Although the above methods are described for applications in the yeast system, the present invention is not limited to detecting protein-protein interactions using yeast, but also includes similar methods that can be used in detecting protein-protein interactions in, for example, mammalian systems as described, for example in Takacs et al., Proc. Natl. Acad. Sci., USA, 90 (21):10375-79 (1993) and Vasavada et al., Proc. Natl. Acad. Sci., USA, 88 (23):10686-90 (1991), as well as a bacterial two-hybrid system as described in Karimova et al (1998), W099/28746, WO 00/66722 and Legrain et al FEBS Letters, 480 pgs. 32-36 (2000).

The above-described methods are limited to the use of yeast, mammalian cells and Escherichia coli cells, the present invention is not limited in this manner. Consequently, mammalian and typically human cells, as well as bacterial, yeast, fungus, insect, nematode and plant cells are encompassed by the present invention and may be transfected by the nucleic acid or recombinant vector as defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli, (e.g., strain DH5-α), Bacillus subtilis, Salmonella typhimurium, or strains of the genera of Pseudomonas, Streptomyces and Staphylococcus.

Further suitable cells that can be used in the present invention include yeast cells such as those of Saccharomyces such as Saccharomyces cerevisiae.

The bait polynucleotide, as well as the prey polynucleotide can be prepared according to the methods known in the art such as those described above in the publications and patents reciting the known method per se.

The bait polynucleotide of the present invention is obtained from Shigella flexneri (see Table I). The prey polynucleotide is obtained form a human placenta cDNA or variants thereof and fragments from the genome or transcriptome of human placenta ranging from about 12 to about 5,000, or about 12 to about 10,000 or from about 12 to about 20,000. The prey polynucleotide is then selected, sequenced and identified.

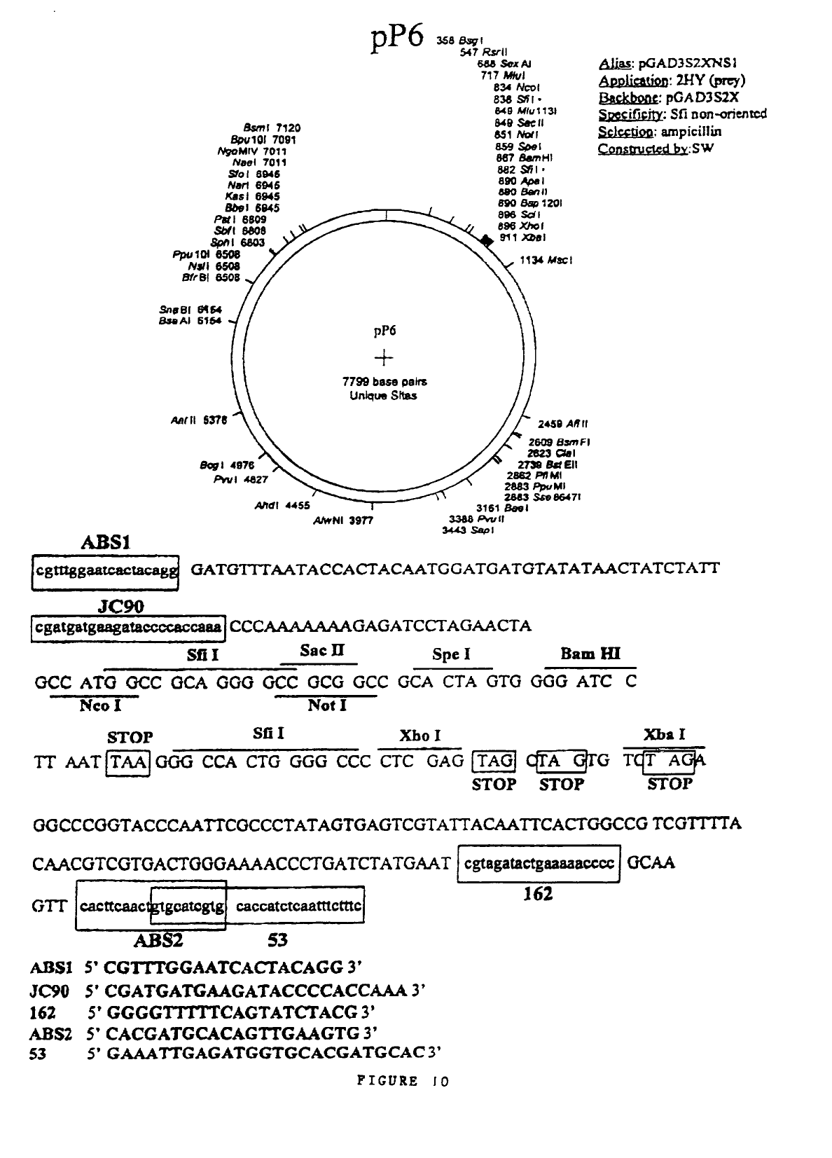

A human placenta cDNA prey library is prepared from global human placenta and constructed in the specially designed prey vector pP6 as shown in FIG. 10 after ligation of suitable linkers such that every cDNA fragment insert is fused to a nucleotide sequence in the vector that encodes the transcription activation domain of a reporter gene. Any transcription activation domain can be used in the present invention. Examples include, but are not limited to, Gal4,YP16, B42, His and the like. Toxic reporter genes, such as CAT^{R}, CYH2, CYH1, URA3, bacterial and fungi toxins and the like can be used in reverse two-hybrid systems.

The polypeptides encoded by the nucleotide inserts of the human placenta cDNA prey library thus prepared are termed “prey polypeptides” in the context of the presently described selection method of the prey polynucleotides.

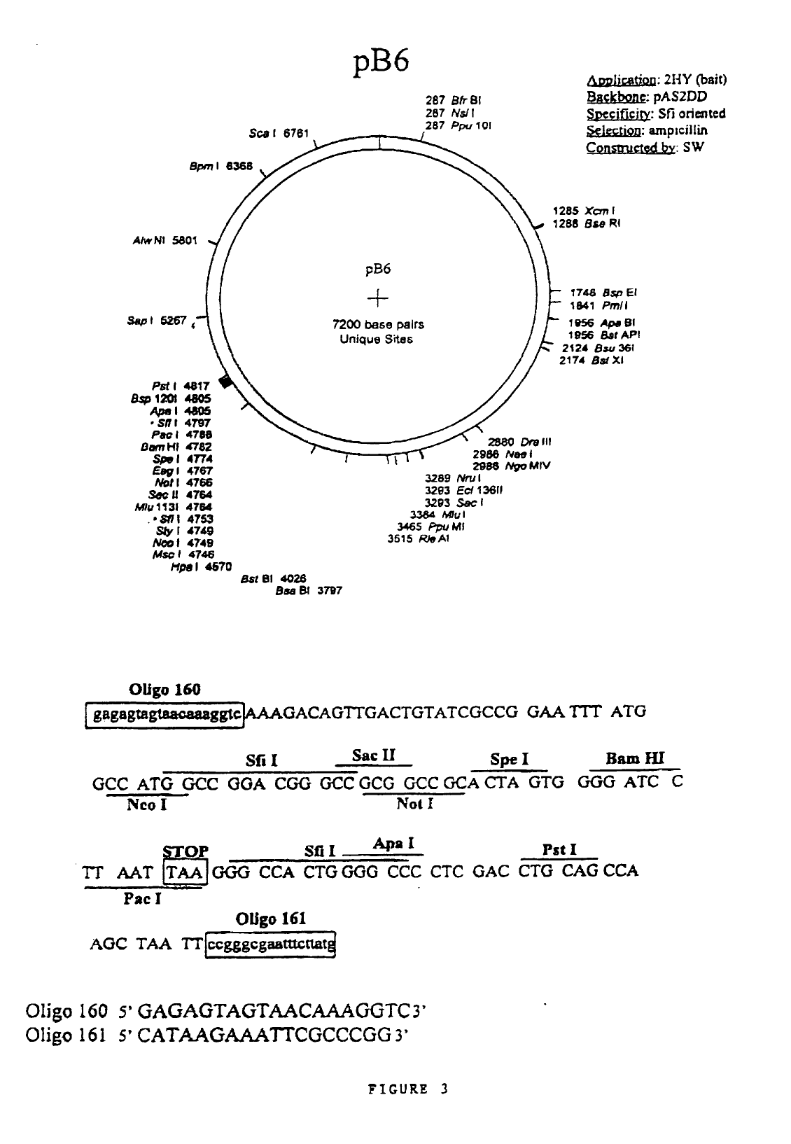

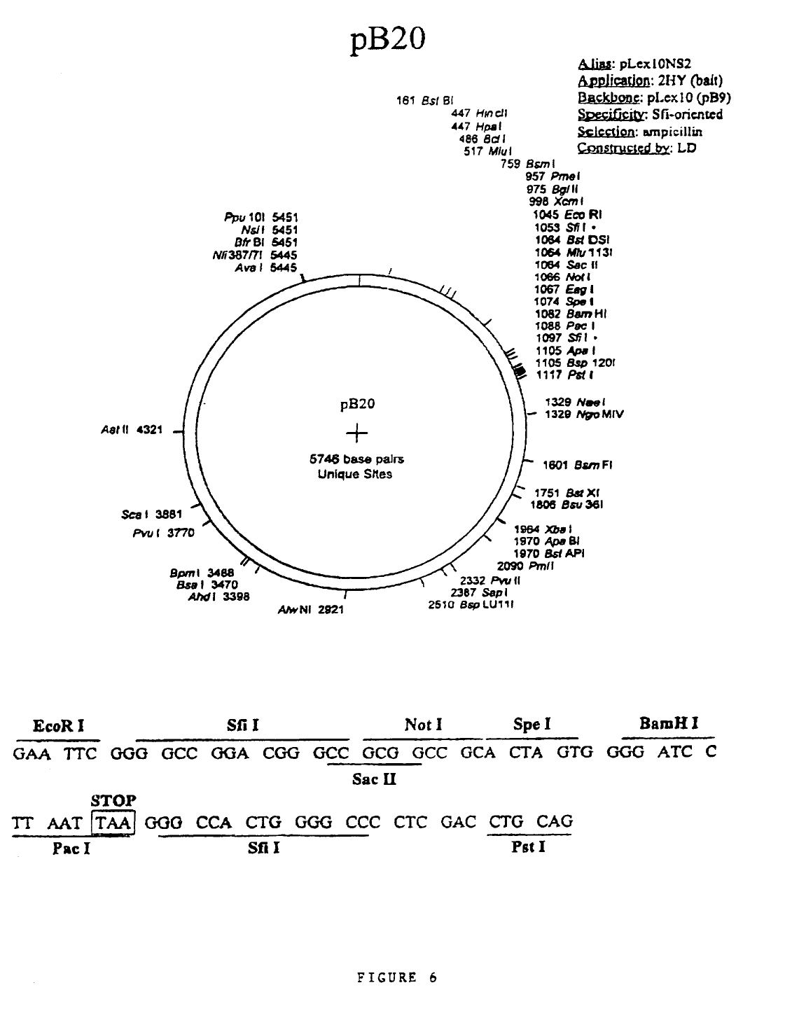

The bait polynucleotide can be inserted in bait plasmid pB6 or pB20 as illustrated in FIG. 3 or 6 respectively. The bait polynucleotide insert is fused to a polynucleotide encoding the binding domain of, for example, the Gal4 DNA binding domain and the shuttle expression vector is used to transform cells. The bait polynucleotides used in the present invention are describes in Table I. As stated above, any cells can be utilized in transforming the bait and prey polynucleotides of the present invention including mammalian cells, bacterial cells, yeast cells, insect cells and the like.

In an embodiment, the present invention identifies protein-protein interactions in yeast. In using known methods a prey positive clone is identified containing a vector which comprises a nucleic acid insert encoding a prey polypeptide which binds to a bait polypeptide of interest. The method in which protein-protein interactions are identified comprises the following steps:

mating at least one first haploid recombinant yeast cell clone from a recombinant yeast cell clone library that has been transformed with a plasmid containing the prey polynucleotide to be assayed with a second haploid recombinant yeast cell clone transformed with a plasmid containing a bait polynucleotide encoding for the bait polypeptide;

cultivating diploid cell clones obtained in step i) on a selective medium; and

selecting recombinant cell clones which grow on the selective medium.

This method may further comprise the step of:

iv) characterizing the prey polynucleotide contained in each recombinant cell clone which is selected in step iii).

In yet another embodiment of the present invention, in lieu of yeast, Escherichia coli is used in a bacterial two-hybrid system, which encompasses a similar principle to that described above for yeast, but does not involve mating for characterizing the prey polynucleotide.

In yet another embodiment of the present invention, mammalian cells and a method similar to that described above for yeast for characterizing the prey polynucleotide are used.

By performing the yeast, bacterial or mammalian two-hybrid system it is possible to identify for one particular bait an interacting prey polypeptide. The prey polypeptide that has been selected by testing the library of preys in a screen using the two-hybrid, two plus one hybrid methods and the like, encodes the polypeptide interacting with the protein of interest.

The present invention is also directed, in a general aspect, to a complex of polypeptides, polynucleotides encoding the polypeptides composed of a bait polypeptide or bait polynucleotide encoding the bait polypeptide and a prey polypeptide or prey polynucleotide encoding the prey polypeptide capable of interacting with the bait polypeptide of interest. These complexes are identified in Table II, as the bait amino acid sequences and the prey amino acid sequences, as well as the bait and prey nucleic acid sequences.

In another aspect, the present invention relates to a complex of polynucleotides consisting of a first polynucleotide, or a fragment thereof, encoding a prey polypeptide that interacts with a bait polypeptide and a second polynucleotide or a fragment thereof. This fragment has at least 12 consecutive nucleotides, but can have between 12 and 5,000 consecutive nucleotides, or between 12 and 10,000 consecutive nucleotides or between 12 and 20,000 consecutive nucleotides.

The polypeptides of column 1 and 3 from Table II according to the present invention and the complexes of these two polypeptides also form part of the present invention. More specifically, the polypeptides of SEQ ID NOS. 1 to 7 are part of the present invention and their complexes with the polypeptides of Column 3, Table II.

In yet another embodiment, the present invention relates to an isolated complex of at least two polypeptides encoded by two polynucleotides wherein said two polypeptides are associated in the complex by affinity binding and are depicted in columns 1 and 3 of Table II.

In yet another embodiment, the present invention relates to an isolated complex comprising at least a polypeptide as described in column 1 of Table II and a polypeptide as described in column 3 of Table II. The present invention is not limited to these polypeptide complexes alone but also includes the isolated complex of the two polypeptides in which fragments and/or homologous polypeptides exhibiting at least 95% sequence identity, as well as from 96% sequence identity to 99.999% sequence identity.

Also encompassed in another embodiment of the present invention is an isolated complex in which SID® of the prey polypeptides encoded by SEQ ID Nos. 15 to 215 in Table III form the isolated complex.

Besides the isolated complexes described above, nucleic acids coding for a Selected Interacting Domain (SID®) polypeptide or a variant thereof or any of the nucleic acids set forth in Table III can be inserted into an expression vector which contains the necessary elements for the transcription and translation of the inserted protein-coding sequence. Such transcription elements include a regulatory region and a promoter. Thus, the nucleic acid which may encode a marker compound of the present invention is operably linked to a promoter in the expression vector. The expression vector may also include a replication origin.

A wide variety of host/expression vector combinations are employed in expressing the nucleic acids of the present invention. Useful expression vectors that can be used include, for example, segments of chromosomal, non-chromosomal and synthetic DNA sequences. Suitable vectors include, but are not limited to, derivatives of SV40 and pcDNA and known bacterial plasmids such as col EI, pCR1, pBR322, pMal-C2, pET, pGEX as described by Smith et al [need cite 1988], pMB9 and derivatives thereof, plasmids such as RP4, phage DNAs such as the numerous derivatives of phage I such as NM989, as well as other phage DNA such as M13 and filamentous single stranded phage DNA; yeast plasmids such as the 2 micron plasmid or derivatives of the 2m plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like.

For example in a baculovirus expression system, both non-fusion transfer vectors, such as, but not limited to pVL941 (BamHI cloning site Summers, pVL1393 (BamHI, SmaI, XbaI, EcoRI, NotI, XmaIII, BgIII and PsfI cloning sites; Invitrogen) pVL1392 (BgIII, PstI, NotI, XmaIII, EcoRI, XbaII, SmaI and BamHI cloning site; Summers and Invitrogen) and pBlueBacIII (BamHI, BgAlII, PstI, NcoI and HindIII cloning site, with blue/white recombinant screening, Invitrogen), and fusion transfer vectors such as, but not limited to, pAc700(BamHI and KpnI cloning sites, in which the BamHI recognition site begins with the initiation codon; Summers), pAc701 and pAc70-2 (same as pAc700, with different reading frames), pAc360 (BamHI cloning site 36 base pairs downstream of a polyhedrin initiation codon; Invitrogen (195)) and pBlueBacHisA, B, C (three different reading frames with BamHI, BglII, PstI, NcoI and HindIII cloning site, an N-terminal peptide for ProBond purification and blue/white recombinant screening of plaques; Invitrogen (220) can be used.

Mammalian expression vectors contemplated for use in the invention include vectors with inducible promoters, such as the dihydrofolate reductase promoters, any expression vector with a DHFR expression cassette or a DHFR/methotrexate co-amplification vector such as pED (PsfI, SalI, SbaI, SmaI and EcoRI cloning sites, with the vector expressing both the cloned gene and DHFR; Kaufman, 1991). Alternatively a glutamine synthetase/methionine sulfoximine co-amplification vector, such as pEE14 (HindIII, XbalI, SmaI, SbaI, EcoRI and BclI cloning sites in which the vector expresses glutamine synthetase and the cloned gene; Celltech). A vector that directs episomal expression under the control of the Epstein Barr Virus (EBV) or nuclear antigen (EBNA) can be used such as pREP4 (BamHI, SfiI, XhoI, NotI, NheI, HindIII, NheI, PvuII and KpnI cloning sites, constitutive RSV-LTR promoter, hygromycin selectable marker; Invitrogen) pCEP4 (BamHI, SfiI, XhoI, NotI, NheI, HindIII, NheI, PvuII and KpnI cloning sites, constitutive hCMV immediate early gene promoter, hygromycin selectable marker; Invitrogen), pMEP4 (KpnI, PvuI, NheI, HindIII, NotI, XhoI, SfiI, BamHI cloning sites, inducible methallothionein IIa gene promoter, hygromycin selectable marker, Invitrogen), pREP8 (BamHI, XhoI, NotI, HindIII, NheI and KpnI cloning sites, RSV-LTR promoter, histidinol selectable marker; Invitrogen), pREP9 (KpnI, NheI, HindIII, NotI, XhoI, SfiI, BamHI cloning sites, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen).

Selectable mammalian expression vectors for use in the invention include, but are not limited to, pRc/CMV (HindIII, BstXI, NotI, SbaI and ApaI cloning sites, G418 selection, Invitrogen), pRc/RSV (HindII, SpeI, BstXI, NotI, Xbal cloning sites, G418 selection, Invitrogen) and the like. Vaccinia virus mammalian expression vectors (see, for example Kaufman 1991 that can be used in the present invention include, but are not limited to, pSC11 (SmaI cloning site, TK- and β-gal selection), pMJ601 (SalI, SmaI, AflI, NarI, BspMII, BamHI, ApaI, NheI, SacII, KpnI and HindIII cloning sites; TK- and β-gal selection), pTKgptF1S (EcoRI, PstI, SalII, AccI, HindII, SbaI, BamHI and Hpa cloning sites, TK or XPRT selection) and the like.

Yeast expression systems that can also be used in the present include, but are not limited to, the non-fusion pYES2 vector (XbaI, SphI, ShoI, NotI, GstXI, EcoRI, BstXI, BamHI, SacI, KpnI and HindIII cloning sites, Invitrogen), the fusion pYESHisA, B, C (XbalI, SphI, ShoI, NotI, BstXI, EcoRI, BamHI, SacI, KpnI and HindIII cloning sites, N-terminal peptide purified with ProBond resin and cleaved with enterokinase; Invitrogen), pRS vectors and the like.

Consequently, mammalian and typically human cells, as well as bacterial, yeast, fungi, insect, nematode and plant cells an used in the present invention and may be transfected by the nucleic acid or recombinant vector as defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli, (e.g., strain DH5-β), Bacillus subtilis, Salmonella typhimurium, or strains of the genera of Pseudomonas, Streptomyces and Staphylococcus.

Further suitable cells that can be used in the present invention include yeast cells such as those of Saccharomyces such as Saccharomyces cerevisiae.

Besides the specific isolated complexes, as described above, the present invention relates to and also encompasses SID® polynucleotides. As explained above, for each bait polypeptide, several prey polypeptides may be identified by comparing and selecting the intersection of every isolated fragment that are included in the same polypeptide. Thus the SID® polynucleotides of the present invention are represented by the shared nucleic acid sequences of SEQ ID Nos. 15 to 215 encoding the SID® polypeptides of SEQ ID Nos. 216 to 416 in columns 5 and 7 of Table III, respectively.

The present invention is not limited to the SID® sequences as described in the above paragraph, but also includes fragments of these sequences having at least 12 consecutive nucleic acids, between 12 and 5,000 consecutive nucleic acids and between 12 and 10,000 consecutive nucleic acids and between 12 and 20,000 consecutive nucleic acids, as well as variants thereof. The fragments or variants of the SID® sequences possess at least the same affinity of binding to its protein or polypeptide counterpart, against which it has been initially selected. Moreover this variant and/or fragments of the SID® sequences alternatively can have between 95% and 99.999% sequence identity to its protein or polypeptide counterpart.

According to the present invention the variants can be created by known mutagenesis techniques either in vitro or in vivo. Such a variant can be created such that it has altered binding characteristics with respect to the target protein and more specifically that the variant binds the target sequence with either higher or lower affinity.

Polynucleotides that are complementary to the above sequences which include the polynucleotides of the SID®'s, their fragments, variants and those that have specific sequence identity are also included in the present invention.

The polynucleotide encoding the SID® polypeptide, fragment or variant thereof can also be inserted into recombinant vectors which are described in detail above.

The present invention also relates to a composition comprising the above-mentioned recombinant vectors containing the SID® polypeptides in Table III, fragments or variants thereof, as well as recombinant host cells transformed by the vectors. The recombinant host cells that can be used in the present invention were discussed in greater detail above.

The compositions comprising the recombinant vectors can contain physiological acceptable carriers such as diluents, adjuvants, excipients and any vehicle in which this composition can be delivered therapeutically and can include, but is are not limited to sterile liquids such as water and oils.

In yet another embodiment, the present invention relates to a method of selecting modulating compounds, as well as the modulating molecules or compounds themselves which may be used in a pharmaceutical composition. These modulating compounds may act as a cofactor, as an inhibitor, as antibodies, as tags, as a competitive inhibitor, as an activator or alternatively have agonistic or antagonistic activity on the protein-protein interactions.

The activity of the modulating compound does not necessarily, for example, have to be 100% activation or inhibition. Indeed, even partial activation or inhibition can be achieved that is of pharmaceutical interest.

The modulating compound can be selected according to a method which comprises:

cultivating a recombinant host cell with a modulating compound on a selective medium and a reporter gene the expression of which is toxic for said recombinant host cell wherein said recombinant host cell is transformed with two vectors:

wherein said first vector comprises a polynucleotide encoding a first hybrid polypeptide having a DNA binding domain;

wherein said second vector comprises a polynucleotide encoding a second hybrid polypeptide having a transcriptional activating domain that activates said toxic reporter gene when the first and second hybrid polypeptides interact;

selecting said modulating compound which inhibits or permits the growth of said recombinant host cell.

Thus, the present invention relates to a modulating compound that inhibits the protein-protein interactions between Shigella flexneri polypeptide and human placenta polypeptide of columns 1 and 3 of Table II, respectively. The present invention also relates to a modulating compound that activates the protein-protein interactions between Shigella flexneri polypeptide and human placenta polypeptide of columns 1 and 3 of Table II, respectively.

In yet another embodiment, the present invention relates to a method of selecting a modulating compound, which modulating compound inhibits the interaction between Shigella flexneri polypeptide and human placenta polypeptide of columns 1 and 3 of Table II, respectively. This method comprises:

(a) cultivating a recombinant host cell with a modulating compound on a selective medium and a reporter gene the expression of which is toxic for said recombinant host cell wherein said recombinant host cell is transformed with two vectors:

(i) wherein said first vector comprises a polynucleotide encoding a first hybrid polypeptide having a first domain of an enzyme;

(ii) wherein said second vector comprises a polynucleotide encoding a second hybrid polypeptide having an enzymatic transcriptional activating domain that activates said toxic reporter gene when the first and second hybrid polypeptides interact;

(b) selecting said modulating compound which inhibits or permits the growth of said recombinant host cell.

In the two methods described above any toxic reporter gene can be utilized including those reporter genes that can be used for negative selection including the URA3 gene, the CYH1 gene, the CYH2 gene and the like.

In yet another embodiment, the present invention provides a kit for screening a modulating compound. This kit comprises a recombinant host cell which comprises a reporter gene the expression of which is toxic for the recombinant host cell. The host cell is transformed with two vectors. The first vector comprises a polynucleotide encoding a first hybrid polypeptide having a DNA binding domain; and a second vector comprises a polynucleotide encoding a second hybrid polypeptide having a transcriptional activating domain that activates said toxic reporter gene when the first and second hybrid polypeptides interact.

In yet another embodiment a kit is provided for screening a modulating compound by providing a recombinant host cell, as described in the paragraph above, but instead of a DNA binding domain, the first vector comprises a first hybrid polypeptide containing a first domain of a protein. The second vector comprises a second polypeptide containing a second part of a complementary domain of a protein that activates the toxic reporter gene when the first and second hybrid polypeptides interact.

In the selection methods described above, the activating domain can be p42 Gal 4, YP16 (HSV) and the DNA-binding domain can be derived from Gal4 or Lex A. The protein or enzyme can be adenylate cyclase, guanylate cyclase, DHFR and the like.

Examples of modulating compounds are set forth in Table III.

In yet another embodiment, the present invention relates to a pharmaceutical composition comprising the modulating compounds for preventing or treating bacillary dysentery in a human or animal, most preferably in a mammal.

This pharmaceutical composition comprises a pharmaceutically acceptable amount of the modulating compound. The pharmaceutically acceptable amount can be estimated from cell culture assays. For example, a dose can be formulated in animal models to achieve a circulating concentration range that includes or encompasses a concentration point or range having the desired effect in an in vitro system. This information can thus be used to accurately determine the doses in other mammals, including humans and animals.

The therapeutically effective dose refers to that amount of the compound that results in amelioration of symptoms in a patient. Toxicity and therapeutic efficacy of such compounds can be determined by standard pharmaceutical procedures in cell cultures or in experimental animals. For example, the LD50 (the dose lethal to 50% of the population) as well as the ED50 (the dose therapeutically effective in 50% of the population) can be determined using methods known in the art. The dose ratio between toxic and therapeutic effects is the therapeutic index which can be expressed as the ratio between LD 50 and ED50 compounds that exhibit high therapeutic indexes.

The data obtained from the cell culture and animal studies can be used in formulating a range of dosage of such compounds which lies preferably within a range of circulating concentrations that include the ED50 with little or no toxicity.

The pharmaceutical composition can be administered via any route such as locally, orally, systemically, intravenously, intramuscularly, mucosally, using a patch and can be encapsulated in liposomes, microparticles, microcapsules, and the like. The pharmaceutical composition can be embedded in liposomes or even encapsulated.

Any pharmaceutically acceptable carrier or adjuvant can be used in the pharmaceutical composition. The modulating compound will be preferably in a soluble form combined with a pharmaceutically acceptable carrier. The techniques for formulating and administering these compounds can be found in “Remington's Pharmaceutical Science” Mack Publication Co., Easton, Pa., latest edition.

The mode of administration optimum dosages and galenic forms can be determined by the criteria known in the art taken into account the seriousness of the general condition of the mammal, the tolerance of the treatment and the side effects.

The present invention also relates to a method of treating or preventing bacillary dysentery in a human or mammal in need of such treatment. This method comprises administering to a mammal in need of such treatment a pharmaceutically effective amount of a modulating compound which binds to a targeted Shigella protein. In a preferred embodiment, the modulating compound is a polynucleotide which may be placed under the control of a regulatory sequence which is functional in the mammal or human.

In yet another embodiment, the present invention relates to a pharmaceutical composition comprising a SID® polypeptide, a fragment or variant thereof. The SID® polypeptide, fragment or variant thereof can be used in a pharmaceutical composition provided that it is endowed with highly specific binding properties to a bait polypeptide of interest.

The original properties of the SID® polypeptide or variants thereof interfere with the naturally occurring interaction between a first protein and a second protein within the cells of the organism. Thus, the SID® polypeptide binds specifically to either the first polypeptide or the second polypeptide.

Therefore, the SID® polypeptides of the present invention or variants thereof interfere with protein-protein interactions between Shigella or Escherichia polypeptides or between a mammal polypeptide.

Thus, the present invention relates to a pharmaceutical composition comprising a pharmaceutically acceptable amount of a SID® polypeptide or variant thereof, provided that the variant has the above-mentioned two characteristics; i.e., that it is endowed with highly specific binding properties to a bait polypeptide of interest and is devoid of biological activity of the naturally occurring protein.

In yet another embodiment, the present invention relates to a pharmaceutical composition comprising a pharmaceutically effective amount of a polynucleotide encoding a SID® polypeptide or a variant thereof wherein the polynucleotide is placed under the control of an appropriate regulatory sequence. Appropriate regulatory sequences that are used are polynucleotide sequences derived from promoter elements and the like.

Polynucleotides that can be used in the pharmaceutical composition of the present invention include the nucleotide sequences of SID®s of SEQ ID Nos. 15 to 215.

Besides the SID® polypeptides and polynucleotides, the pharmaceutical composition of the present invention can also include a recombinant expression vector comprising the polynucleotide encoding the SID® polypeptide, fragment or variant thereof.

The above described pharmaceutical compositions can be administered by any route such as orally, systemically, intravenously, intramuscularly, intradermally, mucosally, encapsulated, using a patch and the like. Any pharmaceutically acceptable carrier or adjuvant can be used in this pharmaceutical composition.

The SID® polypeptides as active ingredients will be preferably in a soluble form combined with a pharmaceutically acceptable carrier. The techniques for formulating and administering these compounds can be found in “Remington's Pharmaceutical Sciences” supra.

The amount of pharmaceutically acceptable SID® polypeptides can be determined as described above for the modulating compounds using cell culture and animal models.

Such compounds can be used in a pharmaceutical composition to treat or prevent bacillary dysentery.

Thus, the present invention also relates to a method of preventing or treating bacillary dysentery in a mammal said method comprising the steps of administering to a mammal in need of such treatment a pharmaceutically effective amount of a recombinant expression vector comprising a polynucleotide encoding a SID® polypeptide which binds to a either to a Shigella flexneri protein or to a human placenta protein involved in a protein-protein interaction between a Shigella flexneri protein and an human placenta protein.More specifically, the present invention relates to a method of preventing or treating bacillary dysentery in a mammal said method comprising the steps of administering to a mammal in need of such treatment a pharmaceutically effective amount of:

(1) a SID® polypeptide of SEQ ID Nos. 216 to 416 or a variant thereof which binds to a targeted Shigella flexneri protein or human placenta protein; or

(2) a SID® polynucleotide encoding a SID® polypeptide of SEQ ID Nos. 15 to 215 or a variant or a fragment thereof wherein said polynucleotide is placed under the control of a regulatory sequence which is functional in said mammal; or

(3) a recombinant expression vector comprising a polynucleotide encoding a SID® polypeptide which binds either to a Shigella flexneri protein or to a human placenta protein involved in a protein-protein interaction between a Shigella flexneri protein and an human placenta protein.

In another embodiment the present invention nucleic acids comprising a sequence of SEQ ID Nos. 15 to 215 which encodes the protein of sequence SEQ ID Nos. 216 to 416 and/or functional derivatives thereof are administered to modulate complex (from Table II) function by way of gene therapy. Any of the methodologies relating to gene therapy available within the art may be used in the practice of the present invention such as those described by Goldspiel et al Clin. Pharm. 12 pgs. 488-505 (1993).

Delivery of the therapeutic nucleic acid into a patient may be direct in vivo gene therapy (i.e., the patient is directly exposed to the nucleic acid or nucleic acid-containing vector) or indirect ex vivo gene therapy (i.e., cells are first transformed with the nucleic acid in vitro and then transplanted into the patient).

For example for in vivo gene therapy, an expression vector containing the nucleic acid is administered in such a manner that it becomes intracellular; i.e., by infection using a defective or attenuated retroviral or other viral vectors as described, for example in U.S. Pat. No. 4,980,286 or by Robbins et al., Pharmacol. Ther. , 80 No. 1 pgs. 35-47 (1998).

The various retroviral vectors that are known in the art are such as those described in Miller et al., Meth. Enzymol. 217 pgs. 581-599 (1993) which have been modified to delete those retroviral sequences which are not required for packaging of the viral genome and subsequent integration into host cell DNA. Also adenoviral vectors can be used which are advantageous due to their ability to infect non-dividing cells and such high-capacity adenoviral vectors are described in Kochanek, Human Gene Therapy, 10, pgs. 2451-2459 (1999). Chimeric viral vectors that can be used are those described by Reynolds et al., Molecular Medecine Today, pgs. 25 -31 (1999). Hybrid vectors can also be used and are described by Jacoby et al., Gene Therapy, 4, pgs. 1282-1283 (1997).

Direct injection of naked DNA or through the use of microparticle bombardment (e.g., Gene Gun®; Biolistic, Dupont). or by coating it with lipids can also be used in gene therapy. Cell-surface receptors/transfecting agents or through encapsulation in liposomes, microparticles or microcapsules or by administering the nucleic acid in linkage to a peptide which is known to enter the nucleus or by administering it in linkage to a ligand predisposed to receptor-mediated endocytosis ( See, Wu & Wu, J. Biol. Chem., 262 pgs. 4429-4432 (1987)) can be used to target cell types which specifically express the receptors of interest.

In another embodiment a nucleic acid ligand compound may be produced in which the ligand comprises a fusogenic viral peptide designed so as to disrupt endosomes, thus allowing the nucleic acid to avoid subsequent lysosomal degradation. The nucleic acid may be targeted in vivo for cell specific endocytosis and expression by targeting a specific receptor such as that described in WO92/06180, WO93/14188 and WO 93/20221. Alternatively the nucleic acid may be introduced intracellularly and incorporated within the host cell genome for expression by homologous recombination. See, Zijlstra et al., Nature, 342, pgs. 435-428 (1989).

In ex vivo gene a gene is transferred into cells in vitro using tissue culture and the cells are delivered to the patient by various methods such as injecting subcutaneously, application of the cells into a skin graft and the intravenous injection of recombinant blood cells such as hematopoietic stem or progenitor cells.

Cells into which a nucleic acid can be introduced for the purposes of gene therapy include, for example, epithelial cells, endothelial cells, keratinocytes, fibroblasts, muscle cells, hepatocytes and blood cells. The blood cells that can be used include, for example, T-lymphocytes, B-lymphocytes, monocytes, macrophages, neutrophils, eosinophils, megakaryotcytes, granulocytes, hematopoietic cells or progenitor cells and the like.

In yet another embodiment the present invention relates to protein chips or protein microarrays. It is well known in the art that microarrays can contain more than 10,000 spots of a protein that can be robotically deposited on a surface of a glass slide or nylon filter. The proteins attach covalently to the slide surface, yet retain their ability to interact with other proteins or small molecules in solution. In some instances the protein samples can be made to adhere to glass slides by coating the slides with an aldehyde-containing reagent that attaches to primary amines. A process for creating microarrays is described, for example by MacBeath and Schreiber in Science, Volume 289, Number 5485, pgs, 1760-1763 (2000) or Service, Science, Vol, 289, Number 5485 pg. 1673 (2000). An apparatus for controlling, dispensing and measuring small quantities of fluid is described, for example, in U.S. Pat. No. 6,112,605.

The present invention also provides a record of protein-protein interactions, PIM®'s, SID®'s and any data encompassed in the following Tables. It will be appreciated that this record can be provided in paper or electronic or digital form.

In order to fully illustrate the present invention and advantages thereof, the following specific examples are given, it being understood that the same are intended only as illustrative and in no way limitative.

### EXAMPLES

### Example 1

### Preparation of a Collection of Random-primed cDNA Fragments

1.A. Collection Preparation and Transformation in Escherichia coli

1.A.1. Random-primed cDNA Fragment Preparation

For the human placenta mRNA sample, random-primed cDNA was prepared from 5 μg of polyA+ mRNA using a TimeSaver cDNA Synthesis Kit (Amersham Pharmacia Biotech) and with 5 μg of random N9-mers according to the manufacturer's instructions. Following phenolic extraction, the cDNA was precipitated and resuspended in water. The resuspended cDNA was phosphorylated by incubating in the presence of T4 DNA Kinase (Biolabs) and ATP for 30 minutes at 37° C. The resulting phosphorylated cDNA was then purified over a separation column (Chromaspin TE 400, Clontech), according to the manufacturer's protocol.

1.A.2. Ligation of Linkers to Blunt-ended cDNA

Oligonucleotide HGX931 (5′ end phosphorylated) 1 μg/μl and HGX932 1 μg/μl.

Sequence of the oligo HGX931: 5′-GGGCCACGAA-3′ (SEQ ID NO. 417) Sequence of the oligo HGX932: 5′-TTCGTGGCCCCTG-3′ (SEQ ID NO. 418)

Linkers were preincubated (5 minutes at 95° C., 10 minutes at 68° C., 15 minutes at 42° C.) then cooled down at room temperature and ligated with cDNA fragments at 16° C. overnight.

Linkers were removed on a separation column (Chromaspin TE 400, Clontech), according to the manufacturer's protocol.

1.A.3. Vector Preparation

Plasmid pP6 (see FIG. 10) was prepared by replacing the SpeI/XhoI fragment of pGAD3S2X with the double-stranded oligonucleotide:

(SEQ ID NO. 419)

5′ CTAGCCATGGCCGCAGGGGCCGCGGCCGCACTAGTGGGGATCCTTAA
TTAAAGGGCCACTGGGGCCCCCGGTACCGGCGTCCCCGGCGCCGGCGTGA

TCACCCCTAGGAATTAATTTCCCGGTGACCCCGGGGGAGCT 3′

The pP6 vector was successively digested with Sfi1 and BamHI restriction enzymes (Biolabs) for 1 hour at 37° C., extracted, precipitated and resuspended in water. Digested plasmid vector backbones were purified on a separation column (Chromaspin TE 400, Clontech), according to the manufacturer's protocol.

1.A.4. Ligation Between Vector and Insert of cDNA

The prepared vector was ligated overnight at 15° C. with the blunt-ended cDNA described in section 2 using T4 DNA ligase (Biolabs). The DNA was then precipitated and resuspended in water.

1.A.5. Library Transformation in Escherichia coli

The DNA from section 1.A.4 was transformed into Electromax DH10B electrocompetent cells (Gibco BRL) with a Cell Porator apparatus (Gibco BRL). 1 ml SOC medium was added and the transformed cells were incubated at 37° C. for 1 hour. 9 mis of SOC medium per tube was added and the cells were plated on LB+ampicillin medium. The colonies were scraped with liquid LB medium, aliquoted and frozen at −80° C.

The obtained collection of recombinant cell clones is named HGXBPLARP1.

1.B. Collection Transformation in Saccharomyces cerevisiae

The Saccharomyces cerevisiae strain (Y187 (MATα Gal4Δ Gal8Δ ade2-101, his3, leu2-3, -112, trp1-901, ura3-52 URA3::UASGAL1-LacZ Met)) was transformed with the cDNA library.

The plasmid DNA contained in E. coli were extracted (Qiagen) from aliquoted E. coli frozen cells (1.A.5.). Saccharomyces cerevisiae yeast Y187 in YPGlu were grown.

Yeast transformation was performed according to standard protocol (Giest et al. Yeast, 11, 355-360, 1995) using yeast carrier DNA (Clontech). This experiment leads to 10⁴ to 5×10⁴ cells/μg DNA. 2×10⁴ cells were spread on DO-Leu medium per plate. The cells were aliquoted into vials containing 1 ml of cells and frozen at −80° C.

The obtained collection of recombinant cell clones is named HGXYPLARP1 (placenta).

1.C. Construction of Bait Plasmids

For fusions of the bait protein (listed in Table II) to the DNA-binding domain of the GAL4 protein of S. cerevisiae, bait fragments were cloned into plasmid pB6. For fusions of the bait protein to the DNA-binding domain of the LexA protein of E. coli, bait fragments were cloned into plasmid pB20.

Plasmid pB6 (see FIG. 3) was prepared by replacing the Nco1/Sa1/ polylinker fragment of pASΔΔ with the double-stranded DNA fragment:

(SEQ ID NO. 420)
5′ CATGGCCGGACGGGCCGCGGCCGCACTAGTGGGGATCCTTAATTAAA
GGGCCACTGGGGCCCCC 3′
(SEQ ID NO. 421)
3′ CGGCCTGCCCGGCGCCGGCGTGATCACCCCTAGGAATTAATTTCCCG
GTGACCCCGGGGGAGCT 5′

Plasmid pB20 (see FIG. 6) was prepared by replacing the EcoRIPstI polylinker fragment of pLex10 with the double-stranded DNA fragment:

(SEQ ID NO. 422)
5′ AATTCGGGGCCGGACGGGCCGCGGCCGCACTAGTGGGGATCCTTAAT
TAAGGGCCACTGGGGCCCCTCGACCTGCA 3′
(SEQ ID NO. 423)
3′ GCCCCGGCCTGCCCGGCGCCGGCGTGATCACCCCTAGGAATTAATTC
CCGGTGACCCCGGGGAGCTGG 5′

The amplification of the bait ORF was obtained by PCR using the Pfu proof-reading Taq polymerase (Stratagene), 10 pmol of each specific amplification primer and 200 ng of plasmid DNA as template.

The PCR program was set up as follows:

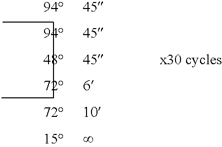

The amplification was checked by agarose gel electrophoresis.

The PCR fragments were purified with Qiaquick column (Qiagen) according to the manufacturer's protocol.

Purified PCR fragments were digested with adequate restriction enzymes. The PCR fragments were purified with Qiaquick column (Qiagen) according to the manufacturer's protocol.

The digested PCR fragments were ligated into an adequately digested and dephosphorylated bait vector (pB6 or pB20) according to standard protocol (Sambrook et al.) and were transformed into competent bacterial cells. The cells were grown, the DNA extracted and the plasmid was sequenced.

### Example 2

### Screening the Collection with the Two-hybrid in Yeast System

2.A. The Mating Protocol

The mating two-hybrid in yeast system (as described by Legrain et al., Nature Genetics, vol.16, 277-282 (1997), Toward a functional analysis of the yeast genome through exhaustive two-hybrid screens) was used for its advantages but one could also screen the cDNA collection in classical two-hybrid system as described in Fields et al. or in a yeast reverse two-hybrid system.

The mating procedure allows a direct selection on selective plates because the two fusion proteins are already produced in the parental cells. No replica plating is required.

This protocol was written for the use of the library transformed into the Y187 strain.

For bait proteins fused to the DNA-binding domain of GAL4, bait-encoding plasmids were first transformed into S. cerevisiae (CG1945 strain (MATa Gal4-542 Gal180-538 ade2-101 his3Δ200, leu2-3,112, trp1-901, ura3-52, lys2-801, URA3::GAL4 17mers (X3)-CyC1TATA-LacZ, LYS2::GAL1UAS-GAL1TATA-HIS3 CYH^{R})) according to step 1.B. and spread on DO-Trp medium.

For bait proteins fused to the DNA-binding domain of LexA, bait-encoding plasmids were first transformed into S. cerevisiae (L40Δgal4 strain (MATa ade2, trp1-901, leu2 3,112, lys2-801, his3Δ200, LYS2::(lexAop)₄-HIS3, ura3-52::URA3 (lexAop)₈-LacZ, GAL4::Kan^{R})) according to step 1.B. and spread on DO-Trp medium.

Day 1, Morning: Preculture

The cells carrying the bait plasmid obtained at step 1.C. were precultured in 20 ml DO-Trp medium and grown at 30° C. with vigorous agitation.

Day 1, Late Afternoon: Culture

The OD_{600 nm} of the DO-Trp pre-culture of cells carrying the bait plasmid pre-culture was measured. The OD_{600 nm} must lie between 0.1 and 0.5 in order to correspond to a linear measurement.50 ml DO-Trp at OD_{600 nm} 0.006/ml was inoculated and grown overnight at 30° C. with vigorous agitation.

Day 2: mating

Medium and Plates

1 YPGlu 15cm plate

50 ml tube with 13 ml DO-Leu-Trp-His

100 ml flask with 5 ml of YPGlu

8 DO-Leu-Trp-His plates

2 DO-Leu plates

2 DO-Trp plates

2 DO-Leu-Trp plates

The OD_{600 nm} of the DO-Trp culture was measured. It should be around 1.

For the mating, twice as many bait cells as library cells were used. To get a good mating efficiency, one must collect the cells at 10⁸ cells per cm².

The amount of bait culture (in ml) that makes up 50 OD_{600 nm} units for the mating with the prey library was estimated.

A vial containing the HGXYCDNA1 library was thawed slowly on ice. 1.0 ml of the vial was added to 5 ml YPGlu. Those cells were recovered at 30° C., under gentle agitation for 10 minutes.

Mating

The 50 OD_{600 nm} units of bait culture was placed into a 50 ml falcon tube.

The HGXYCDNA1 library culture was added to the bait culture, then centrifuged, the supernatant discarded and resuspended in 1.6 ml YPGlu medium.

The cells were distributed onto two 15 cm YPGlu plates with glass beads. The cells were spread by shaking the plates. The plate cells-up at 30° C. for 4h30min were incubated.

Collection of Mated Cells

The plates were washed and rinsed with 6 ml and 7 ml respectively of DO-Leu-Trp-His. Two parallel serial ten-fold dilutions were performed in 500 μl DO-Leu-Trp-His up to 1/10,000. 50 μl of each 1/10000 dilution was spread onto DO-Leu and DO-trp plates and 50 μl of each 1/1000 dilution onto DO-Leu-Trp plates. 22.4 ml of collected cells were spread in 400 μl aliquots on DO-Leu-Trp-His+Tet plates.

Day 4

Clones that were able to grow on DO-Leu-Trp-His+Tetracyclin were then selected. This medium allows one to isolate diploid clones presenting an interaction.

The His+ colonies were counted on control plates.

The number of His+ cell clones will define which protocol is to be processed:

Upon 60.106 Trp+Leu+ colonies:

if the number His+ cell clones<285 then use the process luminometry protocol on all colonies

if the number of His+cell clones>285 and <5000: then process via overlay and then luminometry protocols on blue colonies (2.B and 2.C).

if number of His+cell clones >5000: repeat screen using DO-Leu-Trp-His+Tetracyclin plates containing 3-aminotriazol.

2.B. The X-Gal Overlay Assay

The X-Gal overlay assay was performed directly on the selective medium plates after scoring the number of His⁺ colonies.

Materials

A waterbath was set up. The water temperature should be 50° C.

0.5 M Na₂HPO₄ pH 7.5.

1.2% Bacto-agar.

2% X-Gal in DMF.

Overlay mixture: 0.25 M Na₂HPO₄ pH7.5, 0.5% agar, 0.1% SDS, 7% DMF (LABOSI), 0.04%

X-Gal (ICN). For each plate, 10 ml overlay mixture are needed.

DO-Leu-Trp-His plates.

Sterile toothpicks.

Experiment

The temperature of the overlay mix should be between 45° C. and 50° C. The overlay-mix was poured over the plates in portions of 10 ml. When the top layer was settled, they were collected. The plates were incubated overlay-up at 30° C. and the time was noted. Blue colonies were checked for regularly. If no blue colony appeared, overnight incubation was performed. Using a pen the number of positives was marked. The positives colonies were streaked on fresh DO-Leu-Trp-His plates with a sterile toothpick.

2.C. The Luminometry Assay

His+ colonies were grown overnight at 30° C. in microtiter plates containing DO-Leu-Trp-His+Tetracyclin medium with shaking. The day after, the overnight culture was diluted 15 times into a new microtiter plate containing the same medium and was incubated for 5 hours at 30° C. with shaking. The samples were diluted 5 times and read OD_{600 nm}. The samples were diluted again to obtain between 10,000 and 75,000 yeast cells/well in 100 μl final volume.

Per well, 76 μl of One Step Yeast Lysis Buffer (Tropix) was added, 20 μl Sapphirell Enhancer (Tropix), 4 μl Galacton Star (Tropix) and incubated 40 minutes at 30° C. The β-Gal read-out (L) was measured using a Luminometer (Trilux, Wallach). The value of (OD_{600 nm}×L) was calculated and interacting preys having the highest values were selected.

At this step of the protocol, diploid cell clones presenting interaction were isolated. The next step was now to identify polypeptides involved in the selected interactions.

### Example 3

### Identification of Positive Clones

3.A. PCR on Yeast Colonies

Introduction

PCR amplification of fragments of plasmid DNA directly on yeast colonies is a quick and efficient procedure to identify sequences cloned into this plasmid. It is directly derived from

a published protocol (Wang H. et al., Analytical Biochemistry, 237, 145-146, (1996)). However, it is not a standardized protocol and it varies from strain to strain and it is dependent of experimental conditions (number of cells, Taq polymerase source, etc). This protocol should be optimized to specific local conditions.

Materials

For 1 well, PCR mix composition was:

32.5 μl water,

5 μl 10×PCR buffer (Pharmacia),

1 μl dNTP 10 mM,

0.5 μl Taq polymerase (5u/μl) (Pharmacia),

0.5 μl oligonucleotide ABS1 10 pmole/μl: 5′-GCGTTTGGAATCACTACAGG-3′,(SEQ ID NO. 424)

0.5 μl oligonucleotide ABS2 10 pmole/μl: 5′-CACGATGCACGTTGAAGTG-3′.(SEQ ID NO. 425)

1 N NaOH.

Experiment

The positive colonies were grown overnight at 30° C. on a 96 well cell culture cluster (Costar), containing 150 μl DO-Leu-Trp-His+Tetracyclin with shaking. The culture was resuspended and 100 μl was transferred immediately on a Thermowell 96 (Costar) and centrifuged for 5 minutes at 4,000 rpm at room temperature. The supernatant was removed. 5 μl NaOH was added to each well and shaken for 1 minute.

The Thermowell was placed in the thermocycler (GeneAmp 9700, Perkin Elmer) for 5 minutes at 99.9° C. and then 10 minutes at 4° C. In each well, the PCR mix was added and shaken well.

The PCR program was set up as followed:

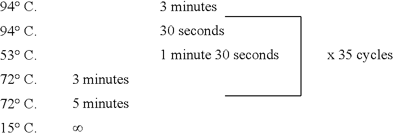

The quality, the quantity and the length of the PCR fragment was checked on an agarose gel. The length of the cloned fragment was the estimated length of the PCR fragment minus 300 base pairs that corresponded to the amplified flanking plasmid sequences.

3.B. Plasmids Rescue from Yeast by Electroporation

Introduction

The previous protocol of PCR on yeast cell may not be successful, in such a case, plasmids from yeast by electroporation can be rescued. This experiment allows the recovery of prey plasmids from yeast cells by transformation of E. coli with a yeast cellular extract. The prey plasmid can then be amplified and the cloned fragment can be sequenced.

Materials

Plasmid Rescue

Glass beads 425-600 μm (Sigma)Phenol/chloroform (1/1) premixed with isoamyl alcohol (Amresco)

Extraction buffer: 2% Triton X100, 1% SDS, 100 mM NaCl, 10 mM TrisHCl pH 8.0, 1 mM EDTA pH 8.0.

Mix ethanol/NH₄Ac: 6 volumes ethanol with 7.5 M NH₄ Acetate, 70% Ethanol and yeast cells in patches on plates.

Electroporation

SOC medium

M9 medium

Selective plates: M9-Leu+Ampicillin

2 mm electroporation cuvettes (Eurogentech)

Experiment

Plasmid Rescue

The cell patch on DO-Leu-Trp-His was prepared with the cell culture of section 2.C. The cell of each patch was scraped into an Eppendorf tube, 300 μl of glass beads was added in each tube, then, 200 μl extraction buffer and 200 μl phenol:chloroform:isoamyl alcohol (25:24:1) was added.

The tubes were centrifuged for 10 minutes at 15,000 rpm.

180 μl supernatant was transferred to a sterile Eppendorf tube and 500 μl each of ethanol/NH₄Ac was added and the tubes were vortexed. The tubes were centrifuged for 15 minutes at 15,000 rpm at 4° C. The pellet was washed with 200 μl 70% ethanol and the ethanol was removed and the pellet was dried. The pellet was resuspended in 10 μl water. Extracts were stored at −20° C.

Electroporation

Materials

Electrocompetent MC1066 cells prepared according to standard protocols (Sambrook et al. supra).

1 μl of yeast plasmid DNA-extract was added to a pre-chilled Eppendorf tube, and kept on ice.

1 μl plasmid yeast DNA-extract sample was mixed and 20 μl electrocompetent cells was added and transferred in a cold electroporation cuvette. Set the Biorad electroporator on 200 ohms resistance, 25 μF capacity; 2.5 kV. Place the cuvette in the cuvette holder and electroporate.

1 ml of SOC was added into the cuvette and the cell-mix was transferred into a sterile Eppendorf tube. The cells were recovered for 30 minutes at 37° C., then spun down for 1 minute at 4,000×g and the supernatant was poured off. About 100 μl medium was kept and used to resuspend the cells and spread them on selective plates (e.g., M9-Leu plates). The plates were then incubated for 36 hours at 37° C.

One colony was grown and the plasmids were extracted. Check for the presence and size of the insert through enzymatic digestion and agarose gel electrophoresis. The insert was then sequenced.

### Example 4

### Protein-protein Interaction

For each bait, the previous protocol leads to the identification of prey polynucleotide sequences. Using a suitable software program (e.g., Blastwun, available on the Internet site of the University of Washington: http://bioweb.pasteur.fr/seqanal/interfaces/blastwu.html) the identity of the mRNA transcript that is encoded by the prey fragment may be determined and whether the fusion protein encoded is in the same open reading frame of translation as the predicted protein or not.

Alternatively, prey nucleotide sequences can be compared with one another and those which share identity over a significant region (60nt) can be grouped together to form a contiguous sequence (Contig) whose identity can be ascertained in the same manner as for individual prey fragments described above.

### Example 5

### Identification of SID®

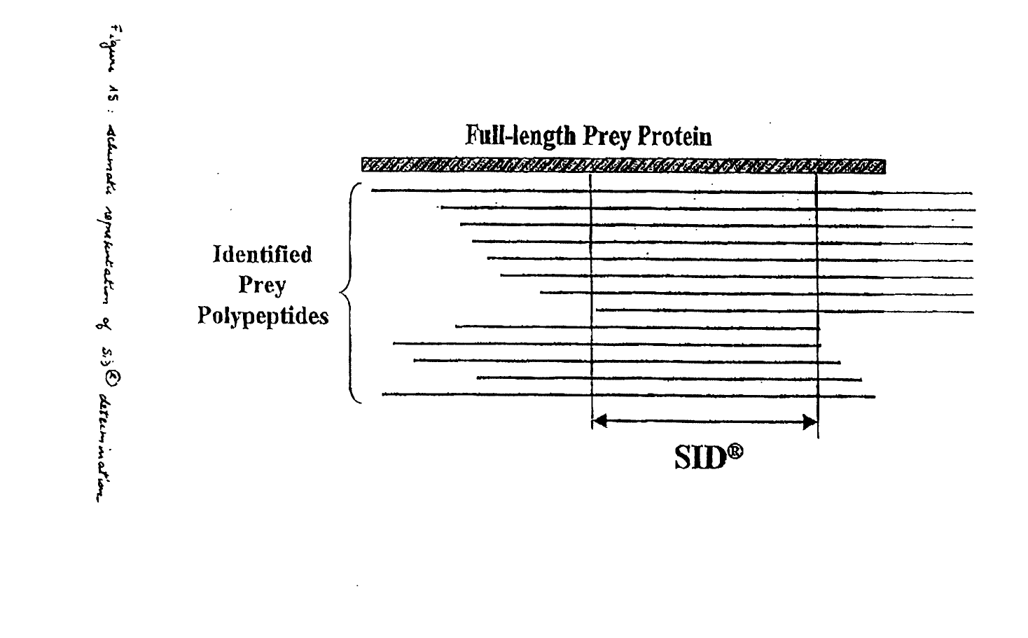

By comparing and selecting the intersection of all isolated fragments that are included in the same polypeptide, one can define the Selected Interacting Domain (SID®) as illustrated in FIG. 15. The SID® is illustrated in Table III.

### Example 6

### Identification of PIM®

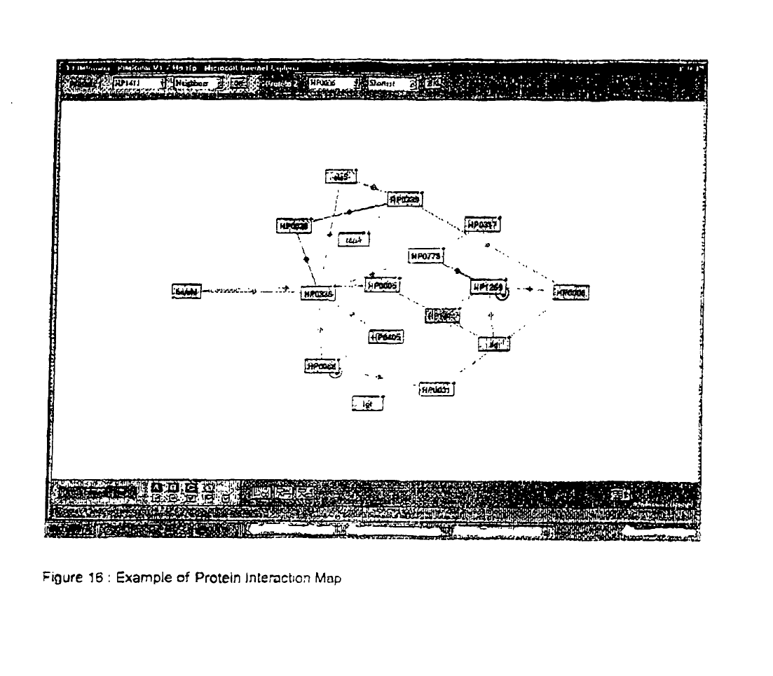

The PIM® is then constructed using methods known in the art as exemplified in FIG. 16.

### Example 7

### Making of Polyclonal and Monoclonal Antibodies

The protein-protein complex of columns 1 and 3 of Table II was injected into mice and polyclonal and monoclonal antibodies were made following the procedure set forth in Sambrook et al. (supra).

More specifically, mice are immunized with an immunogen comprising Table II complexes conjugated to keyhole limpet hemocyanin using glutaraldehyde or EDC as is well known in the art. The complexes can also be stabilized by crosslinking as described in WO 00/37483. The immunogen is then mixed with an adjuvant. Each mouse receives four injections of 10 ug to 100 ug of immunogen, and after the fourth injection, blood samples are taken from the mice to determine if the serum contains antibodies to the immunogen. Serum titer is determined by ELISA or RIA. Mice with sera indicating the presence of antibody to the immunogen are selected for hybridoma production.

Spleens are removed from immune mice and single-cell suspension is prepared (Harlow et al 1988). Cell fusions are performed essentially as described by Kohler et al (1976). Briefly, P365.3 myeloma cells (ATTC Rockville, Md.) or NS-1 myeloma cells are fused with spleen cells using polyethylene glycol as described by Harlow et al (1989). Cells are plated at a density of 2×10⁵ cells/well in 96-well tissue culture plates. Individual wells are examined for growth and the supernatants of wells with growth are tested for the presence of the complex-specific antibodies by ELISA or RIA using one of the proteins set forth in Table II as a target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to one of the proteins in Table II, to determine which are specific for the Table II complexes as opposed to those that bind to the individual proteins. More specifically, antibodies are tested for binding to bait polypeptide of column 1 of Table II alone or to prey polypeptide of column 3 of Table II alone, to determine which are specific for the protein-protein complex of columns 1 and 3 of Table II as opposed to those that bind to the individual proteins.

Monoclonal antibodies against each of the complexes set forth in columns 1 and 3 of Table II are prepared in a similar manner by mixing specified proteins together, immunizing an animal, fusing spleen cells with myeloma cells and isolating clones which produce antibodies specific for he protein complex, but not for individual proteins.

### Example 8

### Modulating Compounds/PIM Screening

Each specific protein-protein complex of columns 1 and 3 of Table II may be used to screen for modulating compounds.

One appropriate construction for this modulating compound screening may be:

bait polynucleotide inserted in pB6 or pB20;- prey polynucleotide inserted in pP6;

transformation of these two vectors in a permeable yeast cell;

growth of the transformed yeast cell on medium containing compound to be tested;

and observation of the growth of the yeast cells.

The following results obtained from these Examples, as well as the teachings in the specification are set forth in the Tables below.

While the invention has been described in terms of the various preferred embodiments, the skilled artisan will appreciate that various modifications, substitutions, omissions and changes may be made without departing from the scope thereof. Accordingly, it is intended that the present invention be limited by the scope of the following claims, including equivalents thereof.

All patent and non-patent publications cited in this specification, including the websites set forth on pages 8, 13 and 33, are indicative of the level of skill of those skilled in the art to which this invention pertains. All these publications and patent applications are herein incorporated by reference to the same extent as if each individual publication or patent application was specifically and individually indicated to be incorporated herein by reference.

TABLE I
Bait sequences

| | 2: Nucleic | | | 5: Amino- | |
|---|---|---|---|---|---|
| 1: Bait | acid | | 4: Nucleic | acid | |
| name | ID No. | 3: Nucleic acid sequence | Positions | ID No. | 6: Amino-acid sequence |
| Shigella | 1 | ATGAATTTAGATGGTGTTAGACCATACTGTAGAATAGTCAATAAAAAGAATGAAAGCATATCAGAT | [1-888] | 8 | MNLDGVRPYCRIVNKKNESIS |
| ospB | | ATTGCATTTGCACATATAATAAAAAGGGTAAAAAATTCATCATGTACTCACCCAAAAGCAGCATTG | | | DIAFAHIIKRVKNSSCTHPKAAL |
| | | GTTTTTTTAGGAGAGAAAGGTTTTTGTGATAGCAATGATGTTCTATCTATTATGGGACAACAAATA | | | VFLGEKGFCDSNDVLSIMGQQ |
| | | CCAAGAGTATTTAAGAACAAGATGTTATATGATTATGTTTTTAAAAATGAAAAAAGTAAAAATGATT | | | IPRVFKNKMLYDYVFKNEKSK |
| | | TTCTAAAAATGGCTGAATCATGGCTACCACAGAGTGAACCAATAGTAATAAATAATGATGATGAC | | | NDFLKMAESWLPQSEPIVINN |
| | | GCATTGAATGCTGCTGCTTATTTTTCTGTAAAAAAAGCGAAAATAAAAACAGTAAACGATACTGAT | | | DDDALNAAAYFSVKKAKIKTV |
| | | TTTAAAGAGTATAATAAGGTTTATATTCTTGGGCACGGTAGTCCTGGTTCTCATCAATTAGGCCTT | | | NDTDFKEYNKVYILGHGSPGS |
| | | GGTTCGGAACTTATTGATGTACAAACAATCATTTCAAGAATGAAAGACTGTGGTATTCTAAATGTG | | | HQLGLGSELIDVQTIISRMKDC |
| | | AAAGATATCCGTTTTACTTCATGCGGCTCCGCTGATAAAGTGGCTCCTAAAAATTTTAACAATGC | | | GILNVKDIRFTSCGSADKVAPK |
| | | CCCTGCTGAAAGTCTTTCTTGTATCCTTAACTCTCTGCCTTTTTTTAAGGAAAAAGAATCTTTGCT | | | NFNNAPAESLSCILNSLPFFKE |
| | | AGAGCAGATAAAAAAACACCTTGAAAACGATGAGTCATTGAGTGATGGTCTAAAAATATCCGGCT | | | KESLLEQIKKHLEDESLSDGL |
| | | ATCATGGATATGGAGTTCACTATGGTCAAGAGCTTTTTCCCTACTCACATTATCGTTCAACTTCAA | | | KISGYHGYGVHYGQELFPYSH |
| | | TTCCTGCTGATCCGGAGCATACAGTAAAAAGAAGCTCTCAGAAAAAGACTTTTATTATTAATAAAG | | | YRSTSIPADPEHTVKRSSQKK |
| | | AACTGGATTAGTATAAAATTTTTAACCTATAG | | | TFIINKELD*YKIFNL* |
| Shigella | 2 | ATGTCAATAAATAACTATGGATTACATCCAGCAAACAACAAAAATATGCACCTAATAATAGGCAGC | [1-711] | 9 | MSINNYGLHPANNKNMHLIIGS |
| ospD1 | | AATACTGCTAATGAAAATAAAGGAATGAAAAATAATATCATTAACGTGACAAATACCGCTATATCC | | | NTANENKGMKNNIINVTNTAIS |
| | | CACGCCATCAATGAAGAAAAATCAGGGGGGGGATATAGTGGTGTTTCTTTCAGAAAATTGGCCA | | | HAINEEKSGGGYSGVSFRKLA |
| | | AAATACAGAACATATCCATTCCGACAAAGAATAATAAGGAGTATAACCGCCATAATTTGTTTTCAT | | | KIQNISIPTKNNKEYNRHNLFS |
| | | TGATTTGGCATGGAAATGCCGATGCAGCGCGTAAATACAGTGAATCGCTGTTGGCAGCCGAAAT | | | LIWHGNADAARKYSESLLAAEI |
| | | ACCCAAAGAGGAAAAACTAGAAGTTCTTGCAGCACGAAATAATGCTGGGGAATCTGCTTTGTTCA | | | PKEEKLEVLAARNNNAGESALFI |
| | | TAGCTCTTCAAGAAGGTCATTCCGCTGCGATTCAAGCTTATGGAGATTTTATTAAAACTTTTGATT | | | ALQEGHSAAIQAYGDFIKFDLK |
| | | TATCACCAAAAGAAACGATTAAACTATTGGATGTAAGAGATAATGAGGGGTTACCAGGATTATTT | | | SPKETIKLLDVRDNEGLPGLFL |
| | | CTGGCCGCAGGGAAAGGGAATATCGAGGCTATGATGGCATATATAAATATATGCCATCATAGTG | | | AAGKGNIEAMMAYINICHHSGI |
| | | GGATAAAACTTACAGAAATAGCAGACAGACTTAACAATAATGAACAAGACATGTTTAATATTATTT | | | KLTEIADRLNNNEQDMFNIISD |
| | | CTGACAAAATACAAGAGTTGTTTTAAGTGTGCTAAATAGCTGCAAAGAATTGCACTTAG | | | KIQELF*VC*IAAKNCT |
| Shigella | 3 | ATGAATATATCAGAAACACTGAACTCAGCAAATACCCAATGCAATATAGATTCTATGGATAACAGA | [1-1434] | 10 | MNISETLNSANTQCNIDSMDN |
| ospC1 | | TTACATACATTGTTTCCAAAAGTGACATCAGTGCGAAACGCTGCACAACAAACTATGCCAGATGA | | | RLHTLFPKVTSVRNAAQQTMP |
| | | AAAAAATTTAAAAGATAGTGCAAATATTATTAAAGATTTCTTTAGGAAAACTATAGCAGCACAGAG | | | DEKNLKDSANIIKDFFRKTIAA |
| | | TTATAGTAGAATGTTCTCTCAAGGCTCTAACTTTAAATCTTTAAATATAGCAATTGATGCACCATCA | | | QSYSRMFSQGSNFKSLNIAID |
| | | GACGCTAAAGCCTCATTTAAGGCTATTGAGCACCTTGACAGATTATCGAAGCATTATATATCTGA | | | APSDAKASFKAIEHLDRLSKHY |
| | | AATAAGGGAAAAACTTCATCCTCTTTCTGCAGAGGAACTCAATTTGCTTTCGCTAATTATTAATTC | | | ISEIREKLHPLSAEELNLLSLIIN |
| | | TGATTTAATCTTCAGACATCAAAGTAATTCTGATTTGTCTGATAAAATTTTAAACATTAAGTCATTC | | | SDLIFRHQSNSDLSDKILNIKSF |
| | | AATAAAATTCAGTCTGAAGGAATATGCACAAAACGAAACACATACGCTGATGATATAAAAAAAATA | | | NKIQSEGICTKRNTYADDIKKIA |
| | | GCTAATCATGACTTTGTGTTTTTTGGCGTTGAAATCTCTAACCATCAGAAAAAACACCCCCTGAAT | | | NHDFVFFGVEISNHQKKHPLN |
| | | ACAAAACATCACACTGTTGATTTTGGTGCAAATGCGTATATCATTGATCATGACTCTCCATATGGA | | | TKHHTVDFGANAYIIDHDSPY |
| | | TATATGACATTAACCGATCACTTTGATAATGCTATTCCACCTGTTTTTTACCATGAGCACCAATCA | | | GYMTLTDHFDNAIPPVFYHEH |
| | | TTTTTAGATAAATTTTCAGAGGTTAATAAAGAAGTTAGTCGATACGTACATGGAAGTAAAGGAATT | | | QSFLDKFSEVNKEVSRYVHGS |
| | | ATAGATGTACCAATATTCAATACTAAAGATATGAAGTTAGGGCTCGGATTATACCTGATTGACTTT | | | KGIIDVPIFNTKDMKLGLGLYLI |
| | | ATTAGAAAAAGTGAAGACCAAAGCTTCAAGGAGTTTTGCTATGGAAAAAATCTTGCCCCTGTGGA | | | DFIRKSEDQSFKEFCYGKNLA |
| | | TCTGGATAGAATCATAAACTTTGTTTTTCAGCCAGAGTACCATATACCTAGGATGGTAAGTACAG | | | PVDLDRIINFVFQPEYHIPRMV |
| | | AAAACTTCAAAAAAGTTAAGATTAGAGAAATATCCTTAGAGGAGGCTGTTACAGCATCTAATTACG | | | STENFKKVKIREISLEEAVTAS |
| | | AAGAAATTAACAAGCAGGTCACTAACAAAAAAATTGCTCTCCAGGCTCTTTTTCTTTCGATTACTA | | | NYEEINKQVTNKKIALQALFLSI |
| | | ATCAAAAAGAGGATGTCGCCTTATATATATTATCTAATTTTGAGATAACTAGACAAGATGTTATTTC | | | TNQKEDVALYILSNFEITRQDVI |
| | | CATAAAGCATGAGTTGTATGATATTGAGTATCTACTTAGCGCTCATAATTCAAGCTGTAAAGTACT | | | SIKHELYDIEYLLSAHNSSCKV |
| | | TGAGTATTTTATCAATAAGGGATTGGTTGATGTAAACACAAAGTTCAAAAAAACTAATAGTGGGGA | | | LEYFINKGLVDVNTKFKKTNSG |
| | | TTGTATGTTGGATAACGCAATAAAATATGAGAATGCAGAAATGATAAAACTATTATTGAAATATGG | | | DCMLDNAIKYENAEMIKLLLKY |
| | | TGCAACATCTGACAATAAATATATTTAATCAAAATTGAATATCGTTTAG | | | GATSDNKYI*SKLNIV* |
| Shigella | 4 | ATGAATATAACAACTCTGACTAATAGTATTTCCACCTCATCATTCAGTCCAAACAATACCAACGGT | [1-1005] | 11 | MNITTLTNSISTSSFSPNNTNG |
| ipaD | | TCATCAACCGAAACAGTTAATTCTGATATAAAAACAACGACCAGTTCTCATCCTGTAAGTTCCCTT | | | SSTETVNSDIKTTTSSHPVSSL |
| | | ACTATGCTCAACGACACCCTTCATAATATCAGAACAACAAATCAGGCATTAAAGAAAGAGCTTTC | | | TMLNDTLHNIRTTNQALKKELS |
| | | ACAAAAAACGTTGACTAAAACATCGCTAGAAGAAATAGCATTACATTCATCTCAGATTAGCATGG | | | QKTLTKTSLEEIALHSSQISMD |
| | | ATGTAAATAAATCCGCTCAACTATTGGATATTCTTTCCAGGAACGAATATCCAATTAATAAAGACG | | | VNKSAQLLDILSRNEYPINKDA |
| | | CAAGAGAATTATTACATTCAGCCCCGAAAGAAGCCGAGCTTGATGGAGATCAAATGATATCTCAT | | | RELLHSAPKEAELDGDQMISH |
| | | AGAGAACTGTGGGCTAAAATTGCAAACTCCATCAATGATATTAATGAACAGTATCTGAAAGTATAT | | | RELQAKIANSINDINEQYLKVY |
| | | GAACATGCCGTTAGTTCATATACTCAAATGTATCAAGATTTTAGCGCTGTTCTTTCCAGTCTTGCC | | | EHAVSSYTQMYQDFSAVLSSL |
| | | GGCTGGATCTCTCCCGGAGGTAACGACGGAAACTCCGTGAAATTACAAGTCAACTCGCTTAAAA | | | AGWISPGGNDGNSVKLQVNS |
| | | AGGCATTGGAAGAACTCAAGGAAAAATATAAAGATAAACCGCTATATCCAGCAAATAATACTGTT | | | LKKALEELKEKYKDKPLYPAN |
| | | AGTCAGGAACAAGCAAATAAATGGCTTACAGAATTAGGTGGAACAATCGGCAAGGTATCTCAAAA | | | NTVSQEQANKWLTELGGTIGK |
| | | AAACGGGGGATATGTTGTCAGTATAAACATGACCCCAATAGACAATATGTTAAAAAGCTTAGATA | | | VSQKNGGYVVSINMTPIDNML |
| | | ATCTAGGTGGAAATGGCGAGGTTGTGCTAGATAATGCAAAATATCAGGCATGGAATGCCGGATT | | | KSLDNLGGNGEVVLDNAKYQ |
| | | CTCTGCCGAAGATGAAACAATGAAAAATAATCTTCAAACTTTAGTTCAAAAATACAGTAATGCCAA | | | AWNAGFSAEDETMKNNLQTL |
| | | TAGTATTTTTGATAATTTAGTAAAGGTTTTGAGTAGTACAATAAGCTCATGTACAGATACAGATTAA | | | VQKYSNANSIFDNLVKVLSSTI |
| | | ACTTTTTCTCCATTTCTGAGGTGCG | | | SSCTDTDKLFLHF*GA |
| Shigella | 5 | ATGTTGCAAAAGCAATTTTGCAACAAACTACTGCTTGATACAAATAAGGAGAATGTTATGGAAATT | [1-1149] | 12 | MLQKQFCNKLLLDTNKENVME |
| ipaC | | CAAAACACAAAACCAACCCAGACTTTATATACAGATATATCCACAAAACAAACTCAAAGTTCTTCC | | | IQNTKPTQTLYTDISTKQTQSS |
| | | GAAACACAAAAATCACAAAATTATCAGCAGATTGCAGCGCATATTCCACTTAATGTCGGTAAAAAT | | | SETQKSQNYQQIAAHIPLNVG |
| | | CCCGTATTAACAACCACATTAAATGATGATCAACTTTTAAAGTTATCAGAGCAGGTTCAGCATGAT | | | KNPVLTTTLNDDQLLKLSEQV |
| | | TCAGAAATCATTGCTCGCCTTACTGACAAAAAGATGAAAGATCTTTCAGAGATGAGTCACACCCT | | | QHDSEIIARLTDKKMKDLSEM |
| | | TACTCCAGAGAACACTCTGGATATTTCCAGTCTTTCTTCTAATGCTGTTTCTTTAATTATTAGTGTA | | | SHTLTPENTLDISSLSSNAVSLI |
| | | GCCGTTCTACTTTCTGCTCTCCGCACTGCAGAAACTAAATTGGGCTCTCAATTGTCATTGATTGC | | | ISVAVLLSALRTAETKLGSQLS |
| | | GTTCGATGCTACAAAATCAGCTGCAGAGAACATTGTTCGGCAAGGCCTGGCAGCCCTATCATCA | | | LIAFDATKSAAENIVRQGLAAL |
| | | AGCATTACTGGAGCAGTCACACAAGTAGGTATAACGGGTATCGGTGCCAAAAAAACGCATTCAG | | | SSSITGAVTQVGITGIGAKKTH |
| | | GGATTAGCGACCAAAAAGGAGCCTTAAGAAAGAACCTTGCCACTGCTCAATCTCTTGAAAAAGA | | | SGISDQKGALRKNLATAQSLE |
| | | GGTTGCAGGTTCTAAATTAGGGTTAAATAAACAAATAGATACAAATATCACCTCACCACAAACTAA | | | KELAGSKLGLNKQIDTNITSPQ |
| | | CTCTAGCACAAAATTTTTAGGTAAAAATAAACTGGCGCCAGATAATATATCCCTGTCAACTGAACA | | | TNSSTKFLGKNKLAPDNISLST |
| | | TAAAACTTCTCTTAGTTCTCCCGATATTTCTTTGCAGGATAAAATTGACACCCAGAGAAGAACTTA | | | EHKTSLSSPDISLQDKIDTQRR |
| | | CGAGCTCAATACCCTTTCTGCGCAGCAAAAACAAAACATTGGCCGTGCAACAATGGAAACATCA | | | TYELNTLSAQQKQNIGRATME |
| | | GCCGTTGCTGGTAATATATCCACATCAGGAGGGCGTTATGCATCTGCTCTTGAAGAAGAAGAAC | | | TSAVAGNISTSGGRYASALEE |
| | | AACTAATCAGTCAGGCCAGCAGTAAACAAGCAGAGGAAGCATCCCAAGTATCTAAAGAAGCATC | | | EEQLISQASSKQAEEASQVSK |
| | | CCAAGCGACAAATCAATTAATACAAAAATTATTGAATATAATTGACAGCATCAACCAATCAAAGAA | | | EASQATNQLIQKLLNIIDSINQS |
| | | TTCGGCAGCCAGTCAGATTGCTGGTAACATTCGAGCTTAA | | | KNSAASQIAGNIRA* |
| Shigella | 6 | ATGTTACCGATAAATAATAACTTTTCATTGCCCCAAAATTCTTTTTATAACACTATTTCCGGTACAT | [1-1022] | 13 | MLPINNNFSLPQNSFYNTISGT |
| ipaH9.8 | | ATGCTGATTACTTTTCAGCATGGGATAAATGGGAAAAACAAGCGCTCCCCGGTGAAGAGCGTGA | | | YADYFSAQDKQEKQALPGEE |
| | | TGAGGCTGTCTCCCGACTTAAAGAATGTCTTATCAATAATTCCGATGAACTTCGACTGGACCGTT | | | RDEAVSRLKECLINNSDELRL |
| | | TAAATCTGTCCTCGCTACCTGACAACTTACCAGCTCAGATAACGCTGCTCAATGTATCATATAATC | | | DRLNLSSLPDNLPAQITLLNVS |
| | | AATTAACTAACCTACCTGAACTGCCTGTTACGCTAAAAAAATTATATTCCGCCAGCAATAAATTAT | | | YNQLTNLPELPVTLKKLYSASN |
| | | CAGAATTGCCCGTGCTACCTCCTGCGCTGGAGTCACTTCAGGTACAACACAATGAGCTGGAAAA | | | KLSELPVLPPALESLQVQHNE |
| | | CCTGCGAGGTTTACCCGATTCGTTATTGACTATGAATATCAGCTATAACGAAATAGTCTCCTTACC | | | LENLPALPDSLLTMNISYNEIV |
| | | ATCGGTCCCACAGGCTCTTAAAAATCTCAGAGGGACCCGTAATTTCCTCACTGAGCTACCAGCAT | | | SLPSLPQALKNLRATRNFLTEL |
| | | TTTCTGAGGGAAATAATCCCGTTGTCAGAGAGTATTTTTTTGATAGAAATCAGATAAGTCATATCC | | | PAFSEGNNPVVREYFFDRNQI |
| | | CGGAAAGCATTCTTAATCTGAGGAATGAATGTTCAATACATATTAGTGATAACCCATTATCATCCC | | | SHIPESILNLRNECSIHISDNPL |
| | | ATGCTCTGCAAGCCCTGCAAAGATTAACCTCTTCGCCGGACTACCACGGCCCACGGATTTACTT | | | SSHALQALQRLTSSPDYHGPR |
| | | CTCCATGAGTGACGGACAACAGAATACACTCCATCGCCCCCTGGCTGATGCCGTGACAGCATG | | | IYFSMSDGQQNTLHRPLADAV |
| | | GTTCCCGGAAAACAAACAATCTGATGTATCACAGATATGGCATGCTTTTGAACATGAAGAGCATG | | | TAQFPENKQSDVSQIWHAFE |
| | | CCAACACCTTTTCCGCGTTCCTTGACCGCCTTTCCGATACCGTCTCTGCACGCAATACCTCCGG | | | HEEHANTFSAFLDRLSDTVSA |
| | | ATTCCGTGAACAGGTCGCTGCATGGCTGGAAAAACTCAGTGCCTCTGCGGAGCTTCGACAGCA | | | RNTSGFREQVAAQLEKLSAS |
| | | GTCTTTCGCTGTTGCTGCTGATGCCACTGAGAGCTGTGAGGACCGTGT | | | AELRQQSFAVAADATESCEDR |
| Shigella | 7 | ATGAAAATAACATCTACCATTATTCAAACACCTTTTCCATTTGAGAATAATAATTCTCATGCTGGCA | [1-612] | 14 | MKITSTIIQTPFPFENNNSHAGI |
| ospG | | TAGTAACGGAGCCCATTCTCGGTAAGTTAATAGGTCAGGGGTCGACAGCAGAAATCTTTGAAGA | | | VTEPILGKLIGQGSTAEIFEDV |
| | | TGTGAATGATTCATCTGCTTTGTATAAAAAGTATGATCTTATTGGCAACCAGTACAATGAGATTCT | | | NDSSALYKKYDLIGNQYNEILE |
| | | GGAAATGGCTTGGCAAGAATCTGAGCTTTTTAATGCTTTTTATGGCGATGAAGCATCCGTTGTTA | | | MAWQESELFNAFYGDEASVVI |
| | | TACAGTATGGCGGAGATGTGTACCTCCGAATGCTGCGCGTGCCTGGGACTCCCCTTAGTGACAT | | | QYGGDVYLRMLRVPGTPLSDI |
| | | TGATACAGCTGATATCCCTGATAATATAGAGAGCCTTTATCTACAGTTGATATGTAAATTGAATGA | | | DTADIPDNIESLYLQLICKLNEL |
| | | GTTGAGTATAATCCATTACGATCTTAATACAGGTAATATGCTGTATGATAAAGAAAGTGAAAGTTT | | | SIIHYDLNTGNMLYDKESESLF |
| | | ATTCCCAATAGATTTTCGCAATATTTATGCTGAATATTACGCTGCAACCAAAAAAGATAAAGAGAT | | | PIDFRNIYAEYYAATKKDKEIID |
| | | TATCGACCGACGATTACAAATGCGTACAAATGATTTTTATTCGTTATTAAACAGGAAATATTTATA | | | RRLQMRTNDFYSLLNRKYL*T |
| | | GACGTATTTGTTGATGCTATAA | | | YLLML* |

TABLE II
Bait-prey interactions

| | 2: Bait nucleic | |
|---|---|---|
| 1: Bait name | acid SEQ ID No. | 3: Prey name |
| Shigella ospB | 1 | prey44074 (JM5; prey44078) hJM5 |
| Shigella ospB | 1 | prey67804 (LOC91851) hhypothetical proteinXP_041083 |
| Shigella ospB | 1 | prey67806 |
| Shigella opsB | 1 | prey67810 (FBXO3 FBX3 DKFZp564B092 FBA) hFBXO3 |
| Shigella ospB | 1 | prey5237 (NONO NRB54 NMT55 P54NRB) hNONO |
| Shigella ospB | 1 | prey67661 (CAPN2 CANPL2 CANPML) hCAPN2 |
| Shigella ospB | 1 | prey34730 (LMO4; prey34731) hLMO4 |
| Shigella ospB | 1 | prey33141 (ZIN; prey33142) hZIN |
| Shigella ospB | 1 | prey67575 (LOC136773) hsimilar to 3-HYDROXYISOBUTYRATE DEHYDROGENASE, MITOCHONDRIAL |
| | | PRECURSOR (HIBADH) (H. sapiens) |
| Shigella ospB | 1 | prey67608 (MGC4126) hMGC4126 |
| Shigella ospB | 1 | prey67637 (LOC90706) hhypothetical proteinXP_033663 |
| Shigella ospB | 1 | prey12713 (LMO2 RBTNL1 RHOM2 TTG2 RBTN2; prey12714) hLMO2 hTTG-2a/RBTN-2a |
| Shigella ospB | 1 | prey67836 (MYO9A) hMYO9A |
| Shigella ospB | 1 | prey700 (RANBP9 RANBPM RANBP9-PENDING; prey701) hRANBP9 hRanBPM |
| Shigella ospB | 1 | prey67844 |
| Shigella ospB | 1 | prey67853 |
| Shigella ospB | 1 | prey66272 (FLJ20254) hFLJ20254 |
| Shigella ospD1 | 2 | prey700 (RANBP9 RANBPM RANBP9-PENDING; prey701) hRANBP9 hRanBPM |
| Shigella ospD1 | 2 | prey2492 (FLJ11026; prey2493) hFLJ11026 |
| Shigella ospD1 | 2 | prey67651 putative homolog of prey064241-Mouse |
| Shigella ospD1 | 2 | prey67653 putative homolog of prey067652- |
| Shigella ospD1 | 2 | prey67667 (PACSIN2) hPACSIN2 |
| Shigella ospD1 | 2 | prey67657 hUnknown (protein forMGC: 16824) |
| Shigella ospD1 | 2 | prey67501 (LOC51667) hLOC51667 |
| Shigella ospD1 | 2 | prey67678 (LOC90410) hhypothetical proteinXP_031534 |
| Shigella ospD1 | 2 | prey67578 (LOC121052) hhypothetical proteinXP_035313 |
| Shigella ospD1 | 2 | prey67580 (DKFZp586I021) hDKFZp586I021 |
| Shigella ospD1 | 2 | prey3160 (KIF5B UKHC KNS KNS1 U-KHC KINH; prey3161) hKIF5B hkinesin heavychain |
| Shigella ospD1 | 2 | prey50427 (KIAA0419; prey50428) hKIAA0419 |
| Shigella ospD1 | 2 | prey63765 (LIM; prey63767) hLIM |
| Shigella ospD1 | 2 | prey67623 (LDB2 CLIM1) hLDB2 |
| Shigella ospD1 | 2 | prey7315 (LDB1 CLIM2 NLI; prey7316) hLDB1 hCLIM2 |
| Shigella ospD1 | 2 | prey67601 (ATIP1 KIAA1288 DKFZp586D1519 FLJ14295) hATIP1 |
| Shigella ospD1 | 2 | prey53735 (TLN1 TLN KIAA1027) hTLN1 |
| Shigella ospD1 | 2 | prey67630 |
| Shigella ospD1 | 2 | prey12665 (CREBL1 CREB-RP G13; prey12666) hCREBL1 hG13 |
| Shigella ospD1 | 2 | prey67631 (FLJ21742) hFLJ21742 |
| Shigella ospD1 | 2 | prey20143 (SYNCOILIN; prey20144) hSYNCOILIN |
| Shigella ospD1 | 2 | prey1418 (NR1H2 UNR NER NER-I RIP15 LXR-B; prey1419) hNR1H2 hNer-I |
| Shigella ospD1 | 2 | prey67642 (ALDH3B2 ALDH3B2-PENDING ALDH8) hALDH3B2 |
| Shigella ospD1 | 2 | prey67648 (PON2) hPON2 |
| Shigella ospC1 | 3 | prey67266 |
| Shigella ospC1 | 3 | prey67267 |
| Shigella ospC1 | 3 | prey50590 (TID1; prey48229) hTID1 |
| Shigella ospC1 | 3 | prey9822 |
| Shigella ospC1 | 3 | prey67268 |
| Shigella ospC1 | 3 | prey67270 |
| Shigella ospC1 | 3 | prey67271 (STAT5B STAT5) hSTAT5B |
| Shigella ospC1 | 3 | prey700 (RANBP9 RANBPM RANBP9-PENDING; prey701) hRANBP9 hRanBPM |
| Shigella ospC1 | 3 | prey3486 (PM5; prey3487) hPM5 hpM5 |
| Shigella ospC1 | 3 | prey14801 (KIAA0321) hKIAA0321 |
| Shigella ospC1 | 3 | prey67279 |
| Shigella ospC1 | 3 | prey67280 |
| Shigella ospC1 | 3 | prey49194 (KIAA0211; prey49195) hKIAA0211 |
| Shigella ospC1 | 3 | prey67287 |
| Shigella ospC1 | 3 | prey19931 (HEF1 CAS-L) hHEF1 |
| Shigella ospC1 | 3 | prey67290 |
| Shigella ospC1 | 3 | prey67291 |
| Shigella ospC1 | 3 | prey67294 |
| Shigella ospC1 | 3 | prey67296 |
| Shigella ospC1 | 3 | prey67299 |
| Shigella ospC1 | 3 | prey4637 (TAF2A BA2R CCG1 CCGS NSCL2 TAFII250; prey4638 prey4639) hTAF2A |
| Shigella ospC1 | 3 | prey67316 |
| Shigella ospC1 | 3 | prey67318 |
| Shigella ospC1 | 3 | prey7144 (IMMT P87/89 HMP; prey7145) hIMMT hp87/89 |
| Shigella ospC1 | 3 | prey67328 (TSC22) hTSC22 |
| Shigella ospC1 | 3 | prey37430 (WASL N-WASP; prey37432) hWASL hN-WASP |
| Shigella ospC1 | 3 | prey67351 |
| Shigella ospC1 | 3 | prey67353 |
| Shigella ospC1 | 3 | prey25185 hHSPC272 |
| Shigella ospC1 | 3 | prey4411 (ZNF147 EFP TRIM25 Z147) hZNF147 |
| Shigella ospC1 | 3 | prey2686 (VRP AD3; prey2687) hVRP |
| Shigella ospC1 | 3 | prey67368 (LOC92609) hhypothetical proteinXP_053074 |
| Shigella ospC1 | 3 | prey67371 |
| Shigella ospC1 | 3 | prey4005 (KIAA0141; prey4006; prey8649; prey44107) hKIAA0141 |
| Shigella ospC1 | 3 | prey67380 |
| Shigella ospC1 | 3 | prey3296 (FHOS; prey3297) hFHOS |
| Shigella ospC1 | 3 | prey2108 (prey2101; prey2104; prey2107; prey2102; prey2103) hSimilar to COP9 (constitutive |
| | | photomorphogenic), subunit 5(Arabidopsis) hsimilar to COP9 (constitutive photomorphogenic, Arabidopsis, |
| | | homolog) subunit 5 (H. sapiens) hCOPS5 hsimilar to COP9 (constitutive photomorphogenic, Arabidopsis, |
| | | homolog) subunit 5 (H. sapiens) hCOPS5 hsimilar to COP9 (constitutive photomorphogenic, Arabidopsis, |
| | | homolog) subunit 5 (H. sapiens) |
| Shigella ospC1 | 3 | prey67403 |
| Shigella ospC1 | 3 | prey67405 |
| Shigella ospC1 | 3 | prey14400 (prey14399; prey14401) hprotein phosphatase 5, catalyticsubunit hPPP5C hPPP5C |
| Shigella ospC1 | 3 | prey50029 |
| Shigella ipaD | 4 | prey67563 (PRSC1) hPRSC1 |
| Shigella ipaD | 4 | prey2109 (COPS5 JAB1 SGN5 MOV-34; prey2110) hCOPS5 h38 kDa Mov34homolog |
| Shigella ipaD | 4 | prey25185 hHSPC272 |
| Shigella ipaD | 4 | prey53990 (TNFRSF1A CD120a TNF-R TNF-R-I TNF-R55 TNFAR TNFR60 TNFR1 p55-R p55) hTNFRSF1A |
| Shigella ipaD | 4 | prey9120 (VIM; prey9122) hVIM hvimentin |
| Shigella ipaD | 4 | prey67571 |
| Shigella ipaD | 4 | prey67572 |
| Shigella ipaD | 4 | prey65696 (KARS KIAA0070; prey65697) hKARS hLysyl tRNASynthetase |
| Shigella ipaD | 4 | prey8889 (PLCB3) hPLCB3 |
| Shigella ipaD | 4 | prey700 (RANBP9 RANBPM RANBP9-PENDING; prey701) hRANBP9 hRanBPM |
| Shigella ipaD | 4 | prey2694 (INDO IDO; prey2696; prey2693) hINDO hINDO |
| Shigella ipaD | 4 | prey53735 (TLN1 TLN KIAA1027) hTLN1 |
| Shigella ipaD | 4 | prey67574 |
| Shigella ipaC | 5 | prey67509 (POLR2A RPOL2 POLR2 POLRA hRPB220 hsRPB1 RPO2 RpIILS RPBh1 RPB1) hPOLR2A |
| Shigella ipaC | 5 | prey67514 |
| Shigella ipaC | 5 | prey2926 (FLJ23153; prey2927) hFLJ23153 |
| Shigella ipaC | 5 | prey4458 (RRBP1 ES130 ES/130; prey4459) hRRBP1 hES/130 |
| Shigella ipaC | 5 | prey4458 (RRBP1 ES130 ES/130; prey4459) hRRBP1 hES/130 |
| Shigella ipaC | 5 | prey67522 |
| Shigella ipaC | 5 | prey527 (CLTC CLTCL2 KIAA0034; prey528) hCLTC hKIAA0034 |
| Shigella ipaC | 5 | prey53735 (TLN1 TLN KIAA1027) hTLN1 |
| Shigella ipaC | 5 | prey53735 (TLN1 TLN KIAA1027) hTLN1 |
| Shigella ipaC | 5 | prey67546 (LOC128116) hsimilar to phosphodiesterase 4D interacting protein (myomegalin) (H. sapiens) |
| Shigella ipaC | 5 | prey4671 (KIAA0454) hKIAA0454 |
| Shigella ipaC | 5 | prey67550 (LOC92689) hhypothetical proteinXP_046663 |
| Shigella ipaC | 5 | prey8889 (PLCB3) hPLCB3 |
| Shigella ipaC | 5 | prey11375 (HSPBP1; prey11376) hHSPBP1 hHsp70 binding proteinHsBP1 |
| Shigella ipaC | 5 | prey67473 (GALE) hGALE |
| Shigella ipaC | 5 | prey8929 (KIAA0728 FLJ21489) hKIAA0728 |
| Shigella ipaC | 5 | prey3488 (ACF7 ABP620 KIAA1251 KIAA0465) hACF7 |
| Shigella ipaC | 5 | prey3514 (SNX1; prey3515) hSNX1 |
| Shigella ipaC | 5 | prey5814 (USP9X DFFRX) hUSP9X |
| Shigella ipaC | 5 | prey5814 (USP9X DFFRX) hUSP9X |
| Shigella ipaC | 5 | prey67479 |
| Shigella ipaC | 5 | prey700 (RANBP9 RANBPM RANBP9-PENDING; prey701) hRANBP9 hRanBPM |
| Shigella ipaC | 5 | prey67481 (GDBR1 GBDR1) hGDBR1 |
| Shigella ipaC | 5 | prey67488 (LOC126257) hsimilar to putative (H. sapiens) |
| Shigella ipaC | 5 | prey51967 (UBQLN1 DSK2 PLIC-1 DA41 XDRP1) hUBQLN1 |
| Shigella ipaC | 5 | prey67491 (KIAA1007 AD-005) hKIAA1007 |
| Shigella ipaC | 5 | prey323 (CSH1 CSMT CSA PL; prey324; prey325) hCSH1 |
| Shigella ipaC | 5 | prey67495 |
| Shigella ipaC | 5 | prey67506 (LOC126083) hdynamin2 |
| Shigella ipaC | 5 | prey4578 (PSAP SAP1 GLBA; prey5664) hPSAP hGLBA |
| Shigella ipaC | 5 | prey1135 (PSMD1 P112 S1; prey1136) hPSMD1 hproteasome subunitp112 |
| Shigella ipaC | 5 | prey67465 (COL4A2 FLJ22259) hCOL4A2 |
| Shigella ipaC | 5 | prey28880 (KPNA4; prey28881) hKPNA4 hQIP1 |
| Shigella ipaC | 5 | prey3599 (TRIP12 KIAA0045; prey3600) hTRIP12 hKIAA0045 |
| Shigella ipaH9.8 | 6 | prey67717 |
| Shigella ipaH9.8 | 6 | prey700 (RANBP9 RANBPM RANBP9-PENDING; prey701) hRANBP9 hRanBPM |
| Shigella ipaH9.8 | 6 | prey67718 (KIAA1715) hKIAA1715 |
| Shigella ipaH9.8 | 6 | prey2530 harrestin, beta1 |
| Shigella ipaH9.8 | 6 | prey67731 (LOC126896) hsimilar to Gene 33/Mig-6 (H. sapiens) |
| Shigella ipaH9.8 | 6 | prey7155 (CSH2 CSB) hCSH2 |
| Shigella ipaH9.8 | 6 | prey1687 (DCTN1) hDCTN1 |
| Shigella ipaH9.8 | 6 | prey67734 (FLJ10618) hFLJ10618 |
| Shigella ipaH9.8 | 6 | prey2694 (INDO IDO; prey2696; prey2693) hINDO hINDO |
| Shigella ipaH9.8 | 6 | prey67740 |
| Shigella ipaH9.8 | 6 | prey67703 (PPP2R4 PTPA) hPPP2R4 |
| Shigella ipaH9.8 | 6 | prey67741 |
| Shigella ipaH9.8 | 6 | prey67742 (FLJ20313) hFLJ20313 |
| Shigella ipaH9.8 | 6 | prey67339 (MMP19 RASI-1 MMP18) hMMP19 |
| Shigella ipaH9.8 | 6 | prey67337 (MMP19 RASI-1 MMP18) hMMP19 |
| Shigella ipaH9.8 | 6 | prey67746 (FBXO25 FBX25) hFBXO25 |
| Shigella ipaH9.8 | 6 | prey54430 (PSG4 PSG9) hPSG4 |
| Shigella ipaH9.8 | 6 | prey67749 |
| Shigella ipaH9.8 | 6 | prey67751 |
| Shigella ipaH9.8 | 6 | prey8739 (MLL2 ALR; prey8742) hMLL2 hALR |
| Shigella ipaH9.8 | 6 | prey18232 (CCT3 TRIC5 CCTG; prey18233) hCCT3 hCctg |
| Shigella ipaH9.8 | 6 | prey66739 (EIF2B1 EIF2B EIF-2B) hEIF2B1 |
| Shigella ipaH9.8 | 6 | prey67769 (PP2135 FLJ00041) hPP2135 |
| Shigella ipaH9.8 | 6 | prey13613 (KIAA0970) hKIAA0970 |
| Shigella ipaH9.8 | 6 | prey3337 (LMNA LMN1 EMD2 FPL LFP LDP1 FPLD CMD1A; prey14196) hLMNA |
| Shigella ipaH9.8 | 6 | prey67774 (LOC119758) hsimilar to REGULATOR OF PRESYNAPTIC ACTIVITY AEX-3 (H. sapiens) |
| Shigella ipaH9.8 | 6 | prey67776 |
| Shigella ipaH9.8 | 6 | prey4758 (DKFZP761L0424 KIAA1217) hDKFZP761L0424 |
| Shigella ipaH9.8 | 6 | prey67781 putative homolog of prey046760-Mouse Fmnl |
| Shigella ipaH9.8 | 6 | prey2109 (COPS5 JAB1 SGN5 MOV-34; prey2110) hCOPS5 h38 kDa Mov34homolog |
| Shigella ipaH9.8 | 6 | prey4060 (KIAA0155; prey4061; prey4062) hKIAA0155 |
| Shigella ipaH9.8 | 6 | prey49284 (SLC7A8 LAT2) hSLC7A8 |
| Shigella ipaH9.8 | 6 | prey67686 |
| Shigella ipaH9.8 | 6 | prey66872 (MRPS9) hMRPS9 |
| Shigella ipaH9.8 | 6 | prey67690 (RRP4) hRRP4 |
| Shigella ipaH9.8 | 6 | prey67695 (ATP6N1B RDRTA2 RTA1C VPP2 RTADR) hATP6N1B |
| Shigella ipaH9.8 | 6 | prey67336 (MMP19 RASI-1 MMP18) hMMP19 |
| Shigella ipaH9.8 | 6 | prey6299 (KIAA0335; prey6300) hKIAA0335 |
| Shigella ipaH9.8 | 6 | prey6586 (FLNA ABPX ABP-280 FLN FLN1 NHBP; prey6587) hFLNA |
| Shigella ipaH9.8 | 6 | prey56789 (ALDH4 P5CDH; prey56791) hALDH4 hP5CDh |
| Shigella ipaH9.8 | 6 | prey67711 |
| Shigella ipaH9.8 | 6 | prey2118 (RNF2 dinG Bap-1; prey2119) hRNF2 hring finger proteinBAP-1 |
| Shigella ipaH9.8 | 6 | prey3596 (DDX15 HRH2 DBP1; prey3597) hDDX15 hATP-dependent RNA helicase #46 |
| Shigella ipaH9.8 | 6 | prey666 (RANBP16 KIAA0745; prey667; prey665; prey9721) hRANBP16 hRAN binding protein16 hRANBP16 |
| | | hRANBP16 |
| Shigella ospG | 7 | prey3917 (BTBD2 FLJ20386; prey3920; prey3918; prey3921; prey3922; prey3919) hBTBD2 |
| Shigella ospG | 7 | prey63632 (ZNF189; prey63789) hZNF189 |
| Shigella ospG | 7 | prey2109 (COPS5 JAB1 SGN5 MOV-34; prey2110) hCOPS5 h38 kDa Mov34homolog |
| Shigella ospG | 7 | prey54201 (UBE2D3 UBCH5C; prey54202) hUBE2D3 hUBCH5C |
| Shigella ospG | 7 | prey1922 (DLST DLTS; prey1923) hDLST hE2K |
| Shigella ospG | 7 | prey67418 (UBE2L3 UBCH7) hUBE2L3 |
| Shigella ospG | 7 | prey67314 (UBE2L6 UBCH8 RIG-B) hUBE2L6 |
| Shigella ospG | 7 | prey67435 hUnknown (protein forMGC:3432) |
| Shigella ospG | 7 | prey67443 (FLJ11807) hFLJ11807 |
| Shigella ospG | 7 | prey67317 (KIAA1485) hKIAA1485 |
| Shigella ospG | 7 | prey67393 (UBE2D2 UBCH5B UBC4) hUBE2D2 |
| Shigella ospG | 7 | prey700 (RANBP9 RANBPM RANBP9-PENDING; prey701) hRANBP9 hRanBPM |
| Shigella ospG | 7 | prey67411 (UBE2E3 UBCH9) hUBE2E3 |
| Shigella ospG | 7 | prey67423 |
| Shigella ospG | 7 | prey67298 |
| Shigella ospG | 7 | prey67464 |
| Shigella ospG | 7 | prey67320 |
| Shigella ospG | 7 | prey67321 |
| Shigella ospG | 7 | prey35777 (PSG2 PSBG2 PSGGB; prey35778) hPSG2 hPSG1 |
| Shigella ospG | 7 | prey67327 (AKAP13 HT31 BRX) hAKAP13 |
| Shigella ospG | 7 | prey412 (RPN2; prey413) hRPN2 hsignalpeptide |
| Shigella ospG | 7 | prey50598 (PEX10 NALD; prey50599) hPEX10 hperoxisome assembly proteinPEX10 |
| Shigella ospG | 7 | prey67364 |
| Shigella ospG | 7 | prey67367 |
| Shigella ospG | 7 | prey67369 |
| Shigella ospG | 7 | prey67372 (CD63 MLA1 ME491) hCD63 |
| Shigella ospG | 7 | prey67379 |
| Shigella ospG | 7 | prey67381 (LOC131541) hhypothetical proteinXP_059524 |
| ospB | 1 | gb\|AB008515\|AB008515 Homo sapiens mRNA for RanBPM, complete cds. |
| ospB | 1 | gb\|AC005091\|AC005091 Homo sapiens BAC clone CTA-318C11 from 7p14-p15, complete sequence. |
| ospB | 1 | gb\|AF117888\|AF117888 Homo sapiens myosin-IXa mRNA, complete cds. |
| ospB | 1 | gb\|AF141347\|AF141347 Homo sapiens hum-a-tub2 alpha-tubulin mRNA, complete cds. |
| ospB | 1 | gb\|AF176702\|AF176702 Homo sapiens F-box protein FBX3 mRNA, partial cds. |
| ospB | 1 | gb\|AF177198\|AF177198 Homo sapiens talin mRNA, complete cds. |
| ospB | 1 | gb\|AF212940\|AF212940 Homo sapiens zinedin (ZIN) mRNA, complete cds. |
| ospB | 1 | gb\|AF257211\|AF257211 Homo sapiens LMO2b splice variant (LMO2) mRNA, complete cds. |
| ospB | 1 | gb\|AJ005897\|HSA005897 Homo sapiens mRNA for JM5 protein, complete CDS (clone IMAGE 53337, |
| | | LLNLc110F1857Q7 (RZPD Berlin) and LLNLc110G0913Q7 (RZPD Berlin)). |
| ospB | 1 | gb\|AK024239\|AK024239 Homo sapiens cDNA FLJ14177 fis, clone NT2RP2003161 |
| ospB | 1 | gb\|AL049176\|HS141H5 Human DNA sequence from clone 141H5 on chromosome Xq22.1-23. Contains |
| | | parts of a novel Chordin LIKE protein with von Willebrand factor type C domains. Contains ESTs, STSs |
| | | and GSSs, complete sequence. |
| ospB | 1 | gb\|AL122043\|HSM801240 Homo sapiens mRNA; cDNA DKFZp566G1424 (from clone DKFZp566G1424). |
| ospB | 1 | gb\|AL442166\|HSMX1A Homo sapiens chromosome 21 from 5 PACs and 5 Cosmids map |
| | | 21q22.2, D21S349-MX1; segment 1/2, complete sequence. |
| ospB | 1 | gb\|AP002026\|AP002026 Homo sapiens genomic DNA, chromosome 4q22-q24, clone: 429K21, |
| | | complete sequence. |
| ospB | 1 | gb\|D21260\|HUMORFEA Human mRNA for KIAA0034 gene, complete cds. |
| ospB | 1 | gb\|L14599\|HUMPSFHOMO Human mRNA, complete cds. |
| ospB | 1 | gb\|L28809\|HUMAAE Homo sapiens dbpB-like protein mRNA, complete cds. |
| ospB | 1 | gb\|M23254\|HUMCANP Human Ca2-activated neutral protease large subunit (CANP) mRNA, complete cds. |
| ospB | 1 | gb\|U24576\|U24576 Homo sapiens breast tumor autoantigen (LMO4) mRNA, complete cds. |
| ospB | 1 | gb\|X61118\|HSTTG2 Human TTG-2 mRNA for a cysteine rich protein with LIM motif. |
| ospD1 | 2 | gb\|AB007879\|AB007879 Homo sapiens KIAA0419 mRNA, complete cds. |
| ospD1 | 2 | gb\|AB008515\|AB008515 Homo sapiens mRNA for RanBPM, complete cds. |
| ospD1 | 2 | gb\|AB016485\|AB016485 Homo sapiens mRNA for LIM homeobox protein cofactor (CLIM-2), complete cds. |
| ospD1 | 2 | gb\|AB028956\|AB028956 Homo sapiens mRNA for KIAA1033 protein, partial cds. |
| ospD1 | 2 | gb\|AB033114\|AB033114 Homo sapiens mRNA for KIAA1288 protein, partial cds. |
| ospD1 | 2 | gb\|AC003108\|HUAC003108 Human Chromosome 16 BAC clone CIT987Sk-327O24, complete sequence. |
| ospD1 | 2 | gb\|AC008764\|AC008764 Homo sapiens chromosome 19 clone CTD-3222D19, complete sequence. |
| ospD1 | 2 | gb\|AF001601\|AF001601 Homo sapiens paraoxonase (PON2) mRNA, complete cds. |
| ospD1 | 2 | gb\|AF006466\|AF006466 Mus musculus lymphocyte specific formin related protein (Fr1) mRNA, complete cds. |
| ospD1 | 2 | gb\|AF061258\|AF061258 Homo sapiens LIM protein mRNA, complete cds. |
| ospD1 | 2 | gb\|AF068651\|AF068651 Homo sapiens LIM-domain binding factor CLIM1 (CLIM1) mRNA, complete cds. |
| ospD1 | 2 | gb\|AF128536\|AF128536 Homo sapiens cytoplasmic phosphoprotein PACSIN2 mRNA, complete cds. |
| ospD1 | 2 | gb\|AF155099\|AF155099 Homo sapiens NY-REN-18 antigen mRNA, complete cds. |
| ospD1 | 2 | gb\|AF177198\|AF177198 Homo sapiens talin mRNA, complete cds. |
| ospD1 | 2 | gb\|AF265342\|AF265342 Homo sapiens chromosome 8 map 8p BAC 2053N22, complete sequence. |
| ospD1 | 2 | gb\|AK001888\|AK001888 Homo sapiens cDNA FLJ11026 fis, clone PLACE1004104. |
| ospD1 | 2 | gb\|AL121808\|CNS01DSJ Human chromosome 14 DNA sequence *** IN PROGRESS *** BAC C-2313O13 of |
| | | library CalTech-D from chromosome 14 of Homo sapiens (Human), complete sequence. |
| ospD1 | 2 | gb\|AQ628981\|AQ628981 RPCI-11-469I15.TJ RPCI-11 Homo sapiens genomic clone |
| | | RPCI-11-469I15, DNA sequence. |
| ospD1 | 2 | gb\|B88348\|B88348 CIT-HSP-2063N18.TFB CIT-HSP Homo sapiens genomic clone 2063N18, DNA sequence. |
| ospD1 | 2 | gb\|M57298\|HUMGPG25K Human GTP-binding protein G25K mRNA, complete cds. |
| ospD1 | 2 | gb\|M63960\|HUMPRPHOS1 Human protein phosphatase-1 catalytic subunit mRNA, complete cds. |
| ospD1 | 2 | gb\|U07132\|HSU07132 Human steroid hormone receptor Ner-I mRNA, complete cds. |
| ospD1 | 2 | gb\|U31903\|HSU31903 Human CREB-RP (creb-rp) mRNA, complete cds. |
| ospD1 | 2 | gb\|U37519\|HSU37519 Human aldehyde dehydrogenase (ALDH8) mRNA, complete cds. |
| ospD1 | 2 | gb\|X65873\|HSKHCMR H. sapiens mRNA for kinesin (heavy chain). |
| ospD1 | 2 | gb\|X65873\|HSKHCMR H. sapiens mRNA for kinesin (heavy chain). |
| ospD1 | 2 | gb\|X65873\|HSKHCMR H. sapiens mRNA for kinesin (heavy chain). |
| ipaD | 4 | gb\|AB008515\|AB008515 Homo sapiens mRNA for RanBPM, complete cds. |
| ipaD | 4 | gb\|AF161390\|AF161390 Homo sapiens HSPC272 mRNA, partial cds. |
| ipaD | 4 | gb\|AF177198\|AF177198 Homo sapiens talin mRNA, complete cds. |
| ipaD | 4 | gb\|D32053\|D32053 Homo sapiens mRNA for Lysyl tRNA Synthetase, complete cds. |
| ipaD | 4 | gb\|D55696\|D55696 Homo sapiens mRNA for cysteine protease, complete cds. |
| ipaD | 4 | gb\|M14144\|HUMVIM Human vimentin gene, complete cds. |
| ipaD | 4 | gb\|M34455\|HUMIGIIDO Human interferon-gamma-inducible indoleamine 2,3-dioxygenase |
| | | (IDO) mRNA, complete cds. |
| ipaD | 4 | gb\|M63121\|HUMTNFRC Human tumor necrosis factor receptor (TNF receptor) mRNA, complete cds. |
| ipaD | 4 | gb\|U70734\|HSU70734 Homo sapiens 38 kDa Mov34 homolog mRNA, complete cds. |
| ipaD | 4 | gb\|Z26649\|HSPPLCB3 H. sapiens mRNA for phospholipase C-b3. |
| ipaD | 4 | gb\|Z26649\|HSPPLCB3 H. sapiens mRNA for phospholipase C-b3. |
| ipaC | 5 | gb\|AB002366\|AB002366 Human mRNA for KIAA0368 gene, partial cds. |
| ipaC | 5 | gb\|AB002533\|AB002533 Homo sapiens mRNA for Qip1, complete cds. |
| ipaC | 5 | gb\|AB007923\|AB007923 Homo sapiens mRNA for KIAA0454 protein, partial cds. |
| ipaC | 5 | gb\|AB008515\|AB008515 Homo sapiens mRNA for RanBPM, complete cds. |
| ipaC | 5 | gb\|AB018271\|AB018271 Homo sapiens mRNA for KIAA0728 protein, partial cds. |
| ipaC | 5 | gb\|AB020335\|AB020335 Homo sapiens Pancreas-specific TSA305 mRNA, complete cds. |
| ipaC | 5 | gb\|AB023224\|AB023224 Homo sapiens mRNA for KIAA1007 protein, partial cds. |
| ipaC | 5 | gb\|AB029290\|AB029290 Homo sapiens mRNA for actin binding protein ABP620, complete cds. |
| ipaC | 5 | gb\|AB046026\|AB046026 Macaca fascicularis brain cDNA, clone: QccE-16688. |
| ipaC | 5 | gb\|AC003991\|AC003991 Human BAC clone CTB-167B5 from 7q21, complete sequence. |
| ipaC | 5 | gb\|AC005578\|AC005578 Homo sapiens chromosome 19, cosmid F20887, complete sequence. |
| ipaC | 5 | gb\|AF006751\|AF006751 Homo sapiens ES/130 mRNA, complete cds. |
| ipaC | 5 | gb\|AF006751\|AF006751 Homo sapiens ES/130 mRNA, complete cds. |
| ipaC | 5 | gb\|AF006751\|AF006751 Homo sapiens ES/130 mRNA, complete cds. |
| ipaC | 5 | gb\|AF006751\|AF006751 Homo sapiens ES/130 mRNA, complete cds. |
| ipaC | 5 | gb\|AF100153\|AF100153 Homo sapiens connector enhancer of KSR-like protein CNK1 mRNA, complete cds. |
| ipaC | 5 | gb\|AF176069\|AF176069 Homo sapiens ubiquilin mRNA, complete cds. |
| ipaC | 5 | gb\|AF176069\|AF176069 Homo sapiens ubiquilin mRNA, complete cds. |
| ipaC | 5 | gb\|AF176796\|AF176796 Homo sapiens putative glialblastoma cell differentiation-related protein |
| | | (GBDR1) mRNA, complete cds. |
| ipaC | 5 | gb\|AF176796\|AF176796 Homo sapiens putative glialblastoma cell differentiation-related protein |
| | | (GBDR1) mRNA, complete cds. |
| ipaC | 5 | gb\|AF176796\|AF176796 Homo sapiens putative glialblastoma cell differentiation-related protein |
| | | (GBDR1) mRNA, complete cds. |
| ipaC | 5 | gb\|AF177198\|AF177198 Homo sapiens talin mRNA, complete cds. |
| ipaC | 5 | gb\|AF177198\|AF177198 Homo sapiens talin mRNA, complete cds. |
| ipaC | 5 | gb\|AF187859\|AF187859 Homo sapiens Hsp70 binding protein HspBP2 mRNA, complete cds. |
| ipaC | 5 | gb\|AF189009\|AF189009 Homo sapiens ubiquitin-like product Chap1/Dsk2 mRNA, complete cds. |
| ipaC | 5 | gb\|AK000982\|AK000982 Homo sapiens cDNA FLJ10120 fis, clone HEMBA1002863. |
| ipaC | 5 | gb\|D21260\|HUMORFEA Human mRNA for KIAA0034 gene, complete cds. |
| ipaC | 5 | gb\|D28476\|HUMKG1C Human mRNA for KIAA0045 gene, complete cds. |
| ipaC | 5 | gb\|D44466\|D44466 Homo sapiens mRNA for proteasome subunit p112, complete cds. |
| ipaC | 5 | gb\|J00118\|HUMPLB Human placental lactogen hormone (PL-4) mRNA, complete cds. |
| ipaC | 5 | gb\|J00118\|HUMPLB Human placental lactogen hormone (PL-4) mRNA, complete cds. |
| ipaC | 5 | gb\|J04164\|HUM927A Human interferon-inducible protein 9-27 mRNA, complete cds. |
| ipaC | 5 | gb\|L36983\|HUMDNM Homo sapiens dynamin (DNM) mRNA, complete cds. |
| ipaC | 5 | gb\|L41498\|HUMPTI1B Homo sapiens longation factor 1-alpha 1 (PTI-1) mRNA, complete cds. |
| ipaC | 5 | gb\|L41668\|HUMGALE Homo sapiens UDP-galactose-4-epimerase (GALE) mRNA, complete cds. |
| ipaC | 5 | gb\|M24766\|HUMCOL4A2P Human (clone pHAIV2-12) alpha-2 collagen type IV (COL4A2) mRNA, 3′ end. |
| ipaC | 5 | gb\|M81355\|HUMSPHINO Homo sapiens sphingolipid activator proteins 1 and 2 processed |
| | | mutant mRNA, complete cds. |
| ipaC | 5 | gb\|U02389\|HSU02389 Human hLON ATP-dependent protease mRNA, nuclear gene encoding mitochondrial |
| | | protein, complete cds. |
| ipaC | 5 | gb\|U53225\|HSU53225 Human sorting nexin 1 (SNX1) mRNA, complete cds. |
| ipaC | 5 | gb\|X05610\|HSC4A2 Human mRNA for type IV collagen alpha (2) chain. |
| ipaC | 5 | gb\|X63564\|HSRPIILS H. sapiens mRNA for RNA polymerase II largest subunit. |
| ipaC | 5 | gb\|X98296\|HSUBIQHYD H. sapiens mRNA for ubiquitin hydrolase. |
| ipaC | 5 | gb\|Z26649\|HSPPLCB3 H. sapiens mRNA for phospholipase C-b3. |
| ipaH9.8 | 6 | dbj\|AB001636.1\|AB001636 Homo sapiens mRNA for ATP-dependent RNA helicase #46, complete cds |
| ipaH9.8 | 6 | dbj\|AB002333.1\|AB002333 Human mRNA for KIAA0335 gene, complete cds |
| ipaH9.8 | 6 | dbj\|AB008515.1\|AB008515 Homo sapiens mRNA for RanBPM, complete cds |
| ipaH9.8 | 6 | dbj\|AB023187.1\|AB023187 Homo sapiens mRNA for KIAA0970 protein, complete cds |
| ipaH9.8 | 6 | dbj\|AB033043.1\|AB033043 Homo sapiens mRNA for KIAA1217 protein, partial cds |
| ipaH9.8 | 6 | dbj\|AK001451.1\|AK001451 Homo sapiens cDNA FLJ10589 fis, clone NT2RP2004389, weakly similar |
| | | to PROBABLE MITOCHONDRIAL 40S RIBOSOMAL PROTEIN S9 PRECURSOR |
| ipaH9.8 | 6 | dbj\|AK024449.1\|AK024449 Homo sapiens mRNA for FLJ00041 protein, partial cds |
| ipaH9.8 | 6 | dbj\|D63875.1\|D63875 Human mRNA for KIAA0155 gene, complete cds |
| ipaH9.8 | 6 | emb\|AL034405.16\|HS537K23 Human DNA sequence from clone RP4-537K23 on chromosome Xq25-26.1, |
| | | complete sequence [Homo sapiens] |
| ipaH9.8 | 6 | emb\|AL034417.14\|HS215D11 Human DNA sequence from clone 215D11 on chromosome 1p36.12-36.33 |
| | | Contains a gene for RNA-binding protein regulatory subunit, a gene similar to rat gene 33, a |
| | | pseudogene similar to PLA-X, ESTs, STSs, GSSs and CpG |
| | | islands, complete sequence [Homo sapie |
| ipaH9.8 | 6 | emb\|AL050313.6\|HSBK754D9 Human DNA sequence from clone CTA-754D9 on chromosome 22 |
| | | Contains GSSs, complete sequence [Homo sapiens] |
| ipaH9.8 | 6 | emb\|AL117448.1\|HSM800958 Homo sapiens mRNA; cDNA DKFZp586B1417 |
| | | (from clone DKFZp586B1417); partial cds |
| ipaH9.8 | 6 | emb\|AL137068.10\|AL137068 Human DNA sequence from clone RP11-165P4 on chromosome |
| | | 9q34.11-34.13, complete sequence [Homo sapiens] |
| ipaH9.8 | 6 | emb\|X53416.1\|HSABP280 Human mRNA for actin-binding protein (filamin) (ABP-280) |
| ipaH9.8 | 6 | emb\|X73478.1\|HSPTPAA H. sapiens hPTPA mRNA |
| ipaH9.8 | 6 | emb\|X74801.1\|HSHUMAPC H. sapiens Cctg mRNA for chaperonin |
| ipaH9.8 | 6 | emb\|X95648.1\|HSEIF2BAS H. sapiens mRNA for eIF-2B alpha subunit |
| ipaH9.8 | 6 | gb\|AC005392.1\|AC005392 Homo sapiens chromosome 19, CIT-HSP BAC 490g23 |
| | | (BC338531), complete sequence |
| ipaH9.8 | 6 | gb\|AC005833.1\|AC005833 Homo sapiens 12p13.3 BAC RPCI11-234B2 (Roswell Park Cancer Institute Human |
| | | BAC Library) complete sequence |
| ipaH9.8 | 6 | gb\|AC005881.3\|AC005881 citb_79_e_16, complete sequence [Homo sapiens] |
| ipaH9.8 | 6 | gb\|AC020663.1\|A0020663 Homo sapiens chromosome 16 clone RPCI-11_127I20, complete sequence |
| ipaH9.8 | 6 | gb\|AF006466.1\|AF006466 Mus musculus lymphocyte specific formin related protein (Fr1) mRNA, complete cds |
| ipaH9.8 | 6 | gb\|AF010404.1\|AF010404 Homo sapiens ALR mRNA, complete cds |
| ipaH9.8 | 6 | gb\|AF064729.1\|AF064729 Homo sapiens RAN binding protein 16 mRNA, complete cds |
| ipaH9.8 | 6 | gb\|AF084940.1\|AF084940 Homo sapiens beta-arrestin 1B mRNA, complete cds |
| ipaH9.8 | 6 | gb\|AF135159.1\|AF135159 Homo sapiens GMP reductase mRNA, complete cds |
| ipaH9.8 | 6 | gb\|AF139184.1\|AF139184 Homo sapiens Sec31 protein mRNA, complete cds |
| ipaH9.8 | 6 | gb\|AF141327.1\|AF141327 Homo sapiens ring finger protein BAP-1 mRNA, complete cds |
| ipaH9.8 | 6 | gb\|AF171669.1\|AF171669 Homo sapiens glycoprotein-associated amino acid transporter LAT2 |
| | | (LAT2) mRNA, complete cds |
| ipaH9.8 | 6 | gb\|AF174605.1\|AF174605 Homo sapiens F-box protein Fbx25 (FBX25) mRNA, partial cds |
| ipaH9.8 | 6 | gb\|AF207661.1\|AF207661 Homo sapiens sodium bicarbonate cotransporter-like protein mRNA, partial cds |
| ipaH9.8 | 6 | gb\|AF245517.1\|AF245517 Homo sapiens vacuolar proton pump 116 kDa accessory subunit |
| | | (ATP6N1B) mRNA, complete cds, alternatively spliced |
| ipaH9.8 | 6 | gb\|AF249874.1\|AF249874 Homo sapiens vacuolar proton pump 116 kDa accessory subunit gene, exon 3 and |
| | | 5′ untranslated region, partial sequence |
| ipaH9.8 | 6 | gb\|J00118.1\|HUMPLB Human placental lactogen hormone (PL-4) mRNA, complete cds |
| ipaH9.8 | 6 | gb\|L14283.1\|HUMPROKINC Human protein kinase C zeta mRNA, complete cds |
| ipaH9.8 | 6 | gb\|L25286.1\|HUMCOLXVA1 Homo sapiens alpha-1 type XV collagen mRNA, complete cds |
| ipaH9.8 | 6 | gb\|M13451.1\|HUMLAMC Human lamin C mRNA, complete cds |
| ipaH9.8 | 6 | gb\|M21616.1\|HUMPDGFR Human platelet-derived growth factor (PDGF) receptor mRNA, complete cds |
| ipaH9.8 | 6 | gb\|M32053.1\|HUMH19 Human H19 RNA gene, complete cds |
| ipaH9.8 | 6 | gb\|M34455.1\|HUMIGIIDO Human interferon-gamma-inducible indoleamine 2,3-dioxygenase (IDO) |
| | | mRNA, complete cds |
| ipaH9.8 | 6 | gb\|M94890.1\|HUMPSBG11 Human pregnancy-specific beta-1-glycoprotein 11 (PSG11) mRNA, complete cds |
| ipaH9.8 | 6 | gb\|M98478.1\|HUMTGH1A Human transglutaminase mRNA, complete cds |
| ipaH9.8 | 6 | gb\|U24267.1\|HSU24267 Human pyrroline-5-carboxylate dehydrogenase (P5CDh) mRNA, short form, |
| | | complete cds |
| ipaH9.8 | 6 | gb\|U37791.1\|HSU37791 Homo sapiens clone rasi-1 matrix metalloproteinase RASI-1 mRNA, complete cds |
| ipaH9.8 | 6 | gb\|U38431.1\|HSU38431 Human clone rasi-6 matrix metalloproteinase RASI-1 mRNA, splice variant, |
| | | complete cds |
| ipaH9.8 | 6 | gb\|U65928.1\|HSU65928 Human Jun activation domain binding protein mRNA, complete cds |
| ipaH9.8 | 6 | ref\|NM_014285.1\|Homo sapiens homolog of Yeast RRP4 (ribosomal RNA processing 4), |
| | | 3′-5′-exoribonuclease (RRP4), mRNA |
| ipaH9.8 | 6 | ref\|NM_017762.1\|Homo sapiens hypothetical protein FLJ20313 (FLJ20313), mRNA |
| ipaH9.8 | 6 | ref\|NM_018155.1\|Homo sapiens hypothetical protein FLJ10618 (FLJ10618), mRNA |
| ospG | 7 | gb\|AB008515\|AB008515 Homo sapiens mRNA for RanBPM, complete cds. |
| ospG | 7 | gb\|AB013818\|AB013818 Homo sapiens PEX10 mRNA for peroxisome biogenesis factor (peroxin) |
| | | 10, complete cds. |
| ospG | 7 | gb\|AB033054\|AB033054 Homo sapiens mRNA for KIAA1228 protein, partial cds. |
| ospG | 7 | gb\|AB033054\|AB033054 Homo sapiens mRNA for KIAA1228 protein, partial cds. |
| ospG | 7 | gb\|AB040918\|AB040918 Homo sapiens mRNA for KIAA1485 protein, partial cds. |
| ospG | 7 | gb\|AC005281\|AC005281 Homo sapiens PAC clone RP4-722F20 from 7q31.1-q31.3, complete sequence. |
| ospG | 7 | gb\|AE003603\|AE003603 Drosophila melanogaster genomic scaffold 142000013386043 section 4 of 8, |
| | | complete sequence. |
| ospG | 7 | gb\|AF033095\|AF033095 Homo sapiens testis enhanced gene transcript protein (TEGT) mRNA, complete cds. |
| ospG | 7 | gb\|AF035121\|AF035121 Homo sapiens KDR/flk-1 protein mRNA, complete cds. |
| ospG | 7 | gb\|AF061736\|AF061736 Homo sapiens ubiquitin-conjugating enzyme RIG-B mRNA, complete cds. |
| ospG | 7 | gb\|AF085362\|AF085362 Homo sapiens UbcM2 mRNA, complete cds. |
| ospG | 7 | gb\|AF104913\|AF104913 Homo sapiens eukaryotic protein synthesis initiation factor mRNA, complete cds. |
| ospG | 7 | gb\|AF155238\|AF155238 Homo sapiens BAC 180i23 chromosome 8 map 8q24.3 beta-galactoside |
| | | alpha-2,3-sialytransferase (SIAT4A) gene, complete sequence. |
| ospG | 7 | gb\|AJ000519\|HSUBICONJ Homo sapiens mRNA for ubiquitin-conjugating enzyme UbcH7. |
| ospG | 7 | gb\|AK000393\|AK000393 Homo sapiens cDNA FLJ20386 fis, clone KAIA4184. |
| ospG | 7 | gb\|AK001311\|AK001311 Homo sapiens cDNA FLJ10449 fis, clone NT2RP1000947, highly similar to |
| | | Human E2 uibiquitin conjugating enzyme UbcH5B mRNA. |
| ospG | 7 | gb\|AL050321\|HSJ717M23 Human DNA sequence from clone RP4-717M23 on chromosome 20, |
| | | complete sequence. |

TABLE III
SID®

| | | | | | | |
|---|---|---|---|---|---|---|
| | 2: Bait | | 4: SID | | 6: SID | |
| | nucleic | | nucleic | | amino- | |
| | acid | | acid | | acid | |
| 1: Bait name | SEQ ID No. | 3: Prey name | ID No. | 5:SID nucleic acid sequence | ID No. | 7:SID amino-acid sequence |

| | | | | | | | |
|---|---|---|---|---|---|---|---|
| Shigella | 1 | prey44074 | 15 | CTTCAGCCACGACTCCTCCTTCCTCTGCGCTTCCAGTGATAAGGGTACTGTC | 216 | FSHDSSFLCASSDKGTVHI | |
| ospB | | | | CATATCTTTGCTCTCAAGGATACCCGCCTCAACCGCCGCTCCGCGCTGGCTC | | FALKDTRLNRRSALARVGK | |
| | | | | GCGTGGGCAAGGTGGGGCCTATGATTGGGCAGTACGTGGACTCTCAGTGGA | | VGPMIGQYVDSQWSLASF | |
| | | | | GCCTGGCGAGCTTCACTGTGCCTGCTGAGTCAGCTTGCATCTGCGCCTTCG | | TVPAESACICAFGRNTSKN | |
| | | | | GTCGCAATACTTCCAAGAACGTCAACTCTGTCATTGCCATCTGCGTAGATGG | | VNSVIAICVDGTFHKYVFTP | |
| | | | | GACCTTCCACAAATATGTCTTCACTCCTGATGGAAACTGCCAACAGAGAGGCT | | DGNCNREAFDVYLDICDDD | |
| | | | | TTCGACGTGTACCTTGACATCTGTGATGATGATGACTTTAA | | DF* | |
| Shigella | 1 | prey67804 | 16 | GACCAGCAAGTCTTGCGAGTACAATGGGACAACTTACCAACATGGAGAGCT | 217 | TSKSCEYNGTTYQHGELFV | |
| ospB | | | | GTTCGTAGCTGAAGGGCTCTTTCAGAATCGGCAACCCAATCAATGCACCCAG | | AEGLFQNRQPNQCTQCSC | |
| | | | | TGCAGCTGTTCGGAGGGAAACGTGTATTGTGGTCTCAAGACTTGCCCCAAAT | | SEGNVYCGLKTCPKLTCAF | |
| | | | | TAACCTGTGCCTTCCCAGTCTCTGTTCCAGATTCCTGCTGCCGGGTATGCAG | | PVSVPDSCCRVCRGDGFI | |
| | | | | AGGAGATGGAGAACTGTCATGGGAACATTCTGATGGTGATATCTTCCGGCAA | | SWEHSDGDIFRQPANREA | |
| | | | | CCTGCCAACAGAGAAGCAAGACATTCTTACCACCGCTCTCACTATGATCCTC | | RHSYHRSHYDPPPSRQAG | |
| | | | | CACCAAGCCGACAGGCTGGAGGTCTGTCCCGCTTTCCTGGGGCCAGAAGTC | | GLSRFPGARSHRGALMDS | |
| | | | | ACCGGGGAGCTCTTATGGATTCCCAGCAAGCATCAGGAACCATTGTGCAAAT | | QQASGTIVQIVINNKHKHG | |
| | | | | TGTCATCAATAACAAACACAAGCATGGACAAGTGTGTGTTTCCAATGGAAAG | | QVCVSVGKTYSHGESQHP | |
| | | | | ACCTATTCTCATGGCGAGTCCTGGCACCCAAACCTCCGGGCATTTGGCATTG | | NLRAFGIVECVLCTCNVTK | |
| | | | | TGGAGTGTGTGCTATGTACTTGTAATGTCACCAAGCAAGAGTGTAAGAAAAT | | QECKKIHCPNRYPCKYPQK | |
| | | | | CGACTGCCCCAATCGATACCCCTGCAAGTATCCTCAAAAAATAGACGGAAAA | | IDGKCCKVCPGKKAKELPG | |
| | | | | TGCTGCAAGGTGTGTCCAGGTAAAAAAGCAAAAGAACTtCCAGGCCAAAGCT | | QSFDNKGYFCGEETMPVY | |
| | | | | TTGACAATAAAGGCTACTTCTGCGGGGAAGAAACGATGCCTGTGTATGAGTC | | ESVFMEDGETTRKIALETE | |
| | | | | TGTATTCATGGAGGATGGGGAGACAACCAGAAAAATAGCACTGGAGACTGA | | RPPQVEVHVQtIRKGILQH | |
| | | | | GAGACCACCTCAGGTAGAGGTCCACGTTTGGACTATTCGAAAGGGCATTCTC | | FHIEKISKRMFEELPHFKLV | |
| | | | | CAGCACTTCCATATTGAGAAGATCTCCAAGAGGATGTTTGAGGAGCTTCCTC | | TRTTLSQWKIFTEGEAQISQ | |
| | | | | ACTTCAAGCTGGTGACCAGAACAACCCTGAGCCAGTGGAAGATCTTCACCGA | | MCSSRVCRTELEDLVKVLY | |
| | | | | AGGAGAAGCTCAGATCAGCCAGATGTGTTCAAGTCGTGTATGCAGAACAGA | | LERSEKGHC* | |
| | | | | GCTTGAAGATTTAGTCAAGGTTTTGTACCTGGAGAGATCTGAAAAGGGCCAC | | | |
| | | | | TGTTAG | | | |
| Shigella | 1 | prey67806 | 17 | NCTNCCCTGNGCGNGACCAGCCTGGTNANCTTACCNGGANCCACNGGATGT | | XXLXXTSLVXLPGXTGCXV | |
| ospB | | | | NGTGTANCTGTGCTCTGCGCTTGCCATGATGACTTNTGGGAGCTGCANCCG | | XVLCACHDDXWELXPSRX | |
| | | | | TCGCGTTTNTGNNNCGTNGTTGGTGNCNGGCCTCCNTANGNTGTGNNACGA | | XXVVGXXPPXXVXRRLXFA | |
| | | | | AGACTGTTNTTTGCTAAGGACCTGCNGTNTGCTGCTTCATTNGGNGAGNTTT | | KDLXXAASXGEXXLGGXLX | |
| | | | | NNTTAGGGGGNGNNTTATTNCTAAAATNTTGGGACTCTTAAGTTTTNGNTGN | | LKXWDS*VXXXVFXXK | |
| | | | | GGTTTTTNTNGNNAAGAA | | | |
| Shigella | 1 | prey67810 | 18 | GGCGGCCATGGAGACCGAGACGGCGCCGCTGACCCTAGAGTCGCTGCCCA | 219 | AAMETETAPLTLESLPTDPL | |
| ospB | | | | CCGATCCCCTGCTCCTCATGTTATCCTTTTTGGACTATCCGGGATCTAATCAAC | | LLILSFLDYRDLINCCYVSR | |
| | | | | TGTTGTTATGTCAGTCGAAGACTTAGCCAGCTATCAAGTCATGATCCGCTGT | | RLSQLSSHDPLQRRHCKK | |
| | | | | GGAGAAGACATTGCAAAAAATACTGGCTGATATCTGAGGAAGAGAAAACACA | | YWLISEEEKTQKNQCWKSL | |
| | | | | GAAGAATCAGTGTTGGAAATCTCTCTTGATAGATACTTACTCTGATGTAGGAA | | FIDTYSDVGRYIDHYAAIKK | |
| | | | | GATACATTGACCATTATGCTGCTATTAAAAAGGCCTGGGATGATCTCAAGAAA | | AWDDLKKYLEPRCPRMVL | |
| | | | | TATTTGGAGCCCAGGTGTCCTCGGATGGTTTTATCTCTGAAAGAGGGTGCTC | | SLKEGAREEDLDAVEAQIG | |
| | | | | GAGAGGAAGACCTCGATGCTGTGGAAGCGCAGATTGGCTGCAAGCTTCCTG | | CKLPDDYRCSYRIHNGQKL | |
| | | | | ACGATTATCGATGTTCATACCGAATTCACAATGGACAGAAGTTAGTGGTTCCT | | VVPGLLGSMALSNHYRSED | |
| | | | | GGGTTATTGGGAAGCATGGCACTGTCTAATCACTATCGTTCTGAAGATTTGTT | | LLDVDTAAGGFQQRQGLK | |
| | | | | AGACGTOGATACAGCTGCCGGAGGATTCCAGCAGAGACAGGGACTGAAATA | | YCLPLTFCIHTGLSQYIAVE | |
| | | | | CTGTCTCCCTTTAACTTTTTGCATACATACTGGTTTGAGTCAGTACATAGCAG | | AAEGRNKNEVFYQCPDQM | |
| | | | | TGGAAGCTGCAGAGGGCCGAAACAAAAATGAAGTTTTCTACCAATGTCCAGA | | ARNPAAIDMFIIGATFTDWF | |
| | | | | CCAAATGGCTCGAAATCCAGCTGCTATTGACATGTTTATTATAGGTGCTACTT | | TSYVKNVVSGGFPIIRDQIF | |
| | | | | TTACTGACTGGTTTAOCTCTTATGTCAAAAATGTTGTATCAGGTGGCTTCCCC | | RYVHDPECVATTGDITVSV | |
| | | | | ATCATCAGAGACCAAATTTTCAGATATGTTCACGATCCAGAATGTGTAGCAAC | | STSFLPELSSVHPPHYFFTY | |
| | | | | AACTGGGGATATTACTGTGTCAGTTTCCACATCGTTTCTGCCAGAACTTAGCT | | RIRIEMSKDALPEKACQLDS | |
| | | | | CTGTAGATCCACCCCACTATTTCTTCACATACCGAATCAGGATTGAAATGTCA | | RYWRITNAKGDVEEVQGP | |
| | | | | AAAGATGCACTTCCTGAGAAGGCCTGTCAGTTGGACAGTCGCTATTGGAGAA | | GVVGEFPIISPGRVYEYTSC | |
| | | | | TAACAAATGCTAAGGGTGACGTGGAAGAAGTTCAAGGACCTGGAGTAGTTG | | TTFSTTSGTMEGYYTFHFL | |
| | | | | GTGAATTTCCAATCATCAGCCCAGGTCGGGTATATGAATACACAAGCTGTAC | | YFKDKIFNVAIPRFHMACPT | |
| | | | | CACATTCTCTACAACATCAGGATACATGGAAGGATATTATACCTTCCATTTTC | | FRVSIARLVS* | |
| | | | | TTTACTTTAAAGACAAGATCTTTAATGTTGCCATTCCCCGATTCCATATGGCAT | | | |
| | | | | GTGCAACATTCAGGGTGTCTATAGCCCGATTGGTAAGTTAA | | | |
| Shigella | 1 | prey5237 | 19 | GCAGCAACAGCAGCAGCCGCCACCACCGCCAATACCTGCAAATGGGCAACA | 220 | QQQQQPPPPPIPANGQQA | |
| ospB | | | | GGCCAGCAGCCAAAATGAAGGCTTGACTATTGACCTGACCTGAAGAATTTTAGAAAA | | SSQNEGLTIDLKNFRKPGE | |
| | | | | CCAGGAGAGAAGACCTTCACCCAACGAAGCCGTCTTTTTGTGGGAAATGTTC | | KTFTQRSRLFVGNLPPDITE | |
| | | | | CTCCCGACATCACTGAGGAAGAAATGAGGAAACTATTTGAGAAATATGGAAA | | EEMRKLFEKYGKAGEVFIH | |
| | | | | GGCAGGCGAAGTCTTCATTCATAAGGATAAAGGATTTGGCTTTATCCGCTTG | | KDKGFGFIRLETRTLAEIAK | |
| | | | | GAAACGCGAACCCTAGCGGAGATTGCCAAAGTGGAGCCTGGACAATATGCCA | | VELDNMPLRGKQLRVRFA | |
| | | | | CTCCGTGGAAAGCAGCTGCGTGTGCGCTTTGCCTGCCATAGTGCATCCCTTA | | CHSASLTVRNLPQYVSNEL | |
| | | | | CAGTTCGAAACCTTCCTCAGTATGTGTCCAACGAACTGCTGGAAGAAGCCTT | | LEEAFSVFGQVERAVVIVD | |
| | | | | TTCTGTGTTTGGCCAGGTAGAGAGGGCTGTAGTCATTGTGGATGATCGAGGA | | DRGRPSGKGIVEFSGKPAA | |
| | | | | AGGCCCTCAGGAAAAGGCATTGTTGAGTTCTCAGGGAAGCCAGCTGCTCGG | | RKALDRCSEGSFLLTTFRP | |
| | | | | AAAGCTCTGGACAGATGCAGTGAAGGCTCCTTCCTGCTAACCACATTTCCTC | | PVTVEPMDQLDDEEGLPEK | |
| | | | | GTCCTGTGACTGTGGAGCCCATGGACCAGTTAGATGATGAAGAGGGACTTC | | LVIKNQQFHKEREQPPRFA | |
| | | | | CAGAGAAGCTGGTTATAAAAAACCAGCAATTTCACAAGGAACGAGAGCAGCC | | QPGSFEYEYAMRWKALIE | |
| | | | | ACCCAGATTTGCACAGCCTGGCTCCTTTGAGTATGAATATGCCATGCGCTGG | | MEKQQQDQVDRNIKE | |
| | | | | AAGGCACTCATTGAGATGGAGAAGCAGCAGCAGGACCAAGTGGACCGCAAC | | | |
| | | | | ATCAAGGAGGC | | | |
| Shigella | 1 | prey67661 | 20 | TGGGGATTTCTGCATCCGGGTCTTTTCTGAAAAGAAAGCTGACTACCAAGCT | 221 | GDFCIRVFSEKKADYQAVD | |
| ospB | | | | GTCGATGATGAAATCGAGGCCAATCTTGAAGAGTTCGACATCAGCGAGGATG | | DEIEANLEEFDISEDDIDDG | |
| | | | | ACATTGATGATGGAGTCAGGAGACTGTTTGCCCAGTTGGCAGGAGAGGATG | | VRRLFAQLAGEDAEISAFEL | |
| | | | | CGGAGATCTCTGCCTTTGAGCTGCAGACCATCCTGAGAAGGGTTCTAGCAAA | | QTILRRVLAKRQDIKSKGFS | |
| | | | | GCGCCAAGATATCAAGTCAGATGGCTTCAGCATCGAGACATGCAAAATTATG | | IETCKIMVDMLDSDGSGKL | |
| | | | | GTTGACATGCTAGATTCGGACGGGAGTGGCAAGCTGGGGCTGAAGGAGTTC | | GLKEFYILWTKIQKYQKIYR | |
| | | | | TACATTCTCTGGACGAAGATTCAAAAATACCAAAAAATTTACCGAGAAATCGA | | EIDVDRSGTMNSYEMRKAL | |
| | | | | CGTTGACAGGTCTGGTACCATGAATTCCTATGAAATGCGGAAGGCATTAGAA | | EEAGFKMPCQLHQVIVARF | |
| | | | | GAAGCAGGTTTCAAGATGCCCTGTCAACTCCACCAAGTCATCGTTGCTCGGT | | ADDQLIIDFDNFVRCLVRLE | |
| | | | | TTGCAGATGACCAGCTCATCATCGATTTTGATAATTTTGTTCGGTGTTTGGTT | | TLFKIFKQLDPENTGTIELDL | |
| | | | | CGGCTGGAAACGCTATTCAAGATATTTAAGCAGCTGGATCCCGAGAATACTG | | ISWLCFSVL* | |
| | | | | GAACAATAGAGCTCGACCTTATCTCTTGGCTCTGTTTCTCAGTACTTTGA | | | |
| Shigella | 1 | prey34730 | 21 | ATGGTGAATCCGGGCAGCAGCTCGCAGCCGCCCCCGGTGACGGCCGGCTC | 222 | MVNPGSSSQPPPVTAGSL | |
| ospB | | | | CCTCTCCTGGAAGCGGTGCGCAGGCTGCGGGGGCAAGATTGCGGACCGCT | | SWKRCAGCGGKIADRFLLY | |
| | | | | TTCTGCTCTATGCCATGGACAGCTATTGGCACAGCCGGTGCCTCAAGTGCTC | | AMDSYWHSRCLKCSCCQA | |
| | | | | CTGCTGCCAGGCGCAGCTGGGCGACATGGGCACGTCCTGTTACACCAAAAG | | QLGDIGTSCYTKSGMILCR | |
| | | | | TGGCATGATCCTTTGCAGAAATGACTACATTAGGTTATTTGGAAATAGCGGTG | | NDYIRLFGNSGACSACGQS | |
| | | | | CTTGCAGCGCTTGCGGAGAGTCGATTCCTGCGAGTGAACTCGTCATGAGGG | | IPASELVMRAQGNVYHLKC | |
| | | | | CGCAAGGCAATGTGTATCATCTTAAGTGTTTTACATGCTCTACCTGCCGGAAT | | FTCSTCRNRLVPGDRFHYI | |
| | | | | CGCCTGGTCCCGGGAGATCGGTTTCACTACATCAATGGCAGTTTATTTTGTG | | NGSLFCEHDRPTALINGHI | |
| | | | | AACATGATAGACCTACAGCTCTCATCAATGGCCATTTGAATTCACTTCAGAGC | | NSLQSNP | |
| | | | | AATCCACT | | | |
| Shigella | 1 | prey33141 | 22 | CCTGAGCCTGCCGGGGATCCTGCACTTTATCCAGCACGAGTGGGCGCGCTT | 223 | LSLPGILHFIQHEWARFEAE | |
| ospB | | | | CGAAGCCGAGAAAGCCCGCTGGGAGGCCGAGCGCGCCGAGTTACAGGCTC | | KARWEAERAELQAQVAFL | |
| | | | | AGGTGGCCTTCCTTCAGGGAGAGAGGAAAGGGCAGGAGAATCTAAAGACGG | | QGERKGQENLKTDLVRRIK | |
| | | | | ACCTGGTGCGGCGGATCAAGATGCTAGAGTATGCGCTGAAGCAGGAAAGGG | | MLEYALKQERAKYHKLKFG | |
| | | | | CCAAATATCATAAACTGAAGTTTGGGACAGACCTGAACCAGGGGGAGAAGAA | | TDLNQGEKKADVSEQVSN | |
| | | | | AGCAGATGTGTCAGAACAAGTCTCCAATGGCCCCGTGGAATCGGTCACCCT | GPVESVTLENSPLVWKEG | | |
| | | | | GGAGAACAGCCCGTTGGTGTGGAAGGAGGGGCGGCAGCTTCTCCGACAGT | | RQLLRQYLE | |
| | | | | ACCTGGAAG | | | |
| Shigella | 1 | prey67575 | 23 | ATGGCAGCCTCCTTACGGCTCCTCGGAGCTGCCTCCGGTCTCCGGTACTGG | 224 | MAASLRLLGAASGLRYWS | |
| ospB | | | | AGCCGGCGGCTGCGGCCGGCAGCCGGCAGCTTTGCAGCGGTGTGTTCTAG | | RRLRPAAGSFAAVCSRSVA | |
| | | | | GTCAGTGGCTTCAAAGACTCCAGTTGGATTCATTGGACTGGGCAACATGGG | | SKTPVGFIGLGNMGNPMAK | |
| | | | | GAATCCAATGGCAAAAAATCTCATGAAACATGGCTATCCACTTATTATTTATG | | NLMKHGYPLIIYDVFPDACK | |
| | | | | ATGTGTTCCCTGATGCCTGCAAAGAGTTTCAAGATGCAGGTGAACAGGTAGT | | EFQDAGEQVVSSPADVAE | |
| | | | | ATCTTCCCCAGCAGATGTTGCTGAAAAAGCTGACAGAATTATTACAATGCTGC | | KADRIITMLPTSINAIEAYSG | |
| | | | | CCACCAGTATCAATGCAATAGAAGCTTATTCCGGAGCAAATGGGATTCTAAA | | ANGILKKVKKGSLLIDSSTID | |
| | | | | AAAAGTGAAGAAGGGCTCATTATTAATAGATTCCAGCACTATTGATCCTGCAG | | PAVSKELAKEVEKMGAVEM | |
| | | | | TTTCAAAAGAATTGGCCAAAGAAGTTGAGAAAATGGGAGCAGTTTTCATGGA | | DAPVSGGVGAARSGNLTF | |
| | | | | TGCCCCTGTTTCTGGTGGTGTAGGAGCTGCACGATCTGGGAACCTCACGTTT | | MVGGVEDEFAAAQELLGC | |
| | | | | ATGGTGGGAGGAGTTGAAGATGAATTTGCTGCTGCCCAAGAGTTGCTGGGG | | MGSNVVYCGAVGTGQAAK | |
| | | | | TGCATGGGCTCCAACGTGGTGTACTGTGGAGCTGTTGGGACTGGGCAGGCG | | ICNNMLLAISMIGTAEAMNL | |
| | | | | GCAAAGATCTGCAACAACATGCTGTTAGCTATTAGTATGATTGGAACTGCTGA | | GIRLGLDPKLLAKILNMSSG | |
| | | | | AGCTATGAATCTTGGAATCAGGTTAGGGCTTGACCCAAAACTACTGGCTAAA | | RCWSSDTYNPVPGVMDGV | |
| | | | | ATCCTAAATATGAGCTCAGGACGGTGTTGGTCAAGTGACACTTATAATCCTGT | | PSANNYQGGFGTTLMAKD | |
| | | | | ACCTGGAGTGATGGATGGCGTTCCCTCGGCTAATAACTATCAGGGTGGATTT | | LGLAQDSATSTKSPILLGSL | |
| | | | | GGAACAACACTCATGGCTAAGGATCTGGGATTGGCACAAGACTCTGCTACCA | | AHQIYRMMCAKGYSKKDE | |
| | | | | GCACAAAGAGCCCAATCCTTCTTGGCAGTCTGGCCCATCAGATCTACAGGAT | | SSVFQFLREEETF* | |
| | | | | GATGTGTGCAAAGGGCTACTCAAAGAAAGACTTCTCATCCGTGTTCCAGTTC | | | |
| | | | | CTACGAGAGGAGGAGACCTTCTGA | | | |
| Shigella | 1 | prey67608 | 24 | CGCAGAGGAAGAGGAGGCCGAGGTGAGACAGCCCAAGGGACCAGACCCAG | 225 | AEEEEAEVRQPKGPDPDSL | |
| ospB | | | | ACAGCCTTAGTTCACAGTTTATGGCGTATATTGAACAGCGGCGAATCTCTCAG | | SSQFMAYIEQRRISHEGSP | |
| | | | | GAGGGTTCACCAGTAAAGCCAGTAGCCATTAGGGAGTTTCAAAAAACAGAAG | | VKPVAIREFQKTEDMRRYL | |
| | | | | ATATGAGAAGATACTTACATCAAAACAGGGTTGCAGCTGAGCCATCTTCCCT | | HQNRVPAEPSSLLSLSASH | |
| | | | | CCTGTCACTATCAGCAAGTCACAATCAGCTGTCACACACAGACCTGGAACTT | | NQLSHTDLELHQRREQLVE | |
| | | | | CATCAGAGAAGGGAGCAGTTAGTAGAGCGCACTCGGAGAGAGGCTCAGCTT | | RTRREAQLAALQYEEEKIR | |
| | | | | GCTGCCCTGCAGTATGAGGAGGAGAAAATAAGGACCAAGCAGATCCAGAGA | | TKQIQRDAVLDFVKQKASQ | |
| | | | | GATGCTGTCCTGGAGTTTGTCAAACAAAAAGCATCACAAAGTCCACAAAAAC | | SPQKQHPLLDGVDGECPF | |
| | | | | AGCACCCGCTCCTAGATGGCGTAGATGGTGAGTGCCCCTTCCCATCCAGAA | | PSRRSQHTDDSALCMSLS | |
| | | | | GGTCTCAGCACACTGATGATAGTGCCTTGTGCATGTCGCTGTCAGGGTTGAA | | GLNQVGCAATLPHSSAFTP | |
| | | | | TCAAGTGGGCTGTGCTGCTACCCTGCCTCATTCTTCTGCCTTCACGCCTCTT | | LKSDDRPNALLSSPATETV | |
| | | | | AAGAGTGATGACAGACCTAATGCTCTATTAAGTTCACCTGCAACAGAAACAG | | HHSPAYSFPAAIQRNQPQR | |
| | | | | TTCATCATTCCCCTGCATATTCTTTTCCTGCTGCTATCCAGAGAAATCAGCCT | | P | |
| | | | | CAGCGCCCT | | | |
| Shigella | 1 | prey67637 | 25 | ATGATACTACAGGAGTTACCAGATTTGGAGGAGCTCTTCCTGTGCCTTAATG | 226 | MILQELPDLEELFLCLNDYE | |
| ospB | | | | ACTATGAAACAGTGTCTTGTCCTTCTATTTGCTGTCATTCTCTTAAGCTACTAC | 226 | MILQELPDLEELFLCLNDYE | |
| | | | | ATATAACAGACAATAACCTCCAAGACTGGACTGAAATACGAAAGTTAGGAGTT | | NLQDWTEIRKLGVMFPSLD | |
| | | | | ATGTTTCCTTCACTGGATACCCTCGTCCTGGCCAACAATCATTTGAATGCTAT | | TLVLANNHLNAIEEPDDSLA | |
| | | | | TGAGGAGCCTGATGATTCATTGGCCAGGTTGTTTCCTAATCTTCGATCCATCA | | RLFPNLRSISLHKSGLQSW | |
| | | | | GCCTCGACAAGTCAGGTTTGCAGTCCTGGGAAGACATTGATAAACTAAATTC | | EDIDKLNSFPKLEEVRLLGI | |
| | | | | ATTTCCCAAACTGGAAGAAGTGAGATTGTTAGGAATTCCTCTTCTGCAGCCAT | | PLLQPYTTEERRKLVIARLP | |
| | | | | ATACCACCGAGGAGCGAAGGAAATTGGTAATAGCCAGATTGCCATCAGTTTC | | SVSKLNGSVVTDGEREDSE | |
| | | | | CAAACTTAATGGCAGCGTTGTTACTGATGGTGAACGAGAAGATTCTGAGAGA | | RFFIRYYVDVPQEEVPFRY | |
| | | | | TTTTTTATTCGTTAOTATGTGGATGTTCCACAGGAAGAAGTGCCATTCAGGTA | | HELITKYGKLEPLAEVDLRP | |
| | | | | TCATGAACTGATCACTAAATATGGGAAGTTGGAGCCTTTGGCAGAAGTGGAC | | QSSAKVEVHFNDQVEEMSI | |
| | | | | CTAAGACCCCAGAGCAGTGCAAAAGTAGAAGTCCACTTTAACGATCAGGTGG | | RLDQTVAELKKQLKTLVQL | |
| | | | | AAGAAATGAGCATTCGTCTGGACCAAACAGTGGCAGAACTAAAGAAACAGTT | | | |
| | | | | AAAAACTCTAGTACAATTACC | | | |
| Shigella | 1 | prey12713 | 26 | AGTGGATGAGGTGCTGCAGATCCCCCCATCCCTGCTGACATGCGGCGGCTG | 227 | VDEVLQIPPSLLTCGGCQQ | |
| ospB | | | | CCAGCAGAACATCGGGGACCGCTACTTCCTGAAGGCCATCGACCAGTACTG | | NIGDRYFLKAIDQYWHEDC | |
| | | | | GCACGAGGACTGCCTGAGCTGCGACCTCTGTGGCTGCCGGCTGGGTGAGG | | LSCDLCGCRLGEVGRRLYY | |
| | | | | TGGGGCGGCGCCTCTACTACAAACTGGGCCGGAAGCTCTGCCGGAGAGAC | | KLGRKLCRRDYRLFGQD | |
| | | | | TATCTCAGGCTTTTTGGGCAAGACGGTCTCTGCGCATCCTGTGACAAGCGGA | | GLCASCDKRIRAYEMTMRV | |
| | | | | TTCGTGCCTATGAGATGACAATGCGGGTGAAAGACAAAGTGTATCAGGTGGA | | KDKVYHLECFKCAACQKHF | |
| | | | | ATGTTTCAAGTGCGCCGCCTGTCAGAAGCATTTCTGTGTAGGTGACAGATAC | | CVGDRYLLINSDIVCEQDIY | |
| | | | | CTCCTCATCAACTCTGACATAGTGTGCGAACAGGACATCTACGAGTGGACTA | | EWTKINGMI* | |
| | | | | AGATCAATGGGATGATATAG | | | |
| Shigella | 1 | prey67836 | 27 | CCTGAAGACAGCTGGCAAGTCTGAACCTTCCAGCAAGTTGCGAAAGCAACTT | 228 | LKtAGKSEPSSKLRKQLKK | |
| ospB | | | | AAAAAGCAGCAAGACTCTTTAGATGTCGTGGACTCTTCGGTCTCCTCTTTATG | | QQDSLDVVDSSVSSLCLSN | |
| | | | | TCTGTCTAACACGGCATCATCTCATGGGACCAGAAAACTATTTCAGATTTATT | | TASSHGTRKLFQIYSKSPFY | |
| | | | | CCAAATCTCCATTCTACCGAGCTGCCTCAGGTAATGAGGCCCTGGGAATGGA | | RAASGNEALGMEGPLGQT | |
| | | | | AGGACCATTGGGCCAGACCAAATTCCTGGAAGACAAGCCTCAGTTCATCAGC | | KFLEDKPQFISRGTFNPEK | |
| | | | | AGAGGAACCTTCAACCCGGAAAAGGGCAAACAAAAATTAAAGAATGTGAAAA | | GKQKLKNVKNSPQKTKETP | |
| | | | | ACTCACCTCAGAAAACCAAAGAGACCCCAGAGGGGACAGTCATGTCTGGCC | | EGTVMSGRRKTVDPDCTS | |
| | | | | GCAGAAAAACTGTGGACCCAGACTGCACCTCCAACCAACAGC | | | |
| Shigella | 1 | prey700 | 28 | ATGGGAATTGGTCTTTCTGCTCAAGGTGTGAACATGAATAGACTACCAGGTT | 229 | MGIGLSAQGVNMNRLPGW | |
| ospB | | | | GGGATAAGCATTCATATGGTTACCATGGGGATGATGGACATTCGTTTTGTTCT | | DKHSYGYHGDDGHSFCSS | |
| | | | | TCTGGAACTGGACAACCTTATGGACCAACTTTCACTACTGGTGATGTCATTG | | GTGQPYGPTFTTGDVIGCC | |
| | | | | GCTGTTGTGTTAATCTTATCAACAATACCTGCTTTTACACCAAGAATGGACAT | | VNLINNTCFYTKNGHSLGIA | |
| | | | | AGTTTAGGTATTGCTTTCACTGACCTACCGCCAAATTTGTATCCTACTGTGGG | | FTDLPPNLYPTVGLQTPGE | |
| | | | | GCTTCAAACACCAGGAGAAGTGGTCGATGCCAATTTTGGGCAACATCCTTTC | | VVDANFGQHPFVFDIEDYM | |
| | | | | GTGTTTGATATAGAAGACTATATGCGGGAGTGGAGAACCAAAATCCAGGCAC | | REWRTKIQAQIDRFPIGDR | |
| | | | | AGATAGATCGATTTCCTATCGGAGATCGAGAAGGAGAAATGGCAGACCATGAT | | EGEWQTMIQKMVSSYLVH | |
| | | | | ACAAAAAATGGTTTCATCTTATTTAGTCCACCATGGGTACTGTGCCACAGCAG | | HGYCATAEAFARSTDQTVL | |
| | | | | AGGCCTTTGCCAGATCTACAGACCAGACCGTTCTAGAAGAATTAGCTTCCAT | | EELASIKNRQRIQKLVLAGR | |
| | | | | TAAGAATAGACAAAGAATTCAGAAATTGGTATTAGCAGGAAGAATGGGAGAA | | MGEAIETTQQLYPSLLE | |
| | | | | GCCATTGAAACAACACAACAGTTATACCCAAGTTTACTTGAAAG | | | |
| Shigella | 1 | prey67844 | 29 | TTCCATACAGGAACCCCATCTGAAGGTCACCAACATCAAAGACCAAAGGTAG | 230 | FHTGTPSEGHQHQRPKVD | |
| ospB | | | | ATAAATCCACGAAGTTGAGGAAAAACCAGTGCAAAAAGGCTGAGAATTCCAA | | KSTKLRKNQCKKAENSKN | |
| | | | | AAACCAGAAAGGCTCTTCTCCTCCAAAGGATCAAAACTCCTCGCCAGCAAGG | | QKGSSPPKDQNSSPAREQ | |
| | | | | GAACAAAACCAGATGGAGAATGAGTTTGATGAATTGACAGAAGTAGGCTTCA | | NQMENEFDELTEVGFRRW | |
| | | | | GAAGGTGGGTAATAACAAGTAAGCTAAAGGAGCATGTTCTAACCCAATGCAA | | VITSKLKEHVLTQCKEVKNL | |
| | | | | GGAAGTTAAGAACCTTGAAAAAAGGTTATG | | | |
| Shigella | 1 | prey67853 | 30 | GCCGTGGACGGTGAGGGTGCCGGCCTCACCTCGGAGGCATGGAAGTACCA | 231 | AVDGEGAGLTSEAWKYQV | |
| ospB | | | | GGTTACTTCACATCGAGAGGACCGTTTTCCTCTTTCCAGTCGGCTGCGGTTG | | TSHREDRFPLSSRLRLALK | |
| | | | | GCACTGAAGAATCTTGGTGCTGACAGACACAGAGCAGGGTCTCTCGTGGAA | | NLGADRHRAGSLVEQELS | |
| | | | | CAGGAGTTGTCTGGTCTGTTCAGTTTGATGAGTGGCAGAAAATGAGACGATG | | GLFSLMSGRK*DDGKCVC | |
| | | | | GGAAGTGTGTGTGTGGGCCTNTTTTTNGGTGCTNNGGNNGNNN | | GPXFXCXGX | |
| Shigella | 1 | prey66272 | 31 | ATGTGGGCCCTGGGTCAAGGAGGTTTTGCCAACCTCACCGAGGGACTGAAA | 232 | MWALGQAGFANLTEGLKV | |
| ospB | | | | GTGTGGCTGGGGATCATGCTGCCTGTGCTGGGCATCAAGTCTCTGTCTCCC | | WLGIMLPVLGIKSLSPFAITY | |
| | | | | TTTGCCATCACATACCTGGATCGGCTGCTCCTGATGCATCCCAACCTTACCA | | LDRLLLMHPNLTKGFGMIG | |
| | | | | AGGGCTTCGGCATGATTGGCCCCAAGGACTTCTTCCCACTTCTGGACTTTGC | | PKDFFPLLDFAYMPNNSLT | |
| | | | | CTATATGCCGAACAACTCCCTGACACCCAGCCTGCAGGAGCAGCTGTGTCA | | PSLQEQLCQLYPRLKVLAF | |
| | | | | GCTCTACCCCCGACTGAAAGTGCTGGCATTTGGAGCAAAGCCGGATTCCAC | | GAKPDSTLHTYFPSFLSRA | |
| | | | | CCTGCATACCTACTTCCCTTCTTTCCTGTCCAGAGCCACCCCTAGCTGTCCC | | TPSCPPEMKKELLSSLTEC | |
| | | | | CCTGAGATGAAGAAAGAGCTCCTGAGCAGCCTGACTGAGTGCCTGACGGTG | | LTVDPLSASVWRQLYPKHL | |
| | | | | GACCCCCTCAGTGCGAGCGTCTGGAGGCAGCTGTACCCTAAGCACCTGTCA | | SQSSLLLEHLLSSQEQIPKK | |
| | | | | CAGTCCAGCCTTCTGCTGGAGCACTTGCTCAGCTCCTGGGAGCAGATTCCC | | VQKSLQETIQSLKLTNQELL | |
| | | | | AAGAAGGTACAGAAGTCTTTGCAAGAAACCATTCAGTCCCTCAAGCTTACCA | | RKGSSNNQDVVTCDMACK | |
| | | | | ACCAGGAGCTGCTGAGGAAGGGTAGCAGTAACAACCAGGATGTCGTCACCT | | GLLQQVQGPRLPWTBLLLL | |
| | | | | GTGACATGGCCTGCAAGGGCCTGTTGCAGCAGGTTCAGGGTCCTCGGCTGC | | LLVFAVGFLCHDLRSHSSF | |
| | | | | CCTGGACGCGGCTCCTCCTGTTGCTGCTGGTCTTCGCTGTAGGCTTCCTGT | | QASLTGRLLRSSGFLPASQ | |
| | | | | GCCATGACCTCCGGTCACACAGGTCCTTCCAGGCCTCCCTTACTGGCCGGT | | QACAKLYSYSLQGYSWLG | |
| | | | | TGCTTCGATCATCTGGCTTCTTACCTGCTAGCCAACAAGCGTGTGCCAAGCT | | ETLPLWGSHLLTVVRPSLQ | |
| | | | | CTACTCCTACAGTCTGCAAGGCTACAGCTGGCTGGGGGAGACACTGCCGCT | | LAWAHTNATVSELSAHCAS | |
| | | | | CTGGGGCTCCCACCTGCTCACCGTGGTGCGGCCCAGCTTGCAGCTGGCCT | | HLAWFGDSLTSLSQBLQIQ | |
| | | | | GGGCTCACACCAATGCCACAGTCAGCTTCCTTTCTGCCCACTGTGCCTCTCA | | LPDSVNQLLRYLRELPLLFH | |
| | | | | CCTTGCGTGGTTTGGTGACAGTCTCACCAGTCTCTCTCAGAGGCTACAGATC | | QNVLLPLWHLLLEALAWAQ | |
| | | | | CAGCTCCCCGATTCCGTGAATCAGCTACTCCGCTATCTGAGAGAGCTGCCC | | EHCHEACRGEVTWDCMKT | |
| | | | | CTGCTTTTCCACCAGAATGTGCTGCTGCCACTGTGGCACCTCTTGCTTGAGG | | QLSEAVHWTWLCLQDITVA | |
| | | | | CCCTGGCCTGGGCCCAGGAGCACTGCCATGAGGCATGCAGAGGTGAGGTG | | FLDWALALISQQ* | |
| | | | | ACCTGGGACTGCATGAAGACACAGCTCAGTGAGGCTGTCCACTGGACCTGG | | | |
| | | | | CTTTGCCTACAGGACATTACAGTGGCTTTCTTGGACTGGGCACTTGCCCTGA | | | |
| | | | | TATCCCAGCAGTAG | | | |
| Shigella | 2 | prey700 | 32 | ATGGGAATTGGTCTTTCTGCTCAAGGTGTGAACATGAATAGACTACCAGGTT | 233 | MGIGLSAQGVNMNRLPGW | |
| ospD1 | | | | GGGATAAGCATTCATATGGTTACCATGGGGATGATGGACATTCGTTTTGTTCT | | DKHSYGYHGDDGHSFCSS | |
| | | | | TCTGGAACTGGACAACCTTATGGACCAACTTTCACTACTGGTGATGTCATTG | | GTGQPYGPTFTTGDVIGCC | |
| | | | | GCTGTTGTGTTAATCTTATCAACAATACCTGCTTTTACACCAAGAATGGACAT | | VNLINNTCFYTKNGHSLGIA | |
| | | | | AGTTTAGGTATTGCTTTCACTGACCTACCGCCAAATTTGTATCCTACTGTGGG | | FTDLLPPNLYPTVGLQTYGE | |
| | | | | GCTTCAAACACCAGGAGAAGTGGTCGATGCCAATTTTGGGCAACATCCTTTC | | VVDANFGQHPFVFDIEDYM | |
| | | | | GTGTTTGATATAGAAGACTATATGCGGGAGTGGAGAACCAAAATCCAGGCAC | | REWRTKIQAQIDRFPIGDR | |
| | | | | AGATAGATCGATTTCCTATCGGAGATCGAGAAGGAGAAtGGCAGACCATGAT | | EGEWQTMIQKMVSSYLVH | |
| | | | | ACAAAAAATGGTTTCATCTTATTTAGTCCACCATGGGTACTGTGCCACAGCAG | | HGYCATAE | |
| | | | | AGGC | | | |
| Shigella | 2 | prey2492 | 33 | CACCAACCTAAAGAGACAGGCTAACAAGAAGAGTGAGGGCAGCCTGGCCTA | 234 | TNLKRQANKKSEGSLAYVK | |
| ospD1 | | | | TGTGAAAGGCGGTCTCAGTACATTCTTCGAAGCACAGGATGCCCTCTCAGCC | | GGLSTFFEAQDALSAIHQK | |
| | | | | ATCCATCAAAAACTAGAAGCAGATGGAACGGAAAAAGTAGAAGGATCCATGA | | LEADGTEKVEGSMTQKLEN | |
| | | | | CGCAGAAACTGGAGAATGTTCTGAACAGAGCAAGTAATACTGCAGACACATT | | VLNRASNTADTLEQEVLGR | |
| | | | | GTTTCAAGAAGTATTAGGTCGGAAAGACAAGGCAGATTCCACTAGAAATGCA | | KDKADSTRNALNVLQRFKF | |
| | | | | CTCAATGTGCTTCAGCGATTTAAGTTTCTTTTCAACCTTCCTCTAAATATTGAA | | FLNLPLNIERNIQKGDYDVV | |
| | | | | AGGAATATTCAAAAGGGTGATTATGATGTGGTTATTAATGATTATGAAAAGGC | | INDYEKAKSLFGKTEVQVF | |
| | | | | CAAGTCACTTTTTGGGAAAACGGAGGTGCAAGTTTTCAAGAAATATTATGCTG | | KKYYAEVETRIEALRELLLD | |
| | | | | AAGTAGAAACAAGGATTGAAGCTTTAAGAGAATTACTTCTGGATAAATTGCTT | | KLLETPSTLHDQKRYIRYLS | |
| | | | | GAGACACCATCAACTTTACATGACCAAAAACGTTACATAAGGTACCTGTCTGA | | DLHASGDPAWQCIGAQHK | |
| | | | | CCTTCATGCGTCTGGTGACCCTGCTTGGCAATGCATTGGAGCCCAACACAAG | | WILQLNHSCKEGYVKDLKG | |
| | | | | TGGATCCTTCAGCTCATGCACAGTTGCAAAGAGGGCTACGTGAAAGATCTGA | | NPGLHSPMLDLDNDTYPSY | |
| | | | | AAGGTAACCCAGGCCTGCACAGTCCCATGTTGGATCTTGATAATGATACACG | | LGHLSQTASLKRGSSFQSG | |
| | | | | TCCCTCAGTGTTGGGCCATCTCAGTCAGACAGCGTCCCTGAAGAGGGGCAG | | RDDTWRYKTPHRVAFVEK | |
| | | | | CAGCTTTCAGTCTGGTCGAGACGACACGTGGAGATACAAAACTCCCCACAG | | LTKLVLSQLPNFWKLWISY | |
| | | | | GGTGGCCTTTGTTGAAAAATTGAGAAAACTCGTCTTGAGCCAGCTGCCTAAC | | VNGSLFSETAEKSGQIERS | |
| | | | | TTCTGGAAACTCTGGATCTCCTACGTTAATGGAAGCCTCTTCAGTGAGACTG | | KNVRQRQNDFKKMIQEVM | |
| | | | | GTGAGAAGTCAGGCCAGATTGAAAGATCAAAGAATGTAAGGCAAAGACAAAA | | HSLVKLTRGALHPLSIRDGE | |
| | | | | TGATTTTAAGAAAATGATTCAGGAAGTAATGCACTCCCTGGTGAAGCTTACCC | | AKQYGGWEVKCELSGQWL | |
| | | | | GCGGAGCCCTGCATCCCCTCAGCATCCGGGATGGGGAAGCCAAGCAGTAC | | AHAIQTVRLTHESLTALEIP | |
| | | | | GGAGGCTGGGAGGTGAAGTGCGAGCTCTCCGGACAGTGGCTCGCTCACGC | | NDLLQTIQDLILDLRVRCVM | |
| | | | | CATCCAGACTGTAAGACTTACTCATGAATCGTTGACTGCCCTTGAAATTCCTA | | ATLQHTAEEIKRLAEKEDWI | |
| | | | | ATGACCTGTTACAGACTATCCAGGATCTCATCTTGGATCTCCGAGTACGTTG | | VDNEGLTSLPCQFEQCIVC | |
| | | | | CGTAATGGCCACGTTGCAGCACACGGCGGAAGAAATAAAGAGATTAGCTGA | | SLQSLKGVLECKPGEASVF | |
| | | | | AAAAGAAGACTGGATTGTTGACAATGAAGGACTGACTTCTCTACCATGTCAG | | QQPKTQEEVCQLSINIMQV | |
| | | | | TTTGAACAGTGCATCGTGTGTTGTCTGCAGTCACTGAAGGGGGTTCTGGAGT | | FIYCLEQLSTKPDADIDTTH | |
| | | | | GCAAGCCGGGAGAGGCTAGTGTCTTCCAACAACCTAAAACACAGGAGGAGG | | LSVDVSSPDLKGSIHEDFSI | |
| | | | | TTTGCCAGCTAAGCATCAATATAATGCAGGTTTTTATATACTGTCTGGAACAG | | TSEQR | |
| | | | | TTGAGCACCAAGCCTGATGCAGATATAGATACTACACATCTCTCTGTTGATGT | | | |
| | | | | TTCTTCCCCTGACTTGTTTGGAAGTATCCATGAAGACTTCAGCTTGACCTCAG | | | |
| | | | | AACAGCGCC | | | |
| Shigella | 2 | prey67651 | 34 | CAGTATAAGAAGGCCTTAGAGAATGAAACAAATGAGGAGAAATCTGGCACAC | 235 | QYKKALENETNEEKSGTPG | |
| ospD1 | | | | CAGGAGCTGATAAAGCAGAAAAAAGATATAAGTATACAGTTAAGCTCANCCC | | ADKAEKRYKYTVKLXPVSL | |
| | | | | AGTCTCGTTGTACTCTTCTAGAGAAGCAACTAGAATATACAAAGAGAATGGTT | | YSSREATRIYKENGSQRRS | |
| | | | | CTCAACGTAGGAGCGAGAAAAGAACATGATCCTAGAACAACAGGCCCAGCTT | | EKRT*S*NNRPSFRGKKNKI | |
| | | | | CAGAGGGAAAAAGAACAAGATCAGATGAAGCTGTATGCAAAACTTGAAAAGC | | R*SCMQNLKSLMSXKKSVS | |
| | | | | TTGATGTCTTANAAAAAGAGTGTTTCAGACTTACAACAACAACTCAGN | | DLQQLX | |
| Shigella | 2 | prey67653 | 35 | CCCTGAAATCTGCAAAATGGCTGATAATTTGGATGAATTTATTGAAGAGCAAA | 236 | PEICKMADNLDEFIEEQKAR | |
| ospD1 | | | | AAGCCAGATTGGCCGAAGACAAAGCAGAGTTGGAAAGTGATCCACCTTACAT | | LAEDKAELESDPPYMEMK | |
| | | | | GGAAATGAAGGGAAAGTTGTCAGCGAAGCTTTCTGAAAACAGTAAGATACTG | | GKLSAKLSENSKILISMAKE | |
| | | | | ATCTCTATGGCTAAGGAAAACATACCACCAAATAGTCAACAGACCAGGGGTT | | NIPPNSQQTRGSLGIDYGI | |
| | | | | CCTTAGGAATTGATTATGGATTAAGTTTACCACTTGGAGAAGACTATGAACGG | | SLPLGEDYERKKHKLKEEL | |
| | | | | AAGAAACATAAATTAAAAGAAGAATTGCG | | | |
| Shigella | 2 | prey67667 | 36 | CGACCAGGGCACACCCCAGTACATGGAGAAGATGGAGCAGGTGTTTGAGCA | 237 | DQGTPQYMENMEQVFEQC | |
| ospD1 | | | | GTGCCAGCAGTTCGAGGAGAAACGCCTTCGCTTCTTCCGGGAGGTTCTGCT | | QQFEEKRLRFFREVLLEVQ | |
| | | | | GGAGGTTCAGAAGCACCTAAACCTGTCCAATGTGGCTGGTTACAAAGCCATT | | KHLNLSNVAGYKAIYHDLE | |
| | | | | TACCATGACCTGGAGCAGAGCATCAGAGCAGCTGATGCAGTGGAGGACCTG | | QSIRAADAVEDLRWFRANH | |
| | | | | AGGTGGTTCCGAGCCAATCACGGGCCAGGCATGGCCATGAACTGGCCGCA | | GPGMAMNWPQFEEWSAD | |
| | | | | GTTTGAGGAGTGGTCCGCAGACCTGATTCGAACCCTCAGCCGGAGAGAGAA | | LIRTLSRREKKKATDGFTLT | |
| | | | | GAAGAAGGCCACTGACGGCTTCACCCTGACGGGCATCAACCAGACAGGCGA | | GINQTGDQFLPSKPSS | |
| | | | | CCAGTTTTTGCCGAGTAAGCCCAGCAGCAC | | | |
| Shigella | 2 | prey67657 | 37 | CCCGCCTGCCATGGACTGGATCTTCCAGTGCATCTCCTACCATGCCCCCGA | 238 | PPAMDWIFQCISYHAPEAL | |
| ospD1 | | | | GGCTCTGCTGACCGAGATGATGGAAAGGTGTAAGAAACTAGGAAACAATGC | | LTEMMERCKKLGNNALLLN | |
| | | | | CTTGCTGTTGAATTCTGTGATGTCTGCCTTCCGGGCTGAGTTCATCGCCACA | | SVMSAFRAEFIATRSMDFIG | |
| | | | | AGGTCTATGGATTTCATTGGCATGATTAAAGAGTGTGATGAATCTGGTTTCCC | | MIKECDESGFPKHLLFRSI | |
| | | | | CAAGCATCTTCTTTTTCGATCACTGGGATTAAACTTGGCCTTGGCTGATCCTC | | GLNLALADPPESDBLQILNE | |
| | | | | CTGAGAGTGACCGACTTCAGATTCTCAACGAAGCTTGGAAAGTCATCACTAA | | AWKVITKLKNPQDYINCAE | |
| | | | | GCTGAAGAACCCACAGGACTACATTAATTGTGCCGAAGTGTGGGTGGAATAC | | VWVEYTCKHFTKREVNTVL | |
| | | | | ACCTGCAAGCATTTCACGAAACGAGAGGTGAATACCGTTTTGGCAGATGTCA | | ADVIKHMTPDRAFEDSYPQ | |
| | | | | TCAAGCACATGACTCCAGATCGTGCATTTGAAGATTCCTACCCCCAGCTTCA | | LQLIIKKVIAHFHDFSVLFSY | |
| | | | | GTTAATAATTAAGAAAGTTATTGCCCACTTCCATGACTTCTCAGTTCTTTTCTC | | EKFLPELDMFQKESVRVEV | |
| | | | | AGTGGAAAAATTTCTGCCGTTTCTGGACATGTTCCAAAAAGAGAGTGTGCGG | | CKCIMDAFIKHQQEPTKD | |
| | | | | GTGGAGGTTTGCAAATGCATCATGGACGCCTTTATCAAGCATCAACAAGAGC | | | |
| | | | | CCACCAAGGACC | | | |
| Shigella | 2 | prey67501 | 38 | CTTCCGCCTGGAACAGCTGGAATGCCTTGATGATGCAGAAAAAAAATTAAAC | 239 | FRLEQLECLDDAEKKLNLA | |
| ospD1 | | | | TTGGCCCAGAAATGCTTTAAAAATTGTTACGGAGAAAATCATCAGAGACTGGT | | QKCFKNCYGENHQRLVHIK | |
| | | | | CCACATAAAAGGAAATTGTGGGAAAGAGAAGGTACTGTTTCTAAGACTCTAC | | GNCGKEKVLFLRLYLLQGI | |
| | | | | TTACTTCAAGGGATCCGAAACTATCACAGTGGAAATGATGTAGAGGCTTATG | | RNYHSGNDVEAYEYLNRH | |
| | | | | AGTATCTTAACAGGCACGTCAGCTCTTTAAAGAGCTATATATTGATCCATCAA | | VSSLKSYILIHQKWTICCSW | |
| | | | | AAGTGGACAATTTGTTGCAGTTGGGGTTTACTGCCCAGGAAGCACCGGCTTG | | GLLPRKHRLGLRACDGNV | |
| | | | | GCCTGAGGGCGTGTGATGGGAACGTGGATCATGCGGCCACTCATATTACCA | | DHAATHITNRREELAQIRKE | |
| | | | | ACCGCAGAGAGGAACTGGCCCAAATAAGGAAGGAGGAAAAAGAGAAGAAAA | | EKEKKRRRLENIRFLKGMG | |
| | | | | GACGCCGCCTCGAGAACATCAGGTTTCTGAAAGGGATGGGCTACTCCACGC | | YSTH | |
| | | | | ACG | | | |
| Shigella | 2 | prey67678 | 39 | GAACAAGCTGAGGGTGTTGGACCCAGAGGTTACCCAGCAGACCATAGAGCT | 240 | NKLRVLDPEVTQQTIELKEE | |
| ospD1 | | | | GAAGGAAGAGTGCAAAGACTTTGTGGACAAAATTGGCCAGTTTCAGAAAATA | | CKDFVDKIGQFQKIVGGLIE | |
| | | | | GTTGGTGGTTTAATTGAGCTTGTTGATCAACTTGCAAAAGAAGCAGAAAATGA | | LVDQLAKEAENEKMKAIGA | |
| | | | | AAAGATGAAGGCCATCGGTGCTCGGAACTTGCTCAAATCTATAGCAAAGCAG | | RNLLKSIAKQREAQQQQLQ | |
| | | | | AGAGAAGCTCAACAGCAGCAACTTCAAGCCCTAATAGCAGAAAAGAAAATGC | | ALIAEKKMQLERYRVEYEA | |
| | | | | AGCTAGAAAGGTATCGGGTTGAATATGAAGCTTTGTGTAAAGTAGAAGCAGA | | LCKVEAEQNEFIDQFIFQK* | |
| | | | | ACAAAATGAATTTATTGACCAATTTATTTTTCAGAAATGA | | | |
| Shigella | 2 | prey67578 | 40 | ATGGCGGTGGAGACTCTGTCCCCGGACTGGGAGTTTGACCGCGTTGACGAC | 241 | MAVETLSPDWEFDRVDDG | |
| ospD1 | | | | GGCTCGCAGAAAATTCATGCCGAAGTCCAACTTAAGAATTATGGGAAATTTCT | | SQKIHAEVQLKNYGKFLEE | |
| | | | | TGAGGAGTATACCTCTCAACTGAGAAGAATTGAGGACGCTCTGGATGACTCA | | YTSQLRRIEDALDDSIGDV | |
| | | | | ATTGGAGATGTTTGGGATTTCAATCTTGATCCTATAGCATTAAAGCTTTTGCC | | WDFNLDPIALKLLPYEQSSI | |
| | | | | TTATGAACAGTCCTCTCTTTTGGAACTCATAAAGACTGAAAACAAGGTCTTAA | | LELIKTENKVLNKVITVYAAL | |
| | | | | ACAAAGTCATCACTGTTTATGCTGCACTTTGTTGTGAAATCAAGAAATTAAAAT | | CCEIKKLKYEAETKFYNGLL | |
| | | | | ATGAGGCTGAAACTAAATTTTACAATGGTCTCTTGTTTTATGGAGAAGGAGCT | | EYGEGATDASMVEGDCQI | |
| | | | | ACAGATGCCAGCATGGTGGAAGGTGATTGCCAAATTCAAATGGGGAGATTTA | | QMGRFISFLQELSCFVTRC | |
| | | | | TTTCATTCTTACAGGAACTGTCTTGCTTTGTTACGAGGTGCTATGAAGTGGTG | | YEVVMNVVHQLAALYISNKI | |
| | | | | ATGAACGTAGTCCACCAGTTGGCTGCCCTCTATATCAGTAACAAGATTGCAC | | APKIIETTGVHFQTMYEHLG | |
| | | | | CCAAAATTATAGAGACAACTGGAGTTCATTTTCAGACTATGTATGAGCACTTG | | ELLTVLLTLDEIIDNHITLKD | |
| | | | | GGAGAACTGCTAACAGTTTTGCTCACCCTGGATGAAATTATTGATAATCATAT | | HWTMYKRLLKSVHHNPSK | |
| | | | | CACACTGAAAGACCACTGGACTATGTACAAAAGGTTACTGAAATCTGTCCAT | | FGIQEEKLKPFEKFLLKLEG | |
| | | | | CACAATCCTTCAAAATTTGGAATTCAGGAAGAAAAATTAAGCCATTTGAAAA | | QLLDGMIFQACIEQQFDSL | |
| | | | | GTTCTTGCTGAAGCTAGAAGGGCAATTACTGGATGGAATGATATTCCAGGCC | | NGGVSVSKNSTFAEEFAHS | |
| | | | | TGTATAGAACAACAATTTGATTCTCTCAATGGAGGAGTATCTGTGTCAAAAAA | | IRSIFANVEAKLGEPSEIDQ | |
| | | | | TAGTACTTTTGCTGAGGAATTTGCACATAGTATTCGGTCAATTTTTGCAAATG | | RDKYVGICGLFVLHFQIFRT | |
| | | | | TAGAAGCCAAACTTGGAGAACCTTCTGAAATTGACCAGAGAGACAAGTATGT | | IDKKEYKSLLD | |
| | | | | TGGAATTTGTGGACTCTTTGTATTGCACTTTCAGATTTTTCGAACTATTGATAA | | | |
| | | | | AAAGTTTTATAAGTCTTTATTGGAC | | | |
| Shigella | 2 | prey67580 | 41 | GCACTCCCCGCCGGTCCGACTCCGCCATCTCTGTCCGCTCCCTGCACTCAG | 242 | TPRRSDSAISVRSLHSESS | |
| ospD1 | | | | AGTCCAGCATGTCTCTGCGCTCCACATTCTCACTGCCCGAGGAGGAGGAGG | | MSLRSTFSLPEEEEEPEPL | |
| | | | | AGCCGGAGCCACTGGTGTTTGCGGAGCAGCCCTCGGTGAAGCTGTGCTGTC | | VFAEQPSVKLCCQLCCSVF | |
| | | | | AGCTCTGCTGCAGCGTCTTCAAAGACCCCGTGATCACCACGTGTGGGCACA | | KDPVITTCGHTECRRCALK | |
| | | | | CGTTCTGTAGGAGATGCGCCTTGAAGTCAGAGAAGTGTCCCGTGGACAACG | | SEKCPVDNVKLTVVVNNIA | |
| | | | | TCAAACTGACCGTGGTGGTGAACAACATCGCGGTGGCCGAGCAGATCGGGG | | VAEQIQELFIHCRHGCRVA | |
| | | | | AGCTCTTCATCCACTGCCGGCACGGCTGCCGGGTAGCGGGCAGCGGGAAG | | GSGKPPIFEVDPRGCPFTIK | |
| | | | | CCCCCCATCTTTGAGGTGGACCCCCGAGGGTGCCCCTTCACCATCAAGCTC | | LSARKDHEGSCKYRPVRC | |
| | | | | AGCGCCCGGAAGGACCACGAGGGCAGCTGTGACTACAGGCCTGTGCGGTG | | PNNPSCPPLLRMNLEAHLK | |
| | | | | TCCCAACAACCCCAGCTGCCCCCCGCTGCTCAGGATGAACCTGGAGGCCCA | | ECEHIKCPHSKYGCTFIGN | |
| | | | | CCTCAAGGAGTGCGAGCACATCAAATGCCCCCACTCCAAGTACGGGTGCAC | | QDTYETHLETCRFEGLKEF | |
| | | | | GTTCATCGGGAACCAGGACACTTACGAGACCCACCTGGAGACTTGCCGCTT | | LQQTDDRFHEMHVALAQK | |
| | | | | CGAGGGGGTGAAGGAGTTTCTGCAGCAGACGGATGACCGCTTCCACGAGAT | | DQEIAFLRSMLGKLSEKID | |
| | | | | GCACGTGGCTCTGGCCCAGAAGGACCAGGAGATCGCCTTCCTGCGCTCCAT | | | |
| | | | | GCTGGGAAAGCTCTCGGAGAAGATCGACC | | | |
| Shigella | 2 | prey3160 | 42 | CAGAAAACTACATGAACTTACGGTTATGCAAGATAGACGAGAACAAGCAAGA | 243 | RKLHELTVMQDRREQARQ | |
| ospD1 | | | | CAAGACTTGAAGGGTTTGGAAGAGACAGTGGCAAAAGAACTTCAGACTTTAC | | DLKGLEETVAKELQTLHNL | |
| | | | | ACAACCTGCGCAAACTCTTTGTTCAGGACCTG | | RKLFVQDL | |
| Shigella | 2 | prey50427 | 43 | ATGGAGGAGTATGAGAAGTTCTGTGAAAAAAGTCTTGCCAGAATACAAGAAG | 244 | MEEYEKFCEKSLARIQEAS | |
| ospD1 | | | | CATCACTATCCACAGAGAGCTTTCTCCCTGCTCAGTCTGAAAGTATCTCACTT | | LSTESFLPAQSESISLIRFH | |
| | | | | ATTCGCTTTCATGGAGTGGCTATCCTTTCTCCACTGCTTAACATTGAGAAAAG | | GVAILSPLLNIEKRKEMQQE | |
| | | | | AAAGGAAATGCAACAAGAAAAGCAGAAAGCACTTGATGTAGAAGCAAGAAAG | | KQKALDVEARKQVNRKKAL | |
| | | | | CAGGTTAACAGGAAGAAAGCTTTACTGACTCGTGTCCAGGAGATTCTTGACA | | LTRVQEILDNVQVRKAPNA | |
| | | | | ATGTTCAGGTTAGAAAAGCACCTAATGCCAGTGATTTTGATCAGTGGGAGAT | | SDFDQWEMETVYSNSEVR | |
| | | | | GGAAACAGTTTACTCTAATTCAGAAGTCAGAAACTTGAATGTTCCTGCTACAT | | NLNVPATFPNSFPSHTEHS | |
| | | | | TTCCAAATAGCTTTCCAAGCCATACGGAACACTCTACTGCAGCAAAGCTTGAT | | TAAKLDKIAGILPLDNEDQC | |
| | | | | AAGATAGCTGGGATTTTGCCATTGGATAATGAGGAGCAATGTAAAACTGATG | | KTDGIDLARDSEGFNSPKQ | |
| | | | | GAATAGACTTAGCTAGAGATTCAGAAGGATTTAATTCTCCGAAGCAATGTGAT | | CDSSNISHVENEAFPKTSS | |
| | | | | AGTTCCAATATTAGTCATGTAGAAAATGAAGCTTTTCCAAAGACCTCTTCAGC | | ATPQETLISDGPFSVNEQQ | |
| | | | | AACCCCACAAGAAACTCTTATTTCTGATGGTCCCTTCTCAGTAAATGAACAAC | | DLPLLAEVIPDPYVMSLQNL | |
| | | | | AGGATCTACCACTTTTGGCAGAAGTCATCCCAGATCCCTATGTAATGAGTCTT | | MKKSKEYIEREQSRRSLRG | |
| | | | | CAGAATCTGATGAAAAAGTCAAAGGAATATATAGAAAGAGAACAATCTAGAC | | SMNRIVNESHLDKEHDAVE | |
| | | | | GCAGTCTGAGAGGTAGTATGAACAGAATTGTTAATGAGAGTCATTTAGACAA | | VADCVKEKGQLTGKHCVS | |
| | | | | AGAACATGATGCTGTTGAAGTGGCTGACTGTGTAAAAGAGAAAGGCCAGTTG | | VIPDKPSLNKSNVLLQGAST | |
| | | | | ACAGGCAAACACTGTGTCTCAGTTATTCCTGACAAACCAAGCCTTAATAAATC | | QASSMSMPVLASFSKVDIPI | |
| | | | | AAATGTTCTTCTCCAAGGTGCTTCCACTCAAGCAAGCAGCATGAGTATGCCA | | RTGHPTVLESNSDFKVIPTI | |
| | | | | GTTTTAGCTAGCTTTTCGAAAGTGGACATACCTATACGAACTGGCCATCCCA | | VTENNVIKSLTGSYAKLPSP | |
| | | | | CTGTTCTAGAGTCTAATTCTGATTTTAAAGTTATTCCCACTATTGTTACCGAAA | | EPSMSPKMHRRR | |
| | | | | ATAATGTTATCAAAAGTCTTACAGGTTCATATGCCAAATTACCTAGTCCAGAG | | | |
| | | | | CCAAGTATGAGTCCTAAAATGCACCGAAGACGT | | | |
| Shigella | 2 | prey63765 | 44 | GGACAGCCCAACCTCTGGCAGACCAGGGGTTACCAGCCTCACAACTGCAGC | 245 | DSPTSGRPGVTSLTTAAAF | |
| ospD1 | | | | TGCCTTCAAGCCTGTAGGATCCACTGGCGTCATCAAGTCACCAAGCTGGCAA | | KPVGSTGVIKSPSWQRPN | |
| | | | | CGGCCAAACCAAGGAGTACCTTCCACTGGAAGAATCTCAAACAGCGCTACTT | | QGVPSTGRISNSATYSGSV | |
| | | | | ACTCAGGATCAGTGGCACCAGCCAACTCAGCTTTGGGACAAACCCAGCCAA | | APANSALGQTQPSDQDTLV | |
| | | | | GTGACCAGGACACTTTAGTGCAAAGAGCTGAGCACATTCCAGCAGGGAAAC | | QRAEHIPAGKRTPMCAHC | |
| | | | | GAACTCCGATGTGCGCCCATTGTAACCAGGTCATCAGAGGACCATTCTTAGT | | NQVIRGPFLVALGKSWHPE | |
| | | | | GGCACTGGGGAAATCTTGGCACCCAGAAGAATTCAACTGCGCTCACTGCAA | | EFNCAHCKNTMAYIGFVEE | |
| | | | | AAATACAATGGCCTACATTGGATTTGTAGAGGAGAAAGGAGCCCTGTATTGT | | KGALYCELCYEKFFAPECG | |
| | | | | GAGCTGTGCTATGAGAAATTCTTTGCCCCTGAATGTGGTCGATGCCAAAGGA | | RCQRKILGEVINALKQTWH | |
| | | | | AGATCCTTGGAGAAGTCATCAATGCGTTGAAACAAACTTGGCATGTTTCCTGT | | VSCFVCVACGKPIRNNVFH | |
| | | | | TTTGTGTGTGTAGCCTGTGGAAAGCCCATTCGGAACAATGTTTTTCACTTGGA | | LEDGEPYCETDYYALFGTI | |
| | | | | GGATGGTGAACCCTACTGTGAGACTGATTATTATGCCCTCTTTGGTACTATAT | | CHGCEFPIEAGDMFLEALG | |
| | | | | GCCATGGATGTGAATTTCCCATAGAAGCTGGTGACATGTTCCTGGAAGCTCT | | YTWHDTCFVCSVCCESLE | |
| | | | | GGGCTACACCTGGCATGACACTTGCTTTGTATGCTCAGTGTGTTGTGAAAGT | | GQTFFSKKDKPLCKKHAHS | |
| | | | | TTGGAAGGTCAGACCTTTTTCTCCAAGAAGGACAAGCCCCTGTGTAAGAAAC | | VNF* | |
| | | | | ATGCTCATTCTGTGAATTTTTGA | | | |
| Shigella | 2 | prey67623 | 45 | ATTTTATAGGAGGCATACACCATACATGGTACAGCCAGAGTACCGAATCTAT | 246 | FYRRHTPYMVQPEYRIYEM | |
| ospD1 | | | | GAGATGAACAAGAGACTGCAGTCTCGCACAGAGGATAGTGACAACCTCTGG | | NKRLQSRTEDSDNLWWDA | |
| | | | | TGGGACGCCTTTGCCACTGAATTTTTTGAAGATGACGCCACATTAACCCTTTC | | FATEFFEDDATLTLSFCLED | |
| | | | | ATTTTGTTTGGAAGATGGACCAAAGCGATACACTATCGGCAGGACCCTCATC | | GPKRYTIGRTLIPRYFSTVF | |
| | | | | CCCCGTTACTTTAGCACTGTGTTTGAAGGAGGGGTGACCGACCTGTATTACA | | EGGVTDLYYILKHSKESYH | |
| | | | | TTCTCAAACACTCGAAAGAGTCATACCACAACTCATCCATCACGGTGGACTG | | NSSITVDCDQCTMVTQHGK | |
| | | | | CGACCAGTGTACCATGGTCACCCAGCACGGGAAGCCCATGTTTACCAAGGT | | PMFTKVCTEGRLILEFTFDD | |
| | | | | ATGTACAGAAGGCAGACTGATCTTGGAGTTCACCTTTGATGATCTCATGAGA | | LMRIKTWHFTIRQYRELVP | |
| | | | | ATCAAAACATGGCACTTTACCATTAGACAATACCGAGAGTTAGTCCCGAGAA | | RSILAMHAQDPQVLDQLSK | |
| | | | | GCATCCTAGCCATGCATGCACAAGATCCTCAGGTCCTGGATCAGCTGTCCAA | | NITRMGLTNFTLNYLRLCVI | |
| | | | | AAACATCACCAGGATGGGGCTAACAAACTTCACCCTCAACTACCTCAGGTTG | | LEPMQELMSRHKTYNLSPR | |
| | | | | TGTGTAATATTGGAGCCAATGCAGGAACTGATGTCGAGACATAAAACTTACA | | DCLKTCLFQKWQRMVAPP | |
| | | | | ACCTCAGTCCCCGAGACTGCCTGAAGACCTGCTTGTTTCAGAAGTGGCAGA | | AEPTRQP | |
| | | | | GGATGGTGGCTCCGCCAGCAGAACCCACAAGGCAACCAA | | | |
| Shigella | 2 | prey7315 | 46 | ATGCTGGATAGGGATGTGGGCCCAACTCCCATGTATCCGCCTACATACCTG | 247 | MLDRDVGPTPMYPPTYLEP | |
| ospD1 | | | | GAGCCAGGGATTGGGAGGCACACACCATATGGCAACCAAACTGACTACAGA | | GIGRHTPYGNQTDYRIFEL | |
| | | | | ATATTTGAGCTTAACAAACGGCTTCAGAACTGGACAGAGGTGTGACAATC | | NKRLQNWTEECDNLWWD | |
| | | | | TCTGGTGGGATGCATTCACGACTGAGTTCTTTGAGGATGATGCCATGTTGAC | | AFTTEFFEDDAMLTITFCLE | |
| | | | | CATCACTTTGTGCCTGGAGGATGGACCAAAGAGATATACCATTGGCCGGACC | | DGPKRYTIGRTLIPRYFRSI | |
| | | | | CTGATCCCACGCTACTTCCGCAGCATCTTTGAGGGGGGTGCTACGGAGCTG | | FEGGATELYYVLKHPKEAF | |
| | | | | TACTATGTTCTTAAGCACCCCAAGGAGGCATTCCACAGCAACTTTGTGTCCC | | HSNFVSLDCDQGSMVTQH | |
| | | | | TCGACTGTGACCAGGGCAGCATGGTGACCCAGCATGGCAAGCCCATGTTCA | | GKPMFTQVCVEGRLYLEF | |
| | | | | CCCAGGTGTGTGTGGAGGGCCGGTTGTACCTGGAGTTCATGTTTGACGACA | | MFDDMMRIKTWHFSIRQH | |
| | | | | TGATGCGGATAAAGACGTGGCACTTCAGCATCCGGCAGCACCGAGAGCTCA | | RELIPRSILAMHAQDPQML | |
| | | | | TCCCCCGCAGCATCCTTGCCATGCATGCCCAAGACCCCCAGATGTTGGATC | | DQLSKNITRCGLSNSTLNYL | |
| | | | | AGCTCTCCAAAAACATCACTCGGTGTGGGCTGTCCAATTCCACTCTCAACTA | | RLCVILEPMQELMSRHKTY | |
| | | | | CCTCCGACTCTGTGTGATACTCGAGCCCATGCAAGAGCTCATGTCACGCCAC | | S | |
| | | | | AAGACCTACAGC | | | |
| Shigella | 2 | prey67601 | 47 | AGTCACTGCTTCAACCACCTGTGAGAAATTAGAAAAAGCCAGGAATGAGTTA | 248 | VTASTTCEKLEKARNELQT | |
| ospD1 | | | | CAAACAGTGTATGAAGCATTCGTCCAGCAGCACCAGGCTGAAAAAACAGAAC | | VYEAFVQQHQAEKTEREN | |
| | | | | GAGAGAATCGGCTTAAAGAGTTTTACACCAGGGAGTATGAAAAGCTTCGGGA | | RLKEFYTREYEKLRDTYIEE | |
| | | | | CACTTACATTGAAGAAGCAGAGAAGTACAAAATGCAATTGCAAGAGCAGTTT | | AEKYKMQLQEQFDNLNAA | |
| | | | | GACAACTTAAATGCTGCGCATGAAACCTCTAAGTTGGAAATTGAAGCTAGCC | | HETSKLEIEASHSEKLELLK | |
| | | | | ACTCAGAGAAACTTGAATTGCTAAAGAAGGCCTATGAAGCCTCCCTTTCAGA | | KAYEASLSEIKKGHEIEKKS | |
| | | | | AATTAAGAAAGGCCATGAAATAGAAAAGAAATCGCTTGAAGATTTACTTTCTG | | LEDLLSEKQESLEKQINDLK | |
| | | | | AGAAGCAGGAATCGCTAGAGAAGCAAATCAATGATCTGAAGAGTGAAAATGA | | SENDALNEKLKSEEQKRRA | |
| | | | | TGCTTTAAATGAAAAATTGAAATCAGAAGAACAAAAAAGAAGAGCAAGAGAAA | | REKANLKNPQIMYLEQELE | |
| | | | | AAGCAAATTTGAAAAATCCTCAGATCATGTATGTAGAACAGGAGTTAGAAAGC | | SLKAVLEIKNEKLHQQDIKL | |
| | | | | CTGAAAGCTGTGTTAGAGATCAAGAATGAGAAACTGCATCAACAGGACATCA | | MKMEKLVDNNTALVDKLKR | |
| | | | | AGTTAATGAAAATGGAGAAACTGGTGGACAACAACACAGCATTGGTTGACAA | | FQQENEELKARMDKHMAIS | |
| | | | | ATTGAAGCGTTTCCAGCAGGAGAATGAAGAATTGAAAGCTCGGATGGACAAG | | RQLSTEQAVLQESLEKESK | |
| | | | | CACATGGCAATCTCAAGGCAGCTTTCCACGGAGCAGGCTGTTCTGCAAGAG | | VNKRLSMENEELLWKLHN | |
| | | | | TCGCTGGAGAAGGAGTCGAAAGTCAACAAGCGACTCTCTATGGAAAACGAG | | GDKCSPKRSPTSSAIPLQS | |
| | | | | GAGCTTCTGTGGAAACTGCACAATGGGGACCTGTGTAGCCCCAAGAGATCC | | PRNSGSFPSPSISPR* | |
| | | | | CCCACATCCTCCGCCATCCCTTTGCAGTCACCAAGGAATTCGGGCTCCTTCC | | | |
| | | | | CTAGCCCCAGCATTTCACCCAGATGA | | | |
| Shigella | 2 | prey53735 | 48 | CTCGCTTCCTCCTAGCACTGGGACATTTCAAGAAGCTCAGAGCCGGTTGAAT | 249 | SLPPSTGTFQEAQSRLNEA | |
| ospD1 | | | | GAAGCTGCTGCTGGGCTGAATCAGGCAGCCACAGAACTGGTGCAGGCCTCT | | AAGLNQAATELVQASRGTP | |
| | | | | CGGGGAACCCCTCAGGACCTGGCTCGAGCCTCAGGCCGATTTGGACAGGA | | QDLARASGRFGQDFSTFLE | |
| | | | | CTTCAGCACCTTCCTGGAAGCTGGTGTGGAGATGGCAGGCCAGGCTCCGAG | | AGVEMAGQAPSQEDRAQV | |
| | | | | CCAGGAGGACCGAGCCCAAGTTGTGTCCAACTTGAAGGGCATCTCCATGTC | | VSNLKGISMSSSKLLLAAKA | |
| | | | | TTCAAGGAAACTTCTTCTGGCTGCCAAGGCCCTGTCCACGGACCCTGCTGCC | | LSTDPAAPNLKSQLAAAAR | |
| | | | | CCTAACCTCAAGAGTCAGCTGGCTGGAGCTGCCAGGGCAGTAACTGACAGC | | AVTDSINQLITMCTQQAPG | |
| | | | | ATCAATCAGCTCATCACTATGTGCACCCAGCAGGCACCCGGCCAGAAGGAG | | QKECDNALRELETVRELLE | |
| | | | | TGTGATAACGCCCTGGGGGAATTGGAGACGGTCCGGGAACTCCTGGAGAAC | | NPVQPINDMSYFGCLDSVM | |
| | | | | CCAGTCCAGCCCATCAATGACATGTCCTACTTTGGTTGCCTGGACAGTGTAA | | ENSKVLGEAMTGISQNAKN | |
| | | | | TGGAGAACTCAAAGGTGCTGGGCGAGGCCATGACTGGCATCTCCCAAAATG | | GNLPEFGDAISTASKALCG | |
| | | | | CCAAGAACGGAAAGCTGCCAGAGTTTGGAGATGCCATTTCCACAGCCTCAAA | | FTEAAAQAAYLVGVSDPNS | |
| | | | | GGCACTTTGTGGCTTCACCGAGGCAGCTGCACAGGCTGCATATCTGGTTGG | | QAGQQGLVEPTQFARANQ | |
| | | | | TGTCTCTGACCCCAATAGCCAAGCTGGACAGCAAGGGCTAGTGGAGCCCAC | | AIQMACQSLGEPGCTQAQ | |
| | | | | ACAGTTTGCCCGTGCAAAGCAGGCAATTCAGATGGCCTGCCAGAGTTTGGG | | VLSAATIVAKHTSALCNSCR | |
| | | | | AGAGCCTGGCTGTACCCAGGCCCAGGTGCTCTCTGCAGCCACCATTGTGGC | | LASARTTNPTAKRQFVQSA | |
| | | | | TAAACACACCTCTGCACTGTGTAACAGCTGTCGCCTGGCTTCTGCCCGTACC | | KEVANSTANLVKTIKALDGA | |
| | | | | ACCAATCCTACTGCCAAGCGCCAGTTTGTACAGTCAGCCAAGGAGGTGGCC | | FTEENRAQCRAATAPLLEA | |
| | | | | AACAGCACAGCTAATCTTGTCAAGACCATCAAGGCGCTAGATGGGGCCTTCA | | VDNLSAFASNPEFSSIPAQI | |
| | | | | CAGAGGAGAACCGTGCCCAGTGCCGAGCAGCAACAGCCCCTCTGCTGGAG | | SPEGRAAMEPIVIS | |
| | | | | GGTGTGGACAATCTGAGTGCCTTTGCGTCCAACCCTGAGTTCTCCAGCATTC | | | |
| | | | | CTGCCCAGATCAGCCCTGAGGGTCGGGCTGGCATGGAGCCCATTGTGATCT | | | |
| | | | | CTGC | | | |
| Shigella | 2 | prey67630 | 49 | GAGGACCTGCAGCCACCCAGCGCCCTGTCGGCCCCCTTCACCAACAGCCTC | 250 | EDLQPPSALSAPFTNSLAR | |
| ospD1 | | | | GCTCGCTCTGCGCGCCAGTCTGTGCTCCGGTATAGCACTCTCCCTGGGCGC | | SARQSVLRYSTLPGRRALK | |
| | | | | AGGGCCCTGAAGAACTCCCGCCTAGTGAGCCAGAAGGATGACGTCCACGTC | | NSRLVSQKDDVHVCILCLR | |
| | | | | TGTATCCTTTGTCTCAGAGCCATCATGAACTATCAGTACGGATTCAACCTGGT | | AIMNYQYGFNLVMSHPHAV | |
| | | | | CATGTCCCACCCCCATGCTGTCAATGAGATTGCACTTAGCCTCAATAACAAG | | NEIALSLNNKNPRTKALVLE | |
| | | | | AATCCAAGGACCAAAGCCCTTGTCTTAGAGCTTCTGGCAN | | LLA | |
| Shigella | 2 | prey12665 | 50 | GAAGCGGCACGAGCGAATGATCAAGAACCGGGAGTCAGCCTGCCAGTCCC | 251 | KRHERMIKNRESACQSRR | |
| ospD1 | | | | GGAGAAAGAAGAAAGAGTATCTGCAGGGACTGGAGGCTCGGCTGCAAGCAG | | KKKEYLQGLEARLQAVLAD | |
| | | | | TACTGGCTGACAACCAGCAGCTCCGCCGAGAGAATGCTGCCCTCCGGCGGC | | NQQLRRENAALRRRLEALL | |
| | | | | GGCTGGAGGCCCTGCTGGCTGAAAACAGCGAGCTCAAGTTAGGGTCTGGAA | | AENSELKLGSGNRKVVCIM | |
| | | | | ACAGGAAGGTGGTCTGCATCATGGTCTTCCTTCTCTTCATTGCCTTCAACTTT | | VELLFIAFNFGPVSISEPPSA | |
| | | | | GGACCTGTCAGCATCAGTGAGCCTCCTTCAGCTCCCATCTCTCCTCGGATGA | | PISPRMNKGEPQPRRHLLG | |
| | | | | ACAAGGGGGAGCCTCAACCCCGGAGACACTTGCTGGGGTTCTCAGAGCAAG | | FSEQEPVQGVEPLQGSSQ | |
| | | | | AGCGAGTTCAGGGAGTTGAACCTCTCCAGGGGTCCTCCCAGGGCCCTAAGG | | GPKEPQPSPTDQPSFSNLT | |
| | | | | AGCCCCAGCCCAGCCCCACAGACCAGCCCAGTTTCAGCAACCTGACAGCCT | | AFPGGAKELLLRDLDQLFL | |
| | | | | TCCCTGGGGGCGCCAAGGAGCTACTACTAAGAGACCTAGACCAGCTCTTCC | | SSDCRHFNRTESLRLADEL | |
| | | | | TCTCCTCTGATTGCCGGCACTTCAACCGCACTGAGTCCCTGAGGCTTGCTGA | | SGWVQRHQRGRRKIPQRA | |
| | | | | CGAGTTGAGTGGCTGGGTCCAGCGCGACCAGAGAGGCCGGAGGAAGATCC | | QERQKSQPRKKSPPVKAV | |
| | | | | CTCAGAGGGCCCAGGAGAGACAGAAGTCTCAGCCACGGAAGAAGTCACCTC | | PI | |
| | | | | CAGTTAAGGCAGTCCCCATCC | | | |
| Shigella | 2 | prey67631 | 51 | TGAGAGCGAGGTCTCGGAGCATCTCAGTGCCAGCTCGGCTTCTGCCATCCA | 252 | ESEVSEHLSASSASAIQQD | |
| ospD1 | | | | GCAGGACAGCACTTCCAGCATGCAGCCACCATCTGAAGCCCCCATGGTGAA | | STSSMQPPSEAPMVNTVS | |
| | | | | CACAGTCAGCTCAGCTTATTCGGAGGATTTTGAAAACTCTCCAAGTCTGACA | | SAYSEDFENSPSLTASEPT | |
| | | | | GCATCTGAGCCAACCGCCGATTCCAAGGAGTCTCTTGACAGAACACTGGAC | | AHSKESLDRTLDALSESSS | |
| | | | | GCTTTGTCTGAATCCTCTTCAAGTGTGAAGACAGACCTTCCACAAACAGCCG | | SVKTDLPQTAESRKKSGRH | |
| | | | | AGTCTAGGAAAAAGTCGGGCAGGCACGTGACAAGAGTGCTTGTGAAGGACA | | VTRVLVKDTAVQTPDPAFT | |
| | | | | CAGCTGTGCAGACGCCAGATCCTGCCTTCACCTACGAGTGGACCAAGGTGG | | YEWTKVASMAAMGPALGG | |
| | | | | CCAGCATGGCAGCCATGGGGCCTGCCCTGGGAGGCGCCTACGTGGACCCG | | AYVDPTPIANHVISADAIEAL | |
| | | | | ACACCCATCGCCAATCATGTTATCAGTGCAGATGCAATAGAAGCCCTGACCG | | TAYSPAVLALHDVLKQQLS | |
| | | | | CTTACAGCCCGGCCGTGCTGGCACTCCATGATGTGCTGAAGCAGCAGCTGA | | LTQQFIQASRHLHASLLRSL | |
| | | | | GCCTGACGCAGCAGTTCATCCAGGCGAGCCGGCACCTGCACGCCTCCCTCC | | DADSFHYHTLEEAKEYIRC | |
| | | | | TGCGCTCCCTGGACGCGGACTCCTTCCACTACCACACCCTGGAGGAAGCCA | | HRPAPLTMEDALEEVNKEL | |
| | | | | AAGAGTACATTAGGTGCGACAGACCTGCCCCACTGACCATGGAGGATGCCC | | * | |
| | | | | TGGAGGAGGTGAACAAGGAGCTGTGA | | | |
| Shigella | 2 | prey20143 | 52 | ATGGGAGAGAGCCGCCAGGACCTGGAGGAGGAGTATGAGCCTCAGTTCCTG | 253 | MAESRQDLEEEYEPQFLRL | |
| ospD1 | | | | CGGCTCCTAGAGAGGAAAGAAGCTGGGACCAAAGCTCTGCAGAGAACCCAG | | LERKEAGTKALQRTQAEIQ | |
| | | | | GCTGAGATCCAGGAAATGAAGGAGGCTCTGAGACCCCTGCAAGCAGAGGCC | | EMKEALRPLQAEARQLRLQ | |
| | | | | CGGCAGCTCCGCCTGCAAAACAGGAACCTGGAGGACCAGATCGCACTTGTG | NRNLEDQIALVRQKRDEEV | | |
| | | | | AGGCAAAAACGAGATGAAGAGGTGCAGCAGTACAGGGAACAGCTGGAGGAA | | QQYREQLEEMEERQRQLR | |
| | | | | ATGGAAGAACGCCAGAGGCAGTTAAGAAATGGGGTGCAACTCCAGCAACAG | | NGVQLQQQKNKEMEQLRL | |
| | | | | AAGAACAAAGAGATGGAACAGCTAAGGCTCAGTCTTGCTGAAGAGCTCTCTA | | SLAEELSTYKAMLLPKSLE | |
| | | | | CTTATAAGGCTATGCTACTACCCAAGAGCCTGGAACAGGCTGATGCTCCCAC | | QADAPTSQAGGMETQSQG | |
| | | | | TTCTCAGGCAGGTGGAATGGAGACACAGTCTCAAGGGGCTGTTTAG | | AV* | |
| Shigella | 2 | prey1418 | 53 | CTGGGTCATCCCAGATCCCGAAGAGGAACCAGAGCGCAAGCGAAAGAAGG | 254 | WVIPDPEEEPERKRKKGPA | |
| ospD1 | | | | GCCCAGCCCCGAAGATGCTGGGCCACGAGGTTTGCCGTGTCTGTGGGGAC | | PKMLGHELCRVCGDKASG | |
| | | | | AAGGCCTCCGGCTTCCACTACAACGTGCTCAGCTGCGAAGGCTGCAAGGGC | | FHYNVLSCEGCKGFFRRSV | |
| | | | | TTCTTCCGGCGCAGTGTGGTCCGTGGTGGGGCGAGGCGCTATGCCTGCCG | | VRGGARRYACRGGGTCQ | |
| | | | | GGGTGGCGGAACCTGCCAGATGGACGCTTTCATGCGGCGCAAGTGCCAGC | | MDAFMRRKCQQCRLRKCK | |
| | | | | AGTGCCGGCTGCGCAAGTGCAAGGAGGCAGGGATGAGGGAGCAGTGCGTC | | EAGMREQCVLSEEQIRKKK | |
| | | | | CTTTCTGAAGAACAGATCCGGAAGAAGAAGATTCGGAAACAGCAGCAGCAG | | IRKQQQQESQSQSQSPVG | |
| | | | | GAGTCACAGTCACAGTCGCAGTCACCTGTGGGGCCGCAGGGCAGCAGCAG | | PQGSSSSASGPGASPGGS | |
| | | | | CTCAGCCTCTGGGCCTGGGGCTTCCCCTGGTGGATCTGAGGCAGGCAGCC | | EAGSQGSGEGEGVQLTAA | |
| | | | | AGGGGTCCGGGGAAGGCGAGGGTGTCCAGCTAACAGCGGCTCAAGAACTA | | QELMIQQLVAAQLQCNKRS | |
| | | | | ATGATCCAGCAGTTGGTGGCGGCCCAACTGCAGTGCAACAAACGCTCCTTC | | FSDQPKVTPWPLGADPQS | |
| | | | | TCCGACCAGCCCAAAGTCACGCCCTGGCCCCTGGGCGCAGACCCCCAGTC | | RDARQQRFAHFTELAIISVQ | |
| | | | | CCGAGATGCCCGCCAGCAACGCTTTGCCCACTTCACGGAGCTGGCCATCAT | | EIVDFAKQVPGFLQLGRED | |
| | | | | CTCAGTCCAGGAGATCGTGGACTTCGCTAAGCAAGTGCCTGGTTTCCTGCA | | QIALLKASTIEIMLLETARRY | |
| | | | | GCTGGGCCGGGAGGACCAGATCGCCCTCCTGAAGGCATCCACTATCGAGAT | | NHE | |
| | | | | CATGCTGCTAGAGACAGCCAGGCGCTACAACCACGAGA | | | |
| Shigella | 2 | prey67642 | 54 | ATGAAGGATGAACCACGGTCCACGAACCTGTTCATGAAGCTGGACTCGGTCT | 255 | MKDEPRSTNLFMKLDSVFI | |
| ospD1 | | | | TCATCTGGAAGGAACCCTTTGGCCTGGTCCTCATCATCGCACCCTGGAACTA | | WKEPFGLVLIIAPWNYPLNL | |
| | | | | CCCATTGAACCTGACCCTGGTGCTCCTGGTGGGCACCCTCCCCGCAGGGAA | | TLVLLVGTLPAGNCVVLKP | |
| | | | | TTGCGTGGTGCTGAAGCCGTCAGAAATCAGCCAGGGCACAGAGAAGGTCCT | | SEISQGTEKVLAEVLPQYLD | |
| | | | | GGCTGAGGTGCTGCCCCAGTACCTGGACCAGAGCTGCTTTGCCGTGGTGCT | | QSCFAVVLGGPQETGQLLE | |
| | | | | GGGCGGACCCCAGGAGACAGGGCAGCTGCTAGAGCACAAGTTGGACTACA | | HKLDYIFFTGSPRVGKIVMT | |
| | | | | TCTTCTTCACAGGGAGCCCTCGTGTGGGCAAGATTGTCATGACTGCTGCCAC | | AATKHLTPVTLEL | |
| | | | | CAAGCACCTGACGCCTGTCACCCTGGAGCTGGG | | | |
| Shigella | 2 | prey67648 | 55 | GCTGGGGATCGCGCTGGCGCTCGTGGGCGAGAGGCTTCTGGCACTCAGAA | 256 | LGIALALLGERLLALRNRLK | |
| ospD1 | | | | ATCGACTTAAAGCCTCCAGAGAAGTAGAATCTGTAGACCTTCCACACTGCCA | | ASREVESVDLPHCHLIKGIE | |
| | | | | CCTGATTAAAGGAATTGAAGCTGGCTCTGAAGATATTGACATACTTCCCAATG | | AGSEDIDILPNGLAFFSVGL | |
| | | | | GTCTGGCTTTTTTTAGTGTGGGTCTAAAATTGCCAGGACTCCACAGCTTTGCA | | KEPGLHSFAPDKPGGILMM | |
| | | | | CCAGATAAGCCTGGAGGAATACTAATGATGGATCTAAAGAAGAAAAACCAA | | DLKEEKPRARELRISRGFD | |
| | | | | GGGCACGGGAATTAAGAATCAGTCGTGGGTTTGATTTGGCCTCATTCAATCC | | LASFNPHGISTFIDNDD | |
| | | | | ACATGGCATCAGGACTTTCATAGACAACGATGAC | | | |
| Shigella | 3 | prey67266 | 56 | NNNNNNNNNNNNNNNNNNNNNNNNNNNNNNNNNNNNNNNNNNNNNNNNNN | 257 | XXXXXXXXXXXXXXXXXXX | |
| ospC1 | | | | NNNNNNNNNNNNNNNNNNNNNNNNNNNNNNNNNNNNNNNNNNNNNNNNNN | | XXXXXXXXXXXXXXXXXXX | |
| | | | | NNNNNNNNNNNNNNNNNNNNNNNNNNNNNNNGGTTCTTAATCTTTGCTCTC | | XXXXXXVLNLCSPDPFTLIKI | |
| | | | | CTGACCCTTTTACTCTCATAAAAATTATTNGAGGACTCCAAATATAATAGCTTT | | IXGLQI**LLFMYVITLDTILEL | |
| | | | | TATTTATGTATGTNATAACTTTGGATACTATATTAGAATTAAAACTGAGAAANT | | KLRXLKGLIKK*LCTC* | |
| | | | | TAAAGGGCTTAATTAAAAAATAACTCTGTACATGTTAAAN | | | |
| Shigella | 3 | prey67267 | 57 | TACTATTTACTGGATGTCTCAGTAGGCATAGTTTAGAGATACGTTGTGTGCAA | 258 | YYLLDVSVGIV*RYVVCNRH | |
| ospC1 | | | | TAGACATATAAATGATTTGTTTACATTACTCCATATAAATGATTTGTTTGTTTAA | | INKLFTLLHINDLFV*CLGNY | |
| | | | | TGTCTTGGAAATTATTTTATGTCTTATGGTTGCTGATTCTGTTGCCATCNGATT | | FMSYGC*FCCHXITTIXKIXX | |
| | | | | ACTACGATAAANAAGATCTGNGNCTANNGANGGGNCTTNTTTGAACTGNTNC | | XXXXXFESXLXXXXGXXCX | |
| | | | | TNNGGCNTNGGNTNGGGNGCNTNTGTNTNGNCNNNGTTTGTTGNGNNNANG | | XXVVXXXAXXXXVDXQVW | |
| | | | | GCGNGNNCGGNGNCNGTTGATTNNCAGGTNTGGNNNNGNTGGNGGCNCNT | | XXWXXXAPXX | |
| | | | | GGCNCCTNGCATNTN | | | |
| Shigella | 3 | prey50590 | 58 | GTTTGATCAGCCTCAGGAATACTTCATGGAGTTGACATTCAATCAAGCTGCAA | 259 | FDQPQEYFMELTFNQAAK | |
| ospC1 | | | | AGGGGGTCAACAAGGAGTTCACCGTGAACATCATGGACACGTGTGAGCGCT | | GVNKEFTVNIMDTCERCNG | |
| | | | | GCAACGGCAAGGGGAACGAGCCCGGCACCAAGGTGCAGCATTGCCACTAC | | KGNEPGTKVQHCHYCGGS | |
| | | | | TGTGGCGGCTCCGGCATGGAAACCATCAACAGAGGCCCTTTTGTGATGCGT | | GMETINTGPFVMRSTCRRC | |
| | | | | TCCACGTGTAGGAGATGTGGTGGCCGCGGCTCCATCATCATATCGCCCTGT | | GGRGSIIISPCVVCRGAGQ | |
| | | | | GTGGTCTGCAGGGGAGCAGGACAAGCCAAGCAGAAAAAGCGAGTGATGATC | | AKQKKRVMIPVPAGVEDG | |
| | | | | CCTGTGCCTGCAGGAGTCGAGGATGGCCAGACCGTGAGGATGCCTGTGGG | | QTVRMPVGKREIFITFRVQ | |
| | | | | AAAAAGGGAAATTTTCATTACGTTCAGGGTGCAGAAAAGCCCTGTGTTCCGG | | KSPVFRRDGADIHSDLFISI | |
| | | | | AGGGACGGCGCAGACATCCACTCCGACCTGTTTATTTCTATAGCTCAGGCTC | | AQALLGGTARAQGLYETIN | |
| | | | | TTCTTGGGGGAACAGCCAGAGCCCAGGGCCTGTACGAGACGATCAACGTGA | | VTIPPGTQTDQKIRMGGKGI | |
| | | | | CGATCCCCCCTGGGACTCAGACAGACCAGAAGATTCGGATGGGTGGGAAAG | | PRINSYGYGDHYIHIKIRVP | |
| | | | | GCATCCCCCGGATTAACAGCTACGGCTACGGAGACCAACTACATCCACATCAA | | KRLTSRQQSLILSYAEDETD | |
| | | | | GATACGAGTTCCAAAGAGGCTAACGAGCCGGCAGCAGAGCCTGATCCTGAG | | VEGTVNGVTLTSSGGSTM | |
| | | | | CTACGCCGAGGACGAGACAGATGTGGAGGGGACGGTGAACGGCGTCACCC | | DSSAGSKARREAGEDEEG | |
| | | | | TCACCAGCTCTGGTGGCAGCACCATGGATAGCTCCGCAGGAAGCAAGGCTA | | FLSKLKKMFTS* | |
| | | | | GGCGTGAGGCTGGGGAGGACGAGGAGGGATTCCTTTCCAAACTTAAGAAAA | | | |
| | | | | TGTTTACCTCATGA | | | |
| Shigella | 3 | prey9822 | 59 | ATGGCGGACCTTGATTCGCCTCCGAAGCTGTCAGGGGTGCAGCAGCCGTCT | 260 | MADLDSPPKLSGVQQPSE | |
| ospC1 | | | | GAGGGGGTGGGAGGTGGCCGCTGCTCCGAAATCTCCGCTGAGCTCATTCG | | GVGGGRCSEISAELIRSLTE | |
| | | | | CTCCCTGACAGAGCTGCAGGAGCTGGAGGCTGTATACGAACGGCTCTGCGG | | LQELEAVYERLCGEEKVVE | |
| | | | | CGAGGAGAAAGTGGTGGAGAGAGAGCTGGATGCTCTTTTGGAACAGCAAAA | | RELDALLEQQNTIESKMVTL | |
| | | | | CACCATTGAAAGTAAGATGGTCACTCTCCACCGAATGGGTCCTAATCTGCAG | | HRMGPNLQLIEGDAKQLAG | |
| | | | | CTGATTGAGGGAGATGCAAAGCAGCTGGCTGGAATGATCACCTTTACCTGCA | | MITFTCNLAENVSSKVRQL | |
| | | | | ACCTGGCTGAGAATGTGTCCAGCAAAGTTCGTCAGCTTGACCTGGCCAAGAA | | DLAKNRLYQAIQRADDILDL | |
| | | | | CCGCCTCTATCAGGCCATTCAGAGAGCTGATGACATCTTGGACCTGAAGTTC | | KFCMDGVQTALRSEDYEQ | |
| | | | | TGCATGGATGGAGTTCAGACTGCTTTGAGGAGTGAAGATTATGAGCAGGCTG | | AAAHIHRYLCLDKSVIELSR | |
| | | | | CAGCACATATTCATCGCTACTTGTGCCTGGACAAGTCGGTCATTGAGCTCAG | | QGKGGSMIDANLKLLQEAE | |
| | | | | CCGACAGGGCAAAGGGGGGAGCATGATTGATGCCAACCTGAAATTGCTGCA | | QRLKAIVAEKFAIATKEGDL | |
| | | | | GGAAGCTGAGCAACGTCTCAAAGCCATTGTGGCAGAGAAGTTTGCCATTGC | | PQVERFFKIFPLLGLHEEGL | |
| | | | | CACCAAGGAAGGTGATTTGCCCCAGGTGGAGCGCTTCTTCAAGATCTTCCCA | | RRFSEYLCKQVASKAEENL | |
| | | | | CTGCTGGGTTTGCATGAGGAGGGATTAAGAAGGTTCTCGGAGTACCTTTGCA | | LMVLGTDMSDRRAAVIFAD | |
| | | | | AGCAGGTGGCCAGTAAAGCTGAGGAGAATCTGCTCATGGTGCTGGGGACAG | | TLTLLFEGIARIVEAHQPIVE | |
| | | | | ACATGAGTGATCGGAGAGCTGCAGTCATCTTTGCAGATACACTTACTCTTCT | | TYYGPGRLYTLIKYLQVEC | |
| | | | | GTTTGAAGGGATTGCCCGCATTGTGGAGGGCCACCAGCCAATAGTGGAGAC | | DRQVEKVVDKFIKQRDHQ | |
| | | | | CTATTATGGGCCAGGGAGACTCTATACCCTGATCAAATATCTGCAGGTGGAA | | QFRHVQNNLMRNSTTEKIE | |
| | | | | TGTGACAGACAGGTGGAGAAGGTGGTAGACAAGTTCAATCAAGCAAAGGGAC | | PRELDPILTEVTLMNARSEL | |
| | | | | TACCACCAGCAGTTCCGGCATGTTCAGAACAACCTGATGAGAAATTCTACAA | | YLRFLKKRISSDFEVGDSM | |
| | | | | CAGAAAAAATCGAACCAAGAGAACTGGACCCCATCCTGACTGAGGTCACCCT | | ASEEVKQEHQKCLDKLLNN | |
| | | | | GATGAACGCCCGCAGTGAGCTATACTTACGCTTCCTCAAGAAGAGGATTAGC | | CLLSCTMQELIGLYVTMEE | |
| | | | | TCTGATTTTGAGGTGGGAGACTCCATGGCCTCAGAGGAAGTAAAGCAAGAG | | YFMRETVNKAVALDTYEKG | |
| | | | | CACCAGAAGTGTCTGGACAAACTCCTCAATAACTGCCTTTTGAGCTGTACCA | | QLTSSMVDDVFYIVKKCIGR | |
| | | | | TGCAGGAGCTAATTGGCTTATATGTTACCATGGAGGAGTACTTCATGAGGGA | | ALSSSSIDCLCAMINLATTE | |
| | | | | GACTGTCAATAAGGCTGTGGCTCTGGACACCTATGAGAAGGGCCAGCTGAC | | LESDFRDVLCNKLRMGFPA | |
| | | | | ATCCAGCATGGTGGATGATGTCTTCTACATTGTTAAGAAGTGCATTGGGCGG | | TTFQDIQRGVTSAVNIMHS | |
| | | | | GCTCTGTCCAGCTCCAGCATTGACTGTCTCTGTGCCATGATCAACCTCGCCA | | SLQQGKFDTKGIESTDEAK | |
| | | | | CCACAGAGCTGGAGTCTGACTTCAGGGATGTTCTGTGTAATAAGCTGCGGAT | | MSFLVTLNNVEVCSENISTL | |
| | | | | GGGCTTTCCTGCCACCACCTTCCAGGACATCCAGCGCGGGGTGACAAGTGC | | KKTLESDCTKLFSQGIGGE | |
| | | | | CGTGAACATCATGCACAGCAGCCTCCAGCAAGGCAAATTTGACACAAAAGGC | | QAQAKFDGCLSDLAAVSNK | |
| | | | | ATCGAGAGTACTGACGAGGCGAAGATGTCCTTCCTGGTGACTCTGAACAAC | | FRDLLQEGLTELNSTAIKTQ | |
| | | | | GTGGAAGTCTGCAGTGAAAACATCTCCACTCTGAAGAAGACACTGGAGAGTG | | VQPWINSFFSVSHNIEEEEF | |
| | | | | ACTGCACCAAGCTCTTCAGCCAGGGCATTGGAGGGGAGCAGGCCCAGGCC | | NDYEANDPWVQQFILNLEQ | |
| | | | | AAGTTTGACGGCTGCCTTTCTGACTTGGCCGCCGTGTCCAACAAATTCCGAG | | QMAEFKASLSPVIYDSLTGL | |
| | | | | ACCTCTTGCAGGAAGGGCTGACGGAGCTCAACAGCACAGCCATCAAGCCAC | | MTSLVAVELEKVVLKSTFN | |
| | | | | AGGTGCAGGCTTGGATCAACAGCTTTTTCTCCGTCTCCCACAACATCGAGGA | | RLGGLQFDKELRSLIAYLTT | |
| | | | | GGAAGAATTCAATGACTATGAGGCCAACGACCCTTGGGTACAACAGTTCATC | | VTTWTIRDKFARLSQMATIL | |
| | | | | CTTAACCTGGAGCAGCAAATGGCAGAGTTCAAGGCCAGCCTGTCCCCGGTC | | NLERVTEILDYWGPNSGPL | |
| | | | | ATCTACGACAGCCTAACCGGCCTCATGACTAGCCTTGTTGCCGTCGAGTTGG | | TWRLTPAEVRQVLALRIDF | |
| | | | | AGAAAGTGGTGCTGAAATCCACCTTTAACCGGCTGGGTGGTCTGCAGTTTGA | | RSEDIKRLRL* | |
| | | | | CAAGGAGCTGAGGTCGCTCATTGCCTACCTTACCACGGTGACCACCTGGAC | | | |
| | | | | CATCCGAGACAAGTTTGCCCGGCTCTCCCAGATGGCCACCATCCTCAATCTG | | | |
| | | | | GAGCGGGTGACCGAGATCCTCGATTACTGGGGACCCAATTCCGGCCCATTG | | | |
| | | | | ACGTGGCGCCTCACCCCTGCTGAAGTGCGCCAGGTGCTGGCCCTGCGGAT | | | |
| | | | | AGACTTCCGCAGTGAAGATATCAAGAGGCTGCGCCTGTAG | | | |
| Shigella | 3 | prey67268 | 60 | CCGTGTCTTGGCTGGCTCATTTATCAGGGTTGTCTTTCTCTTTGTCTTTGACT | 261 | PCLGWLIYQGCLSLCL*LGY | |
| ospC1 | | | | AGGCTATTTTACTACTCTATAGAGATAGAAATTTGTTTACAGTGCACTAATACT | | FTTL*R*KFVYSALILM*IIPV | |
| | | | | GATGTAAATAATTCCTGTTCATAAAACTGCAAATTATATCATTGAATGCAATTG | | HKTANYIIECN*LQPCRHSR | |
| | | | | ATTATGGCCCTGTAGACATTCAAGAGTTTTGCCAGTTTGCACCCATTTGTAAA | | VLPVCTHL*MCFSISYLTINV | |
| | | | | TGTGTTTTAGCATCTCTTATCTGACTATAAATGTGCTGCTTTTGATTTATCTTA | | LLLIYLTNHLS | |
| | | | | CAAACCATTTGTCACN | | | |
| Shigella | 3 | prey67270 | 61 | NCNGGTGNGTGNAGANGGAGTNNANCTNTGCCACTGCATGNTGTTTTGCTC | 262 | XGXXRXSXXXPLHXVLLRX | |
| ospC1 | | | | AGGCANGATNNATGATGCTTGACTTTTATGAAGTTCCANNATTCAAATGGATN | | DX*CLTFMKFXXSNGXDA* | |
| | | | | TGATGCNTAACCTTCCCCATGTANTNGTTGTACATGTTCATGNGGGCTGGNN | | PSPCXXCTCSXGLXXLXXL | |
| | | | | TNNCTNNTNNTTCTATNGNTCATTAGATNNNNNNNCACTCTTGNACTCTCNCT | | XXIRXXXTLXLSLXLPSCH* | |
| | | | | NTANTTACCCTCATGCCATTGANNAATCTGTCNTTCTCATTNATGATCCCNTA | | XICXSHX*SXXXXPXIS | |
| | | | | NNNNCTGNCCANNGATCTCTC | | | |
| Shigella | 3 | prey67271 | 62 | GCAGGAGCTGCAGAAGAAGGCAGAGCACCAGGTGGGGGAAGATGGGTTTT | 263 | QELQKKAEHQVGEDGFLLK | |
| ospC1 | | | | TACTGAAGATCAAGCTGGGGCACTATGCCACACAGCTCCAGAACACGTATGA | IKLGHYATQLQNTYDRCPM | | |
| | | | | CCGCTGCCCCATGGAGCTGGTCCGCTGCATCCGCCATATATTGTACAATGAA | | ELVRCIRHILYNEQRLVREA | |
| | | | | CAGAGGTTGGTCCGAGAAGCCAACAATGGTAGCTCTCCAGCTGGAAGCCTT | | NNGSSPAGSLADAMSQKH | |
| | | | | GCTGATGCCATGTCCCAGAAACACCTCCAGATCAACCAGACGTTTGAGGAG | | LQINQTFEELRLVTQDTENE | |
| | | | | CTGCGACTGGTCACGCAGGACACAGAGAATGAGTTAAAAAAGCTGCAGCAG | | LKKLQQTQEYFIIQYQESLR | |
| | | | | ACTCAGGAGTACTTCATCATCCAGTACCAGGAGAGCCTGAGGATCCAAGCTC | | IQAQFGPLAQLSPQERLSR | |
| | | | | AGTTTGGCCCGCTGGCCCAGCTGAGCCCCCAGGAGCGTCTGAGCCGGGAG | | ETALQQKQVSLEAWLQRE | |
| | | | | ACGGCCCTCCAGCAGAAGCAGGTGTCTCTGGAGGCCTGGTTGCAGCGTGA | | AQTLQQYRVELPEKHQKTL | |
| | | | | GGCACAGACACTGCAGCAGTACCGCGTGGAGCTGCCCGAGAAGCACCAGA | | QLLRKQQTIILDDELIQWKR | |
| | | | | AGACCCTGCAGCTGCTGCGGAAGCAGCAGAGCATCATCCTGGATGACGAGC | | RQQLAGNGGPPEGSLDVL | |
| | | | | TGATCCAGTGGAAGCGGCGGCAGCAGCTGGCCGGGAACGGCGGGCCCCC | | QSWCEKLAEIIWQNRQQIR | |
| | | | | CGAGGGCAGCCTGGACGTGCTACAGTCCTGGTGTGAGAAGTTGGCGGAGAT | | RAEHLCQQLPIPGPVEEML | |
| | | | | CATCTGGCAGAACCGGCAGCAGATCCGCAGGGCTGAGCACCTCTGCCAGCA | | AEVNATITDIISALVTSTFIIE | |
| | | | | GCTGCCCATCCCCGGCCCAGTGGAGGAGATGCTGGCCGAGGTCAACGCCA | | KQPPQVLKTQTKFAATVRL | |
| | | | | CCATCACGGACATTATCTCAGCCCTGGTGACCAGCACGTTCATCATTGAGAA | | LVGGKLNVHMNPPQVKATII | |
| | | | | GCAGCCTCCTCAGGTCCTGAAGACCCAGACCAAGTTTGCAGCCACTGTGCG | | SEQQAKSLLKNENTRNDYS | |
| | | | | CCTGCTGGTGGGCGGGAAGCTGAACGTGCACATGAACCCCCCCCAGGTGA | | GEILNNCCVMEYHQATGTL | |
| | | | | AGGCCACCATCATCAGTGAGCAGCAGGCCAAGTCTCTGCTCAAGAACGAGA | | SAHFRNMSLKRIKRSDRRG | |
| | | | | ACACCCGCAATGATTACAGTGGCGAGATCTTGAACAACTGCTGCGTCATGGA | | AESVTEEKFTILFESQFSVG | |
| | | | | GTACCACCAAGCCACAGGCACCCTTAGTGCCCACTTCAGGAATATGTCCCTG | | GNELVFQVKTLSLPVVVIVH | |
| | | | | AAACGAATTAAGAGGTCAGACCGTCGTGGGGCAGAGTCGGTGACAGAAGAA | | GSQDNNATATVLWDNAFA | |
| | | | | AAATTTACAATCCTGTTTGAATCCCAGTTCAGTGTTGGTGGAAATGAGCTGGT | | EPGRVPFAVPDKVLWPQL | |
| | | | | TTTTCAAGTCAAGACCCTGTCCCTGCCAGTGGTGGTGATCGTTCATGGCAGC | | CEALNMKFKAEVQSNRGLT | |
| | | | | CAGGACAACAATGCGACGGCCACTGTTCTCTGGGACAATGCTTTTGCAGAG | | KENLVFLAQKLFNNSSSHL | |
| | | | | CCTGGCAGGGTGCCATTTGCCGTGCCTGACAAAGTGCTGTGGCCACAGCTG | | EDYSGLSVSWSQFNRENL | |
| | | | | TGTGAGGCGCTCAACATGAAATTCAAGGCCGAAGTGCAGAGCAACCGGGGC | | PGRNYTFWQWFDGVMEVL | |
| | | | | CTGACCAAGGAGAACCTCGTGTTCCTGGCGCAGAAACTGTTCAACAACAGCA | | KKHLKPHWNDGAILGFVNK | |
| | | | | GCAGCCACCTGGAGGACTACAGTGGCCTGTCTGTGTCCTGGTCCCAGTTCA | | QQAHDLLINKPDGTFLLRFS | |
| | | | | ACAGGGAGAATTTACCAGGACGGAATTACACTTTCTGGCAATGGTTTGACGG | | DSEIGGITIAWKFDSQERMF | |
| | | | | TGTGATGGAAGTGTTAAAAAAACATCTCAAGCCTCATTGGAATGATGGGGCC | | WNLMPFTTRDFSIRSLADR | |
| | | | | ATTTTGGGGTTTGTAAACAAGCAACAGGCCCATGACCTACTGATTAACAAGC | | LGDLNYLIYVFPDRPKDEVY | |
| | | | | CAGATGGGACCTTGCTCCTGAGATTCAGTGACTCAGAAATTGGCGGCATCAC | | SKYYTPVPCESATAKAVDG | |
| | | | | CATTGCTTGGAAGTTTGATTCTCAGGAAAGAATGTTTTGGAATCTGATGCCTT | | YVKPQIKQVVPEFVNASAD | |
| | | | | TTACCACCAGAGACTTCTCCATCAGGTCCCTAGCCGACCGCTTGGGAGACTT | | AGGGSATYMDQAPSPAVC | |
| | | | | GAATTACCTTATCTACGTGTTTCCTGATCGGCCAAAAGATGAAGTATACTCCA | | PQAHYNMYPQNPDSVLDT | |
| | | | | AATACTACACACCAGTTCCCTGCGAGTCTGCTACTGCTAAAGCTGTTGATGG | | DGDFDLEDTMDVARRVEE | |
| | | | | ATACGTGAAGCCACAGATCAAGCAAGTGGTCCCTGAGTTTGTGAACGCATCT | | LLGRPMDSQWIPHAQS* | |
| | | | | GCAGATGCCGGGGGCGGCAGCGCCACGTACATGGACCAGGCCCCCTCCCC | | | |
| | | | | AGCTGTGTGTCCCCAGGCTCACTATAACATGTACCCACAGAACCCTGACTCA | | | |
| | | | | GTCCTTGACACCGATGGGGACTTCGATCTGGAGGACACAATGGACGTAGCG | | | |
| | | | | CGGCGTGTGGAGGAGCTCCTGGGCCGGCCAATGGACAGTCAGTGGATCCC | | | |
| | | | | GCACGCACAATCGTGA | | | |
| Shigella | 3 | prey700 | 63 | ATGGGAATTGGTCTTTCTGCTCAAGGTGTGAACATGAATAGACTACCAGGTT | 264 | MGIGLSAQGVNMNRLPGW | |
| ospC1 | | | | GGGATAAGCATTCATATGGTTACCATGGGGATGATGGACATTCGTTTTGTTCT | | DKHSYGYHGDDGHSFCSS | |
| | | | | TCTGGAACTGGACAACCTTATGGACCAACTTTCACTACTGGTGATGTCATTG | | GTGQPYGPTFTTGDVIGCC | |
| | | | | GCTGTTGTGTTAATCTTATCAACAATACCTGCTTTTACACCAAGAATGGACAT | | VNLINNTCFYTKNGHSLGIA | |
| | | | | AGTTTAGGTATTGCTTTCACTGACCTACCGCCAAATTTGTATCCTACTGTGGG | | FTDLPPNLYPTVGLQTPGE | |
| | | | | GCTTCAAACACCAGGAGAAGTGGTCGATGCCAATTTTGGGCAACATCCTTTC | | VVDANFGQHPFVFDIEDYM | |
| | | | | GTGTTTGATATAGAAGACTATATGCGGGAGTGGAGAACCAAAATCCAGGCAC | | REWRTKIQAQIDRFPIGDR | |
| | | | | AGATAGATCGATTTCCTATCGGAGATCGAGAAGGAGAATGGCAGACCATGAT | | EGEWQTMIQKMVSSYLVH | |
| | | | | ACAAAAAATGGTTTCATCTTATTTAGTCCACCATGGGTACTGTGCCACAGCAG | | HGYCATAEAFARSTDQTVL | |
| | | | | AGGCCTTTGCCAGATCTACAGACCAGACCGTTCTAGAAGAATTAGCTTCCAT | | EELASIKNRQRIQKLVLAGR | |
| | | | | TAAGAATAGACAAAGAATTCAGAAATTGGTATTAGCAGGAAGAATGGGAGAA | | MGEAIETTQQLYPSLLERN | |
| | | | | GCCATTGAAACAACACAACAGTTATACCCAAGTTTACTTGAAAGAAATCCTAA | | PNLLFTLKVRQFIEMVNGT | |
| | | | | TCTCCTTTTCACATTAAAAGTGCGTCAGTTTATAGAAATGGTGAATGGTACAG | | DSEVRCLGGRSPKSQDSY | |
| | | | | ATAGTGAAGTACGATGTTTGGGAGGCCGAAGTCCAAAGTCTCAAGACAGTTA | | PVSPRPFSSPSMSPSHGM | |
| | | | | TCCTGTTAGTCCTCGACCTTTTAGTAGTCCAAGTATGAGCCCCAGCCATGGA | | NIHNLASGKGSTAHFSGFE | |
| | | | | ATGAATATCCACAATTTAGCATCAGGCAAAGGAAGCACCGCACATTTTTCAG | | SCSNGVISNKAHQSYCHSN | |
| | | | | GTTTTGAAAGTTGTAGTAATGGTGTAATATCAAATAAAGCACATCAATCATATT | | KHQSSNLNVPELNSINMSR | |
| | | | | GCCATAGTAATAAACACCAGTCATCCAACTTGAATGTACCAGAACTAAACAGT | | SQQVNNFTSNDVDMETDH | |
| | | | | ATAAATATGTCAAGATCACAGCAAGTTAATAACTTCACCAGTAATGATGTAGA | | YSNGVGETSSNGFLNGSS | |
| | | | | CATGGAAACAGATCACTACTCCAATGGAGTTGGAGAAACTTCATCCAATGGT | | KHDHEMEDCDTEMEVDSS | |
| | | | | TTCCTAAATGGTAGCTCTAAACATGACCACGAAATGGAAGATTGTGACACCG | | QLRRQLCGGSQAAIERMIH | |
| | | | | AAATGGAAGTTGATTCAAGTCAGTTGAGACGCCAGTTGTGTGGAGGAAGTCA | | FGRELQAMSEQLRRDCGK | |
| | | | | GGCCGCCATAGAAAGAATGATCCACTTTGGACGAGAGCTGCAAGCAATGAG | | NTANKKMLKDAFSLLAYSD | |
| | | | | TGAACAGCTAAGGAGAGACTGTGGCAAGAACACTGCAAACAAAAAATGTTG | | PWNSPVGNQLDPIQREPV | |
| | | | | AAGGATGCATTCAGTCTACTAGCATATTCAGATCCCTGGAACAGCCCAGTTG | | CSALNSAILETHNLPKQPPL | |
| | | | | GAAATCAGCTTGACCCGATTCAGAGAGAACCTGTGTGCTCAGCTCTTAACAG | | ALAMGQATQCLGLMARSGI | |
| | | | | TGCAATATTAGAAACCCACAATCTGCCAAAGCAACCTCCACTTGCCCTAGCA | | GSCAFATVEDYLH* | |
| | | | | ATGGGACAGGCCACACAATGTGTAGGACTGATGGCTCGATCAGGAATTGGA | | | |
| | | | | TCCTGCGCATTTGCCACAGTGGAAGACTACCTACATTAG | | | |
| Shigella | 3 | prey3486 | 64 | GATCGAGATCCATGGGAAGGCAGGCCTGTTTTTAGAAGGCCAGATCCACCC | 265 | IEIHGKAGLFLEGQIHPELE | |
| ospC1 | | | | CGAGTTGGAAGGAGTCGAGATTGTCATCAGTGAAAAGGGGGCAAGTTCACC | | GVEIVISEKGASSPLITVFTD | |
| | | | | GCTGATCACAGTCTTTACTGATGACAAAGGTGCCTACAGTGTTGGCCCCCTG | | DKGAYSVGPLHSDLEYTVT | |
| | | | | CACAGTGACCTGGAGTACACGGTGACCTCACAGAAGGAGGGCTATGTTCTG | | SQKEGYVLTAVEGTIGDFK | |
| | | | | ACTGCGGTGGAAGGAACCATCGGAGACTTCAAGGCCTATGCCCTGGCAGGC | | AYALAGVSFEIKAEDDQPL | |
| | | | | GTAAGCTTTGAGATAAAAGCTGAGGATGACCAGCCCCTCCCGGGAGTCCTC | | PGVLLSLSGGLFRSNLLTQ | |
| | | | | TTATCCCTGAGCGGTGGCCTGTTTCGTTCCAACCTCTTGACCCAGGACAACG | | DNGILTFSNLSPGQYYFKP | |
| | | | | GCATTCTGACATTCTCAAACCTGAGCCCTGGCCAGTATTACTTCAAACCCAT | | MMKEFRFEPSSQMIEVQE | |
| | | | | GATGAAGGAGTTCCGGTTTGAGCCATCCTCACAGATGATCGAGGTGCAGGA | | GQNLKITITGYRTAYSCYGT | |
| | | | | AGGCCAGAACCTGAAGATCACCATCACGGGGTACCGAACCGCTTACAGTTG | | VSSLNGEPEQGVAMEAVG | |
| | | | | CTATGGCACAGTGTCTTCCTTAAACGGAGAGCCCGAACAAGGGGTTGCCAT | | QNDCSIYGEDTVTDEEGKF | |
| | | | | GGAAGCGGTGGGCCAGAACGACTGCAGCATTTACGGAGAAGACACCGTGAC | | RLRGLLPGCVYHVQLKAEG | |
| | | | | AGACGAAGAGGGCAAGTTCAGATTACGTGGATTGCTGCCGGGATGTGTGTA | | NDHIERALPHHRVIEVGNN | |
| | | | | CCACGTTCAGCTCAAGGCAGAAGGCAACGACCACATTGAGCGGGCGCTCCC | | DIDDVNIIVFRQINQFDLSG | |
| | | | | CCACCATAGGGTGATTGAGGTTGGGAATAATGACATCGATGATGTAAACATC | | NVITSSEYLPTLWVKLYKSE | |
| | | | | ATAGTTTTCCGGCAGATTAATCAATTTGATTTAAGTGGAAATGTGATCACTTC | | NLDNPIQTVSLGQSLFFHFP | |
| | | | | CTCTGAATACCTTCCTACATTATGGGTCAAGCTTTACAAAAGCGAAAACCTCG | | PLLRDGENYVVLLDSTLPR | |
| | | | | ACAATCCAATCCAGACAGTTTCCCTTGGCCAGTCCCTGTTCTTCCATTTCCCC | | SQYDYILPQVSFTAVGYHK | |
| | | | | CCACTGCTCAGAGACGGCGAGAACTATGTTGTGCTTCTGGACTCCACACTCC | | HTTLIFNPTRKLPEQDIAQG | |
| | | | | CCAGATCCCAGTATGACTACATCTTGCCTCAAGTTTCTTTCACCGCAGTGGG | | SYIALPLTLLVLLAGYNHDK | |
| | | | | CTACCATAAACACACCACCTTGATTTTTAATCCCACGAGGAAGCTGCCTGAA | | LIPLLLQLPSRLQGVRALGQ | |
| | | | | CAGGACATCGCACAAGGATCCTACATTGCCCTGCCATTGACGCTGCTGGTTC | | AASDNSGPEDAKRQAKKQ | |
| | | | | TGCTGGCCGGTTACAACCATGACAAGCTCATTCCTTTGCTGCTGCAGTTGAC | | KTRRT* | |
| | | | | AAGCCGGCTACAGGGAGTCCGCGCGCTCGGCCAGGCAGCCTCTGACAATA | | | |
| | | | | GCGGCCCAGAAGATGCAAAGAGACAAGCCAAGAAACAGAAGACAAGGCGGA | | | |
| | | | | CTTGA | | | |
| Shigella | 3 | prey14801 | 65 | CCTGGGCCTACATTCTCCCATTGCCCTAGATGTACTGAGTGAGGCTTTTGAG | 266 | LGLHSPIALDVLSEAFEESL | |
| ospC1 | | | | GAATCCTTGGTGGCCAGAGATTGGTCCCGGGCCCTTCAGCTCACTGAAGTG | | VARDWSRALQLTEVYGRD | |
| | | | | TACGGGCGAGATGTGGACGATTTGAGCAGCATAAAGGATGCAGTCCTGAGC | | VDDLSSIKDAVLSCAVAYD | |
| | | | | TGTGCTGTGGCATATGACAAAGAAGGTTGGCAATACCTGTTTCCCGTGAAGG | | KEGWQYLFPVKDASLRSRL | |
| | | | | ATGCATCTCTGAGAAGTCGGCTGGCCCTACAGTTTGTGGACAGGTGGCCCC | | ALQFVDRWPLESCLEILAY | |
| | | | | TGGAGTCATGCGTGGAGATTCTGGCCTACTGCATTTCAGACACGGCTGTCCA | | CISDTAVQEGLKCELQRKL | |
| | | | | AGAAGGACTAAAGTGTGAGCTACAGAGGAAGCTGGCGGAGCTGCAGGTGTA | | AELQVYQKILGLQSPPVWC | |
| | | | | TCAGAAGATTCTGGGTTTGCAGTCTCCCCCAGTGTGGTGTGACTGGCAGAC | | DWQTLRSCCVEDPSTVMN | |
| | | | | CTTGAGGAGCTGTTGTGTTGAGGACCCATCAACTGTCATGAACATGATTCTA | | MILEAQEYELCEEWGCLYP | |
| | | | | GAAGCACAGGAGTATGAACTGTGTGAAGAGTGGGGCTGCCTGTACCCCATT | | IPREHLISLHQKHLLHLLER | |
| | | | | CCAAGAGAACATTTAATCAGCCTTCATCAAAAGCATCTTCTCCACCTTCTAGA | | RDHDKALQLLRRIPDPTMC | |
| | | | | AAGAAGAGATCATGACAAGGCTCTGCAACTCCTGCGAAGAATCCCTGACCCC | | LEVTEQSLDQHTSLATSHF | |
| | | | | ACCATGTGCCTTGAAGTGACAGAGCAATCCCTCGACCAGCACACTAGCTTGG | | LANYLTTHFYGQLTAVRHR | |
| | | | | CCAGTTCTCACTTCTTGGCCAACTACCTCACCACCCACTTCTATGGACAACTG | | EIQALYVGSKILLTLPEQHR | |
| | | | | ACTGCTGTCCGACACCGTGAAATCCAGGCGCTGTATGTGGGATCCAAGATTC | | ASYSHLSSNPLFMLEQLLM | |
| | | | | TGCTGACCCTGGCTGAGCAGCACCGGGCCAGCTATTCCCACTTGTCCTCTAA | | NMKVDQATVAVQTLQQLL | |
| | | | | CCCCCTGTTCATGCTGGAGCAGCTGCTTATGAACATGAAGGTGGATTGGGC | | VGQEIGFTMDEVDSLLSRY | |
| | | | | CACTGTGGCTGTGCAGACTCTCCAGCAGCTGCTGGTTGGACAGGAGATTGG | | AEKALDFPYPQREKRSDSV | |
| | | | | CTTCACTATGGACGAGGTGGACTCACTGCTTTGCAGATACGCAGAGAAAGCC | | IHLQEIVHQAADPETLPRSP | |
| | | | | CTGGACTTTCCATACCCTCAGAGGGAGAAACGATCAGATTCTGTGATTCACC | | SAEFSPAAPPGISSIHSPSL | |
| | | | | TCCAAGAAATTGTCCACCAGGCTGCAGATCCCGAGACCCTCCCTAGATCACC | | RERSFPPTQPSQEFVPPAT | |
| | | | | ATCAGCAGAGTTCTCTCCTGCTGCTCCTCCTGGTATCTCCAGTATACATTCCC | | PPARHQWVPDETESICMV | |
| | | | | CTAGTCTAAGGGAAAGGAGTTTCCCACCAACCCAGCCCTCACAGGAATTTGT | | CCREFTMFNRRHHCRRC | |
| | | | | GCCCCCAGCGACACCCCCTGCCAGGCACCAGTGGGTACCGGATGAGACTG | | GRLVCSSCSTKKMVVEGC | |
| | | | | AGAGTATCTGCATGGTCTGCTGCAGGGAGCACTTCACCATGTTTAACAGGCG | | RENPARVCDQCYSYCNKD | |
| | | | | TCATCATTGTCGCCGCTGTGGCCGGCTAGTGTGCAGCTCCTGCTCCACTAA | | VPEEPSEKPEALDSSKSES | |
| | | | | GAAAATGGTGGTTGAAGGCTGCAGAGAGAACCCTGCTCGTGTGTGTGATCA | | PPYSFVVRVPKADEVEWIL | |
| | | | | GTGCTATAGTTACTGCAACAAAGATGTACCAGAGGAGCCTTCAGAAAAACCA | | DLKEEENELVRSEFYYEQA | |
| | | | | GAAGCTCTAGACAGCTCCAAGAGTGAAAGCCCTCCATACTCGTTTGTGGTGA | | PSASLCIAILNLHRDSIACG | |
| | | | | GAGTCCCCAAAGCAGATGAGGTGGAATGGATTTTGGATCTCAAAGAGGAGG | | HQLIEHCCRLSKGLTNPEV | |
| | | | | AAAATGAGCTGGTGCGGAGTGAATTTTACTATGAGCAGGCCCCCAGCGCCT | | DAGLLTDIMKQLLFSAKMM | |
| | | | | CCTTGTGCATTGCCATCCTGAATCTGCACCGGGACAGCATTGCCTGTGGTCA | | FVKAGQSQDLALCDSYISK | |
| | | | | CCAGCTGATTGAGCACTGCTGCAGGCTCTCCAAGGGCCTCACCAACCCAGA | | VDVLNILVAAAYRHVPSLD | |
| | | | | GGTGGATGCCGGGCTGCTCACGGACATCATGAAGCAGCTGCTGTTCAGCGC | | QILQPAAVTRLRNQLLEAEY | |
| | | | | CAAGATGATGTTCGTCAAAGCCGGCCAGAGCCAAGACTTGGCTCTTTGTGAC | | YQLGVEVSTKTGLDTTGA | |
| | | | | AGCTACATCAGCAAGGTAGATGTGCTGAATATTTTAGTTGCTGCTGCCTATC | | WHAWGMACLKAGNLTAAR | |
| | | | | GCCACGTGCCATCTTTGGATCAGATCTTGCAGCCAGCTGCAGTAACCAGGCT | | EKFSRCLKPPFDLNQLNHG | |
| | | | | AAGGAACCAGCTTTTGGAAGCCGAGTACTACCAACTGGGCGTTGAGGTCTC | | SRLVQDVVEYLESTVRPFV | |
| | | | | CACAAAGACTGGGCTTGATACCACCGGGGCGTGGCATGCTTGGGGCATGGC | | SLQDDDYFATLRELEATLR | |
| | | | | CTGCCTCAAAGCCGGGAACCTCACTGCTGCACGGGAGAAGTTCAGTCGCTG | | TQSLSLAVIPEGKIMNNTYY | |
| | | | | TCTGAAGCCCCCATTTGACCTCAATCAGCTGAATCATGGCTCAAGGCTGGTG | | QECLFYLHNYSTNLAIISFY | |
| | | | | GAGGATGTGGTTGAGTACCTAGAGTCCACAGTGAGGCCCTTTGTATCCTTGC | | VRHSCLREALLHLLNKESP | |
| | | | | AAGATGACGATTACTTTGCCACCCTGAGGGAACTGGAAGCTACCCTTCGGAC | | PEVFIEGIFQPSYKSGKLHT | |
| | | | | GCAGAGCCTTTCTCTGGCAGTGATTCCTGAAGGGAAAATCATGAACAACACC | | LENLLESIDPTLESWGKYLI | |
| | | | | TACTACCAGGAATGCCTCTTCTACCTGCACAACTATAGCACCAACCTGGCCA | | AACQHLQKKNYYHILYELQ | |
| | | | | TCATCAGCTTCTACGTGAGGCACAGCTGCCTGCGGGAAGCTCTTCTGCACCT | | QFMKDQVRAAMTCIRFFSH | |
| | | | | TCTCAACAAGGAGAGTCCTCCAGAAGTTTTTATAGAAGGCATTTTCCAACCAA | | KAKSYTELGEKLSWLLKAK | |
| | | | | GCTATAAAAGTGGGAAGCTACACACTTTGGAGAACTTGCTAGAATCCATTGA | | DHLKIYLQETSRSSGRKKT | |
| | | | | TCCAACCTTGGAGAGCTGGGGAAAGTACTTGATTGCTGCCTGCCAACATTTA | | TFFRKKMTAADVSRHMNTL | |
| | | | | CAGAAGAAGAACTACTACCACATTCTGTATGAGCTGCAGCAGTTTATGAAGG | | QLQMEVTRFLHRCESAGT | |
| | | | | ACCAAGTTCGGGCCGCCATGACCTGTATTCGGTTCTTCAGTCACAAAGCAAA | | SQITTLPLPTLFGNNHMKM | |
| | | | | GTCATATACAGAACTGGGAGAGAAGCTCTCATGGCTACTTAAGGCCAAGGAC | | DVACKVMLGGKNVEDGFGI | |
| | | | | CACCTGAAGATCTACCTCCAAGAAACATCCCGCAGCTCTGGAAGGAAGAAAA | | AFRVLQDFQLDAAMTYCRA | |
| | | | | CCACATTCTTCAGAAAGAAGATGACTGCAGCTGATGTGTCAAGGCACATGAA | | ARQLVEKEKYSEIQQLLKC | |
| | | | | CACACTTCAGCTGCAGATGGAAGTGACCAGGTTCTTGCATCGGTGCGAAAGT | | VSESGMAAKSDGDTILLNC | |
| | | | | GCTGGGACCTCTCAAATCACCACTTTGCCTCTGCCAACCCTGTTTGGAAATA | | LEAFKRIPPQELEGLIQAIHN | |
| | | | | ACCACATGAAAATGGATGTTGCCTGCAAGGTCATGCTGGGAGGGAAAAATGT | | DDNKVRAYLICCKLRSAYLI | |
| | | | | AGAAGATGGTTTTGGAATTGCTTTCCGTGTTCTGCAGGACTTCCAGCTGGAT | | AVKQEHSRATALVQQVQQ | |
| | | | | GCTGCCATGACCTACTGCAGAGCTGCCCGCCAGTTGGTGGAGAAAGAGAAG | | AAKSSGDAVVQDICAQWLL | |
| | | | | TACAGTGAGATCCAGCAACTGCTCAAATGTGTCAGTGAGTCAGGCATGGCAG | | TSHPRGAHGPGSRK* | |
| | | | | CCAAAAGTGACGGGGACACCATCCTCCTCAACTGCCTGGAAGCGTTCAAGA | | | |
| | | | | GAATTCCGCCCCAGGAGCTGGAGGGCCTGATCCAGGCAATACACAATGATG | | | |
| | | | | ACAACAAGGTTCGGGCCTACCTGATATGTTGCAAACTGCGTTCTGCCTACTT | | | |
| | | | | GATTGCTGTGAAGCAAGAACACTCACGGGCCACAGCCCTTGTCCAGCAGGT | | | |
| | | | | GCAGCAGGCCGCCAAGAGCAGCGGGGATGCAGTAGTGCAAGACATCTGTG | | | |
| | | | | CCCAGTGGCTTCTGACAAGCCACCCCCGGGGTGCCCATGGCCCAGGCTCC | | | |
| | | | | AGGAAGTGA | | | |
| Shigella | 3 | prey67279 | 66 | CTCCCTCTCTGCCTAGCTGGCTTTCTGTAAATAATTATTTGTGTCATAGCTTA | 267 | LPLCLAGFL*IIICGIAYSFLNI | |
| ospC1 | | | | CAGCTTTTTAAACATTTTCACTTTTATTATTTCATTTAATTTTCACACCAGCCCC | | FTFIISFNFHTSPEKCFFHFT | |
| | | | | GAAAAGTGTTTTTTCCACTTTACAAATTAAGATGCAGAAGCTCAGCAATANNN | | N*DAEAQQXXXXXXXXXXX | |
| | | | | NNNNNNNNNNNNNNNNNNNNNNNNNNNNNNNNNNNNNNNNNNNNNNNNNN | | XXXXXXXXXXXXXXXXXXX | |
| | | | | NNNNNNNNNNNNNNNNNNNNNNNNNNNNNNNNNNNGGGGAAGCTCAGCAA | | GEAQQY*MSGPIT*SVS | |
| | | | | TATTAAATGTCTGGGCCAATTACGTAATCAGTAAGC | | | |
| Shigella | 3 | prey67280 | 67 | AATTTCCACCTCCCAAGGGAAGTTTATGTATTTTTCTAGGCCCTTTTCTATGT | 268 | NFHLPREVYVFF*ALFYVFT | |
| ospC1 | | | | CTTTACATCTCTGTCTCACACACACACACGTATACACACACACAGTTTATTTTT | | SLSHTHTRIHTHSLFLIK*DY | |
| | | | | AATAAAATAGGATTATACCACACACATCCTGTCACTTGCTTTTTTGCTTAAGA | | TTHILSLAFLLKSISKRILCVS | |
| | | | | GTATATCTAAGAGAATCCTTTGTGTCAGTGAAGCTGGAGCTACCTCATTCTTT | | EAGATSFF*LAAWRSIECLS | |
| | | | | TAACTGGCTGCGTGGCGTTCCATTGAGTGTCTGTCATCATGTGTTTAGCCGA | | SCV*PSGWIVCLFLVX | |
| | | | | GTGGATGGATAGTCTGCTTGTTTTTAGTTTNTGC | | | |
| Shigella | 3 | prey49194 | 68 | CAACCCCGTGCCCCTCTATGCGCCAAATCTCAGCCCGCCTGCGGACAGCAG | 269 | NPVPLYAPNLSPPADSRIH | |
| ospC1 | | | | GATCCACGTGCCGGCCAGTGGGTACTGCTGCCTGGAGTGTGGAGACGCATT | | VPASGYCCLECGDAFALEK | |
| | | | | TGCCTTAGAGAAGAGCCTGAGCCAGCACTATGGCCGGCGGAGCGTCCACAT | | SLSQHYGRRSVHIEVLCTL | |
| | | | | TGAGGTACTGTGCACACTGTGCTCCAAGACGCTGCTCTTCTTCAACAAGTGC | | CSKTLLFFNKCSLLRHARD | |
| | | | | AGCCTGCTCCGGCACGCCCGTGACCACAAGAGCAAGGGGCTCGTCATGCA | | HKSKGLVMQCSQLLVKPIS | |
| | | | | GTGTTCCCAGCTGCTGGTGAAGCCTATCTCTGCGGACCAAATGTTCGTGTCG | | ADQMFVSAPVNSTAPAAPA | |
| | | | | GCCCCTGTGAACTCCACGGCACCAGCAGCCCCAGCCCCTTCATCCTCTCCC | | PSSSPKHGLTSGSASPPPP | |
| | | | | AAACATGGCCTCACTTCGGGCAGTGCCAGTCCCCCTCCTCCAGCCTTGCCA | | ALPLYPDPVRLIRYSIKCLE | |
| | | | | CTCTACCCAGACCCTGTGAGGCTCATCCGGTACTCAATCAAGTGTCTTGAAT | | CHKQMRDYMVLAAHFQRT | |
| | | | | GTCACAAGCAGATGCGGGACTACATGGTCCTGGCTGCACATTTCCAGAGGA | | TEETEGLTCQVCQMLLPNQ | |
| | | | | CAACAGAGGAGACAGAGGGGCTGACCTGCCAGGTATGCCAGATGCTGCTGC | | CSFCAHQRIHAHKSPYCCP | |
| | | | | CCAACCAGTGCAGTTTCTGTGCCCACCAGCGGATTCATGCACACAAGTCCCC | | ECGVLCRSAYFQTHVKEN | |
| | | | | CTACTGCTGCCCGGAGTGTGGGGTCCTCTGCCGCTCTGCCTACTTCCAGAC | | CLHYARKVGYRCIHCGVVH | |
| | | | | CCATGTAAAGGAGAATTGCCTGCACTATGCCCGCAAGGTGGGCTACAGGTG | | LTLALLKSHIQERHCQVFHK | |
| | | | | CATCCACTGTGGTGTCGTCCACCTGACCTTGGCCTTGCTGAAAAGCCACATC | | CAFCPMAFKTASSTADHSA | |
| | | | | CAGGAGCGACACTGCCAGGTTTTCCACAAATGTGCATTCTGCCCCATGGCCT | | TQHPTQPHRPSQLIYKCSC | |
| | | | | TCAAGACTGCCAGCAGCACTGCAGACCACAGTGCCACCCAGCACCCCACCC | | EMVFNKKRHIQQHFYQNVS | |
| | | | | AGCCCCACAGACCCTCCCAGCTCATTTATAAGTGCTCCTGTGAAATGGTCTT | | KTQVGVFKCPECPLLFVQK | |
| | | | | CAACAAGAAGAGGCACATTCAGCAGCATTTTTACCAGAATGTCAGCAAGACG | | PELMQHVKSTHGVPRNVD | |
| | | | | CAGGTGGGCGTCTTCAAGTGCCCTGAGTGCCCACTCTTGTTCGTGCAGAAG | | ELSNLQSSADTSSSRPGSR | |
| | | | | CCGGAGTTGATGCAACACGTCAAGAGCACCCACGGTGTTCCCCGAAATGTG | | VPTEPPATSVAARSSSLPS | |
| | | | | GACGAGCTGTCAAACCTCCAGTCTTCAGCGGACACATCCTCAAGCCGCCCT | | GRWGRPEAHRRVEARPRL | |
| | | | | GGCTCTCGAGTTCCCACTGAGCCACCAGCCACTAGTGTGGCTGCTCGGAGC | | RNTGWTCQECQEWVPDR | |
| | | | | AGCTCCCTGCCTTCTGGCCGCTGGGGTAGGCCTGAAGCCCACCGCAGGGT | | ESYVSHMKKSHGRTLKRY | |
| | | | | GGAAGCCAGGCCGCGGCTGAGGAACACTGGCTGGACCTGCCAGGAGTGCC | | PCRQCEQSFHTPNSLRKHI | |
| | | | | AGGAGTGGGTTGCAGATCGGGAGAGCTAGGTGTCCCACATGAAAAAGAGCC | | RNNHDTVKKFYTCGYCTE | |
| | | | | ACGGTCGGACATTGAAGCGGTACGCATGCCGGCAGTGTGAACAGTCCTTCC | | DSPSFPRPSLLESHISLMH | |
| | | | | ACACCCCCAACAGCCTGCGCAAACACATCCGCAACAACCATGACACAGTAAA | | GIRNPDLSQTSKVKPPGGH | |
| | | | | GAAGTTCTACACCTGCGGGTACTGCACAGAGGACAGCCCCCAGCTTTCCTCG | | SPQVNHLKRPVSGVGDAP | |
| | | | | GCCCTCCCTTCTGGAGAGCCACATCAGCCTTATGCATGGCATCAGAAACCCT | | GTSNGATVSSTKRHKSLFQ | |
| | | | | GATTTGAGCCAGACGTCCAAAGTGAAACCTCCGGGTGGACATTCCCCTCAG | | CAKCSFATDSGLEFQSHIP | |
| | | | | GTGAACCATCTGAAAAGAOCAGTCAGTGGAGTGGGGGACGCTCCAGGCACC | | QHQVDSSTAQCLLCGLCYT | |
| | | | | AGCAATGGCGCAACTGTCTCTTCCACCAAAAGGCACAAGTCCCTTTTTCAGT | | SASSLSRHLFIVHKVRDQE | |
| | | | | GCGCGAAATGTAGTTTTGCCACAGACTCGGGGCTCGAGTTTCAGAGCCACA | | EEEEEEAAAAEMAVEVAEP | |
| | | | | TACCTCAGCACCAGGTGGACAGCTCCACAGCCCAATGTCTCCTCTGTGGTTT | | EEGSGEEVPMETRENGLE | |
| | | | | GTGCTACACCTCTGCCAGCTCCCTCAGCCGCCACCTCTTCATTGTCCACAAG | | ECAGEPLSADPEARRLLGP | |
| | | | | GTGAGAGACCAGGAGGAGGAGGAGGAAGAGGAGGCGGCGGCAGCGGAGA | | APEDDGGHNDHSQPQASQ | |
| | | | | TGGCAGTGGAGGTGGCAGAGCCAGAGGAGGGCTCCGGGGAGGAGGTGCC | | DQDSHTLSPQV* | |
| | | | | CATGGAGACTAGAGAGAATGGACTGGAAGAATGTGCCGGTGAGCCTTTGTC | | | |
| | | | | AGCTGACCCAGAGGCGAGGAGATTGCTGGGCCCGGCCCCTGAGGACGATG | | | |
| | | | | GTGGCCACAATGATCACAGTCAACCACAGGCCTCTCAGGACCAGGACAGCC | | | |
| | | | | ACACACTGTCCCCTCAGGTGTGA | | | |
| Shigella | 3 | prey67287 | 69 | GAACACTCCTCTAGCTTAGTTATGCTGTTCTTTTAAGTTTGTCTTTGAGTTGG | 270 | EHSSSLVMLFF*VCL*VGKV | |
| ospC1 | | | | GAAAGTAGACCTATTTGGCTTGGCTTAAGGGCTAAATGTCTCCTCTTCACTTG | | DLFGLA*GLNVSSSLGLLILS | |
| | | | | GTCTTCTAATCCTCAGTCCTTCCTGGCTATGTGGCATCATGTCTTTAAAGCAG | | PSWLCGIMSLKQGE*SINIL | |
| | | | | GGAGAGTAAAGTATCAATATTTTAAGAAGGAACATTCTTCCCACTTACGTTTT | | RRNILPTYVFYSSFF*ALSR | |
| | | | | CTATTCTTCTTTCTTTTGAGCCCTTTCTAGAAAGAGTAATGCTCTAGCCTTCAA | | KSNALAFNQK*KVY | |
| | | | | CCAGAAATGAAAAGTCTATG | | | |
| Shigella | 3 | prey19931 | 70 | GGTGCACCAAGTGACAGACCTTTCTAGAAATGCCCAGCTGTTCAAGCGCTCT | 271 | VHQVTDLSRNAQLFKRSLL | |
| ospC1 | | | | TTGCTGGAGATGGCAACGTTCTGA | | | |
| Shigella | 3 | prey67290 | 71 | GGGGGGGTGGGGATGGGGAGGTAATAACNNNATNTTCTTTTGGTANTNATA | 272 | GGVGMGR**XXXLLVXIQC | |
| ospC1 | | | | CAGTGTGGNANTCTCNTNTGAANNNTTCTATNGACNANAAATATCTTTTTTTT | | GXLX*XXLXTXNIFFXSYLS | |
| | | | | NTCTTATCTTTCTNTTGTCTTCTGTGGGAGANGGCTGCTNTNTTTTTTANNGN | | XVFCGRXLLXFLXXLXIFXIS | |
| | | | | CTTTGTNTATTTTTCNTATTAGCAGAATATCAGCNNNNCTGNTNCTNCNATAT | | RISAXLXLXYFMXXXLXXXX | |
| | | | | TTTATGANATANNTGCTTNTAANCNTNTANAATCTGATTAATATTTATNNACTT | | NLINIYXLXLHHIXXIF | |
| | | | | NTTTTACATCATATAGANNATATCTTT | | | |
| Shigella | 3 | prey67291 | 72 | TTTGAAGGGNTCNTANNAACATAGGANAATGTGGCTATAGTTTGGAACCTNC | 273 | FEGXXXT*XNVAIVWNLLHI | |
| ospC1 | | | | TACATATTTGTTGAATGGCTTTGACANACTTGCTGATAGTGATATGAACATTA | C*MALTXLLIVI*TLXSKLRW | | |
| | | | | NNGTCCAAGCTGAGGTGGTCTCAAATGGAGATGAGGAACTTGTTGGGAACT | | SQMEMRNLLGTEXQVTLV | |
| | | | | GAAGNACAGGTGACTCTTGTTATGTTTTANCCAAGACCACTGTCNTCATTTTG | MFXPRPLSSFCLCPXXXW | | |
| | | | | CCTNTGCCCTANANATTTNTGGAACTTTNACNTTGAGANANATGATNCANGAT | | NFXXEXXDXXSWXXXXXX | |
| | | | | CTTGGNNGANGANNTNNNTAANNGNNNTATATTNN | | | |
| Shigella | 3 | prey67294 | 73 | GCAOAAGCCGTCATACCATACCAGGCAGTAAAAATTTACTCCTTAGTTTTCTT | 274 | AQAVIPYQAVKIYSLVFFXK* | |
| ospC1 | | | | CTANAAATAGATTAAGTCTGTGATCCATTTTGGGTTAATTTTTCTGTGATGTAT | | IKSVIHEGLIFL*CILLFEVNF | |
| | | | | ACTATTGTTTGAGGTTAATTTTTTTCTAGTTTTAAAATTTTCATCCAGTTGTTCC | | FLVLKFSSSCSSXXC*ENCX | |
| | | | | AGCNTCNCTTGTTGAGAAAATTGTTNTTCCCATTAANATTACTTTGGATACCT | | SH*XYFGYLX*XXYXXYXVX | |
| | | | | NGNGTGANGNNTATATGNGGNCTATANNGTGTNGNGNAACNCGACGCTGCG | | XNXTLRXVAXRRKXXXXXX | |
| | | | | CAGNGTGGCNTANCGTCGTAAGNNANGTAGNGNANAGNGCCGNGAGA | | RE | |
| Shigella | 3 | prey67296 | 74 | AGAGTGGGGATGGGCTGGGCCTCTGTTCGTCCGTCCGACCCCCCTCATGTG | 275 | RVGMGWASVRPSDPPHVC | |
| ospC1 | | | | TGCTGCCCCAAACCTCGCCGCTCCCTAGTTTGGTATTCTGTGTCCGGCCTGG | | CPKPRRSLVWYSVSGLG** | |
| | | | | GGTAGTAGCTGGACACCAGACTCAATCTTGGGCTCCAGTTCCCGACTTTTCG | | LDTRLNLGLQFPTFRLLWV | |
| | | | | CCTCCTCTGGGTCTGTCCTGGGGTCAGTAATTAACCCGGGTCCCAGGGGTG | | CPGVSN*PGSQGCRLFPP | |
| | | | | TCGTCTTTTCCCTCCAGGGTGGGGCGCTGCCTGTACATGCCAGGATCTTTT | | GWGAACTCQGSFAGLFIXI | |
| | | | | GCAGGGCTTTTCATCCANATTTGCTTCAGGG | | CFR | |
| Shigella | 3 | prey67299 | 75 | CCTCCTCCTCCAACACACGTGCACACAGTGTCTGCCCAATGCCTACTTTTTTT | 276 | PPPPTHVHTVSAQCLLFFF | |
| ospC1 | | | | TTTTAAANGAAANTTTNANTTNGNAANTANAANNNGGNtAAAANGNCNTNNNC | | KXXFXXXXXXXXKXXXXXX | |
| | | | | NTNTANCCTTTTNNNGTTTTTTTTNNTTTTNTTTTTTTNGNTAANNNANNNGTT | | FXXFFXFXFFX*XXXFXKRX | |
| | | | | TTTNAAAAAGGTNNAAAAAAATNTTNACANTTTTNGGGGNTAANCTTTTAATTT | | KKXXTXXGXXLLI*NXXPLN | |
| | | | | AAAACTTNGNCCCCTTAAATTANCCACCNCAANNTANCAAATTTTNAAGGTTT | | XPPQXXKFXRFXKXXLG | |
| | | | | TNAAAAAANNGTTTGGGA | | | |
| Shigella | 3 | prey4637 | 76 | AGCAGAAGGATGATAAAGAACCGCAGCCAGTGAAGAAGACAGTGACAGGAA | 277 | QKDDKEPQPVKKTVTGTD | |
| ospC1 | | | | CAGATGCAGACCTTCGTCGCCTTTCCCTGAAAAATGCCAAGCAACTTCTACG | | ADLRRLSLKNAKQLLRKFG | |
| | | | | TAAATTTGGTGTGCCTGAGGAAGAGATTAAAAAGTTGTCCCGCTGGGAAGTG | | VPEEEIKKLSRWEVIDVVRT | |
| | | | | ATTGATGTGGTGCGCACAATGTCAACAGAACAGGCTCGTTCTGGAGAGGGG | | MSTEQARSGEGPMSKFAR | |
| | | | | CCCATGAGTAAATTTGCCCGTGGATCAAGGTTTTCTGTGGCTGAGCATCAAG | | GSRFSVAEHQERYKEECQ | |
| | | | | AGCGTTACAAAGAGGAATGTCAGOGCATCTTTGACCTACAGAACAAGGTTCT | | RIFDLQNKVLSSTEVLSTDT | |
| | | | | GTCATCAACTGAAGTCTTATOAACTGACACAGACAGCAGCTCAGCTGAAGAT | | DSSSAEDSDFEEMGKNIEN | |
| | | | | AGTGACTTTGAAGAAATGGGAAAGAACATTGAGAACATGTTGCAGAACAAGA | | MLQNKKTSSQLSREREEQ | |
| | | | | AAACCAGCTCTCAGCTTTCACGTGAACGGGAGGAACAGGAGCGGAAGGAAC | | ERKELQRMLLAAGSAASG | |
| | | | | TACAGCGAATGCTACTGGCAGCAGGCTCAGCAGCATCCGGAAACAATCACA | | NNHRDDDTASVTSLNSSAT | |
| | | | | GAGATGATGACACAGCTTCCGTGACTAGCCTTAACTCTTCTGCCACTGGACG | | GRCLKIYRTFRDEEGKEYV | |
| | | | | CTGTCTCAAGATTTATCGCACGTTTCGAGATGAAGAGGGGAAAGAGTATGTT | | RCETVRKPAVIDAYVRIRTT | |
| | | | | CGCTGTGAGACAGTCCGAAAACCAGCTGTCATTGATGCCTATGTGCGCATAC | | KDEEFIRKFALFDEQHREE | |
| | | | | GGACTACAAAAGATGAGGAATTCATTCGAAAATTTGCCCTTTTTGATGAACAA | | MRKERRRIQEQLRRLKRN | |
| | | | | CATCGGGAAGAGATGCGAAAAGAACGGCGGAGGATTCAAGAGCAACTGAGG | | WEKEKLKGPPEKKPKKMKE | |
| | | | | CGGCTTAAGAGGAACCAGGAAAAGGAGAAGCTTAAGGGTCCTCCTGAGAAG | | RPDLKLKCGACGAIGHMRT | |
| | | | | AAGCCCAAGAAAATGAAGGAGCGTCCTGACCTAAAACTGAAATGTGGGGCAT | | NKFCPLYYQTNAPPSNPVA | |
| | | | | GTGGTGCCATTGGACACATGAGGACTAACAAATTCTGCCCCCTCTATTATCA | | MTEEQEEELEKTVIHNDNE | |
| | | | | AACAAATGCGCCACCTTCCAACCCTGTTGCCATGACAGAAGAACAGGAGGA | | ELIKVEGTKIVLGKQLIESAD | |
| | | | | GGAGTTGGAAAAGACAGTCATTCATAATGATAATGAACTTATCAAGGTTG | | EVRRKSLVLKFPKQQLPPK | |
| | | | | AAGGGACCAAAATTGTCTTGGGGAAACAGCTAATTGAGAGTGCGGATGAGG | | KKRRVGTTVHCDYLNRPH | |
| | | | | TTCGCAGAAAATCTCTGGTTCTCAAGTTTCCTAAACAGCAGCTTCCTCCAAAG | | KSIHRRRTDPMVTLSSILESI | |
| | | | | AAGAAACGGCGAGTTGGAACGACTGTTCAGTGTGACTATTTGAATAGACCTC | | INDMRDLPNTYPFHTPVNA | |
| | | | | ATAAGTCCATCCACCGGCGCCGCACAGACCCTATGGTGACGCTGTCGTCCA | | KVVKDYYKIITRPMDLQTLR | |
| | | | | TCTTGGAGTCTATCATCAATGACATGAGAGATCTTCCAAATACATACCCTTTC | | ENVRKRLYPSREEFREHLE | |
| | | | | CACACTCCAGTCAATGCAAAGGTTGTAAAGGACTACTACAAAATCATCACTC | | LIVKNSATYNGPKHSLTQIS | |
| | | | | GGCCAATGGACCTACAAACACTCCGCGAAAACGTGCGTAAACGCCTCTACC | | QSMLDLCDEKLKEKEDKLA | |
| | | | | CATCTCGGGAAGAGTTCAGAGAGCATCTGGAGCTAATTGTGAAAAATAGTGC | | RLEKAINPLLDDDDQVAFSF | |
| | | | | AACCTACAATGGGCCAAAACACTCATTGACTCAGATCTCTCAATCCATGCTG | | ILDNIVTQKMMAVPDSWPF | |
| | | | | GATCTCTGTGATGAAAAACTCAAAGAGAAAGAAGACAAATTAGCTCGCTTAG | | HHPVNKKFVPDYYKVIVNP | |
| | | | | AGAAAGCTATCAACCCCTTGCTGGATGATGATGACCAAGTGGCGTTTTCTTT | | MDLETIRKNISKHKYQSRES | |
| | | | | CATTCTGGACAACATTGTCACCCAGAAAATGATGGCAGTTCCAGATTCTTGG | | FLDDVNLILANSVKYNGPES | |
| | | | | CCATTTCATCACCCAGTTAATAAGAAATTTGTTCCAGATTATTACAAAGTGATT | | QYTKTAQEIVNVCYQTLTE | |
| | | | | GTCAATCCAATGGATTTAGAGACCATACGTAAGAACATCTCCAAGCACAAGT | | YDEHLTQLEKDICTAKEAAL | |
| | | | | ATCAGAGTCGGGAGAGCTTTCTGGATGATGTAAACCTTATTCTGGCCAACAG | | EEAELESLDPMTPGPYTPQ | |
| | | | | TGTTAAGTATAATGGACCTGAGAGTCAGTATACTAAGACTGCCCAGGAGATT | | PPDLYDTNTSLSMSRDASY | |
| | | | | GTGAACGTCTGTTACCAGACATTGACTGAGTATGATGAACATTTGACTCAACT | | FQDESNMSVLDIPSATPEK | |
| | | | | TGAGAAGGATATTTGTACTGCTAAAGAAGCAGCTTTGGAGGAAGCAGAATTA | | QVTQEGEDGDGDLADEEE | |
| | | | | GAAAGCCTGGACCCAATGACCCCAGGGCCCTACACGCCTCAGCCTCCTGAT | | GTVQQPQASVLYEDLLMSE | |
| | | | | TTGTATGATACCAACACATCCCTCAGTATGTCTCGAGATGCCTCTGTATTTCA | | GEDDEEDAGSDEEGDNPF | |
| | | | | AGATGAGAGCAATATGTCTGTCTTGGATATTCCCAGTGCCACTCCAGAAAAG | | SAIQLSESGSDSDVGSGGI | |
| | | | | CAGGTAACACAGGAAGGTGAAGATGGAGATGGTGATCTTGCAGATGAAGAG | | RPKQPRMLQENTRMDMEN | |
| | | | | GAAGGAACTGTACAACAGCCTCAAGCCAGTGTCCTGTATGAGGATTTGCTTA | | EESMMSYEGDGGEASHGL | |
| | | | | TGTCTGAAGGAGAAGATGATGAGGAAGATGCTGGGAGTGATGAAGAAGGAG | | EDSNISYGSYEEPDPKSNT | |
| | | | | ACAATCCTTTCTCTGCTATCCAGCTGAGTGAAAGTGGAAGTGACTCTGATGT | | QDTSFSSIGGYEVSEEEED | |
| | | | | GGGATCTGGTGGAATAAGACCCAAACAACCCCGCATGCTTCAGGAGAACAC | | EEEEEQRSGPSVLSQVHLS | |
| | | | | AAGGATGGACATGGAAAATGAAGAAAGCATGATGTCCTATGAGGGAGACGG | | EDEEDSEDFHSIAGDSDLD | |
| | | | | TGGGGAGGCTTCCCATGGTTTGGAGGATAGCAACATCAGTTATGGGAGCTAT | | SDE* | |
| | | | | GAGGAGCCTGATCCCAAGTCGAACACCCAAGACACAAGCTTCAGCAGCATC | | | |
| | | | | GGTGGGTATGAGGTATCAGAGGAGGAAGAAGATGAGCGAGGAGGAAGAGCA | | | |
| | | | | GCGCTCTGGGCCGAGCGTACTAAGCCAGGTCCACCTGTCAGAGGACGAGG | | | |
| | | | | AGGACAGTGAGGATTTCCACTCCATTGCTGGGGACAGTGACTTGGACTCTGA | | | |
| | | | | TGAATGA | | | |
| Shigella | 3 | prey67316 | 77 | CCACTCTACTCCACAAGGCTCATTCTAACTTCCCCCCTTGCTTATTTGTAACT | 278 | PLYSTRLILTSPLAYL*LFSL | |
| ospC1 | | | | TTTTTCTCTGAGAGTGAGACCCCAACTTTCATTATCTACAACATATCTATCTAT | | RVRPQLSLSTTYLSIYYTCS | |
| | | | | TTATTATACTTGTAGTTTCAAAATTACTGAGAAACAAATTTACTACCTAGAATA | | FKITEKQIYYLEYCVNIQFSL | |
| | | | | CTGTGTTAATATACAATTTTCTTTAGTTTTACAGTATCCAGTCAAAAGGCTGTC | | VLQYPVKRLSSKIA*VSSFS | |
| | | | | TTCCAAAATTGCTTAGGTCAGCTCCTTCTCCATGCAACTCTTTCAGTGAGGCT | | MQLFQ*GXIMRL*YC* | |
| | | | | GNATCATGCGTTTGTAATATTGTTAGAT | | | |
| Shigella | 3 | prey67318 | 78 | CCACCGCACCTGACCTTAGTTTTTTTCTGACGTGGTCCTCTTCTTTTATCTCT | 279 | PPHLTLVFF*RGPLLLSLRL | |
| ospC1 | | | | AAGACTTATGATTGCTAAGACAACAAAAGATACCATCGTTACTGGCCAACCTT | | MIAKTTKDTIVTGQPWNLVL | |
| | | | | GGAATTTGGTCTTGGGAAATGGAGGCCTGTAGTTTGTAACCCATAAGAAGAG | | GNGGL*FVTHKKRLKGPKC | |
| | | | | ACTGAAGGGGCCTAAGTGCAGATGAGAATCCCTGGTGATAGAACAGACAAG | | R*ESLVIEQTRTGDQCQ*FV | |
| | | | | AACTGGAGATCAATGCCAATAGTTTGTGATGAACGTCTTGGGGTTCCTGTGT | | MNVLGFLCDQPVGISV | |
| | | | | GATCAACCTGTTGGGATTTCTGTATT | | | |
| Shigella | 3 | prey7144 | 79 | GGAAGCCAGAAAAGCCCACCAACTCTGGCTTTCAGTGGAGGCATTAAAGTAC | 280 | EARKAHQLWLSVEALKYS | |
| ospC1 | | | | AGCATGAAGACCTCATCTGCAGAAACACCTACTATCCCGCTGGGTAGTGCAG | | MKTSSAETPTIPLGSAVEAI | |
| | | | | TTGAGGCCATCAAAGCCAACTGTTCTGATAATGAATTCACCCAAGCTTTAACC | | KANCSDNEFTQALTAAIPP | |
| | | | | GCAGCTATCCCTCCAGAGTCCCTGACCCGTGGGGTGTACAGTGAAGAGACC | | ESLTRGVYSEETLRARFYA | |
| | | | | CTTAGAGCCCGTTTCTATGCTGTTCAAAAACTGGCCCGAAGGGTAGCAATGA | | VQKLARRVAMIDETRNSLY | |
| | | | | TTGATGAAACCAGAAATAGCTTGTACCAGTACTTCCTCTCCTACCTACAGTCC | | QYFLSYLQSLLLFPPQQLK | |
| | | | | CTGCTCCTATTCCCACCTCAGCAACTGAAGCCGCCCCCAGAGCTCTGCCCT | | PPPELCPEDINTFKLLSYAS | |
| | | | | GAGGATATAAACACATTTAAATTACTGTCATATGCTTCCTATTGCATTGAGCAT | | YCIEHGDLELAAKFVNQLK | |
| | | | | GGTGATCTGGAGCTAGCAGCAAAGTTTGTCAATCAGCTGAAGGGGGAATCC | | GESRRVAQDWLKEARMTL | |
| | | | | AGACGAGTGGCACAGGACTGGCTGAAGGAAGCCCGAATGACCCTAGAAACG | | ETKQIVEILTAYASAVGIGTT | |
| | | | | AAACAGATAGTGGAAATCCTGACAGCATATGCCAGCGCCGTAGGAATAGGAA | | QVQPE* | |
| | | | | CCACTCAGGTGCAGCCAGAGTGA | | | |
| Shigella | 3 | prey67328 | 80 | ATGAAATCCCAATGGTGTAGACCAGTGGCGATGGATCTAGGAGTTTACCAAC | 281 | MKSQWCRPVAMDLGVYQL | |
| ospC1 | | | | TGAGACATTTTTCAATTTCTTTCTTGTCATCCTTGCTGGGGACTGAAAACGCT | | RHFSISFLSSLLGTENASVR | |
| | | | | TCTGTGAGACTTGATAATAGCTCCTCTGGTGCAAGTGTGGTAGCTATTGACA | | LDNSSSGASVVAIDNKIEQA | |
| | | | | ACAAAATCGAGCAAGCTATGGATCTAGTGAAAAGCCATTTGATGTATGCGGT | | MDLVKSHLMYAVREEVEVL | |
| | | | | CAGAGAAGAAGTGGAGGTCCTCAAAGAGCAAATCAAAGAACTAATAGAGAAA | | KEQIKELIEKNSQLEQENNL | |
| | | | | AATTCCCAGCTGGAGCAGGAGAACAATCTGCTGAAGACACTGGCCAGTCCT | | LKTLASPEQLAQFQAQLQT | |
| | | | | GAGCAGCTTGCCCAGTTTCAGGCCCAGCTGCAGACTGGCTCCCCCCCTGCC | | GSPPATTQPQGTTQPPAQ | |
| | | | | ACCACCCAGCCACAGGGCACCACACAGCCCCCCGCCCAGCCAGCATCGCA | | PASQGSGPTA* | |
| | | | | GGGCTCAGGACCAACCGCATAG | | | |
| Shigella | 3 | prey37430 | 81 | GTGGGAACAAGAGCTATACAATAACTTTGTATATAATAGTCCTAGAGGATATT | 282 | WEQELYNNFVYNSPRGYF | |
| ospC1 | | | | TTCATACCTTTGCTGGAGATACTTGTCAAGTTGCTCTTAATTTTGCCAATGAA | | HTFAGDTCQVALNFANEEE | |
| | | | | GAAGAAGCAAAAAAATTTCGAAAAGCAGTTACAGACCTTTTGGGCCGTCGAC | | AKKFRKAVTDLLGRRQRKS | |
| | | | | AAAGGAAATCTGAGAAAAGACGAGATCCCCCAAATGGTCCTAATCTACCCAT | | EKRRDPPNGPNLPMATVDI | |
| | | | | GGCTACAGTTGATATAAAAAATCCAGAAATCACAACAAATAGATTTTATGGTC | | KNPEITTNRFYGPQVNNISH | |
| | | | | CACAAGTCAACAACATCTCCCATACCAAAGAAAAGAAGAAGGGAAAAGCTAA | | TKEKKKGKAKKKRLTKGDI | |
| | | | | AAAGAAGAGATTAACCAAGGGAGATATAGGAACACCAAGCAATTTCCAGCAC | | GTPSNFQHIGHVGWDPNT | |
| | | | | ATTGGACATGTTGGTTGGGATCCAAATACAGGCTCTGATCTGAATAATTTGGA | | GSDLNNLDPELKNLFDMCG | |
| | | | | TCCAGAATTGAAGAATCTTTTTGATATGTGTGGAATCTTAGAGGCACAACTTA | | ILEAQLKERETLKVIYDFIEK | |
| | | | | AAGAAAGAGAAACATTAAAAGTTATATATGACTTTATTGAAAAAACAGGAGGT | | TGGVEAVKNELRRQAPPP | |
| | | | | GTTGAAGCTGTTAAAAATGAACTGCGGAGGCAAGCACCACCACCACCTCCACCAC | | PPPSRGGPPPPPPPPHSS | |
| | | | | CATCAAGGGGAGGGCCACCTCCTCCTCCTCCCCCTCCACATAGCTCGGGTC | | GPPPPPARGRGAPPPPPS | |
| | | | | CTCCTCCTCCTCCTGCTAGGGGAAGAGGCGCTCCTCCCCCACCACCTTCAA | | RAPTAAPPPPPPSRPSVEV | |
| | | | | GAGCTCCCACAGCTGCACCTCCACCACCGCCTCCTTCCAGGCCAAGTGTAG | | PPPPPNRMYPPPPPALPSS | |
| | | | | AAGTCCCTCCACCACCGCCAAATAGGATGTACCCTCCTCCACCTCCAGCCCT | | APSGPPPPPPSVLGVGPVA | |
| | | | | TCCCTCCTCAGCACCTTCAGGGCCTCCACCACCACCTCCATCTGTGTTGGG | | PPPPPPPPPPPGPPPPPGL | |
| | | | | GGTAGGGCCAGTGGCACCACCCCCACCGCCTCCACCTCCACCTCCTCCTGG | | PSDGDHQVPTTAGNKAALL | |
| | | | | GCCACCGCCCCCGCCTGGCCTGCCTTCTGATGGGGACCATCAGGTTCCAAC | | DQIREGAQLKKVEQNSRPY | |
| | | | | TACTGCAGGAAACAAAGCAGCTCTTTTAGATCAAATTAGAGAGGGTGCTCAG | | SCSGRDALLDQIRQGIQLK | |
| | | | | CTAAAAAAAGTGGAGCAGAACAGTCGGCCAGTGTCCTGCTCTGGACGAGAT | | SVADGQESTPPTPAPTSGI | |
| | | | | GCACTGTTAGACCAGATACGACAGGGTATCCAACTAAAATCTGTGGCTGATG | | VGALMEVMQKRSKAIHSSD | |
| | | | | GCCAAGAGTCTACACCACCAACACCTGCACCCACTTCAGGAATTGTGGGTG | | EDEDEDDEEDFEDDDEWE | |
| | | | | CATTAATGGAAGTGATGCAGAAAAGGAGCAAAGCCATTCATTCTTCAGATGA | | D* | |
| | | | | AGATGAAGATGAAGATGATGAAGAAGATTTTGAGGATGATGATGAGTGGGAA | | | |
| | | | | GACTGA | | | |
| Shigella | 3 | prey67351 | 82 | ATTGCCTTCCATGTCTACTGTGATTCAGCTTTGGGAAGATATTTTCTGTTCCT | 283 | IAFHVYCDSALGRYFLFLLL | |
| ospC1 | | | | TTTGCTGCTTTGACTCCCTGCCGCGCCCCCCTTACTTACGCTTCAAATCTGC | | L*LPAAPPLLTLQICLPGFPF | |
| | | | | CTACGAGGTTTTCCATTTCCAGGCAGTCTTTTCTAATTTTTTCCACCTGGAAG | | PGSLF*FFPPGRNFLFSEFV | |
| | | | | AAACTTTCTTTTCTCTGAGTTCGTAATCTTATAATAAGTACCTATTTTTCTCTTC | | IL**VPIFLFF*RI*NVLSDVSS | |
| | | | | TTCTAGCGTATATAAAATGTATTATCTGACGTGTCAAGTGAGTTAATGCATTTA | | ELMHLKSLGMVP | |
| | | | | AAGAGCCTAGGAATGGTACCTAC | | | |
| Shigilla | 3 | prey67353 | 83 | GGAGAAGAGAGGGAGCAACTCGGTATTTGTCCACAAAAAGAGTATTATTCCA | 284 | EKRGSNSVFVHKKSIIPEEE | |
| ospC1 | | | | GAGGAAGAGTGTTATATAAATTGTGTTTTCCAATAAAAATAGTGATGTCTATC | | CYINCVFQ*K**CLSVQCTW | |
| | | | | AGTTCAGTGTACATGGACCTTTGCAGTGAGTCAGAGATTTGGCCTAGGCCTG | | RFAVSQRFGLGLWGISLGE | |
| | | | | TGGGGGATATCCCTGGGAGAAACTGTCTTGTCAAAGGAAGTTAGCATTTGAG | | TVLSKEVSI*DDGMIFAHLS | |
| | | | | ACGATGGCATGATCTTTGCCCACTTATCCCATCAAAAAGAGTTTTGAAAGGAT | | HQKEF*KDSXEALI*XATL | |
| | | | | AGCANGGAAGCATTGATATGANAGGCTACTCTCA | | | |
| Shigella | 3 | prey25185 | 84 | GGCTGCCCTGCCTGATGACATCCGTCGGGAAGTTCTACAGAACCAGCTAGG | 285 | AALPDDIRREVLQNQLGIRP | |
| ospC1 | | | | CATTCGTCCACCAACCCGGACTGCCCCCTCCACAAATAGCTCAGCGCCTGC | | PTRTAPSTNSSAPAVVGNP | |
| | | | | AGTGGTGGGGAATCCTGGTGTGACTGAAGTGAGCCCTGAGTTTCTGGCTGC | | GVTEVSPEFLAALPPAIQEE | |
| | | | | CCTGCCTCCAGCCATTCAGGAGGAAGTACTGGCACAGCAGAGAGCTGAGCA | | VLAQQRAEQQRRELAQNA | |
| | | | | GCAGCGACGAGAACTAGCACAGAATGCCAGCTCAGACACCCCTATGGACCC | | SSDTPMDPVTFIQTLPSDL | |
| | | | | TGTGACCTTCATCCAGACTCTGCCCTCAGACCTGCGCCGTAGTGTCCTAGAG | | RRSVLEDMEDSVLAVMPP | |
| | | | | GATATGGAGGACAGTGTGTTAGCTGTGATGCCACCTGACATTGCAGCTGAG | | DIAAEAQALRREQEARQRQ | |
| | | | | GGTCAAGCCCTGAGACGAGAGCAAGAAGCCCGGCAGCGACAGCTCATGCAT | | LMHERLFGHSSTSALSAILR | |
| | | | | GAGCGTCTGTTTGGGCACAGTAGCACCTCCGCACTCTCTGCTATTCTCCGAA | | SPAFTSRLSGNRGVQYTRL | |
| | | | | GCCCGGCTTTCACCAGTCGCTTAAGTGGCAACCGTGGGGTCCAGTATACTC | | AVQRGGTFQMGGSSSHNR | |
| | | | | GCCTTGCTGTGCAGAGAGGTGGCACCTTCCAGATGGGGGGTAGCAGCAGC | | PSGSNVDTLLRLRGRLLLD | |
| | | | | CATAACAGGCCTTCTGGCAGTAATGTAGATACTCTCCTCCGCCTCCGAGGAC | | HEALSCLLVLLFVDEPKLNT | |
| | | | | GGCTCCTTCTGGACCACGAAGCCCTTTCTTGTCTCTTGGTCCTACTTTTTGTG | | SRLHRVLRNLCYHAQTRH | |
| | | | | GATGAGCCAAAGCTCAATACTAGCCGTCTACACCGAGTACTGAGAAATCTCT | | WVIRSLLSILQRSSESELCIE | |
| | | | | GCTACCATGCCCAGACCCGCCACTGGGTCATCCGCAGTCTGCTCTCCATCTT | | TPKLTTSEEKGKKSSKSCG | |
| | | | | GCAGCGCAGCAGTGAGAGTGAGCTATGCATTGAAACACCCAAACTCACTACA | | SSSHENRPLDLLHKMESKS | |
| | | | | AGTGAGGAAAAGGGCAAAAAGTCGAGCAAGAGCTGTGGGTCAAGTAGCCAT | | SNQLSWLSVSMDAALGCR | |
| | | | | GAGAACCGTCCCCTGGACCTGCTACACAAGATGGAGTCAAAGAGCTCCAAC | | TNIFQIQRSGGRKHTEKHA | |
| | | | | CAGCTTTCCTGGCTCTCAGTATCCATGGATGCAGCCCTAGGCTGCAGGACTA | | SGGSTVHIHPQAAPVVCRH | |
| | | | | ATATATTTCAGATCCAGCGTTCAGGGGGGCGTAAACATACCGAGAAGCATGC | | VLDTLIQLAKVFPSHFTQQR | |
| | | | | AAGCGGTGGCTCCACCGTCCACATCCATCCCCAAGCTGCTCCTGTTGTCTG | | TKETNCESDRERGNKACS | |
| | | | | CAGACACGTTTTGGATACACTCATTCAATTGGCCAAGGTATTTCCCAGCCACT | | PCSSQSSSSGICTDFWDLL | |
| | | | | TCACACAGCAGCGGACCAAAGAAACAAACTGTGAGAGTGATCGGGAAAGGG | | VKLDNMNVSRKGKNSVKS | |
| | | | | GCAATAAGGCCTGTAGCCCATGCTCCTCACAGTCCTCCAGCAGTGGCATTTG | | VPVSAGGEGETSPYSLEAS | |
| | | | | CACAGACTTCTGGGACTTATTGGTAAAACTGGACAACATGAATGTCAGCCGG | | PLGQLMNMLSHPVIRRSSL | |
| | | | | AAAGGCAAGAACTCCGTGAAGTCAGTGCCAGTGAGCGCTGGCGGTGAGGG | | LTEKLLRLLSLISIALPENKV | |
| | | | | GGAAACCTCTCCATACAGCCTCGAGGCCTCTCCACTGGGGCAGCTCATGAA | | SEAQANSGSGASSTTTATS | |
| | | | | CATGTTGTCACACCCAGTCATCCGCCGGAGCTCTCTCTTAACTGAGAAACTC | | TTSTTTTTAASTTPTPPTAP | |
| | | | | CTCAGACTCCTTTCTCTCATCTCAATTGCTCTCCCAGAAAACAAGGTGTCAGA | | TPVTSAPALVAATAISTIVVA | |
| | | | | AGCACAGGCTAATTCTGGCAGCGGTGCTTCCTCCACCACCACTGCCACCTC | | ASTTVTTPTTATTTVSISPT | |
| | | | | AACCACATCTACCACCACCACCACTGCCGCCTCCACCACGCCCACACCCCC | | TKGSKSPAKVSDGGSSST | |
| | | | | TACTGCACCCACCCCTGTCACTTCTGCTCCAGCCCTGGTTGCTGCCACGGCT | | DFKMVSSGLTENQLQLSVE | |
| | | | | ATTTCCACCATTGTCGTAGCTGCTTCGACCACAGTGACTACCCCCACGACTG | | VLTSHSCSEEGLEDAANVL | |
| | | | | CTACCACTACTGTTTCAATTTCTCCCACTACTAAGGGCAGCAAATCTCCAGCG | | LQLSRGDSGTRDTVLKLLL | |
| | | | | AAGGTGAGTGATGGGGGCAGCAGCAGTACAGACTTTAAGATGGTGTCCTCT | | NGARHLGYTLCKQIGTLLA | |
| | | | | GGCCTCACTGAAAACCAGCTACAGCTCTCTGTAGAGGTGTTGACATCCCACT | | ELREYNLEQQRRAQCETLS | |
| | | | | CTTGTTCTGAGGAAGGCTTAGAGGATGCAGCCAACGTACTACTGCAGCTCTC | | PDGLPEEQPQTTKLKGKM | |
| | | | | CCGGGGGGACTCTGGGACCCGGGACACTGTTCTCAAGCTGCTACTGAATGG | | QSRFDMAENVVIVASQKRP | |
| | | | | AGCCCGCCATCTGGGTTATACCCTTTGTAAACAAATAGGTACCCTGCTGGCC | | LGGRELQLPSMSMLTSKTS | |
| | | | | GAGCTGCGGGAATACAACCTCGAGCAGCAGCGGCGAGCCCAATGTGAAACC | | TQKFFLRVLQVIIQLRDDTR | |
| | | | | CTCTCTCCTGATGGCCTGCCTGAGGAGCAGCCACAGACCACCAAGCTGAAG | | RANKKAKQTGRLGSSGLG | |
| | | | | GGCAAAATGCAGAGCAGGTTTGACATGGCTGAGAATGTGGTAATTGTGGCAT | | SASSIQAAVRQLEAEADAII | |
| | | | | CTCAGAAGCGACCTTTGGGTGGCCGGGAGCTCCAGCTGCCTTCTATGTCCA | | QMVREGQRARRQQQAAT | |
| | | | | TGTTGACATCCAAGACATCTACCCAGAAGTTCTTCTTGAGGGTACTACAGGT | | SESSQSEASVRREESPMD | |
| | | | | CATCATCCAGCTCCGGGACGACACGCGCCGGGCTAACAAGAAAGCCAAGCA | | VDQPSPSAQDTQSIASDGT | |
| | | | | GACAGGCAGGCTAGGTTCCTCCGGTTTAGGCTCAGCTAGCAGCATCCAGGC | | PQGEKEKEERPPELPLLSE | |
| | | | | AGCTGTTCGGCAGCTGGAGGCTGAGGCTGATGCCATTATACAAATGGTACG | | QLSLDELWDMLGECLKELE | |
| | | | | TGAGGGTCAAAGGGCGCGGAGACAGCAACAAGCAGCAACGTCGGAGTCTA | | ESHDQHAVLVLQPAVEAFF | |
| | | | | GCCAGTCAGAGGCGTCTGTCCGGAGGGAGGAATCACCCATGGATGTGGAC | | LVHATERESKPPVRDTRES | |
| | | | | CAGCCATCTCCCAGTGCTCAAGATACTCAATCCATTGCCTCCGATGGAACCC | | QLAHIKDEPPPLSPAPLTPA | |
| | | | | CACAGGGGGAGAAGGAAAAGGAAGAAAGACCACCTGAGTTACCCCTGCTCA | | TPSSLDPFFSREPSSMHIS | |
| | | | | GCGAGCAGCTGAGTTTGGACGAGCTGTGGGACATGCTTGGGGAGTGTCTAA | | SSLPPDTQKFLRFAETHRT | |
| | | | | AGGAACTAGAGGAATCCCATGACCAGCATGCGGTGCTAGTGCTACAGCCTG | | VLNQILRQSTTHLADGPFA | |
| | | | | CTGTCGAGGCCTTCTTTCTGGTCCATGCCACAGAGCGGGAGAGCAAGCCTC | | VLVDYIRVLDFDVKRKYFR | |
| | | | | CTGTCCGAGACACCCGTGAGAGCCAGCTGGCACACATCAAGGACGAGCCTC | | QELERLDEGLRKEDMAVH | |
| | | | | CTCCACTCTCCCCTGCCCCCTTAACCCCAGCCACGCCTTCCTCCCTTGACCC | | VRRDHVFEDSYRELHRKSP | |
| | | | | ATTCTTCTCCCGGGAGCCCTCATCTATGCACATCTCCTCAAGCCTGCCCCCT | | EEMKNRLYIVFEGEEGQDA | |
| | | | | GACACACAGAAGTTCCTTCGCTTTGCAGAGACTCACCGCACTGTGTTAAACC | | GGLLREWYMIISREMFNPM | |
| | | | | AGATCCTACGGCAGTCCACGACCCACCTTGCTGATGGGCCTTTTGCTGTCCT | | YALFRTSPGDRVTYTINPSS | |
| | | | | GGTAGACTACATTCGTGTCCTCGACTTTGATGTCAAGCGCAAATATTTCCGC | | HCNPNHLSYFKFVGRIVAK | |
| | | | | CAAGAGCTGGAGCGTTTAGATGAGGGGCTCCGGAAAGAAGACATGGCTGTG | | AVYDNRLLECYFTRSFYKHI | |
| | | | | CATGTCCGTCGTGACCATGTGTTTGAAGACTCCTATCGTGAGCTGCATCGCA | | LGKSVRYTDMESEDYHFY | |
| | | | | AATCCCCCGAAGAAATGAAGAATCGATTGTATATAGTATTTGAAGGAGAAGAA | | QGLVYLLENDVSTLGYDLT | |
| | | | | GGGCAGGATGCTGGCGGGCTCCTGCGGGAGTGGTATATGATCATCTCTCGA | | FSTEVQEFGVCEVRDLKPN | |
| | | | | GAGATGTTTAACCCTATGTATGCCTTGTTCCGTACCTCACCTGGTGATCGAG | | GANILVTEENKKEYVHLVC | |
| | | | | TCACCTACACCATCAATCCATCTTCCCACTGCAACCCCAACCACCTCAGCTA | | QMRMTGAIRKQLAAFLEGF | |
| | | | | CTTCAAGTTTGTCGGACGCATTGTGGCCAAAGCTGTATATGACAACCGTCTT | | YEIIPKRLISIFTEQELELLIS | |
| | | | | CTGGAGTGCTACTTTACTCGATCCTTTTACAAACACATCTTGGGCAAGTCAGT | | GLPTIDIDDLKSNTEYHKYQ | |
| | | | | CAGATATACAGATATGGAGAGTGAAGATTACCACTTCTACCAAGGTCTGGTTT | | SNSIQIQWFWRALRSFDQA | |
| | | | | ATCTGCTGGAAAATGATGTCTCCACACTAGGCTATGACCTCACCTTCAGCAC | | DRAKFLQFVTGTSKVPLQG | |
| | | | | TGAGGTCCAAGAGTTTGGAGTTTGTGAAGTTCGTGACCTCAAACCCAATGGG | | FAALEGMNGIQKFQIHRDD | |
| | | | | GCCAACATCTTGGTAACAGAGGAGAATAAGAAGGAGTATGTACACCTGGTAT | | RSTDRLPSAHTCFNQLDLP | |
| | | | | GCCAGATGAGAATGACAGGAGCCATCCGCAAGCAGTTGGCGGCTTTCTTAG | | AYESFEKSATCYCWLSRSA | |
| | | | | AAGGCTTCTATGAGATCATTCCAAAGCGCCTCATTTCCATCTTCACTGAGCAG | | LKALGWPNKALPNSVGFFL | |
| | | | | GAGTTAGAGCTGCTTATATCAGGACTGCCCACCATTGACATCGATGATCTGA | | PLLDLGRGELKKEPERNCQ | |
| | | | | AATCCAACACTGAATACCACAAGTACCAGTCCAACTCTATTCAGATCCAGTG | | KPINEIHQLTVCVPAAPSSP | |
| | | | | GTTCTGGAGAGCATTGCGTTCTTTCGATCAAGCTGACCGTGCCAAGTTCCTC | | AHTCSSSHSLPAACFLTFS | |
| | | | | CAGTTTGTCACGGGTACTTCCAAGGTACCCCTGCAAGGCTTTGCTGCCCTCG | | PLSMPSMIPTPCVLKRQ* | |
| | | | | AAGGCATGAATGGCATTCAGAAGTTTCAGATCCATCGAGATGACAGGTCCAC | | | |
| | | | | AGATCGCCTGCCTTCAGCTCACACATGTTTTAATCAGCTGGATCTGCCTGCC | | | |
| | | | | TATGAGAGCTTTGAGAAGTCCGCCACATGCTACTGTTGGCTATCCAGGAGTG | | | |
| | | | | CTCTGAAGGCTTTGGGCTGGCCTAATAAGGCCCTGCCCAACTCCGTGGGGT | | | |
| | | | | TTTTTTTACCATTGTTGGACCTGGGGAGGGGGGAGTTAAAAAAAGAACCAGA | | | |
| | | | | AAGAAATTGTCAAAAACCAATAAATGAAATCCACCAACTCACCGTGTGTGTCC | | | |
| | | | | CAGCTGCCCCATCTTCCCCAGCGCATACCTGTTCCTCTTCTCATTCTCTCCC | | | |
| | | | | CGCCGCCTGTTTCCTCACCTTCTCTCCCCTTTCCATGCCGTCCATGATCCCC | | | |
| | | | | ACCCCATGTGTTTTAAAAAGGCAGTAG | | | |
| Shigella | 3 | prey4411 | 85 | CCGCAAATGTTCCCAGCACAATCGGCTGCGGGAATTTTTCTGCCCCGAGCA | 286 | RKCSQHNRLREFFCPEHS | |
| ospC1 | | | | CAGCGAGTGCATCTGCCACATCTGCCTGGTGGAGCATAAGACCTGCTCTCC | | ECICHICLVEHKTCSPASLS | |
| | | | | CGCGTCCCTGAGCCAGGCCAGCGCCGACCTGGAGGCCACCCTGAGGCACA | | QASADLEATLRHKLTVMYS | |
| | | | | AACTAACTGTCATGTACAGTCAGATCAACGGGGCGTCGAGAGCACTGGATG | | QINGASRALDDVRNRQQD | |
| | | | | ATGTGAGAAACAGGCAGCAGGATGTGCGGATGACTGCAACAGAAAGGTGGA | | VRMTANRKVEQLQQEYTE | |
| | | | | AGCAGCTACAACAAGAATACACGGAAATGAAGGCTCTCTTGGACGCCTCAGA | | MKALLDASETTSTRKIKEEE | |
| | | | | GACCACCTCGACAAGGAAGATAAAGGAAGAGGAGAAGAGGGTCAACAGCAA | | KRVNSKFDTIYQILLKKKSEI | |
| | | | | GTTTGACACCATTTATCAGATTCTCCTCAAGAAGAAGAGTGAGATCCAGACCT | | QTLKEEIEQSLTKRDEFEFL | |
| | | | | TGAAGGAGGAGATTGAACAGAGCCTGACCAAGAGGGATGAGTTCGAGTTTC | | EKASKLRGISTKPVYIPEVE | |
| | | | | TGGAGAAAGCATCAAAACTGCGAGGAATCTCAACAAAGCCAGTCTACATCCC | | LNHKLIKGIHQSTIDLKNELK | |
| | | | | CGAGGTGGAACTGAACCACAAGCTGATAAAAGGCATCCACCAGAGCACCAT | | QCIGRLQELTPSSGDPGEH | |
| | | | | AGACCTCAAAAACGAGCTGAAGCAGTGCATCGGGCGGCTCCAGGAGCTCAC | | DPASTHKSTRPVKKVSKEE | |
| | | | | CCCCAGTTCAGGTGACCCTGGAGAGCATGACCCAGCGTCCACACACAAATC | | KKSKKPPPVPALPSKLPTF | |
| | | | | CACACGCCCTGTGAAGAAGGTCTCCAAAGAGGAAAAGAAATCCAAGAAACCT | | GAPEQLVDLKQAGLEAAAK | |
| | | | | CCCCCTGTCCCTGCCTTACCCAGCAAGCTTCCCACGTTTGGAGCCCCGGAA | | ATSSHPNSTSLKAKVLETFL | |
| | | | | CAGTTAGTGGATTTAAAACAAGCTGGCTTGGAGGCTGCAGCCAAAGCCACCA | | AKSRPELLEYYIKVILDYNT | |
| | | | | GCTCACATCCGAACTCAACATCTCTCAAGGCCAAGGTGCTGGAGACCTTCCT | | AHNKVALSECYTVASVAEM | |
| | | | | GGCCAAGTCCAGACCTGAGCTCCTGGAGTATTACATTAAAGTCATCCTGGAC | | PQNYRPHPQRFTYCSQVL | |
| | | | | TACAACACCGCCCACAACAAAGTGGCTCTGTCAGAGTGCTATACAGTAGCTT | | GLHCYKKGIHYWEVELQKN | |
| | | | | CTGTGGCTGAGATGCCTCAGAACTACCGGCCGCATCCCCAGAGGTTCACAT | | NFCGVGICYGSMNRQGPE | |
| | | | | ACTGCTCTCAGGTGCTGGGCCTGCACTGCTACAAGAAGGGGATCCACTACT | | SRLGRNSASWCVEWFNTK | |
| | | | | GGGAGGTGGAGCTGCAGAAGAACAACTTCTGTGGGGTAGGCATCTGCTACG | | ISAWHNNVEKTLPSTKATR | |
| | | | | GAAGCATGAACCGGCAGGGCCCAGAAAGCAGGCTCGGCCGCAACAGCGCC | | VGVLLNCDHGFVIFFAVAD | |
| | | | | TCCTGGTGCGTGGAGTGGTTCAACACCAAGATCTCTGCCTGGCACAATAACG | | KVHLMYKFRVDFTEALTPA | |
| | | | | TGGAGAAAACCCTGCCCTCCACCAAGGCCACGCGGGTGGGCGTGCTTCTCA | | FWVFSAGATLSICSPK* | |
| | | | | ACTGTGACCACGGCTTTGTCATCTTCTTCGCTGTTGCCGACAAGGTCCACCT | | | |
| | | | | GATGTATAAGTTCAGGGTGGACTTTACTGAGGCTTTGTACCCGGCTTTCTGG | | | |
| | | | | GTATTTTCTGCTGGTGCCACACTCTCCATCTGCTCCCCCAAGTAG | | | |
| Shigella | 3 | prey2686 | 86 | ATGGAGCAGCTGGCCGACGTGACGCTGCGAAGGCTGCTGGATAATGAGGTC | 287 | MEQLADVTLRRLLDNEVFD | |
| ospC1 | | | | TTTGACCTCGACCCCGATCTGCAGGAGCCGAGCCAGATCACCAAGAGGGAC | | LDPDLQEPSQITKRDLEAR | |
| | | | | CTGGAAGCCAGAGCACAGAATGAGTTCTTCCGGGCTTTCTTCAGGTTGCCGA | | AQNEFFRAFFRLPRKEKLH | |
| | | | | GGAAGGAGAAGCTGCACGCGGTTGTGGACTGTTCGCTCTGGACGCCGTTCA | | AVVDCSLWTPFSRCHTAG | |
| | | | | GTCGCTGTCACACCGCGGGGCGGATGTTCGCCTCTGACAGCTACATCTGCT | | RMFASDSYICFASREDGCC | |
| | | | | TTGCCAGCAGAGAAGATGGCTGCTGTAAGATCATCCTGCCACTCAGAGAGG | | KIILPLREVVSIEKMEDTSLL | |
| | | | | TGGTGAGCATCGAGAAGATGGAGGACACGAGCCTGCTGCCGCATCCCATCA | | PHPIIVSIRSKVAFQFIELRD | |
| | | | | TTGTCAGTATCAGAAGCAAGGTGGCCTTCCAGTTCATTGAGCTCCGGGACCG | | RDSLVEALLARLKQVHANH | |
| | | | | AGACAGCCTGGTGGAGGCGCTGCTTGCGAGGTTGAAGCAGGTCCACGCCA | | PVHYDTSADDDMASLVFHS | |
| | | | | ACCACCCCGTGCACTACGACACCTCTGCGGATGATGACATGGCTTCACTCGT | | TSMCSDHRFGDLEMMSSQ | |
| | | | | GTTTCATTCAACAAGCATGTGCAGTGACCACAGATTTGGGGATCTTGAAATG | | NSEESEKEKSPLMHPDALV | |
| | | | | ATGTCTTCTCAAAATAGCGAGGAGAGTGAGAAAGAGAAGAGCCCGCTGATG | | TAFQQSGSQSPDSRMSRE | |
| | | | | CACCCCGATGCCCTGGTCACCGCCTTCCAGCAGTCAGGCAGCCAGAGCCCT | | QIKISLWNDHFVEYGRTVC | |
| | | | | GACTCCCGAATGTCCAGAGAACAGATAAAAATAAGCCTGTGGAATGACCACT | | MFRTEKIRKLVAMGIPESLR | |
| | | | | TTGTGGAATACGGCAGAACCGTGTGTATGTTTCGCACAGAGAAGATTCGGAA | | GRLWLLFSDAVTDLASHPG | |
| | | | | GCTCGTAGCCATGGGCATCCCTGAATCTTTGCGAGGGAGACTCTGGCTTCT | | YYGNLVEESLGKCCLVTEEI | |
| | | | | CTTCTCAGATGCGGTGACGGATCTTGCCTCACACCCTGGTTACTACGGGAAT | | ERDLHRSLPEHPAFQNETG | |
| | | | | CTGGTGGAGGAGTCCCTGGGGAAATGCTGCCTGGTAACCGAGGAGATAGAA | | IAALRRVLTAYAHRNPKIGY | |
| | | | | CGAGACCTGCACCGCTCCCTGCCAGAGCACCCCGCCTTCCAGAACGAAACG | | CQSMNILTSVLLLYTKEEEA | |
| | | | | GGAATTGCTGCTTTGAGGAGAGTCTTGACGGCCTATGCCCACCGGAACCCC | | FWLLVAVCERMLPDYFNH | |
| | | | | AAGATTGGATACTGCCAGTCCATGAACATCCTGACCTCCGTGCTGCTGCTGT | | RVIGAQVDQSVFEELIKGHL | |
| | | | | ACACCAAGGAGGAGGAAGCCTTCTGGCTGTTGGTTGCTGTGTGTGAGCGGA | | PELAEHMNDLSALASVSLS | |
| | | | | TGCTGCCCGATTACTTCAACCACCGAGTGATCGGGGCACAAGTTGACCAGT | | WFLTLFLSIMPLESAVNVVD | |
| | | | | CTGTCTTCGAGGAGCTCATCAAGGGTCATCTCCCAGAGCTGGCAGAGCACA | | CFFYDGIKAIFQLGLAVLEA | |
| | | | | TGAACGACCTCTCAGCCCTGGCGTCTGTCTCTCTCTCGTGGTTCCTGACCCT | | NAEDLCSSKDDGQALMILS | |
| | | | | GTTCCTCAGCATCATGCCTCTAGAGAGTGCGGTGAATGTGGTAGACTGCTTC | | RFLDHIKNEDSPGPPVGSH | |
| | | | | TTCTATGATGGCATCAAAGCCATCTTCCAGCTGGGACTGGCTGTGCTTGAGG | | HAFFSDDQEPYPVTDISDLI | |
| | | | | CCAATGCTGAGGACCTGTGCAGCAGCAAGGATGATGGCCAGGCCTTGATGA | | RDSYEKFGDQSVEQIEHLR | |
| | | | | TCCTCAGCAGGTTTCTAGATCACATTAAGAATGAGGACAGCCCAGGGCCCCC | | YKHRIRVLQGHEDTTKQNV | |
| | | | | AGTTGGCAGCCACCATGCCTTTTTCTCCGACGACCAGGAGCCCTACCCTGT | | LRVVIPEVSILPEDLEELYDL | |
| | | | | GACTGATATTTCGGACCTGATCCGGGATTCCTATGAGAAATTTGGAGACCAG | | FKREHMMSCYWEQPRPM | |
| | | | | TCTGTGGAGCAGATCGAGCACCTACGTTACAAGCACAGGATCAGGGTCCTC | | ASRHDPSRPYAEQYRIDAR | |
| | | | | CAAGGCCACGAGGACACCACAAAGCAGAACGTGCTTCGAGTCGTTATCCCG | | QFAHLFQLVSPWTCGAHT | |
| | | | | GAAGTCTCAATTCTTCCTGAAGACCTAGAGGAGCTCTACGACTTATTCAAGA | | EILAERTFRLLDDNMDQLIE | |
| | | | | GAGAACATATGATGAGCTGTTACTGGGAGCAGCCCAGGCCCATGGCCTCAC | | FKAFVSCLDIMYNGEMNEK | |
| | | | | GCCACGACCCCAGCCGGCCCTATGCTGAGCAGTACCGCATAGACGCCCGG | | IKLLYRLHIPPALTENDRDS | |
| | | | | CAGTTTGCACACCTGTTTCAGCTAGTCTCGCCCTGGACCTGCGGGGCCCAC | | QSPLRNPLLSTSRPLVFGK | |
| | | | | ACGGAGATCCTCGCCGAAAGGACGTTCAGGCTCTTGGATGACAACATGGAC | | PNGDAVDYQKQLKQMIKDL | |
| | | | | CAGCTCATCGAGTTCAAAGCGTTTGTGAGCTGCCTCGATATTATGTATAATG | | AKEKDKTEKELPKMSQREF | |
| | | | | GAGAAATGAATGAGAAGATTAAACTATTATACAGGCTTCATATCCCTCCAGCA | | IQFCKTLYSMFHEDPEEND | |
| | | | | CTCACTGAAAATGACCGAGACAGCCAGTCGCCGTTGAGGAATCCTCTGTTGT | | LYQAIATVTTLLLQIGEVGQ | |
| | | | | CAACATCGAGACCCCTGGTTTTCGGGAAACCCAATGGTGATGCAGTTGATTA | | RGSSSGSCSQECGEELRA | |
| | | | | TCAGAAACAGCTGAAGCAGATGATTAAGGATTTAGCCAAAGAAAAAGATAAA | | SAPSPEDSVFADTGKTPQD | |
| | | | | ACTGAGAAAGAATTGCCCAAAATGAGCCAGAGAGAATTTATCCAGTTCTGTA | | SQALPEAAERDWTVSLEHI | |
| | | | | AAACTCTGTACAGTATGTTCCATGAAGATCCAGAAGAAAATGATTTGTATCAA | | LASLLTEQSLVNFFEKPLD | |
| | | | | GCCATCGCCACAGTCACCACACTGCTGCTGCAGATCGGGGAGGTGGGGCA | | MKSKLENAKINQYNLKTFE | |
| | | | | GCGAGGCAGCAGCTCTGGAAGCTGCTCCCAGGAGTGTGGGGAGGAGCTGC | | MSHQSQSELKLSNL* | |
| | | | | GGGCTTCAGCTCCTTCTCCTGAGGACTCGGTTTTTGCAGACACTGGGAAGAC | | | |
| | | | | GCCCCAGGACTCCCAGGCACTTCCAGAGGCGGCAGAAAGGGACTGGACTG | | | |
| | | | | TCTCCCTTGAACATATTTTAGCTTCACTTCTGACTGAACAGTCATTAGTCAACT | | | |
| | | | | TTTTTGAAAAGCCACTGGACATGAAATCCAAACTTGAAAATGCCAAGATCAAT | | | |
| | | | | CAGTACAATCTCAAAACTTTTGAAATGAGCCACCAATCACAATCTGAACTTAA | | | |
| | | | | GCTGAGTAACTTGTAG | | | |
| Shigella | 3 | prey67368 | 87 | TCTCCCAGACCCTCTGCAGGAACCGTACTACCAGCCACCCTACACGCTCGTT | 288 | LPDPLQEPYYQPPYTLVLE | |
| ospC1 | | | | TTGGAGCTGACCGGCGTCCTCTTGCATCCTGAGTGGTCGCTGGCCACTGGC | | LTGVLLHPEWSLATGWRFK | |
| | | | | TGGAGGTTTAAGAAGCGCCCAGGCATCGAGACCTTGTTCCAGCAGCTTGCC | | KRPGIETLFQQLAPLYEIVIF | |
| | | | | CCTTTATATGAAATTGTCATCTTTACGTCAGAGACTGGCATGACTGCGTTTCC | | TSETGMTAFPLIDSVDPHG | |
| | | | | ACTCATTGATAGTGTGGACCCCCATGGCTTCATCTCCTACCGCCTATTCCGG | | FISYRLFRDATRYMDGHHV | |
| | | | | GACGCCACAAGATACATGGATGGACACCATGTAAAGGATATTTCATGTCTGA | | KDISCLNRDPARVVVVDCK | |
| | | | | ATCGGGACCCAGCTCGAGTAGTAGTTGTGGACTGCAAGAAGGAAGCCTTCC | | KEAFRLQPYNGVALRPWD | |
| | | | | GCCTGCAGCCCTATAACGGCGTTGCCCTGCGGCCCTGGGACGGCAACTCTG | | GNSDDRVLLDLSAFLKTIAL | |
| | | | | ATGACCGGGTCTTGTTGGATCTGTCTGCCTTCCTCAAGACCATTGCACTGAA | | NGVEDVRTVLEHYALEDDP | |
| | | | | TGGTGTGGAGGACGTGCGAACCGTGCTGGAGCACTATGCCCTGGAGGATGA | | LAAFKQRQSRLEQEEQQR | |
| | | | | CCCGCTGGCGGCTTTCAAACAGCGGCAAAGCCGGCTAGAGCAGGAGGAGC | | LAELSKSNKQNLFLGSLTS | |
| | | | | AGCAGCGCCTGGCCGAGCTCTCCAAGTCCAACAAGCAGAACCTCTTCCTTG | | RLWPRSKQP* | |
| | | | | GCTCCCTCACCAGCCGCTTGTGGCCTCGCTCCAAACAGCCCTGA | | | |
| Shigella | 3 | prey67371 | 88 | TGGGGGGTGGGGATGGGGTTTGTTTNTNNNNCTTNTTTTTNTTNNNNTNCNN | 289 | WGVGMGFVXXXXFXXXXX | |
| ospC1 | | | | ATTGGNNTTTNNTTTNTTTNCTACTATGGACNTGANTGATTTTTTTTTTTCTTAT | | WXXXXXLLWT*XIFFFLXFX | |
| | | | | NTTTNACTTGNNTNCTGTGGGNGAAGGNTGNAAANTATTTTATNTGNNTTANT | | LXXVGEGXKXFYXXXSIFXI | |
| | | | | CAATTTTTCNCATTAGCCGANANTCNNTATCCTGATACTACTTCATTNGATGA | | SRXSXS*YYFIX*XIXLIXXXK | |
| | | | | CNTATTNGNCTTATANTCNTTTNGAAGCNTGATTANGATTTATAANCTNNTTTT | | XDXDL*XXFXXGSXX | |
| | | | | NCATNCGGATCCANTCNTN | | | |
| Shigella | 3 | prey4005 | 89 | CTCACACAACTCTTTGAGAGGAGCTCGTCCTCAGGACCCCTCTGAGGAAGG | 290 | SHNSLRGARPQDPSEEGP | |
| ospC1 | | | | TCCCGGTGATTTTGGCTTCCTGCATGCCAGTAGTAGCATCGAGTCCGAGGCA | | GDFGFLHASSSIESEAKPA | |
| | | | | AAACCAGCCCAGCCTCAGCCCACTGGTGAAAAGGAACAAGATAAATCAAAAA | | QPQPTGEKEQDKSKTLSLE | |
| | | | | CTCTTTCCCTTGAGGAGGCTGTGACTTCCATTCAGCAGCTCTTCCAGCTCAG | | EAVTSIQQLFQLSVSIAFNF | |
| | | | | TGTTTCCATCGCTTTCAACTTCCTGGGAACAGAGAACATGAAGAGTGGCGAC | | LGTENMKSGDHTAAFSYF | |
| | | | | CACACGGCAGCCTTTTCTTACTTCCAGAAAGCTGCAGCCCGCGGCTACAGC | | QKAAARGYSKAQYNAGLC | |
| | | | | AAAGCGCAGTACAATGCGGGCTTGTGTCATGAGCATGGCAGAGGCACCCCC | | HEHGRGTPRDISKAVLYYQ | |
| | | | | AGGGACATTAGCAAGGCGGTCCTTTATTATCAGTTGGCTGCCAGCCAGGGC | | LAASQGHSLAQYRYARCLL | |
| | | | | CACAGCCTGGCTCAGTACCGCTATGCCAGGTGCCTACTACGAGACCCAGCC | | RDPASSWNPERQRAVSLL | |
| | | | | TCTTCGTGGAACCCTGAGCGGCAGAGGGCAGTGTCCTTGCTGAAGCAGGCT | | KQAADSGLREAQAFLGVLF | |
| | | | | GCAGACTCAGGCTTGAGAGAGGCCCAAGCTTTCCTCGGGGTGCTTTTCACC | | TKEPYLDEQRAVKYLWLAA | |
| | | | | AAGGAGCCCTACCTGGATGAGCAGAGAGCTGTGAAATATCTTTGGCTTGCAG | | NNGDSQSRYHLGICYEKGL | |
| | | | | CCAACAATGGGGACTCACAGAGCAGGTACCACCTTGGAATTTGCTATGAGAA | | GVQRNLGEALRCYQQSAA | |
| | | | | AGGCCTTGGTGTGCAGAGGAATCTGGGAGAGGCCTTGAGATGTTACCAGCA | | LGNEAAQERLRALFSMGAA | |
| | | | | GTCAGCCGCTCTGGGAAATGAGGCCGCCCAGGAGAGGCTGCGAGCCCTCT | | APGPSDLTVTGLKSFSSPS | |
| | | | | TTTCCATGGGGGCTGCAGCCCCGGGGCCCAGCGACCTGACAGTTACAGGA | | LCSLNTLLAGTSRLPHASST | |
| | | | | CTGAAGTCTTTCTCCAGCCCCTCCCTCTGCAGCTTGAACACCCTGCTAGCAG | | GNLGLLCRSGHLGASLEAS | |
| | | | | GAACCTCACGCCTACCACATGCCTCGAGCACAGGCAACCTTGGCCTCCTCT | | SRAIPPHPYPLERSVVRLG | |
| | | | | GCAGAAGTGGGCATCTCGGAGCCAGCCTGGAAGCCTCCAGCAGGGCTATTC | | FG* | |
| | | | | CCCCACACCCCTACCCACTGGAAAGGAGTGTTGTAAGACTAGGTTTTGGCTA | | | |
| | | | | A | | | |
| Shigella | 3 | prey67380 | 90 | NNNNNNNNNNNNNNNNNNNNNNNNNNNNNNNNNNNNNNNNNNNNNGTCACT | 291 | XXXXXXXXXXXXXXXSLCIF | |
| ospC1 | | | | ATGTATCTTCTTTTAAATGTAAGTTTTGTGTTTTATAATTTTTCACATCTACTGA | | F*M*VLCFIIFHIY*IKSEQ*LC | |
| | | | | ATTAAATCTGAACAGTGACTTTGTGCAAAATAAATTTTGCTGTCCATTCTTGCC | | AK*ILLSILAKKS*MSRMISP | |
| | | | | AAAAAGTCCTGAATGTCCAGGATGATTTCTCCAGGACATCTCTATTGCTCCCA | | GHLYCSQVSNSFLGAKTSG | |
| | | | | AGTTTCAAACAGTTTTTTGGGAGCCAAAACCTCAGGATTTACCCTANATCTGG | | FTLXLVNILKXYX | |
| | | | | TTAACATTTTGAAAANATACANG | | | |
| Shigella | 3 | prey3296 | 91 | GGACCCTGTCTCAGTGGACACGGCCCGACTGGAACACCTCTTTGAGTCTCG | 292 | DPVSVDTARLEHLFESRAK | |
| ospC1 | | | | TGCCAAAGAGGTGCTGCCCTCCAAGAAAGCTGGAGAGGGCCGCCGGACAAT | | EVLPSKKAGEGRRTMTTVL | |
| | | | | GACCACAGTGCTGGACCCCAAGCGCACGAACGCCATCAACATCGGCCTAAC | | DPKRTNAINIGLTTLPPVHVI | |
| | | | | CACACTGCCACCTGTGCATGTCATTAAGGCTGCTCTGCTCAACTTTGATGAG | | KAALLNFDEFAVSKDGIEKL | |
| | | | | TTTGCTGTCAGCAAGGATGGCATTGAGAAGCTACTGACCATGATGCCCACGG | | LTMMPTEEERQKIEGAQLA | |
| | | | | AGGAAGAGCGGCAGAAGATTGAGGGAGCCCAGCTGGCCAACCCTGACATAC | | NPDIPLGPAENFLMTLASIG | |
| | | | | CCCTGGGCCCAGCCGAGAACTTCCTGATGACTCTTGCCTCCATTGGCGGCC | | GLAARLQLWAFKLDYDSM | |
| | | | | TCGCTGCTCGTCTACAACTCTGGGCCTTCAAGCTGGACTATGACAGCATGGA | | EREIAEPLFDLKVGMEQLV | |
| | | | | GCGGGAAATTGCTGAGCCACTGTTTGACCTGAAAGTGGGTATGGAACAGCT | | QNATFRCILATLLAVGNFLN | |
| | | | | GGTACAGAATGCCACCTTCCGCTGCATCCTGGCTACCCTCCTAGCTGTGGG | | GSQSSGFELSYLEKVSDVK | |
| | | | | CAACTTCCTCAATGGCTCCCAGAGCAGCGGCTTTGAGCTGAGCTACCTGGA | | DTVRRQSLLHHLCSLVLQT | |
| | | | | GAAGGTGTCAGATGTGAAGGACACGGTGCGTCGACAGTCACTGCTACACCA | | RPESSDLYSEIPALTRCAKV | |
| | | | | TCTCTGCTCCCTAGTGCTCCAGACCCGGCCTGAGTCCTCTGACCTCTATTCA | | DFEQLTENLGQLERRSRAA | |
| | | | | GAAATCCCTGCCCTGACCCGCTGTGCCAAGGTGGACTTTGAACAGCTGACT | | EESLRSLAKHELAPALRAR | |
| | | | | GAGAACCTGGGGCAGCTGGAGCGCCGGAGCCGGGCAGCCGAGGAAAGCC | | LTHFLDQCARRVAMLRIVH | |
| | | | | TGCGGAGCTTGGCCAAGCATGAGCTGGCCCCAGCCCTGCGTGCCCGCCTC | | RRVCNRFHAFLLYLGYTPQ | |
| | | | | ACCCACTTCCTGGACCAGTGTGCCCGCCGTGTTGCCATGCTAAGGATAGTG | | AAREVRIMQFCHTLREFAL | |
| | | | | CACCGCCGTGTCTGCAATAGGTTCCATGCCTTCCTGCTCTACCTGGGCTACA | | EYRTCRERVLQQQQKQAT | |
| | | | | CCCCGCAGGCGGCCCGTGAAGTGCGCATCATGCAGTTCTGCCACACGCTGC | | YRERNKTRGRMITETEKFS | |
| | | | | GGGAATTTGCGCTTGAGTATCGGACTTGCCGGGAACGAGTGCTACAGCAGC | | GVAGEAPSNPSVPVAVSS | |
| | | | | AGCAGAAGCAGGCCACATACCGTGAGCGCAACAAGACCCGGGGACGCATG | | GPGRGDADSHASMKSLLT | |
| | | | | ATCACCGAGACAGAGAAGTTCTCAGGTGTGGCTGGGGAAGCCCCCAGCAAC | | SRLEDTTHNRRSRGMVQS | |
| | | | | CCCTCTGTCCCAGTAGCAGTGAGCAGCGGGCCAGGCCGGGGAGATGCTGA | | SSPIMPTVGPSTASPEEPP | |
| | | | | CAGTCATGCTAGTATGAAGAGTCTGCTGACCAGCAGGCTTGAGGACACCAC | | GSSLPSDTSDEIMDLLVQS | |
| | | | | ACACAATCGCCGCAGCAGAGGCATGGTCCAGAGCAGCTCCCCAATCATGCC | | VTKSSPRALAARERKRSRG | |
| | | | | CACAGTGGGGCCCTCCACTGCATCCCCAGAAGAACCCCCAGGCTCCAGTTT | | NRKSLRRTLKSGLGDDLVQ | |
| | | | | ACCCAGTGATACATCAGATGAGATCATGGACCTTCTGGTGCAGTCAGTGACC | | ALGLSKGPGLEV* | |
| | | | | AAGAGCAGTCCTCGTGCCTTAGCTGCTAGGGAACGCAAGCGTTCCCGCGGC | | | |
| | | | | AACCGCAAGTCTTTGAGAAGGACGTTGAAGAGTGGGCTCGGAGATGACCTG | | | |
| | | | | GTGCAGGCACTGGGACTAAGCAAGGGTCCTGGCCTGGAGGTGTGA | | | |
| Shigella | 3 | prey2108 | 92 | GCAGGAAGCTCAGAGTATCGATGAAATCTACAAATACGACAAGAAACAGCAG | 293 | QEAQSIDEIYKYDKKQQQEI | |
| ospC1 | | | | CAAGAAATCCTGGCGGCGAAGCCCTGGACTAAGGATCACCATTACTTTAAGT | | LAAKPWTKDHHYFKYCKIS | |
| | | | | ACTGCAAAATCTCAGCATTGGCTCTGCTGAAGATGGTGATGCATGCCAGATC | | ALALLKMVMHARSGGNLEV | |
| | | | | GGGAGGCAACTTGGAAGTGATGGGTCTGATGCTAGGAAAGGTGGATGGTGA | | MGLMLGKVDGETMIIMDSF | |
| | | | | AACCATGATCATTATGGACAGTTTTGCTTTGCCTGTGGAGGGCACTGAAACC | | ALPVEGTETRVNAQAAAYE | |
| | | | | CGAGTAAATGCTCAGGCTGCTGCATATGAATACATGGCTGCATACATAGAAA | | YMAAYIENAKQVGRLENAI | |
| | | | | ATGCAAAACAGGTTGGCCGCCTTGAAAATGCAATCGGGTGGTATCATAGCCA | | GWYHSHPGYGCWLSGIDV | |
| | | | | CCCTGGCTATGGCTGCTGGCTTTCTGGGATTGATGTTAGTACTCAGATGCTC | | STQMLNQQFQEPFVAVVID | |
| | | | | AATCAGCAGTTCCAGGAACCATTTGTAGCAGTGGTGATTGATCCAACAAGAA | | PTRTISAGKVNLGAFRTYP | |
| | | | | CAATATCCGCAGGGAAAGTGAATCTTGGCGCCTTTAGGACATACCCAAAGGG | | KGYKPPDEGPSEYQTIPLN | |
| | | | | CTACAAACCTCCTGATGAAGGACCTTCTGAGTACCAGACTATTCCACTTAATA | | KIEDFGVHCKQYYALEVSY | |
| | | | | AAATAGAAGATTTTGGTGTACACTGCAAACAATATTATGCCTTAGAAGTCTCA | | FKSSLDRKLLELLWNKYWV | |
| | | | | TATTTCAAATCCTCTTTGGATCGCAAATTGCTTGAGCTGTTGTGGAATAAATA | | NTLSSSSLLTNADYTTGQV | |
| | | | | CTGGGTGAATACGTTGAGTTCTTCTAGCTTGCTTACTAATGCAGACTATACCA | | FDLSEKLEQSEAQLGRGSF | |
| | | | | CTGGTCAGGTCTTTGATTTGTCTGAAAAGTTAGAGCAGTCAGAAGCCCAGCT | | MLGLETHDRKSEDKLAKAT | |
| | | | | GGGACGAGGGAGTTTCATGTTGGGTTTAGAAACGCATGACCGAAAATCAGAA | | RDSCKTTIEAIHGLMSQVIK | |
| | | | | GACAAACTTGCCAAAGCTACAAGAGACAGCTGTAAAACTACCATAGAAGCTA | | DKLFNQINIS* | |
| | | | | TCCATGGATTGATGTCTCAGGTTATTAAGGATAAACTGTTTAATCAAATTAACA | | | |
| | | | | TCTCTTAA | | | |
| Shigella | 3 | prey67403 | 93 | TTGGGGCATCTTGGCAGGAGCTTTGGATTTCTTTAGGGAAATGGCAATCAGA | 294 | LGHLGRSFGFL*GNGNQM | |
| ospC1 | | | | TGGGGCAGAGTGTTTTTTGCTGAGGGAATCAGAATGATCCCTCAAACAGCAC | | GQSVFC*GNQNDPSNSTF | |
| | | | | CTTTGATCTCTATTCTCTGCTAAAGATGGTGCTTCCTCTACTTCCCCAGACCC | | DLYSLLKMVLPLLPQTPVSV | |
| | | | | CCGTGTCTGTTCCATTTCCATGAATTTTTCATCAGGGTCACAGGACAAAGGTT | | PFP*IFHQGHRTKVLVFGSN | |
| | | | | TTAGTCTTTGGTTCTAATGAGACCTCTGACTTGGCTCTGGATGACTATGAAAC | | ETSDLALDDYETSECICLF | |
| | | | | TAGTGAATGCATTTGTCTTTTCTGGAATCCN | | | |
| Shigella | 3 | prey67405 | 94 | GCTAATATGGTAGCTATTGATAGCTTACTATGTATCAGATCCNNNNNNNNNN | 295 | ANMVAIDSLLCIRSXXXXXX | |
| ospC1 | | | | NNNNNNNNNNNNNNNNNNNNNNNNNNNNNNNNNNNNNNNNNNNNNNNNNNNN | | XXXXXXXXXXXXXXXXXXX | |
| | | | | NNNNNNNNNNNNNNNNNNNNNNNNNNNNNNNNNNNNNNNNNNNNNNTGAGTA | | XXXXXXXXXXE*LGLQW*A | |
| | | | | GCTAGGACTACAGTGGTGAGCCACCATGCCCAGCTAATTTTTTTTTTTTTTTN | | TMPS*FFFFFXXKGXXXXX | |
| | | | | NNNAAAAAGGGNNTTNNTTNTTNTNGCCCNGGNNGGTNTNAANCTCNTNNC | | PXXVXXSXPXGIXPPXPPX | |
| | | | | CTNANGGNATTNNCCCNCCTNGNCCNCCAAANGGGCNGGANTT | | GXX | |
| Shigella | 3 | prey14400 | 95 | GGGCGAGAGGACTGAGTGTGCTGAGCCCCCCCGGGACGAACCCCCGGCTG | 296 | GERTECAEPPRDEPPADG | |
| ospC1 | | | | ATGGAGCTCTGAAGCGGGCAGAGGAGCTCAAGACTCAGGCCAATGACTACT | | ALKRAEELKTQANDYFKAK | |
| | | | | TCAAAGCCAAGGACTACGAGAACGCCATCAAGTTCTACAGCCAGGCCATCG | | DYENAIKFYSQAIELNPSNA | |
| | | | | AGCTGAACCCCAGCAATGCCATCTACTATGGCAACCGCAGCCTGGCCTACC | | IYYGNRSLAYLRTECYGYA | |
| | | | | TGCGCACTGAGTGCTATGGCTACGCGCTGGGAGACGCCACGCGGGCCATT | | LGDATRAIELDKKYIKGYYR | |
| | | | | GAGCTGGACAAGAAGTACATCAAGGGTTATTACCGCCGGGCTGCCAGCAAC | | RAASNMALGKFRAALRDYE | |
| | | | | ATGGCACTGGGCAAGTTCCGGGCCGCGCTGCGAGACTACGAGACGGTGGT | | TVVKVKPHDKDAKMKYQE | |
| | | | | CAAGGTGAAGCCCCATGACAAGGATGCCAAAATGAAATACCAGGAGTGCAA | | CNKIVKQKAFERAIAGDEH | |
| | | | | CAAGATCGTGAAGCAGAAGGCCTTTGAGCGGGCCATCGCGGGCGACGAGC | | KRSVVDSLDIESMTIEDEYS | |
| | | | | ACAAGCGCTCCGTGGTGGACTCGCTGGACATCGAGAGCATGACCATTGAGG | | GPKLEDGKVTISFMKELMQ | |
| | | | | ATGAGTACAGCGGACCCAAGCTTGAAGACGGCAAAGTGACAATCAGTTTCAT | | WYKDQKKLHRKCAYQILVQ | |
| | | | | GAAGGAGCTCATGCAGTGGTACAAGGACCAGAAGAAACTGCACCGGAAATG | | VKEVLSKLSTLVETTLKETE | |
| | | | | TGCCTACCAGATTCTGGTACAGGTCAAAGAGGTCCTCTCCAAGCTGAGCACG | | KITVCGDTHGQFYDLLNIFE | |
| | | | | CTCGTGGAAACCACACTCAAAGAGACAGAGAAGATTACAGTATGTGGGGACA | | LNGLPSETNPYIFNGDFVD | |
| | | | | CCCATGGCCAGTTCTATGACCTCCTCAACATATTCGAGCTCAACGGTTTACC | | RGSFSVEVILTLFGFKLLYP | |
| | | | | CTCGGAGACCAACCCCTATATATTTAATGGTGACTTTGTGGACCGAGGCTCC | | DHFHLLRGNHETDNMNQIY | |
| | | | | TTCTCTGTAGAAGTGATCCTCACCCTTTTCGGCTTCAAGCTCCTGTACCCAGA | | GFEGEVKAKYTAQMYELFS | |
| | | | | TCACTTTCACCTCCTTCGAGGCAACCACGAGACAGACAACATGAACCAGATC | | EVFEWLPLAQCINGKVLIM | |
| | | | | TACGGTTTCGAGGGTGAGGTGAAGGCCAAGTACACAGCCCAGATGTACGAG | | HGGLFSEDGVTLDDIRKIER | |
| | | | | CTCTTTAGCGAGGTGTTCGAGTGGCTCCCGTTGGCCCAGTGCATCAACGGC | | NRQPPDSGPMCDLLWSDP | |
| | | | | AAAGTGCTGATCATGCACGGAGGCCTGTTCAGTGAAGACGGTGTCACCCTG | | QPQNGRSISKRGVSCQFG | |
| | | | | GATGACATCCGGAAAATTGAGCGGAATCGACAACCCCCAGATTCAGGGCCC | | PDVTKAFLEENNLDYIIRSH | |
| | | | | ATGTGTGACCTGCTCTGGTCAGATCCACAGCCACAGAACGGGCGCTCGATC | | EVKAEGYEVAHGGRCVTV | |
| | | | | AGCAAGCGGGGCGTGAGCTGTCAGTTTGGGCCTGACGTCACCAAGGCCTTC | | FSAPNYCDQMGNKASYIHL | |
| | | | | TTGGAAGAGAACAACCTGGACTATATCATCCGCAGCCACGAAGTCAAGGCC | | QGSDLRPQFHQFTAVPHP | |
| | | | | GAGGGCTACGAGGTGGCTCACGGAGGCCGCTGTGTCACCGTCTTCTCTGCC | | NVKPMAYANTLLQLGMM* | |
| | | | | CCCAACTACTGCGACCAGATGGGGAACAAAGCCTCCTACATCCACCTCCAG | | | |
| | | | | GGCTCTGACCTACGGCCTCAGTTCCACCAGTTCACAGCAGTGCCTCATCCCA | | | |
| | | | | ACGTCAAGCCCATGGCCTATGCCAACACGCTGCTGCAGCTAGGAATGATGT | | | |
| | | | | GA | | | |
| Shigella | 3 | prey50029 | 96 | CTCACCTCTGAAATTCCACAGCTCAATGACTGGAGGCTCTCTCCCACCCACT | 297 | LTSEIPQLNDWRLSPTHSR | |
| ospC1 | | | | CAAGACATTGCCAGGAACGTCTTAAGACCTCAGGAGACCACTTCTTTAGTAA | | HCQERLKTSGDHFFSKQFF | |
| | | | | GCAATTTTTTAGATGGATTCTCACTCTGTCACTCAGGCTGGAGTGCAGTGGC | | RWILTLSLRLECSGAVSAH | |
| | | | | GCGGTCTCTGCTCACTACACCCTCCCTCTCCTGGCTCCTGCCCGTATGTATT | | YTLPLLAPARMYFSFSPCLL | |
| | | | | TCTCCTTCTCTCCATGCCTGCTCTGTAGGGACCATAGCCTCTGTCCCTGCAT | | CRDHSLCPCIHVGHQSHQ | |
| | | | | ACATGTTGGACATCAATCACATCAGTCCACCAAGTAACTTCATCAAGCACCCA | | STK*LHQAPMYAQHSVPRV | |
| | | | | TGTACGCCCAGCACAGCGTCCCAAGGGTGCCCCACTTACCCACAGAAGAAG | | PHLPTEEERQLW*EI*LLAP | |
| | | | | AAAGGCAACTTTGGTAAGAGATCTGACTTCTAGCTCCAGTTCTGTCTCTAGCT | | VLSLANVRCTRLRAVF*LLK | |
| | | | | AACGTGAGATGCACCCGGTTGAGGGCTGTTTTTTAATTGTTGAAAATGAAGG | | MKD*T*MVQLKCFKMI*FYL | |
| | | | | ACTGAACTTAGATGGTCCAACTGAAATGTTTTAAAATGATATGATTCTACCTTA | | KKRMKF*YIHNTGNP*KRYA | |
| | | | | AAAAGAGAATGAAATTCTGATATATTCACAACACAGGAAACCCTTGAAAACGT | | K*NKGDMKGQIYDSTYVMS | |
| | | | | TATGCTAAATGAAATAAGGGAGACATGAAAGGACAAATATATGACTCCACTTA | | LK*TTT*RQKVDSGC*GLLE | |
| | | | | TGTGATGTCCCTCAAATAGACAACCACATAGAGACAGAAAGTAGACAGTGGG | | GQWRVSV*WVQCHSGCSV | |
| | | | | TGCTAGGGGTTGCTGGAGGGGCAATGGAGAGTTAGTTTTAATGGGTACAGG | | YGVGTLGSLYFSNKLAHT* | |
| | | | | TGTCACAGTGGCTGCTCTGTCTATGGAGTAGGCACTCTTGGGTCTCTTTACT | | KEKALEID | |
| | | | | TCTCTAATAAACTCGCTCACACTTAAAAAGAAAAAGCTCTGGAGATTGATAG | | | |
| Shigella | 4 | prey67563 | 97 | GCTGTGTTGAGAGGCGATGCAGAAGCAGTGAAGGGCATAGGATCCGGCAAA | 298 | AVLRGDAEAVKGIGSGKVL | |
| ipaD | | | | GTCCTGAAGAGTGGCCCCCAGGATCACGTGTTCATTTACTTCACTGACCATG | | KSGPQDHVFIYFTDHGSTG | |
| | | | | GATCTACTGGAATACTGGTTTTTCCCAATGAAGATCTTCATGTAAAGGACCTG | | ILVFPNEDLHVKDLNETIHT | |
| | | | | AATGAGACCATCCATTACATGTACAAACACAAAATGTACCGAAAGATGGTGTT | | MYKHKMYRKMVFYIEACES | |
| | | | | CTACATTGAAGCCTGTGAGTCTGGGTCCATGATGAACCACCTGCCGGATAAC | | GSMMNHLPDNINVYATTAA | |
| | | | | ATCAATGTTTATGCAACTACTGCTGCCAACCCCAGAGAGTCGTCCTACGCCT | | NPRESSYACYYDEKRSTYL | |
| | | | | GTTACTATGATGAGAAGAGGTCCACGTACCTGGGGGACTGGTACAGCGTCA | | GDWYSVNWMEDSDVEDLT | |
| | | | | ACTGGATGGAAGACTCGGACGTGGAAGATCTGACTAAAGAGACCCTGCACA | | KETLHKQYHLVKSHTNTSH | |
| | | | | AGCAGTACCACCTGGTAAAATCGCACACCAACACCAGCCACGTCATGCAGTA | | VMQYGNKTISTMKVMQFQ | |
| | | | | TGGAAACAAAACAATCTCCACCATGAAAGTGATGCAGTTTCAGGGTATGAAA | | GMKRKASSPVPLPPVTHLD | |
| | | | | CGCAAAGCCAGTTCTCCCGTCCCCCTACCTCCAGTCACACACCTTGACCTCA | | LTPSPDVPLTIMKRKLMNT | |
| | | | | CCCCCAGCCCTGATGTGCCTCTCACCATCATGAAAAGGAAACTGATGAACAC | | NDLEESRQLTEEIQRHLDA | |
| | | | | CAATGATCTGGAGGAGTCCAGGCAGCTCACGGAGGAGATCCAGCGGCATCT | | RHLIEKSVRKIVSLLAASEA | |
| | | | | GGATGCCAGGCACCTCATTGAGAAGTCAGTGCGTAAGATCGTCTCCTTGCTG | | EVEQLLSERAPLTGHSCYP | |
| | | | | GCAGCGTCCGAGGCTGAGGTGGAGCAGCTCCTGTCCGAGAGAGCCCCGCT | | EALLHFRTHCFNWHSPTYE | |
| | | | | CACGGGGCACAGCTGCTACCCAGAGGCCCTGCTGCACTTCCGGACCCACTG | | YALRHLYVLVNLCEKPYPL | |
| | | | | CTTCAACTGGCACTCCCCCACGTACGAGTATGCGTTGAGACATTTGTACGTG | | HRIKLSMDHVCLGHY* | |
| | | | | CTGGTCAACCTTTGTGAGAAGCCGTATCCACTTCACAGGATAAAATTGTCCAT | | | |
| | | | | GGACCACGTGTGCCTTGGTCACTACTGA | | | |
| Shigella | 4 | prey2109 | 98 | GACTAAGGATCACCATTACTTTAAGTACTGCAAAATCTCAGCATTGGCTCTTC | 299 | TKDHHYFKYCKISALALLKM | |
| ipaD | | | | TGAAGATGGTGATGCATGCCAGATCGGGAGGCAATTTGGAAGTGATGGGTC | | VMHARSGGNLEVMGLMLG | |
| | | | | TGATGCTAGGAAAGGTGGATGGTGAAACCATGATCATTATGGACAGTTTTGC | | KVDGETMIIMDSFALPVEGT | |
| | | | | TTTGCCTGTGGAGGGCACTGAAACCCGAGTAAATGCTCAGGCTGCTGCATAT | | ETRVNAQAAAYEYMAAYIE | |
| | | | | GAATACATGGCTGCATACATAGAAAATGCAAAACAGGTTGGCCGCCTTGAAA | | NAKQVGRLENAIGWYHSH | |
| | | | | ATGCAATCGGGTGGTATCATAGCCACCCTGGCTATGGCTGCTGGCTTTCTGG | | PGYGCWLSGIDVSTQMLN | |
| | | | | GATTGATGTTAGTACTCAGATGCTCAATCAGCAGTTCCAGGAACCATTTGTAG | | QQFQEPFVAVVIDPTRTISA | |
| | | | | CAGTGGTGATTGATCCAACAAGAACAATATCCGCAGGGAAAGTGAATCTTGG | | GKVNLGAFRTYPKGYKPPD | |
| | | | | CGCCTTTAGGACATACCCAAAGGGCTACAAACCTCCTGATGAAGGACCTTCT | | EGPSEYQTIPLNKIEDFGVH | |
| | | | | GAGTACCAGACTATTCCACTTAATAAAATAGAAGATTTTGGTGTACACTGCAA | | CKQYYALEVSYFKSSLDRK | |
| | | | | ACAATATTATGCCTTAGAAGTCTCATATTTCAAATCCTCTTTGGATCGCAAATT | | LLELLWNKYWVNTLSSSSL | |
| | | | | GCTTGAGCTGTTGTGGAATAAATACTGGGTGAATACGTTGAGTTCTTCTAGCT | | LTNADYTTGQVFDLSEKLE | |
| | | | | TGCTTACTAATGCAGACTATACCACTGGTCAGGTCTTTGATTTGTCTGAAAAG | | QSEAQLGRGSFMLGLETH | |
| | | | | TTAGAGCAGTCAGAAGCCCAGCTGGGACGAGGGAGTTTCATGTTGGGTTTA | | DRKSEDKLAKATRDSCKTT | |
| | | | | GAAACGCATGACCGAAAATCAGAAGACAAACTTGCCAAAGCTACAAGAGACA | | IEAIHGLMSQVIKDKLFNQIN | |
| | | | | GCTGTAAAACTACCATAGAAGCTATCCATGGATTGATGTCTCAGGTTATTAAG | | IS* | |
| | | | | GATAAACTGTTTAATCAAATTAACATCTCTTAA | | | |
| Shigella | 4 | prey25185 | 99 | GGGCAATAAGGCCTGTAGCCCATGCTCCTCACAGTCCTCCAGCAGTGGCAT | 300 | GNKACSPCSSQSSSSGICT | |
| ipaD | | | | TTGCACAGACTTCTGGGACTTATTGGTAAAACTGGACAACATGAATGTCAGC | | DFWDLLVKLDNMNVSRKG | |
| | | | | CGGAAAGGCAAGAACTCCGTGAAGTCAGTGCCAGTGAGCGCTGGCGGTGA | | KNSVKSVPVSAGGEGETS | |
| | | | | GGGGGAAACCTCTCCATACAGCCTCGAGGCCTCTCCACTGGGGCAGCTCAT | | PYSLEASPLGQLMNMLSHP | |
| | | | | GAACATGTTGTCACACCCAGTCATCCGCCGGAGCTCTCTCTTAACTGAGAAA | | VIRRSSLLTEKLLRLLSLISIA | |
| | | | | CTCCTCAGACTCCTTTCTCTCATCTCAATTGCTCTCCCAGAAAACAAGGTGTC | | LPENKVSEAQANSGSGAS | |
| | | | | AGAAGCACAGGCTAATTCTGGCAGCGGTGCTTCCTCCACCACCACTGCCAC | | STTTATSTTSTTTTTAASTT | |
| | | | | CTCAACCACATCTACCACCACCACCACTGCCGCCTCCACCACGCCCACACC | | PTPPTAPTPVTSAPALVAAT | |
| | | | | CCCTACTGCACCCACCCCTGTCACTTCTGCTCCAGCCCTGGTTGCTGCCAC | | AISTIVVAASTTVTTPTTATT | |
| | | | | GGCTATTTCCACCATTGTCGTAGCTGCTTCGACCACAGTGACTACCCCCACG | | TVSISPTTKGSKSPAKVSD | |
| | | | | ACTGCTACCACTACTGTTTCAATTTCTCCCACTACTAAGGGCAGCAAATCTCC | | GGSSSTDFKMVSSGLTEN | |
| | | | | AGCGAAGGTGAGTGATGGGGGCAGCAGCAGTACAGACTTTAAGATGGTGTC | | QLQLSVEVLTSHSCSEEGL | |
| | | | | CTCTGGCCTCACTGAAAACCAGCTACAGCTCTCTGTAGAGGTGTTGACATCC | | EDAANVLLQLSRGDSGTRD | |
| | | | | CACTCTTGTTCTGAGGAAGGCTTAGAGGATGCAGCCAACGTACTACTGCAGC | | TVLKLLLNGARHLGYTLCK | |
| | | | | TCTCCCGGGGGGACTCTGGGACCCGGGACACTGTTCTCAAGCTGCTACTGA | | QIGTLLAELREYNLEQQRR | |
| | | | | ATGGAGCCCGCCATCTGGGTTATACCCTTTGTAAACAAATAGGTACCCTGCT | | AQCETLSPDGLPEEQPQTT | |
| | | | | GGCCGAGCTGCGGGAATACAACCTCGAGCAGCAGCGGCGAGCCCAATGTG | | KLKGKMQSRFDMAENVVIV | |
| | | | | AAACCCTCTCTCCTGATGGCCTGCCTGAGGAGCAGCCACAGACCACCAAGC | | ASQKRPLGGRELQLPSMS | |
| | | | | TGAAGGGCAAAATGCAGAGCAGGTTTGACATGGCTGAGAATGTGGTAATTGT | | MLTSKTSTQKFFLRVLQVII | |
| | | | | GGCATCTCAGAAGCGACCTTTGGGTGGCCGGGAGCTCCAGCTGCCTTCTAT | | QLRDDTRRANKKAKQTGR | |
| | | | | GTCCATGTTGACATCCAAGACATCTACCCAGAAGTTCTTCTTGAGGGTACTA | | LGSSGLGSASSIQAAVRQL | |
| | | | | CAGGTCATCATCCAGCTCCGGGACGACACGCGCCGGGCTAACAAGAAAGCC | | EAEADAIIQMVREGQRARR | |
| | | | | AAGCAGACAGGCAGGCTAGGTTCCTCCGGTTTAGGCTCAGCTAGCAGCATC | | QQQAATSESSQSEASVRR | |
| | | | | CAGGCAGCTGTTCGGCAGCTGGAGGCTGAGGCTGATGCCATTATACAAATG | | EESPMDVDQPSPSAQDTQ | |
| | | | | GTACGTGAGGGTCAAAGGGCGCGGAGACAGCAACAAGCAGCAACGTCGGA | | SIASDGTPQGEKEKEERPP | |
| | | | | GTCTAGCCAGTCAGAGGCGTCTGTCCGGAGGGAGGAATCACCCATGGATGT | | ELPLLSEQLSLDELWDMLG | |
| | | | | GGACCAGCCATCTCCCAGTGCTCAAGATACTCAATCCATTGCCTCCGATGGA | | ECLKELEESHDQHAVLVLQ | |
| | | | | ACCCCACAGGGGGAGAAGGAAAAGGAAGAAAGACCACCTGAGTTACCCCTG | | PAVEAFFLVHATERESKPP | |
| | | | | CTCAGCGAGCAGCTGAGTTTGGACGAGCTGTGGGACATGCTTGGGGAGTGT | | VRDTRESQLAHIKDEPPPL | |
| | | | | CTAAAGGAACTAGAGGAATCCCATGACCAGCATGCGGTGCTAGTGCTACAG | | SPAPLTPATPSSLDPFFSR | |
| | | | | CCTGCTGTCGAGGCCTTCTTTCTGGTCCATGCCACAGAGCGGGAGAGCAAG | | EPSSMHISSSLPPDTQKFL | |
| | | | | CCTCCTGTCCGAGACACCCGTGAGAGCCAGCTGGCACACATCAAGGACGAG | | RFAETHRTVLNQILRQSTT | |
| | | | | CCTCCTCCACTCTCCCCTGCCCCCTTAACCCCAGCCACGCCTTCCTCCCTTG | | HLADGPFAVLVDYIRVLDFD | |
| | | | | ACCCATTCTTCTCCCGGGAGCCCTCATCTATGCACATCTCCTCAAGCCTGCC | | VKRKYFRQELERLDEGLRK | |
| | | | | CCCTGACACACAGAAGTTCCTTCGCTTTGCAGAGACTCACCGCACTGTGTTA | | EDMAVHVRRDHVFEDSYR | |
| | | | | AACCAGATCCTACGGCAGTCCACGACCCACCTTGCTGATGGGCCTTTTGCTG | | ELHRKSPEEMKNRLYIVFE | |
| | | | | TCCTGGTAGACTACATTCGTGTCCTCGACTTTGATGTCAAGCGCAAATATTTC | | GEEGQDAGGLLREWYMIIS | |
| | | | | CGCCAAGAGCTGGAGCGTTTAGATGAGGGGCTCCGGAAAGAAGACATGGCT | | REMFNPMYALFRTSPGDR | |
| | | | | GTGCATGTCCGTCGTGACCATGTGTTTGAAGACTCCTATCGTGAGCTGCATC | | VTYTINPSSHCNPNHLSYF | |
| | | | | GCAAATCCCCCGAAGAAATGAAGAATCGATTGTATATAGTATTTGAAGGAGA | | KFVGRIVAKAVYDNRLLEC | |
| | | | | AGAAGGGCAGGATGCTGGCGGGCTCCTGCGGGAGTGGTATATGATCATCTC | | YFTRSFYKHILGKSVRYTD | |
| | | | | TCGAGAGATGTTTAACCCTATGTATGCCTTGTTCCGTACCTCACCTGGTGATC | | MESEDYHFYQGLVYLLEND | |
| | | | | GAGTCACCTACACCATCAATCCATCTTCCCACTGCAACCCCAACCACCTCAG | | VSTLGYDLTFSTEVQEFGV | |
| | | | | CTACTTCAAGTTTGTCGGACGCATTGTGGCCAAAGCTGTATATGACAACCGT | | CEVRDLKPNGANOLVTEEN | |
| | | | | CTTCTGGAGTGCTACTTTACTCGATCCTTTTACAAACACATCTTGGGCAAGTC | | KKEYVHLVCQMRMTGAIRK | |
| | | | | AGTCAGATATACAGATATGGAGAGTGAAGATTACCACTTCTACCAAGGTCTG | | QLAAFLEGFYEIIPKRLISIFT | |
| | | | | GTTTATCTGCTGGAAAATGATGTCTCCACACTAGGCTATGACCTCACCTTCAG | | EQELELLISGLPTIDIDDLKS | |
| | | | | CACTGAGGTCCAAGAGTTTGGAGTTTGTGAAGTTCGTGACCTCAAACCCAAT | | NTEYHKYQSNSIQIQWFWR | |
| | | | | GGGGCCAACATCTTGGTAACAGAGGAGAATAAGAAGGAGTATGTACACCTG | | ALRSFDQADRAKFLQFVTG | |
| | | | | GTATGCCAGATGAGAATGACAGGAGCCATCCGCAAGCAGTTGGCGGCTTTC | | TSKVPLQGFAALEGMNGIQ | |
| | | | | TTAGAAGGCTTCTATGAGATCATTCCAAAGCGCCTCATTTCCATCTTCACTGA | | KFQIHRDDRSTDRLPSAHT | |
| | | | | GCAGGAGTTAGAGCTGCTTATATCAGGACTGCCCACCATTGACATCGATGAT | | CFNQLDLPAYESFEKSATC | |
| | | | | CTGAAATCCAACACTGAATACCACAAGTACCAGTCCAACTCTATTCAGATCCA | | YCWLSRSALKALGWPNKA | |
| | | | | GTGGTTCTGGAGAGCATTGCGTTCTTTCGATCAAGCTGACCGTGCCAAGTTC | | LPNSVGFFLPLLDLGRGEL | |
| | | | | CTCCAGTTTGTCACGGGTACTTCCAAGGTACCCCTGCAAGGCTTTGCTGCCC | | KKEPERNCQKPINEIHQLTV | |
| | | | | TCGAAGGCATGAATGGCATTCAGAAGTTTCAGATCCATCGAGATGACAGGTC | | CVPAAPSSPAHTCSSSHSL | |
| | | | | CACAGATCGCCTGCCTTCAGCTCACACATGTTTTAATCAGCTGGATCTGCCT | | PAACFLTFSPLSMPSMIPTP | |
| | | | | GCCTATGAGAGCTTTGAGAAGTCCGCCACATGCTACTGTTGGCTATCCAGGA | | CVLKRQ* | |
| | | | | GTGCTCTGAAGGCTTTGGGCTGGCCTAATAAGGCCCTGCCCAACTCCGTGG | | | |
| | | | | GGTTTTTTTTACCATTGTTGGACCTGGGGAGGGGGGAGTTAAAAAAAGAACC | | | |
| | | | | AGAAAGAAATTGTCAAAAACCAATAAATGAAATCCACCAACTCACCGTGTGTG | | | |
| | | | | TCCCAGCTGCCCCATCTTCCCCAGCGCATACCTGTTCCTCTTCTCATTCTCTC | | | |
| | | | | CCCGCCGCCTGTTTCCTCACCTTCTCTCCCCTTTCCATGCCGTCCATGATCC | | | |
| | | | | CCACCCCATGTGTTTTAAAAAGGCAGTAG | | | |
| Shigella | 4 | prey53990 | 100 | CCACCTATACCCCCGGTGACTGTCCCAACTTTGCGGCTCCCCGCAGAGAGG | 301 | TYTPGDCPNFAAPRREVAP | |
| ipaD | | | | TGGCACCACCCTATCAGGGGGCTGACCCCATCCTTGCGACAGCCCTCGCCT | | PYQGADPILATALASDPIPN | |
| | | | | CCGACCCCATCCCCAACCCCCTTCAGAAGTGGGAGGACAGCGCCCACAAGC | | PLQKWEDSAHKPQSLDTD | |
| | | | | CACAGAGCCTAGACACTGATGACCCCGCGACGCTGTACGCCGTGGTGGAGA | | DPATLYAVVENVPPLRWKE | |
| | | | | ACGTGCCCCCGTTGCGCTGGAAGGAATTCGTGCGGCGCCTAGGGCTGAGC | | FVRRLGLSDHEIDRLELQN | |
| | | | | GACCACGAGATCGATCGGCTGGAGCTGCAGAACGGGCGCTGCCTGCGCGA | | GRCLREAQYSMLATWRRR | |
| | | | | GGCGCAATACAGCATGCTGGCGACCTGGAGGCGGCGCACGCCGCGGCGC | | TPRREATLELLGRVLRDMD | |
| | | | | GAGGCCACGCTGGAGCTGCTGGGACGCGTGCTCCGCGACATGGACCTGCT | | LLGCLEDIEEALCGPAALPP | |
| | | | | GGGCTGCCTGGAGGACATCGAGGAGGCGCTTTGCGGCCCCGCCGCCCTCC | | APSLLR* | |
| | | | | CGCCCGCGCCCAGTCTTCTCAGATGA | | | |
| Shigella | 4 | prey9120 | 101 | GCCACGCGCTCCTCTGCCGTGCGCCTGCGGAGCAGCGTGCCCGGGGTGCG | 302 | ATRSSAVRLRSSVPGVRLL | |
| ipaD | | | | GCTCCTGCAGGACTCGGTGGACTTCTCGCTGGCCGACGCCATCAACACCGA | | QDSVDFSLADAINTEFKNT | |
| | | | | GTTCAAGAACACCCGCACCAACGAGAAGGTGGAGCTGCAGGAGCTGAATGA | | RTNEKVELQELNDRFANYI | |
| | | | | CCGCTTCGCCAACTACATCGACAAGGTGCGCTTCCTGGAGCAGCAGAATAA | | DKVRFLEQQNKILLAELEQL | |
| | | | | GATCCTGCTGGCCGAGCTCGAGCAGCTCAAGGGCCAAGGCAAGTCGCGCC | | KGQGKSRLGDLYEEEMRE | |
| | | | | TAGGGGACCTCTACGAGGAGGAGATGCGGGAGCTGCGCCHHCAGGTGGAC | | LRRQVDQLTNDKARVEVE | |
| | | | | CAGCTAACCAACGACAAAGCCCGCGTCGAGGTGGAGCGCGACAACCTGGC | | RDNLAEDIMRLREKLQEEM | |
| | | | | CGAGGACATCATGCGCCTCCGGGAGAAATTGCAGGAGGAGATGCTTCAGAG | | LQREEAENTLQSFRQDVD | |
| | | | | AGAGGAAGCCGAAAACACCCTGCAATCTTTCAGACAGGATGTTGACAATGCG | | NASLARLDLERKVESLQEEI | |
| | | | | TCTCTGGCACGTCTTGACCTTGAACGCAAAGTGGAATCTTTGCAAGAAGAGA | | AFLKKLHEEEIQELQAQIQE | |
| | | | | TTGCCTTTTTGAAGAAACTCCACGAAGAGGAAATCCAGGAGCTGCAGGCTCA | | QHVQIDVDVSKPDLTAALR | |
| | | | | GATTCAGGAACAGCATGTCCAAATCGATGTGGATGTTTCCAAGCCTGACCTC | | DVRQQYESVAAKNLQEAE | |
| | | | | ACGGCTGCCCTGCGTGACGTACGTCAGCAATATGAAAGTGTGGCTGCCAAG | | EWYKSKFADLSEAANRNN | |
| | | | | AACCTGCAGGAGGCAGAAGAATGGTACAAATCCAAGTTTGCTGACCTCTCTG | | DALRQAKQESTEYRRQVQ | |
| | | | | AGGCTGCCAACCGGAACAATGACGCCCTGCGCCAGGCAAAGCAGGAGTCC | | SLTCEVDALKGTNESLERQ | |
| | | | | ACTGAGTACCGGAGACAGGTGCAGTCCCTCACCTGTGAAGTGGATGCCCTT | | MREMEENFAVEAANYQDTI | |
| | | | | AAAGGAACCAATGAGTCCCTGGAACGCCAGATGCGTGAAATGGAAGAGAAC | | GRLQDEIQNMKEEMARHL | |
| | | | | TTTGCCGTTGAAGCTGCTAACTACCAAGACACTATTGGCCGCCTGCAGGATG | | REYQDLLNVKMALDIEIATY | |
| | | | | AGATTCAGAATATGAAGGAGGAAATGGCTCGTCACCTTCGTGAATACCAAGA | | RKLLEGEESRISLPLPNFSS | |
| | | | | CCTGCTCAATGTTAAGATGGCCCTTGACATTGAGATTGCCACCTACAGGAAG | | LNLRETNLDSLPLVDTHSK | |
| | | | | CTGCTGGAAGGCGAGGAGAGCAGGATTTCTCTGCCTCTTCCAAACTTTTCCT | | RTFLIKTVETRDGQVINETS | |
| | | | | CCCTGAACCTGAGGGAAACTAATCTGGATTCACTCCCTCTGGTTGATACCCA | | QHHDDLE* | |
| | | | | CTCAAAAAGGACATTCCTGATTAAGACGGTTGAAACTAGAGATGGACAGGTT | | | |
| | | | | ATCAACGAAACTTCTCAGCATCACGATGACCTTGAATAA | | | |
| Shigella | 4 | prey67571 | 102 | CCNTANTATGGAGACTANCNCCNTGGTCCGCNCTGGAAGGATCACCTTATGT | 303 | PXYGDXXXGPXWKDHLMX | |
| ipaD | | | | NCAGATGCAAGTTCTGATGCAGNAGGTCTGGGCAGANCCCNCNACTCTGCN | | RCKF*CXRSGQXPXLCXSX | |
| | | | | TTTCCNCAGGCTGGCAGTGGTGANGATGCTGCGGTCCAGGCAGGGAGCTG | | GWQW*XCCGPGRELLLQG | |
| | | | | CTTTTGCAGGGTGAGGCGGTGGANGGCTGCAACACNCCCCNGACCCCNTCT | | EAVXGCNTPXTPSPFSNAX | |
| | | | | CCNTTCTCAAATGCTGNGANGACTGGAATNNTCCATAGANNANGTTTCTTTTT | | XTGXXHRXXFFFXXXXXE | |
| | | | | TNTANNNNAAANTNATGAAN | | | |
| Shigella | 4 | prey67572 | 103 | TCCTTTNAGGATGNTGAAAAGANGAATATATGCTTGGGAGCATGNNGTATCT | 304 | SFXDXEKXNICLGAXXIFXV | |
| ipaD | | | | TTNTGGTAGCATNACGCCATGNCCTACTTGTGCTTNNNNCACTTNGTTTNNN | | AXRHXLLVLXXLXXXGLQH | |
| | | | | NGGACTACAACATGGAGGAANTNNACCNNATCTACCCTNTAGGCCTGCTCNT | | GGXXPXLPXRPAXGLLXVS | |
| | | | | GGTCTCCTTGNTGTATCATGCCCTCGCTGGTNTGGAGCCNNNGCGGGNCCT | | CPRWXGAXAGPLXYASXIP | |
| | | | | CTTGANTATGCTTCANCCATACCAACACTGGTTGTATGTACGCGATCGCAAC | | TLVVCTRSQHXMHVCXLLY | |
| | | | | ATCANATGCACGTATGTTNCTTGCTGTACAGACGCTACNAGAGANGGGCTTC | | RRYXRXASLX | |
| | | | | CCTGNATN | | | |
| Shigella | 4 | prey65696 | 104 | TGCTGCTGCCACCAACCACACCACTGATAATGGTGTGGGTCCTGAGGAAGA | 305 | AAATNHTTDNGVGPEEESV | |
| ipaD | | | | GAGCGTGGACCCAAATCAATACTACAAAATCCGCAGTCAAGCAATTCATCAG | | DPNQYYKIRSQAIHQLKVN | |
| | | | | CTGAAGGTCAATGGGGAAGACCCATACCCACACAAGTTCCATGTAGACATCT | | GEDPYPHKFHVDISLTDFIQ | |
| | | | | CACTCACTGACTTCATCCAAAAATATAGTCACCTGCAGCCTGGGGATCACCT | | KYSHLQPGDHLTDITLKVA | |
| | | | | GACTGACATCACCTTAAAGGTGGCAGGTAGGATCCATGCCAAAAGAGCTTCT | | GRIHAKRASGGKLIFYDLR | |
| | | | | GGGGGAAAGCTCATCTTCTATGATCTTCGAGGAGAGGGGGTGAAGTTGCAA | | GEGVKLQVMANSRNYKSE | |
| | | | | GTCATGGCCAATTCCAGAAATTATAAATCAGAAGAAGAATTTATTCATATTAAT | | EEFIHINNKLRRGDIIGVQG | |
| | | | | AACAAACTGCGTCGGGGAGACATAATTGGAGTTCAGGGGAATCCTGGTAAAA | | NPGKTKKGELSIIPYEITLLS | |
| | | | | CCAAGAAGGGTGAGCTGAGCATCATTCCGTATGAGATCACACTGCTGTCTCC | | PCLHMLPHLHFGLKDKETR | |
| | | | | CTGTTTGCATATGTTACCTCATCTTCACTTTGGGCTCAAAGACAAGGAAACAA | | YRQRYLDLILNDFVRQKFII | |
| | | | | GGTATCGCCAGAGATACTTGGACTTGATCCTGAATGACTTTGTGAGGCAGAA | | RSKIITYIRSFLDELGFLEIET | |
| | | | | ATTTATCATCCGCTCTAAGATCATCACATATATAAGAAGTTTCTTAGATGAGCT | | PMMNIIPGGAVAKPFITYHN | |
| | | | | GGGATTCCTAGAGATTGAAACTCCCATGATGAACATCATCCCAGGGGGAGC | | ELDMNLYMRIAPELYHKML | |
| | | | | CGTGGCCAAGCCTTTCATCACTTATCACAACGAGCTGGACATGAACTTATATA | | VVGGIDRVYEIGRQFRNEGI | |
| | | | | TGAGAATTGCTCCAGAACTCTATCATAAGATGCTTGTGGTTGGTGGCATCGA | | DLTHNPEFTTCEFYMAYAD | |
| | | | | CCGGGTTTATGAAATTGGACGCCAGTTCCGGAATGAGGGGATTGATTTGACG | | YHDLMEITEKMVSGMVKHI | |
| | | | | CACAATCCTGAGTTCACCACCTGTGAGTTCTACATGGCCTATGCAGACTATC | | TGSYKVTYHPDGPEGQAY | |
| | | | | ACGATCTCATGGAAATCACGGAGAAGATGGTTTCAGGGATGGTGAAGCATAT | | DVDFTPPFRRINMVEELEK | |
| | | | | TACAGGCAGTTACAAGGTCACCTACCACCCAGATGGCCCAGAGGGCCAAGC | | ALGMKLPETNLFETEETRKI | |
| | | | | CTACGATGTTGACTTCACCCCACCCTTCCGGCGAATCAACATGGTAGAAGAG | | LDDICVAKAVECPPPRTTA | |
| | | | | CTTGAGAAAGCCCTGGGGATGAAGCTGCCAGAAACGAACCTCTTTGAAACTG | | RLLDKLVGEFLEVTCINPTFI | |
| | | | | AAGAAACTCGCAAAATTCTTGATGATATCTGTGTGGCAAAAGCTGTTGAATGC | | CDHPQIMSPLAKWHRSKE | |
| | | | | CCTCCACCTCGGACCACAGCCAGGCTCCTTGACAAGCTTGTTGGGGAGTTC | | GLTERFELFVMKKEICNAYT | |
| | | | | CTGGAAGTGACTTGCATCAATCCTACATTCATCTGTGATCACCCACAGATAAT | | ELNDPMRQRQLFEEQAKA | |
| | | | | GAGCCCTTTGGCTAAATGGCACCGCTCTAAAGAGGGTCTGACTGAGCGCTTT | | KAAGDDEAMFIDENFCTAL | |
| | | | | GAGCTGTTTGTCATGAAGAAAGAGATATGCAATGCGTATACTGAGCTGAATG | | EYGLPPTAGWGMGIDRVA | |
| | | | | ATCCCATGCGGCAGCGGCAGCTTTTTGAAGAACAGGCCAAGGCCAAGGCTG | | MFLTDSNNIKEVLLFPAMKP | |
| | | | | CAGGTGATGATGAGGCCATGTTCATAGATGAAAACTTCTGTACTGCCCTGGA | | EDKKENVATTDTLESTTVG | |
| | | | | ATATGGGCTGCCCCCCACAGCTGGCTGGGGCATGGGCATTGATCGAGTCGC | | TSV* | |
| | | | | CATGTTTCTCACGGACTCCAACAACATCAAGGAAGTACTTCTGTTTCCTGCCA | | | |
| | | | | TGAAACCCGAAGACAAGAAGGAGAATGTAGCAACCACTGATACACTGGAAAG | | | |
| | | | | CACAACAGTTGGCACTTCTGTCTAG | | | |
| Shigella | 4 | prey8889 | 105 | GCTCAAGCCGGAGTTCATGCGGCGGCCGGACAAGTCCTTCGACCCCTTCAC | 306 | LKPEFMRRPDKSFDPFTEV | |
| ipaD | | | | TGAGGTCATCGTGGATGGCATCGTGGCCAATGCCTTGCGGGTCAAGGTGAT | | IVDGIVANALRVKVISGQFL | |
| | | | | CTCAGGGCAGTTCCTGTCCGACAGGAAGGTGGGCATCTACGTGGAGGTGGA | | SDRKVGIYVEVDMFGLPVD | |
| | | | | CATGTTTGGCCTCCCTGTTGATACGCGGCGCAAGTACCGCACCCGGACCTC | | TRRKYRTRTSQGNSFNPV | |
| | | | | TCAGGGGAACTCGTTCAACCCCGTGTGGGACGAAGAGCCCTTCGACTTCCC | | WDEEPFDFPKVVLPTLASL | |
| | | | | CAAGGTGGTGCTGCCCACGCTGGCTTCACTTCGCATTGCAGCCTTTGAGGA | | RIAAFEEGGKFVGHRILPVS | |
| | | | | GGGGGGTAAATTCGTAGGGCACCGGATCCTGCCTGTCTCTGCCATCCGCTC | | AIRSGYHYVCLRNEANQPL | |
| | | | | CGGATACCACTACGTCTGCCTGCGGAACGAGGCCAACCAACCGCTGTGCCT | | CLPALLIYTEASDYIPDDHQ | |
| | | | | GCCGGCCCTGCTCATCTACACCGAAGCCTCGGACTACATTCCTGACGACCA | | DYAEALINPIKHVSLMDQRA | |
| | | | | CCAGGACTATGCGGAGGCCCTGATCAACCCCATTAAGCACGTCAGCCTGAT | | RQLAALIGESEAQAGQETC | |
| | | | | GGACCAGAGGGCCCGGCAGCTGGCCGCCCTCATTGGGGAGAGTGAGGCTC | | QDTQSQQLGSQPSSNPTP | |
| | | | | AGGCTGGCCAAGAGACGTGCCAGGACACCCAGTCTCAGCAGCTGGGGTCT | | SPLDASPRRPPGPTTSPAS | |
| | | | | CAGCCGTCCTCAAACCCCACCCCCAGCCCACTGGATGCCTCCCCCCGCCGG | | TSLSSPGQRDDLIASILSEV | |
| | | | | CCCCCTGGCCCCACCACCTCCCCTGCCAGCACCTCCCTCAGCAGCCCAGG | | APTPLDELRGHKALVKLRS | |
| | | | | GCAGCGTGATGATCTCATCGCCAGCATCCTCTCAGAGGTGGCCCCCACCCC | | RQERDLRELRKKHQRKAV | |
| | | | | GCTGGATGAGCTCCGAGGTCACAAGGCTCTGGTCAAGCTCCGGAGCCGGC | | TLTRRLLDGLAQAQAEGRC | |
| | | | | AAGAGCGAGACCTGCGGGAGCTGCGCAAGAAGCATCAGCGGAAGGCAGTC | | RLRPGALGGAADVEDTKE | |
| | | | | ACCCTCACCCGCCGCCTGCTGGATGGCCTGGCTCAGGCACAGGCTGAGGG | | GEDEAKRYQEFQNRQVQS | |
| | | | | CAGGTGCCGGCTGCGGCCAGGTGCCCTAGGTGGGGCCGCTGATGTGGAGG | | LLELREAQVDAEAQRRLEH | |
| | | | | ACACGAAGGAGGGGGAGGACGAGGCAAAGCGGTATCAGGAGTTCCAGAAC | | LRQALQRLREVVLDANTTQ | |
| | | | | AGACAGGTGCAGAGCCTGCTGGAGCTGCGGGAGGCCCAGGTGGACGCAGA | | FKRLKEMNEREKKELQKIL | |
| | | | | GGCCCAGCGGAGGCTGGAACACCTGAGACAGGCTCTGCAGCGGCTCAGGG | | DRKRHNSISEAKMRDKHKK | |
| | | | | AGGTCGTCCTTGATGCAAACACAACTCAGTTCAAGAGGCTGAAAGAGATGAA | | EAELTEINRRHITESVNSIR | |
| | | | | CGAGAGGGAGAAGAAGGAGCTGCAGAAGATCCTGGACAGAAAGCGCCATAA | | RLEEAQKQRHDRLVAGQQ | |
| | | | | CAGCATCTCGGAGGCCAAGATGAGGGACAAGCATAAGAAGGAGGCGGAACT | | QVLQQLAEEEPKLLAQLAQ | |
| | | | | GACGGAGATTAACCGTCGGCACATCACTGAGTCAGTCAACTCCATCCGTCG | | ECQEQRARLPQEIRRSLLG | |
| | | | | GCTGGAGGAGGCCCAGAAGCAGCGGCATGACCGTCTTGTGGCTGGGCAGC | | EMPEGLGDGPLVACASNG | |
| | | | | AGCAGGTCCTGCAACAGCTGGCAGAAGAGGAGCCCAAGCTGCTGGCCCAG | | HAPGSSGHLSGADSESQE | |
| | | | | CTGGCCCAGGAGTGTCAGGAGCAGCGGGCGAGGCTCCCCCAGGAGATCCG | | ENTQL* | |
| | | | | CCGGAGCCTGCTGGGCGAGATGCCGGAGGGGCTGGGGGACGGGCCTCTG | | | |
| | | | | GTGGCCTGTGCCAGCAACGGTCACGCACCCGGGAGCAGCGGGCACCTGTC | | | |
| | | | | GGGCGCTGACTCGGAGAGCCAGGAGGAGAACACGCAGCTCTGA | | | |
| Shigella | 4 | prey700 | 106 | ATGGGAATTGGTCTTTCTGCTCAAGGTGTGAACATGAATAGACTACCAGGTT | 307 | MGIGLSAQGVNMNRLPGW | |
| paD | | | | GGGATAAGCATTCATATGGTTACCATGGGGATGATGGACATTCGTTTTGTTCT | | DKHSYGYHGDDGHSFCSS | |
| | | | | TCTGGAACTGGACAACCTTATGGACCAACTTTCACTACTGGTGATGTCATTG | | GTGQPYGPTFTTGDVIGCC | |
| | | | | GCTGTTGTGTTAATCTTATCAACAATACCTGCTTTTACACCAAGAATGGACAT | | VNLINNTCFYTKNGHSLGIA | |
| | | | | AGTTTAGGTATTGCTTTCACTGACCTACCGCCAAATTTGTATCCTACTGTGGG | | FTDLPPNLYPTVGLQTPGE | |
| | | | | GCTTCAAACACCAGGAGAAGTGGTCGATGCCAATTTTGGGCAACATCCTTTC | | VVDANFGQHPFVFDIEDYM | |
| | | | | GTGTTTGATATAGAAGACTATATGCGGGAGTGGAGAACCAAAATCCAGGCAC | | REWRTKIQAQIDRFPIGDR | |
| | | | | AGATAGATCGATTTCCTATCGGAGATCGAGAAGGAGAATGGCAGACCATGAT | | EGEWQTMIQKMVSSYLVH | |
| | | | | ACAAAAAATGGTTTCATCTTATTTAGTCCACCATGGGTACTGTGCCACAGCAG | | HGYCATAEAFARSTDQTVL | |
| | | | | AGGCCTTTGCCAGATCTACAGACCAGACCGTTCTAGAAGAATTAGCTTCCAT | | EELASIKNRQRIQKLVLAGR | |
| | | | | TAAGAATAGACAAAGAATTCAGAAATTGGTATTAGCAGGAAGAATGGGAGAA | | MGEAIETTQQLYPSLLERN | |
| | | | | GCCATTGAAACAACACAACAGTTATACCCAAGTTTACTTGAAAGAAATCCTAA | | PNLLFTLKVRQFIEMVNGT | |
| | | | | TCTCCTTTTCACATTAAAAGTGCGTCAGTTTATAGAAATGGTGAATGGTACAG | | DSEVRCLGGRSPKSQDSY | |
| | | | | ATAGTGAAGTACGATGTTTGGGAGGCCGAAGTCCAAAGTCTCAAGACAGTTA | | PVSPRPFSSPSMSPSHGM | |
| | | | | TCCTGTTAGTCCTCGACCTTTTAGTAGTCCAAGTATGAGCCCCAGCCATGGA | | NIHNLASGKGSTAHFSGFE | |
| | | | | ATGAATATCCACAATTTAGCATCAGGCAAAGGAAGCACCGCACATTTTTCAG | | SCSNGVISNKAHQSYCHSN | |
| | | | | GTTTTGAAAGTTGTAGTAATGGTGTAATATCAAATAAAGCACATCAATCATATT | | KHQSSNLNVPELNSINMSR | |
| | | | | GCCATAGTAATAAACACCAGTCATCCAACTTGAATGTACCAGAACTAAACAGT | | SQQVNNFTSNDVDMETDH | |
| | | | | ATAAATATGTCAAGATCACAGCAAGTTAATAACTTCACCAGTAATGATGTAGA | | YSNGVGETSSNGFLNGSS | |
| | | | | CATGGAAACAGATCACTACTCCAATGGAGTTGGAGAAACTTCATCCAATGGT | | KHDHEMEDCDTEMEVDSS | |
| | | | | TTCCTAAATGGTAGCTCTAAACATGACCACGAAATGGAAGATTGTGACACCG | | QLRRQLCGGSQAAIERMIH | |
| | | | | AAATGGAAGTTGATTCAAGTCAGTTGAGACGCCAGTTGTGTGGAGGAAGTCA | | FGRELQAMSEQLRRDCGK | |
| | | | | GGCCGCCATAGAAAGAATGATCCACTTTGGACGAGAGCTGCAAGCAATGAG | | NTANKKMLKDAFSLLAYSD | |
| | | | | TGAACAGCTAAGGAGAGACTGTGGCAAGAACACTGCAAACAAAAAAATGTTG | | PWNSPVGNQLDPIQREPV | |
| | | | | AAGGATGCATTCAGTCTACTAGCATATTCAGATCCCTGGAACAGCCCAGTTG | | CSALNSAILETHNLPKQPPL | |
| | | | | GAAATCAGCTTGACCCGATTCAGAGAGAACCTGTGTGCTCAGCTCTTAACAG | | ALAMGQATQCLGLMARSGI | |
| | | | | TGCAATATTAGAAACCCACAATCTGCCAAAGCAACCTCCACTTGCCCTAGCA | | GSCAFATVEDYLH* | |
| | | | | ATGGGACAGGCCACACAATGTCTAGGACTGATGGCTCGATCAGGAATTGGA | | | |
| | | | | TCCTGCGCATTTGCCACAGTGGAAGACTACCTACATTAG | | | |
| Shigella | 4 | prey2694 | 107 | ATGGCACACGCTATGGAAAACTCCTGGACAATCAGTAAAGAGTACCATATTG | 308 | MAHAMENSWTISKEYHIDE | |
| ipaD | | | | ATGAAGAAGTGGGCTTTGCTCTGCCAAATCCACAGGAAAATCTACCTGATTTT | | EVGFALPNPQENLPDFYND | |
| | | | | TATAATGACTGGATGTTCATTGCTAAACATCTGCCTGATCTCATAGAGTCTGG | | WMFIAKHLPDLIESGQLRE | |
| | | | | CCAGCTTCGAGAAAGAGTTGAGAAGTTAAACATGCTCAGCATTGATCATCTC | | RVEKLNMLSIDHLTDHKSQ | |
| | | | | ACAGACCACAAGTCACAGCGCCTTGCACGTCTAGTTCTGGGATGCATCACCA | | RLARLVLGCITMAYVWGKG | |
| | | | | TGGCATATGTGTGGGGCAAAGGTCATGGAGATGTCCGTAAGGTCTTGCCAA | | HGDVRKVLPRNIAVPYCQL | |
| | | | | GAAATATTGCTGTTCCTTACTGCCAACTCTCCAAGAAACTGGAACTGCCTCCT | | SKKLELPPILVYADCVLANW | |
| | | | | ATTTTGGTTTATGCAGACTGTGTCTTGGCAAACTGGAAGAAAAAGGATCCTAA | | KKKDPNKPLTYENMDVLFS | |
| | | | | TAAGCCCCTGACTTATGAGAACATGGACGTTTTGTTCTCATTTCGTGATGGAG | | FRDGDCSKGFFLVSLLVEIA | |
| | | | | ACTGCAGTAAAGGATTCTTCCTGGTCTCTCTATTGGTGGAAATAGCAGCTGC | | AASAIKVIPTVFKAMQMQE | |
| | | | | TTCTGCAATCAAAGTAATTCCTACTGTATTCAAGGCAATGCAAATGCAAGAAC | | RDTLLKALLEIASCLEKALQ | |
| | | | | GGGACACTTTGCTAAAGGCGCTGTTGGAAATAGCTTCTTGCTTGGAGAAAGC | | VFHQIHDHVNPKAFFSVLRI | |
| | | | | CCTTCAAGTGTTTCACCAAATCCACGATCATGTGAACCCAAAAGCATTTTTCA | | YLSGWKGNPQLSDGLVYE | |
| | | | | GTGTTCTTCGCATATATTTGTCTGGCTGGAAAGGCAACCCCCAGCTATCAGA | | GFWEDPKEFAGGSAGQSS | |
| | | | | CGGTCTGGTGTATGAAGGGTTCTGGGAAGACCCAAAGGAGTTTGCAGGGGG | | VFQCFDVLLGIQQTAGGGH | |
| | | | | CAGTGCAGGCCAAAGCAGCGTCTTTCAGTGCTTTGACGTCCTGCTGGGCAT | | AAQFLQDMRRYMPPAHRN | |
| | | | | CCAGCAGACTGCTGGTGGAGGACATGCTGCTCAGTTCCTCCAGGACATGAG | | FLCSLESNPSVREFVLSKG | |
| | | | | AAGATATATGCCACCAGCTCACAGGAACTTCCTGTGCTCATTAGAGTCAAAT | | DAGLREAYDACVKALVSLR | |
| | | | | CCCTCAGTCCGTGAGTTTGTCCTTTCAAAAGGTGATGCTGGCCTGCGGGAA | | SYHLQIVTKYILIPASQQPKE | |
| | | | | GCTTATGACGCCTGTGTGAAAGCTCTGGTCTCCCTGAGGAGCTACCATCTGC | | NKTSEDPSKLEAKGTGGTD | |
| | | | | AAATCGTGACTAAGTACATCCTGATTCCTGCAAGCCAGCAGCCAAAGGAGAA | | LMNFLKTVRSTTEKSLLKE | |
| | | | | TAAGACCTCTGAAGACCCTTCAAAACTGGAAGCCAAAGGAACTGGAGGCACT | | G* | |
| | | | | GATTTAATGAATTTCCTGAAGACTGTAAGAAGTACAACTGAGAAATCCCTTTT | | | |
| | | | | GAAGGAAGGTTAA | | | |
| Shigella | 4 | prey53735 | 108 | GGGTGAACCAGAAGGTTCCTTCGTGGATTACCAAACAACTATGGTGCGGACA | 309 | GEPEGSFVDYQTTMVRTA | |
| ipaD | | | | GCCAAGGCCATTGCAGTGACCGTTCAGGAGATGGTTACCAAGTCAAACACC | | KAIAVTVQEMVTKSNTSPE | |
| | | | | AGCCCAGAGGAGCTGGGCCCTCTTGCTAACCAGCTGACCAGTGACTATGGC | | ELGPLANQLTSDYGRLASE | |
| | | | | CGTCTGGCCTCGGAGGCCAAGCCTGCAGCGGTGGCTGCTGAAAATGAAGA | | AKPAAVAAENEEIGSHIKHR | |
| | | | | GATAGGTTCCCATATCAAACACCGGGTACAGGAGCTGGGCCATGGCTGTGC | | VQELGHGCAALVTKAGALQ | |
| | | | | CGCTCTGGTCACCAAGGCAGGCGCCCTGCAGTGCAGCCCCAGTGATGCCTA | | CSPSDAYTKKELIECARRV | |
| | | | | CACCAAGAAGGAGCTCATAGAGTGTGCCCGGAGAGTCTCTGAGAAGGTCTC | | SEKVSHVLAALQAGNRGT | |
| | | | | CCACGTCCTGGCTGCGCTCCAGGCTGGGAATCGTGGCACCCAGGCCTGCAT | | QACITAASAVSGIIADLDTTI | |
| | | | | CACAGCAGCCAGCGCTGTGTCTGGTATCATTGCTGACCTCGACACCACCATC | | MFATAGTLNREGTETFADH | |
| | | | | ATGTTCGCCACTGCTGGCACGCTCAATCGTGAGGGTACTGAAACTTTCGCTG | | REGILKTAKVLVEDTKVLVQ | |
| | | | | ACCACCGGGAGGGCATCCTGAAGACTGCGAAGGTGCTGGTGGAGGACACC | | NAAGSQEKLAQAAQSSVA | |
| | | | | AAGGTCCTGGTGCAAAACGCAGCTGGGAGCCAGGAGAAGTTGGCGCAGGC | | TITRLADVVKLGAASLGAED | |
| | | | | TGCCCAGTCCTCCGTGGCGACCATCACCCGCCTCGCTGATGTGGTCAAGCT | | PETQVVLINAVKDVAKALG | |
| | | | | GGGTGCAGCCAGCCTGGGAGCTGAGGACCCTGAGACCCAGGTGGTACTAA | | DLISATKAAAGKVGDDPAV | |
| | | | | TCAACGCAGTGAAAGATGTAGCCAAAGCCCTGGGAGACCTCATCAGTGCAA | | WQLKNSAKVMVTNVTSLLK | |
| | | | | CGAAGGCTGCAGCTGGCAAAGTTGGAGATGACCCTGCTGTGTGGCAGCTAA | | TVKAVEDEATKGTRALEAT | |
| | | | | AGAACTCTGCCAAGGTGATGGTGACCAATGTGACATCATTGCTTAAGACAGT | | TEHIRQELAVFCSPEPPAKT | |
| | | | | AAAAGCCGTGGAAGATGAGGCCACCAAAGGCACTCGGGCCCTGGAGGCAA | | STPEDFIRMTKGITMATAKA | |
| | | | | CCACAGAACACATACGGCAGGAGCTGGCGGTTTTCTGTTCCCCAGAGCCAC | | VAAGNSCRQEDVIATANLS | |
| | | | | CTGCCAAGACCTCTACCCCAGAAGACTTCATCCGAATGACCAAGGGTATCAC | | RRAIADMLRACKEAAYHPE | |
| | | | | CATGGCAACCGCCAAGGCCGTTGCTGCTGGCAATTCCTGTCGCCAGGAAGA | | VAPDVRLRALHYGRECAN | |
| | | | | TGTCATTGCCACAGCCAATCTGAGCCGCCGTGCTATTGCAGATATGCTTCGG | | GYLELLDHVLLTLQKPSPEL | |
| | | | | GCTTGCAAGGAAGCAGCTTACCACCCAGAAGTGGCCCCTGATGTGCGGCTT | | KQQLTGHSKRVAGSVTELI | |
| | | | | CGAGCCCTGCACTATGGCCGGGAGTGTGCCAATGGCTACCTGGAACTGCTG | | QAAEAMKGTEWVDPEDPT | |
| | | | | GACCATGTACTGCTGACCCTGCAGAAGCCAAGCCCAGAACTGAAGCAGCAG | | VIAENELLGAAAAIEAAAKK | |
| | | | | TTGACAGGACATTCAAAGCGTGTGGCTGGTTCCGTCACTGAGCTCATCCAGG | | LEQLKPRAKPKEADESLNF | |
| | | | | CTGCTGAAGCCATGAAGGGAACAGAATGGGTAGACCCAGAGGACCCCACAG | | EEQILEAAKSIAAATSALVK | |
| | | | | TCATTGCTGAGAATGAGCTCCTGGGAGCTGCAGCCGCCATTGAGGCTGCAG | | AASAAQRELVAQGKVGAIP | |
| | | | | CCAAAAAGCTAGAGCAGCTGAAGCCCCGGGCCAAACCCAAGGAGGCAGATG | | ANALDDGQWSQGLISAAR | |
| | | | | AGTCCTTGAACTTTGAGGAGCAGATACTAGAAGCTGCCAAGTCCATTGCAGC | | MVAAATNNLCEAANAAVQ | |
| | | | | AGCCACCAGTGCACTGGTAAAGGCTGCGTCGGCTGCCCAGAGAGAACTAGT | | GHASQEKLISSAKQVAAST | |
| | | | | GGCCCAAGGGAAGGTGGGTGCCATTCCAGCCAATGCACTGGACGATGGGC | | AQLLVACKVKADQDSEAM | |
| | | | | AGTGGTCCCAGGGCCTCATTTCTGCTGCCCGGATGGTGGCTGCGGCCACCA | | KRLQAAGNAVKRASDNLVK | |
| | | | | ACAATCTGTGTGAGGCAGCCAATGCAGCTGTACAAGGCCATGCCAGCCAGG | | AAQKAAAFEEQENETVVVK | |
| | | | | AGAAGCTCATCTCATCAGCCAAGCAGGTAGCTGCCTCCACAGCCCAGCTCC | | EKMVGGIAQIIAAQEEMLRK | |
| | | | | TTGTGGCCTGCAAGGTCAAGGCTGACCAGGACTCGGAGGCAATGAAACGAC | | ERELEEARKKLAQIRQQQY | |
| | | | | TTCAGGCTGCTGGCAACGCAGTGAAGCGAGCCTCAGATAATCTGGTGAAAG | | KFLPSELRDEH* | |
| | | | | CAGCACAGAAGGCTGCAGCCTTTGAAGAGCAGGAGAATGAGACAGTGGTGG | | | |
| | | | | TGAAAGAGAAGATGGTTGGCGGCATTGCCCAGATCATCGCAGCACAGGAAG | | | |
| | | | | AAATGCTTCGGAAGGAACGAGAGCTGGAAGAGGCGCGGAAGAAACTGGCC | | | |
| | | | | CAGATCCGGCAGCAGCAGTACAAGTTTCTGCCTTCAGAGCTTCGAGATGAG | | | |
| | | | | CACTAA | | | |
| Shigella | 4 | prey67574 | 109 | NNACAGGAGANTGAGTTGCAANCGGCGGGTGATGCNNNTCTACCNGNNCGT | 310 | XQEXELQXAGDAXLPXRXR | |
| ipaD | | | | GNACGANCCACAGACGCCNCTNCCTGGGTCCTGGGATNCCAAACNACANNN | | XTDAXXWVLGXQTTXXXTX | |
| | | | | NCATNTACNTTNGTCTNTGTCAGANCANNCTGNGGNTGCACTNCNNNCGTCA | | VXVRXXXGCTXXVIA*XXX | |
| | | | | TTGCTTAACNNNACNAGATGCCNCGTCATTTCNAGNCACNCATACAATACCA | | MPRHFXXXIQYHXXX*FXFX | |
| | | | | CNTGCNTGNGTGATTTNTTTTTTNGANNTGCCAATTNTGATGAAGGGAACATA | | XCQX**REHXXSWELVFLX | |
| | | | | TNTNTTCATGGGAATTGGTCTTTCTGTTNANNGTNTNAACAC | | XVXT | |
| Shigella | 5 | prey67509 | 110 | GCTACTCACCCACCTCTCCCAGCTACTCGCCCACCTCTCCCAGCTATTCGCC | 311 | YSPTSPSYSPTSPSYSPTS | |
| ipaC | | | | CACCTCTCCCAGCTACTCACCCACTTCCCCTAGCTATTCGCCCACTTCCCCT | | PSYSPTSPSYSPTSPSYSP | |
| | | | | AGCTACTCGCCAACGTCTCCCAGCTACTCGCCGACATCTCCCAGCTACTCGC | | TSPSYSPTSPSYSPTSPSY | |
| | | | | CAACTTCACCCAGCTATTCTCCCACTTCTCCCAGCTACTCACCTACCTCTCCA | | SPTSPSYSPTSPSYSPTSP | |
| | | | | AGCTATTCACCCACCTCCCCCAGCTACTCACCCACTTCCCCAAGTTACTCAC | | SYSPTSPSYSPTSPNYSPT | |
| | | | | CCACCAGCCCGAACTATTCTCCAACCAGTCCCAATTACACCCCAACATCACC | | SPNYTPTSPSYSPTSPSYS | |
| | | | | CAGCTACAGCCCGACATCACCCAGCTATTCCCCTACTAGTCCCAACTACACA | | PTSPNYTPTSPNYSPTSPS | |
| | | | | CCTACCAGCCCTAACTACAGCCCAACCTCTCCAAGCTACTCTCCAACATCAC | | YSPTSPSYSPTSPSYSPSS | |
| | | | | CCAGCTATTCCCCGACCTCACCAAGTTACTCCCCTTCCAGCCCACGATACAC | | PRYTPQSPTYTPSSPSYSP | |
| | | | | ACCACAGTCTCCAACCTATACCCCAAGCTCACCCAGCTACAGCCCCAGTTCG | | SSPSYSPTSPKYTPTSPSY | |
| | | | | CCCAGCTACAGCCCAACCTCACCCAAGTACACCCCAACCAGTCCTTCTTATA | | SPSSPEYTPTSPKYSPTSP | |
| | | | | GTCCCAGCTCCCCAGAGTATACCCCAACCTCTCCCAAGTACTCACCTACCAG | | KYSPTSPKYSPTSPTYSPT | |
| | | | | TCCCAAATATTCACCCACCTCTCCCAAGTACTCGCCTACCAGTCCCACCTATT | | TPKYSPTSPTYSPTSPVYT | |
| | | | | CACCCACCACCCCAAAATACTCCCCAACATCTCCTACTTATTCCCCAACCTCT | | PTSPKYSPTSPTYSPTSPK | |
| | | | | CCAGTCTACACCCCAACCTCTCCCAAGTACTCACCTACTAGCCCCACTTACT | | YSPTSPTYSPTSPKGSTYS | |
| | | | | CGCCCACTTCCCCCAAGTACTCGCCCACCAGCCCCACCTACTCGCCCACCT | | PTSPGYSPTSPTYSLTSPAI | |
| | | | | CCCCCAAAGGCTCAACCTACTCTCCCACTTCCCCTGGTTACTCGCCCACCAG | | SPDDSDEEN* | |
| | | | | CCCCACCTACAGTCTCACAAGCCCGGCTATCAGCCCGGATGACAGTGACGA | | | |
| | | | | GGAGAACTGA | | | |
| Shigella | 5 | prey67514 | 111 | ATGCACAAGGAGGAACATGAGGTGGCTGTGCTGGGGGCACCCCCCAGCAC | 312 | MHKEEHEVAVLGAPPSTIL | |
| ipaC | | | | CATCCTTCCAAGGTCCACCGTGATCAACATCCACAGCGAGACCTCCGTGCC | | PRSTVINIHSETSVPDHVV | |
| | | | | CGACCATGTCGTCTGGTCCCTGTTCAACACCCTCTTCTTGAACTGGTGCTGT | | WSLFNTLFLNWCCLGFIAF | |
| | | | | CTGGGCTTCATAGCATTCGCCTACTCCGTGAAGTCTAGGGACAGGAAGATG | | AYSVKSRDRKMVGDVTGA | |
| | | | | GTTGGCGACGTGACCGGGGCCCAGGCCTATGCCTCCACCGCCAAGTGCCT | | QAYASTAKCLNIWALILGIL | |
| | | | | GAACATCTGGGCCCTGATTCTGGGCATCCTCATGACCATTGGATTCATCCTG | | MTIGFILSLVFGSVTVYHUML | |
| | | | | TCACTGGTATTCGGCTCTGTGACAGTCTACCATATTATGTTACAGATAATACA | | QIIQEKRGY* | |
| | | | | GGAAAAACGGGGTTACTAG | | | |
| Shigella | 5 | prey2926 | 112 | ATGGAGAAAACTTGTATAGATGCACTTCCTCTTACTATGAATTCTTCAGAAAA | 313 | MEKTCIDALPLTMNSSEKQ | |
| ipaC | | | | GCAAGAGACTGTATGTATTTTTGGAACTGGTGATTTTGGAAGATCACTGGGA | | ETVCIFGTGDFGRSLGLKM | |
| | | | | TTGAAAATGCTCCAGTGTGGTTATTCTGTTGTTTTTGGAAGTCGAAACCCCCA | | LQCGYSVVFGSRNPQKTTL | |
| | | | | GAAGACCACCCTACTGCCCAGTGGTGCAGAAGTCTTGAGCTATTCAGAAGCA | | LPSGAEVLSYSEAAKKSDIII | |
| | | | | GCCAAGAAGTCTGACATCATAATCATAGCAATCCACAGAGAGCATTATGATTT | | IAIHREHYDFLTELTEVLNG | |
| | | | | TCTCACAGAATTAACTGAGGTTCTCAATGGAAAAATATTGGTAGACATCAGCA | | KILVDISNNLKINQYPESNA | |
| | | | | ACAACCTCAAAATCAATCAATATCCAGAATCTAATGCAGAGTACCTTGCTCAT | | EYLAHLVPGAHVVKAFNTIS | |
| | | | | TTGGTGCCAGGAGCCCACGTGGTAAAAGCATTTAACACCATCTCAGCCTGG | | AWALQSGALDASRQVFVC | |
| | | | | GCTCTCCAGTCAGGAGCACTGGATGCAAGTCGGCAGGTGTTTGTGTGTGGA | | GNDSKAKQRVMDIVRNLGL | |
| | | | | AATGACAGCAAAGCCAAGCAAAGAGTGATGGATATTGTTCGTAATCTTGGAC | | TPMDQGSLMAAKEIEKYPL | |
| | | | | TTACTCCAATGGATCAAGGATCACTCATGGCAGCCAAAGAAATTGAAAAGTA | | QLFPMWRFPFYLSAVLCVF | |
| | | | | CCCCCTGCAGCTATTTCCAATGTGGAGGTTCCCCTTCTATTTGTCTGCTGTG | | LFFYCVIRDVIYPYVYEKKD | |
| | | | | CTGTGTGTCTTCTTGTTTTTCTATTGTGTTATAAGAGACGTAATCTACCCTTAT | | NTFRMAISIPNRIFPITAPYT | |
| | | | | GTTTATGAAAAGAAAGATAATACATTTCGTATGGCTATTTCCATTCCAAATCGT | | ACFGLPPWCYCCHSTTVP | |
| | | | | ATCTTTCCAATAACAGCACCTTACACTGCTTGCTTTGGTTTACCTCCCTGGTG | | RHKIPSIPRLA* | |
| | | | | TTATTGCTGCCATTCTACAACTGTACCGAGGCACAAAATACCGTCGATTCCCA | | | |
| | | | | GACTGGCTTGA | | | |
| Shigella | 5 | prey4458 | 113 | CCAGGACGTCCAGGCCAGCCAGGCGGAGGCTGACCAGCAGCAGACTCGCC | 314 | QDVQASQAEADQQQTRLK | |
| ipaC | | | | TCAAGGAGCTGGAGTCCCAGGTGTCGGGTCTGGAGAAGGAGGCCATCGAG | | ELESQVSGLEKEAIELREAV | |
| | | | | CTCAGGGAGGCCGTCGAGCAGCAGAAAGTGAAGAACAATGACCTCCGGGA | | EQQKVKNNDLREKNWKAM | |
| | | | | GAAGAACTGGAAGGCCATGGAGGCACTGGCCACGGCCGAGCAGGCCTGCA | | EALATAEQACKEKLHSLTQ | |
| | | | | AGGAGAAGCTGCACTCCCTGACCCAGGCCAAGGAGGAATCGGAGAAGCAG | | AKEESEKQLCLIEAQTMEA | |
| | | | | CTCTGTCTGATTGAGGCGCAGACCATGGAGGCCCTGCTGGCTCTGCTCCCA | | LLALLPELSVL | |
| | | | | GAACTCTCTGTCTTGGC | | | |
| Shigella | 5 | prey4458 | 114 | GGCCGAGGAGACGCAGAGCACACTGCAGGCCGAGTGTGACCAGTACCGCA | 315 | AEETQSTLQAECDQYRSIL | |
| ipaC | | | | GCATCCTGGCGGAGACGGAGGGCATGCTCAGAGACCTGCAGAAGAGCGTG | | AETEGMLRDLQKSVEEEE | |
| | | | | GAGGAGGAGGAGCAGGTGTGGAGGGCCAAGGTGGGCGCCGCAGAGGAGG | | QVWRAKVGAAEEELQKSR | |
| | | | | AGCTCCAGAAGTCCCGGGTCACAGTGAAGCATCTCGAAGAGATTGTAG | | VTVKHLEEIV | |
| Shigella | 5 | prey67522 | 115 | GANGAATNCNNTATGCCAAAAGGACAAGGAGGTATTGGTNGCTTANGCTGG | 316 | XEXXMPKGQGGIGXLXWL* | |
| ipaC | | | | CTATGAATACNTCNTTCTGTTTGTGATANTCTATTTCTTACACCNTCNGGCAT | | IXXSVCDXLFLTPSGMVGX | |
| | | | | GGTAGGCAANNGCCACAGTANATGCCACATCTATGAGGCTGNNGCNGCATA | | XHSXCHIYEAXAAYSPCLX | |
| | | | | CTCGCCGTGTCTANCTACATCCTNGTTANNGGNTGNGGCCCGNNCGGTTCC | | TSXLXXXARXVPXDXVXXT | |
| | | | | TNCCGATTNTGTTCNGGNCACAGCCTGGTGTNTGACANCTCGGACCGCGNT | | AWCXTXRTAXTXTSWRTY | |
| | | | | NACTATNACCTCCTGGAGGACCTACCACGAANGCATGCTNACCCTGGTGGG | | HEXMLTLVGRLE | |
| | | | | GAGGCTGGAAGG | | | |
| Shigella | 5 | prey527 | 116 | CATGACTGCAGACCTTCCTAATGAACTCATTGAACTGCTGGAGAAAATTGTC | 317 | MTADLPNELIELLEKIVLDN | |
| ipaC | | | | CTTGATAACTCTGTATTCAGTGAACACAGGAATCTGCAAAACCTCCTTATCCT | | SVFSEHRNLQNLLILTAIKA | |
| | | | | CACTGCAATTAAGGCTGACCGTACACGTGTTATGGAGTATATTAACCGCCTG | | DRTRVMEYINRLDNYDAPD | |
| | | | | GATAATTATGATGCCCCAGATATTGCCAATATCGCCATCAGCAATGAGCTGTT | | IANIAISNELFEEAFAIFRKF | |
| | | | | TGAAGAAGCATTTGCCATTTTCCGGAAATTTGATGTCAATACTTCAGCAGTTC | | DVNTSAVQVLIEHIGNLDRA | |
| | | | | AGGTCTTAATTGAGCATATTGGAAACTTGGATCGGGCATATGAGTTTGCTGA | | YEFAERCNEPAVWSQLAK | |
| | | | | ACGTTGCAATGAACCTGCGGTCTGGAGTCAACTTGCAAAAGCCCAGTTGCAG | | AQLQKGMVKEAIDSYIKAD | |
| | | | | AAAGGAATGGTGAAAGAAGCCATTGATTCTTATATCAAAGCAGATGATCCTTC | | DPSSYMEVVQAANTSGNW | |
| | | | | CTCCTACATGGAAGTTGTTCAGGCTGCCAATACTAGTGGAAACTGGGAAGAA | | EELVKYLQMARKKARESYV | |
| | | | | CTGGTGAAGTACTTGCAGATGGCCCGTAAGAAGGCTCGAGAGTCCTATGTG | | ETELIFALAKTNR | |
| | | | | GAGACAGAACTGATATTCGCACTGGCTAAAACAAACCGC | | | |
| Shigella | 5 | prey53735 | 117 | TGCAGTCCAAGAGATCTCCCATCTCATTGAGCCGCTGGCCAATGCTGCCCG | 318 | AVQEISHLIEPLANAARAEA | |
| ipaC | | | | GGCTGAAGCCTCCCAGCTGGGACACAAGGTGTCCCAGATGGCGCAGTACTT | | SQLGHKVSQMAQYFEPLTL | |
| | | | | TGAGCCGCTCACCCTGGCTGCAGTGGGTGCTGCCTCCAAGACCCTGAGCCA | | AAVGAASKTLSHPQQMALL | |
| | | | | CCCGCAGCAGATGGCACTCCTGGACCAGACTAAAACATTGGCAGAGTCTGC | | DQTKTLAESALQLLYTAKE | |
| | | | | CCTGCAGTTGCTATACACTGCCAAGGAGGCTGGTGGTAACCCAAAGCAAGC | | AGGNPKQAAHTQEALEEA | |
| | | | | AGCTCACACCCAGGAAGCCCTGGAGGAGGCTGTGCAGATGATGACCGAGG | | VQMMTEAVEDLTTTLNEAA | |
| | | | | CCGTAGAGGACCTGACAACAACCCTCAACGAGGCAGCCAGTGCTGCTGGGG | | SAAGVVGGMVDSITQAINQ | |
| | | | | TCGTGGGTGGCATGGTGGACTCCATCACCCAGGCCATCAACCAGCTAGATG | | LDEGPMGEPEGSFVDYQT | |
| | | | | AAGGACCAATGGGTGAACCAGAAGGTTCCTTCGTGGATTACCAAACAACTAT | | TMVRTAKAIAVTVQEMVTK | |
| | | | | GGTGCGGACAGCCAAGGCCATTGCAGTGACCGTTCAGGAGATGGTTACCAA | | SNTSPEELGPLANQLTSDY | |
| | | | | GTCAAACACCAGCCCAGAGGAGCTGGGCCCTCTTGCTAACCAGCTGACCAG | | GRLASEAKPAAVAAENEEI | |
| | | | | TGACTATGGCCGTCTGGCCTCGGAGGCCAAGCCTGCAGCGGTGGCTGCTG | | GSHIKHRVQELGHGCAALV | |
| | | | | AAAATGAAGAGATAGGTTCCCATATCAAACACCGGGTACAGGAGCTGGGCC | | TKAGALQCSPSDAYTKKELI | |
| | | | | ATGGCTGTGCCGCTCTGGTCACCAAGGCAGGCGCCCTGCAGTGCAGCCCC | | ECARRVSEKVSHVLAALQA | |
| | | | | AGTGATGCCTACACCAAGAAGGAGCTCATAGAGTGTGCCCGGAGAGTCTCT | | GNRGTQACITAASAVSGIIA | |
| | | | | GAGAAGGTCTCCCACGTCCTGGCTGCGCTCCAGGCTGGGAATCGTGGCACC | | DLDTTIMFATAGTLNREGT | |
| | | | | CAGGCCTGCATCACAGCAGCCAGCGCTGTGTCTGGTATCATTGCTGACCTC | | ETFADHREGILKTAKVLVED | |
| | | | | GACACCACCATCATGTTCGCCACTGCTGGCACGCTCAATCGTGAGGGTACT | | TKVLVQNAAGSQEKLAQAA | |
| | | | | GAAACTTTCGCTGACCACCGGGAGGGCATCCTGAAGACTGCGAAGGTGCTG | | QSSVATITRLADVVKLGAAS | |
| | | | | GTGGAGGACACCAAGGTCCTGGTGCAAAACGCAGCTGGGAGCCAGGAGAA | | LGAEDPETQVVLINAVKDV | |
| | | | | GTTGGCGCAGGCTGCCCAGTCCTCCGTGGCGACCATCACCCGCCTCGCTGA | | AKALGDLISATKAAAGKVG | |
| | | | | TGTGGTCAAGCTGGGTGCAGCCAGCCTGGGAGCTGAGGACCCTGAGACCC | | DDPAVWQLKNSAKVMVTN | |
| | | | | AGGTGGTACTAATCAACGCAGTGAAAGATGTAGCCAAAGCCCTGGGAGACC | | VTSLLKTVKAVEDEATKGT | |
| | | | | TCATCAGTGCAACGAAGGCTGCAGCTGGCAAAGTTGGAGATGACCCTGCTG | | RALEATTEHIRQELAVFCSP | |
| | | | | TGTGGCAGCTAAAGAACTCTGCCAAGGTGATGGTGACCAATGTGACATCATT | | EPPAKTSTPEDFIRMTKGIT | |
| | | | | GCTTAAGACAGTAAAAGCCGTGGAAGATGAGGCCACCAAAGGCACTCGGGC | | MATAKAVAAGNSCRQEDVI | |
| | | | | CCTGGAGGCAACCACAGAACACATACGGCAGGAGCTGGCGGTTTTCTGTTC | | ATANLSRRAIADMLRACKE | |
| | | | | CCCAGAGCCACCTGCCAAGACCTCTACCCCAGAAGACTTCATCCGAATGAC | | AAYHPEVAPDVRLRALHYG | |
| | | | | CAAGGGTATCACCATGGCAACCGCCAAGGCCGTTGCTGCTGGCAATTCCTG | | RECANGYLELLD | |
| | | | | TCGCCAGGAAGATGTCATTGCCACAGCCAATCTGAGCCGCCGTGCTATTGC | | | |
| | | | | AGATATGCTTCGGGCTTGCAAGGAAGCAGCTTACCACCCAGAAGTGGCCCC | | | |
| | | | | TGATGTGCGGCTTCGAGCCCTGCACTATGGCCGGGAGTGTGCCAATGGCTA | | | |
| | | | | CCTGGAACTGCTGGAC | | | |
| Shigella | 5 | prey53735 | 118 | CAGTGATGTGCTGGACAAGGCCAGCAGCCTCATTGAGGAGGCGAAAAAGGC | 319 | SDVLDKASSLIEEAKKAAG | |
| ipaC | | | | AGCTGGCCATCCAGGGGACCCTGAGAGCCAGCAGCGGCTTGCCCAGGTGG | | HPGDPESQQRLAQVAKAV | |
| | | | | CTAAAGCAGTGACCCAGGCTCTGAACCGCTGTGTCAGCTGCCTACCTGGCC | | TQALNRCVSCLPGQRDVD | |
| | | | | AGCGCGATGTGGATAATGCCCTGAGGGCAGTTGGAGATGCCAGCAAGCGAC | | NALRAVGDASKRLLSDSLP | |
| | | | | TCCTGAGTGACTCGCTTCCTCCTAGCACTGGGACATTTCAAGAAGCTCAGAG | | PSTGTFQEAQSRLNEAAAG | |
| | | | | CCGGTTGAATGAAGCTGCTGCTGGGCTGAATCAGGCAGCCACAGAACTGGT | | LNQAATELVQASRGTPQDL | |
| | | | | GCAGGCCTCTCGGGGAACCCCTCAGGACCTGGCTCGAGCCTCAGGCCGAT | | ARASGRFGQDFSTFLEAGV | |
| | | | | TTGGACAGGACTTCAGCACCTTCCTGGAAGCTGGTGTGGAGATGGCAGGCC | | EMAGQAPSQEDRAQVVSN | |
| | | | | AGGCTCCGAGCCAGGAGGACCGAGCCCAAGTTGTGTCCAACTTGAAGGGCA | | LKGISMSSSKLLLAAKALST | |
| | | | | TCTCCATGTCTTCAAGCAAACTTCTTCTGGCTGCCAAGGCCCTGTCCACGGA | | DPAAPNLKSQLAAAARAVT | |
| | | | | CCCTGCTGCCCCTAACCTCAAGAGTCAGCTGGCTGCAGCTGCCAGGGCAGT | | DSINQLITMCTQQAPGQKE | |
| | | | | AACTGACAGCATCAATCAGCTCATCACTATGTGCACCCAGCAGGCACCCGG | | CDNALRELETVRELLENPV | |
| | | | | CCAGAAGGAGTGTGATAACGCCCTGCGGGAATTGGAGACGGTCCGGGAACT | | QPINDMSYFGCLDSVMENS | |
| | | | | CCTGGAGAACCCAGTCCAGCCCATCAATGACATGTCCTACTTTGGTTGCCTG | | KVLGEAMTGISQNAKNGNL | |
| | | | | GACAGTGTAATGGAGAACTCAAAGGTGCTGGGCGAGGCCATGACTGGCATC | | PEFGDAISTASKALCGFTEA | |
| | | | | TCCCAAAATGCCAAGAACGGAAACCTGCCAGAGTTTGGAGATGCCATTTCCA | | AAQAAYLVGVSDPNSQAG | |
| | | | | GAGCCTCAAAGGCACTTTGTGGCTTCACCGAGGCAGCTGCACAGGCTGCAT | | QQGLVEPTQFARANQAIQ | |
| | | | | ATCTGGTTGGTGTCTCTGACCCCAATAGCCAAGCTGGACAGCAAGGGCTAG | | MACQSLGEPGCTQAQVLS | |
| | | | | TGGAGCCCACACAGTTTGCCCGTGCAAACCAGGCAATTCAGATGGCCTGCC | | AATIVAKHTSALCNSCRLAS | |
| | | | | AGAGTTTGGGAGAGCCTGGCTGTACCCAGGCCCAGGTGCTCTCTGCAGCCA | | ARTTNPTAKRQFVQSAKEV | |
| | | | | CCATTGTGGCTAAACACACCTCTGCACTGTGTAACAGCTGTCGCCTGGCTTC | | ANSTANLVKTIKALDGAFTE | |
| | | | | TGCCCGTACCACCAATCCTACTGCCAAGCGCCAGTTTGTACAGTCAGCCAAG | | ENRAQCRAATAPLLEAVDN | |
| | | | | GAGGTGGCCAACAGCACAGCTAATCTTGTCAAGACCATCAAGGCGCTAGAT | | LSAFASNPEFSSIPAQISPE | |
| | | | | GGGGCCTTCACAGAGGAGAACCGTGCCCAGTGCCGAGCAGCAACAGCCCC | | GRAAMEPIVISAKTMLESA | |
| | | | | TCTGCTGGAGGCTGTGGACAATCTGAGTGCCTTTGCGTCCAACCCTGAGTTC | | GGLIQTARALAVNPRD | |
| | | | | TCCAGCATTCCTGCCCAGATCAGCCCTGAGGGTCGGGCTGCCATGGAGCCC | | | |
| | | | | ATTGTGATCTCTGCCAAGACAATGTTAGAGAGTGCCGGGGGACTCATCCAGA | | | |
| | | | | CAGCCCGGGCCCTCGCAGTCAATCCCCGGGAC | | | |
| Shigella | 5 | prey67546 | 119 | CACAGGGGCTGACCTGCTGGAAGAGCATCTTGGTGAAATCTGGAACCTGCG | 320 | TGADLLEEHLGEIWNLRQR | |
| ipaC | | | | CCAGCGCCTGGAGGAGTCCATCTGCATCAATGACTGCCTACGGGAGCAACT | | LEESICINDCLREQLEHR | |
| | | | | GGAACACCGGC | | | |
| Shigella | 5 | prey4671 | 120 | CCTGGAGAGTCTCATCCAGAGAGTATCCCAGCTGGAGGCCCAGCTCCCAAA | 321 | LESLIQRVSQLEAQLPKNGL | |
| ipaC | | | | AAATGGACTAGAAGAGAAGCTGGCTGAGGAGCTGAGATCAGCCTCGTGGCC | | EEKLAEELRSASWPGKYDS | |
| | | | | TGGGAAATATGATTCCCTGATTCAGGATCAGGCCCGGGAACTGTCTTACCTA | | LIQDQARELSYLRQKIREGR | |
| | | | | CGGCAAAAAATACGAGAAGGGAGAGGTATTTGTTATCTTATCACCCGGCATG | | GICYLITRHAKDTVKSFEDL | |
| | | | | CAAAAGATACAGTAAAATCTTTTGAGGATCTCCTAAGGAGCAATGACATTGAC | | LRSNDIDYYLGQSFREQLA | |
| | | | | TACTACCTGGGACAGAGCTTCCGGGAGCAACTCGCCCAGGGAAGCCAGCTG | | QGSQLTERLTSKLSTKDHK | |
| | | | | ACAGAGAGGCTCACCAGCAAACTCAGCACCAAGGATCATAAAAGTGAGAAA | | SEKDQAGLEPLALRLSREL | |
| | | | | GATCAAGCTGGACTTGAGCCACTGGCCCTCAGGCTCAGCAGGGAGCTGCAG | | QEKEKVIEVLQAKLDARSLT | |
| | | | | GAGAAGGAGAAAGTGATTGAAGTCCTGCAGGCCAAGCTGGATGCTCGGTCC | | PSSSHALSDSHRSPSSTSF | |
| | | | | CTCACACCCTCCAGCAGCCATGCCTTGTCTGACTCCCACCGCTCTCCCAGCA | | LSDELEACSDMDIVSEYTH | |
| | | | | GCACCTCTTTCCTGTCTGATGAACTGGAAGCCTGCTCTGACATGGACATAGT | | YEEKKASPSHSDSIHHSSH | |
| | | | | CAGCGAGTACACACACTATGAAGAGAAGAAAGCTTCTCCCAGTCACTCAGAT | | SAVLSSKPSSTSASQGAKA | |
| | | | | TCCATCCATCATTCGAGTCATTCTGCTGTGTTGTCTTCTAAACCATCATCAAC | | ESNSNPISLPTPQNTPKEA | |
| | | | | CAGTGCATCTCAGGGGGCTAAGGCCGAATCCAACAGCAACCCCATCAGCTT | | NQAHSGFHFHSIPKLASLP | |
| | | | | GCCAACTCCCCAGAATACCCCCAAGGAGGCCAACCAGGCCCATTCAGGCTT | | QAPLPSAPSSFLPFSPTGP | |
| | | | | TCATTTTCACTCCATACCCAAGCTGGCTAGCCTTCCTCAGGCACCATTGCCC | | LLLGCCETPVVSLAEAQQE | |
| | | | | TCAGCTCCATCCAGCTTCCTGCCTTTCAGCCCCACTGGCCCTCTCCTCCTTG | | LQMLQKQLGESASTVPPAS | |
| | | | | GCTGCTGTGAGACACCAGTGGTCTCCTTGGCTGAGGCTCAGCAGGAGCTAC | | TATLLSNDLEADSSYYLNS | |
| | | | | AGATGCTGCAGAAGCAGTTGGGAGAAAGTGCCAGCACTGTTCCTCCTGCTT | | AQPHSPPRGTIELGRILEPG | |
| | | | | CCACAGCTACATTGCTGAGCAACGACTTGGAAGCCGACTCTTCCTACTACCT | | YLGSSGKWDVMRPQKGSV | |
| | | | | CAACTCTGCCCAGCCTCACTCTCCTCCAAGGGGCACCATAGAACTGGGAAG | | SGDLSSGSSVYQLNSKPTG | |
| | | | | AATCCTAGAGCCTGGGTACCTGGGCAGCAGTGGCAAGTGGGATGTGATGAG | | ADLLEEHLGEIRNLRQRLEE | |
| | | | | GCCTCAGAAAGGGAGTGTATCTGGGGACCTATCCTCAGGCTCCTCTGTGTA | | SICINDRLREQLEHR | |
| | | | | CCAGCTTAACTCCAAACCCACAGGGGCTGACCTGCTGGAAGAGCATCTTGG | | | |
| | | | | TGAAATCCGGAACCTGCGCCAGCGCCTGGAGGAGTCCATCTGCATCAATGA | | | |
| | | | | CCGCCTACGGGAGCAACTGGAACACCGGC | | | |
| Shigella | 5 | prey67550 | 121 | ATGCTTACAGAGCTTCTCTTTGAATTACATGTGGCGGCCACACCTGACAAAC | 322 | MLTELLFELHVAATPDKLNK | |
| ipaC | | | | TCAATAAGGCCATGAAGAGGGCTCATGACTGGGTGGAAGAGGATCAAACCG | | AMKRAHDWVEEDQTVVSV | |
| | | | | TGGTGTCAGTAGATGTGGCAAAAGTGTCCGAAGAAGAAACAAAGAAGGAAG | | DVAKVSEEETKKEEKEEKS | |
| | | | | AAAAGGAAGAGAAATCTCAAGACCCTCAAGAAGACAAAAAGGAGGAAAAGAA | | QDPQEDKKEEKKTKTIEEV | |
| | | | | AACTAAGACCATAGAGGAAGTATACATGTCGTCCATTGAAAGTCTGGCGGAG | | YMSSIESLAEVTARCIEQLH | |
| | | | | GTAACAGCGCGCTGTATTGAGCAGCTTCATAAAGTAGCAGAATTAATTCTTCA | | KVAELILHGQEEEKPAQDQ | |
| | | | | TGGACAAGAAGAGGAAAAACCAGCTCAGGACCAAGCAAAAGTTCTAATAAAA | | AKVLIKLTTAMCNEVASLSK | |
| | | | | TTAACTACTGCAATGTGCAATGAAGTGGCCTCTTTATCAAAGAAGTTTACGAA | | KFTNSLTTVGSNKKAEVLN | |
| | | | | TTCTTTAACCACTGTTGGGAGCAACAAGAAGGCCGAGGTCCTTAACCCCATG | | PMISSVLLEGC | |
| | | | | ATCAGTAGTGTATTGTTAGAGGGCTGCA | | | |
| Shigella | 5 | prey8889 | 122 | GTTCCAGAACAGACAGGTGCAGAGCCTGCTGGAGCTGCGGGAGGCCCAGG | 323 | FQNRQVQSLLELREAQVDA | |
| ipaC | | | | TGGACGCAGAGGCCCAGCGGAGGCTGGAACACCTGAGACAGGCTCTGCAG | | EAQRRLEHLRQALQRLREV | |
| | | | | CGGCTCAGGGAGGTCGTCCTTGATGCAAACACAACTCAGTTCAAGAGGCTG | | VLDANTTQFKRLKEMNERE | |
| | | | | AAAGAGATGAACGAGAGGGAGAAGAAGGAGCTGCAGAAGATCCTGGACAGA | | KKELQKILDRKRHNSISEAK | |
| | | | | AAGCGCCATAACAGCATCTCGGAGGCCAAGATGAGGGACAAGCATAAGAAG | | MRDKHKKEAELTEINRRHIT | |
| | | | | GAGGCGGAACTGACGGAGATTAACCGTCGGCACATCACTGAGTCAGTCAAC | | ESVNSIRRLEEAQKQRHDR | |
| | | | | TCCATCCGTCGGCTGGAGGAGGCCCAGAAGCAGCGGCATGACCGTCTTGTG | | LVAGQQQVLQQLAEEEPKL | |
| | | | | GCTGGGCAGCAGCAGGTCCTGCAACAGCTGGCAGAAGAGGAGCCCAAGCT | | LAQLAQECQEQRARLPQEI | |
| | | | | GCTGGCCCAGCTGGCCCAGGAGTGTCAGGAGCAGCGGGCGAGGCTCCCCC | | RRSLLGEMPEGLGDGPLV | |
| | | | | AGGAGATCCGCCGGAGCCTGCTGGGCGAGATGCCGGAGGGGCTGGGGGA | | ACASNGHAPGSSGHLSGA | |
| | | | | CGGGCCTCTGGTGGCCTGTGCCAGCAACGGTCACGCACCCGGGAGCAGCG | | DSESQEENTQL* | |
| | | | | GGCACCTGTCGGGCGCTGACTCGGAGAGCCAGGAGGAGAACACGCAGCTC | | | |
| | | | | TGA | | | |
| Shigella | 5 | prey11375 | 123 | CTCCTCGGCTGGGGGCTCGGGCAATTCCCGGCCCCCACGCAACCTCCAAG | 324 | SSAGGSGNSRPPRNLQGL | |
| ipaC | | | | GCTTGCTGCAGATGGCCATCACCGCGGGCTCTGAAGAGCCAGACCCTCCTC | | LQMAITAGSEEPDPPPEPM | |
| | | | | CAGAACCGATGAGTGAGGAGAGGCGTCAGTGGCTGCAGGAGGCCATGTCG | | SEERRQWLQEAMSAAFRG | |
| | | | | GCTGCCTTCCGAGGCCAGCGGGAGGAGGTGGAGCAGATGAAGAGCTGCCT | | QREEVEQMKSCLRVLSQP | |
| | | | | CCGAGTGCTGTCACAGCCCATGCCCCCCACTGCTGGGGAGGCCGAGCAGG | | MPPTAGEAEQAADQQERE | |
| | | | | CGGCCGACCAGCAAGAGCGAGAGGGGGCCCTGGAGCTGCTGGCCGACCTG | | GALELLADLCENMDNAADF | |
| | | | | TGTGAGAACATGGACAATGCCGCAGACTTCTGCCAGCTGTCTGGCATGCAC | | CQLSGMHLLVGRYLEAGA | |
| | | | | CTGCTGGTGGGCCGGTACCTGGAGGCGGGGGCTGCGGGACTGCGGTGGC | | AGLRWRAAQLIGTCSQNVA | |
| | | | | GGGCGGCACAGCTCATCGGCACGTGCAGTCAGAACGTGGCAGCCATCCAG | | AIQEQVLGLGALRKLLRLLD | |
| | | | | GAGCAGGTGCTGGGCCTGGGTGCCCTGCGTAAGCTGCTGCGGCTGCTGGA | | RDACDTVRVKALFAISCLV | |
| | | | | CCGCGACGCCTGCGACACGGTGCGCGTCAAGGCCCTCTTCGCCATCTCCTG | | REQEAGLLQFLRLDGFSVL | |
| | | | | TCTGGTCCGAGAGCAGGAGGCTGGGCTGCTGCAGTTCCTCCGCCTGGACG | | MRAMQQQVQKLKVKSAFL | |
| | | | | GCTTCTCTGTGTTGATGAGGGCCATGCAGCAGCAGGTGCAGAAGCTCAAGG | | LQNLLVGHPEHKGT | |
| | | | | TCAAATCAGCATTCCTGCTGCAGAACCTGCTGGTGGGCCACCCTGAACACAA | | | |
| | | | | AGGGACCC | | | |
| Shigella | 5 | prey67473 | 124 | ATGGCAGAGAAGGTGCTGGTAACAGGTGGGGCTGGCTACATTGGCAGCCAC | 325 | MAEKVLVTGGAGYIGSHTV | |
| ipaC | | | | ACGGTGCTGGAGCTGCTGGAGGCTGGCTACTTGCCTGTGGTCATCGATAAC | | LELLEAGYLPVVIDNFHNAF | |
| | | | | TTCCATAATGCCTTCCGTGGAGGGGGCTCCCTGCCTGAGAGCCTGCGGCGG | | RGGGSLPESLRRVQELTG | |
| | | | | GTCCAGGAGCTGACAGGCCGCTCTGTGGAGTTTGAGGAGATGGACATTTTG | | RSVEFEEMDILDQGALQRL | |
| | | | | GACCAGGGAGCCCTACAGCGTCTCTTCAAAAAGTACAGCTTTATGGCGGTCA | | FKKYSFMAVIHFAGLKAVG | |
| | | | | TCCACTTTGCGGGGCTCAAGGCCGTGGGCGAGTCGGTGCAGAAGCCTCTG | | ESVQKPLDYYRVNLTGTIQ | |
| | | | | GATTATTACAGAGTTAACCTGACCGGGACCATCCAGCTTCTGGAGATCATGA | | LLEIMKAHGVKNLVFSSSAT | |
| | | | | AGGCCCACGGGGTGAAGAACCTGGTGTTCAGCAGCTCAGCCACTGTGTACG | | VYGNPQYLPLDEA | |
| | | | | GGAACCCCCAGTACCTGCCCCTTGATGAGGCCCA | | | |
| Shigella | 5 | prey8929 | 125 | AAAAGTGGTTCAACGGTTGGTAGAGAGAGGAAGATCTTTGGATGATGCAAGG | 326 | KVVQRLVERGRSLDDARK | |
| ipaC | | | | AAGAGAGCCAAGCAGTTCCATGAAGCTTGGAGTAAACTTATGGAGTGGCTAG | | RAKQFHEAWSKLMEWLEE | |
| | | | | AAGAGTCAGAAAAGTCTTTGGATTCTGAACTGGAAATCGCAAATGATCCAGA | | SEKSLDSELEIANDPDKIKT | |
| | | | | CAAAATAAAAACACAACTTGCACAACATAAGGAGTTTCAGAAATCACTCGGAG | | QLAQHKEFQKSLGAKHSVY | |
| | | | | CCAAGCATTCTGTCTACGACACCACCAACAGGACTGGACGTTCTCTGAAGGA | | DTTNRTGRSLKEKTSLADD | |
| | | | | GAAAACCTCCCTGGCTGATGACAACCTGAAACTGGATGACATGCTGAGTGAA | | NLKLDDMLSELRDKWDTIC | |
| | | | | CTCAGAGACAAATGGGATACCATATGTGGAAAATCTGTGGAAAGACAAAACA | | GKSVERQNKLEEALLFSGQ | |
| | | | | AATTGGAGGAAGCCCTGTTATTTTCTGGACAATTCACAGATGCCCTACAGGC | | FTDALQALIDWLYRVEPQL | |
| | | | | TCTCATTGATTGGTTATATAGAGTTGAACCCCAGCTGGCAGAAGACCAGCCT | | AEDQPVHGDIDLVMNLIDN | |
| | | | | GTTCATGGAGACATTGATTTGGTGATGAATCTGATCGATAATCACAAGGCCTT | | HKAFQKELGKRTSSVQALK | |
| | | | | CCAAAAAGAGTTGGGGAAGAGGACCAGCAGTGTGCAGGCCCTGAAGCGCTC | | RSARELIEGSRDDSSWVKV | |
| | | | | AGCCCGAGAACTCATAGAAGGCAGTCGGGATGACTCCTCCTGGGTCAAGGT | | QMQELSTRWETVCALSISK | |
| | | | | CCAGATGCAGGAATTAAGCACACGCTGGGAGACCGTGTGTGCACTTTCTATA | | QTRLEAALRQAEEFHSVVH | |
| | | | | TCAAAGCAAACACGGTTAGAAGCAGCCCTGCGTCAGGCAGAGGAATTCCAC | | ALLEWLAEAEQTLRFHGVL | |
| | | | | TCGGTGGTACATGCCCTCTTGGAGTGGCTGGCTGAGGCGGAGCAAACCCTG | | PDDEDALRTLIDQHKE | |
| | | | | CGTTTCCATGGTGTCCTCCCAGATGATGAGGATGCTCTCCGGACTCTCATTG | | | |
| | | | | ATCAGCATAAAGAAT | | | |
| Shigella | 5 | prey3488 | 126 | GCTGACTCATACCGAAGAGTTGTTAGATGCTCAGAGACCAATAAGTGGAGAC | 327 | LTHTEELLDAQRPISGDPKV | |
| ipaC | | | | CCAAAAGTCATTGAAGTTGAGCTCGCAAAGCACCATGTCCTAAAAAATGATG | | IEVELAKHHVLKNDVLAHQ | |
| | | | | TTTTGGCTCATCAAGCCACAGTGGAAACAGTCAACAAAGCTGGCAATGAGCT | | ATVETVNKAGNELLESSAG | |
| | | | | TCTTGAATCCAGTGCTGGAGATGATGCCAGCAGCTTAAGGAGCCGTTTGGAA | | DDASSLRSRLEAMNQCWE | |
| | | | | GCCATGAACCAATGCTGGGAGTCAGTGTTACAGAAAACAGAGGAGAGGGAG | | SVLQKTEEREQQLQSTLQQ | |
| | | | | CAGCAGCTTCAGTCAACTCTGCAGCAGGCCCAGGGCTTCCACAGTGAAATT | | AQGFHSEIEDFLLELTRME | |
| | | | | GAAGATTTCCTCTTGGAACTTACTAGAATGGAGAGCCAGCTTTCTGCATCTAA | | SQLSASKPTGGLPETAREQ | |
| | | | | GCCCACAGGAGGACTTCCTGAAACTGCTAGGGAACAGCTTGATACACATATG | | LDTHMELYSQLKAKEETYN | |
| | | | | GAACTCTATTCCCAGCTGAAAGCCAAGGAAGAGACTTATAATCAACTACTTGA | | QLLDKGRLMLLSRDDSGS | |
| | | | | CAAGGGCAGACTCATGCTTCTAAGCCGTGACGACTCTGGGTCTGGCTCCAA | | GSKTEQSVALLEQKWHVV | |
| | | | | GACAGAACAGAGTGTAGCACTTTTGGAGCAGAAGTGGCATGTGGTCAGCAG | | SSKMEERKSKLEEALNLAT | |
| | | | | TAAGATGGAAGAAAGAAAGTCAAAGCTGGAAGAGGCCCTCAACTTGGCAACA | | EFQNSLQEFINWLTLAEQS | |
| | | | | GAATTCCAGAATTCCCTACAAGAATTTATCAACTGGCTCACTCTAGCAGAGCA | | LNIASPPSLILNTVLSQIEEH | |
| | | | | GAGTTTAAACATCGCTTCTCCACCAAGCCTGATTCTAAATACTGTCCTTTCCC | | KVFANEVNAHRDQIIELDQT | |
| | | | | AGATAGAAGAGCACAAGGTTTTTGCTAATGAAGTAAATGCTCATCGAGACCA | | GHQLKFLSQKQDVVLIKNLL | |
| | | | | GATCATTGAGCTGGATCAAACTGGGAATCAATTAAAGTTCCTTAGCCAAAAG | | SVSQSRWEKVVQRSIERG | |
| | | | | CAGGATGTTGTTCTGATCAAGAATTTGTTGGTGAGCGTGCAGTCTCGATGGG | | RSLDDARKRAKQFHEAWK | |
| | | | | AGAAGGTTGTCCAGCGATCTATTGAAAGAGGGCGATCACTAGATGATGCCAG | | KLIDWLEDAESHLDSELEIS | |
| | | | | GAAGCGGGCAAAACAATTCCATGAAGCTTGGAAAAAACTGATTGACTGGCTA | | NDPDKIKLQLSKHKEFQKTL | |
| | | | | GAAGATGCAGAGAGTCACCTGGACTCAGAACTAGAGATATCCAATGACCCAG | | GGKQPVYDTTIRTGRALKE | |
| | | | | ACAAAATTAAACTTCAGCTTTCTAAGCATAAGGAGTTTCAGAAGACTCTTGGT | | KTLLPEDTQKLDNFLGEVR | |
| | | | | GGCAAGCAGCCTGTGTATGATACCACAATTAGAACTGGCAGAGCACTGAAAG | | DKWDTVCGKSVERQHKLE | |
| | | | | AAAAGACTTTGCTTCCCGAAGATACTCAGAAACTTGACAATTTCCTAGGAGAA | | EALLFSGQFMDALQALVD | |
| | | | | GTCAGAGACAAATGGGATACTGTTTGTGGCAAGTCTGTGGAGCGGCAGCAC | | WLYKVEPQLAEDQPVHGD | |
| | | | | AAGTTGGAGGAAGCCCTGCTCTTTTCGGGTCAGTTCATGGATGCTTTGCAGG | | LDLVMNLMDAHKVFQKELG | |
| | | | | CATTGGTTGACTGGTTATACAAGGTGGAGCCACAGCTGGCTGAGGACCAGC | | KRTGTVQVLKRSGRELIEN | |
| | | | | CCGTGCACGGGGACCTTGACCTCGTCATGAACCTCATGGATGCACACAAGG | | SRDDTTWVKGQLQELSTR | |
| | | | | TTTTCCAGAAGGAACTGGGAAAGCGAACAGGAACCGTTCAGGTCCTGAAGC | | WDTVCKLSVSKQSRLEQAL | |
| | | | | GGTCAGGCCGAGAGCTGATTGAGAATAGTCGAGATGACACCACTTGGGTAA | | KQAEVFRDTVHMLLEWLSE | |
| | | | | AAGGACAGCTCCAGGAACTGAGCACTCGCTGGGACACTGTCTGTAAACTCT | | AEQTLRFRGALPDDTEALQ | |
| | | | | CTGTTTCCAAACAAAGCCGGCTTGAGCAGGCCTTAAAACAAGCGGAAGTGTT | | SLIDT | |
| | | | | TCGAGACACAGTCCACATGCTGTTGGAGTGGCTTTCTGAAGCAGAGCAAAC | | | |
| | | | | GCTTCGCTTTCGGGGAGCACTTCCTGATGACACAGAGGCCCTGCAGTCTCT | | | |
| | | | | CATTGACACCC | | | |
| Shigella | 5 | prey3514 | 127 | GGAAAAAGAAGAGCTGCCACGTGCCGTGGGTACCCAGACATTGAGTGGTGC | 328 | EKEELPRAVGTQTLSGAGL | |
| ipaC | | | | TGGTCTCCTCAAGATGTTCAACAAAGCCACAGATGCCGTCAGCAAAATGACC | | LKMFNKATDAVSKMTIKMN | |
| | | | | ATCAAGATGAATGAATCAGACATTTGGTTTGAGGAGAAGCTCCAGGAGGTAG | | ESDIWFEEKLQEVECEEQR | |
| | | | | AGTGTGAGGAGCAGCGCTTACGGAAACTGCATGCTGTTGTAGAAACTCTAGT | | LRKLHAVVETLVNHRKELA | |
| | | | | CAACCATAGGAAAGAGCTAGCGCTGAACACAGCCCAGTTTGCAAAGAGTCTA | | LNTAQFAKSLAMLGSSEDN | |
| | | | | GCCATGCTTGGGAGCTCTGAGGACAACACGGCATTGTCACGGGCACTCTCC | | TALSRALSQLAEVEEKIEQL | |
| | | | | CAGCTGGCTGAGGTGGAAGAAAAAATTGAGCAGCTCCACCAGGAACAGGCC | | HQEQANNDFFLLAELLSDYI | |
| | | | | AACAATGACTTCTTCCTCCTTGCTGAGCTCCTGAGTGACTACATTCGCCTCCT | | RLLAIVRAAFDQRMKTWQR | |
| | | | | GGCCATAGTCCGCGCTGCCTTCGACCAGCGCATGAAGACATGGCAGCGCTG | | WQDAQATLQKKREAEARL | |
| | | | | GCAGGATGCCCAAGCCACACTGCAGAAGAAGCGGGAGGCCGAGGCTCGGC | | LWANKPDKLQQAKDEILEW | |
| | | | | TGCTGTGGGCCAACAAGCCTGATAAGCTGCAGCAGGCCAAGGACGAGATCC | | ESRVTQYERDFERISTVVR | |
| | | | | TCGAGTGGGAGTCTCGGGTGACTCAATATGAAAGGGACTTCGAGAGGATTT | | KEVIRFEKEKSKDFKNHVIK | |
| | | | | CAACAGTGGTCCGAAAAGAAGTGATACGGTTTGAGAAAGAGAAATCCAAGGA | | YLETLLYSQQQLAKYWEAF | |
| | | | | CTTCAAGAACCACGTGATCAAGTACCTTGAGACACTCCTTTACTCACAGCAG | | LPEAKAIS* | |
| | | | | CAGCTGGCAAAGTACTGGGAAGCCTTCCTTCCTGAGGCAAAGGCCATCTCC | | | |
| | | | | TAA | | | |
| Shigella | 5 | prey5814 | 128 | TGATGCCCCACCACAGCTTGAAGATGAGGAACCTGCATTTCCACATACTGAC | 329 | DAPPQLEDEEPAFPHTDLA | |
| ipaC | | | | TTGGCCAAGTTGGATGACATGATCAACAGGCCTCGATGGGTGGTTCCAGTTT | | KLDDMINRPRWVVPVLPKG | |
| | | | | TGCCGAAAGGGGAATTAGAAGTGCTTTTAGAAGCTGCTATTGATCTTAGTAAA | | ELEVLLEAAIDLSKKGLDVL | |
| | | | | AAGGGCCTTGATGTTAAAAGTGAAGCATGTCAGCGATTTTTCCGTGATGGGC | | SEACQRFFRDGLTISFTKIL | |
| | | | | TAACAATATCATTCACTAAAATTCTTACAGATGAAGCAGTGAGTGGCTGGAAG | | TDEAVSGWKFEIHRCLVEL | |
| | | | | TTTGAAATTCATAGGTGTCTGGTGGAGCTATGTGTGGCCAAGTTGTCCCAAG | | CVAKLSQDWFPLLELLAMA | |
| | | | | ACTGGTTTCCACTTTTAGAACTTCTTGCCATGGCCTTAAATCCTCATTGCAAA | | LNPHCKFHIYNGTRPCESV | |
| | | | | TTCCATATCTACAATGGTACACGTCCATGTGAATCAGTTTCCTCAAGTGTTCA | | SSSVQLPEDELFARSPDPR | |
| | | | | GTTGCCTGAAGATGAACTCTTTGCTCGTTCTCCAGATCCTCGATCACCAAAG | | SPKGWLVDLLNKFGTLNGF | |
| | | | | GGTTGGCTAGTGGATCTTCTCAACAAATTTGGCACTTTAAATGGGTTCCAGAT | | QILHDRFINGSALNVQIIAALI | |
| | | | | TTTGCATGATCGTTTTATTAATGGATCAGCATTAAACGTTCAAATAATTGCAGC | | KPFGQCYEFLTLHTVKKYF | |
| | | | | CCTTATTAAACCATTTGGGCAATGCTATGAGTTTCTCACTCTTCATACAGTGA | | LPIIEMVPQFLENLTDEELK | |
| | | | | AAAAGTACTTTCTTCCAATAATAGAAATGGTTCCACAGTTTTTAGAAAACTTAA | | KEAKNEAKNDALSMIIKSLK | |
| | | | | CTGATGAAGAACTGAAAAAAGAAGCAAAGAATGAAGCCAAAAATGATGCTCT | | NLASRVPGQEETVKNLEIF | |
| | | | | TTCAATGATTATTAAATCTTTGAAGAATTTAGCTTCAAGGGTTCCAGGACAAG | | RLKMILRLLQISSFNGKMNA | |
| | | | | AAGAAACTGTTAAAAACTTAGAAATATTTAGGTTAAAAATGATACTTAGATTAT | | LNEVNKVISSVSYYTHRHG | |
| | | | | TGCAAATTTCTTCTTTCAATGGAAAGATGAATGCACTGAATGAAGTTAATAAG | | NPEEEEWLTAERMAEWIQ | |
| | | | | GTGATATCTAGTGTATCATACTATACTCATCGACATGGTAATCCTGAGGAGGA | | QNNILSIVLRDSLHQPQYVE | |
| | | | | AGAGTGGCTCACAGCTGAACGAATGGCAGAATGGATACAGCAGAACAATATC | | KLEKILRFVIKEKALTLQDLD | |
| | | | | TTATCCATAGTGTTGCGAGATAGTCTTCATCAGCCACAGTATGTAGAAAAGTT | | NIWAAQAGKHEAIVKNVHD | |
| | | | | AGAGAAGATTCTTCGTTTTGTCATCAAAGAAAAAGCTCTGACCTTACAGGATC | | LLAKLAWDFSPEQLDHPFD | |
| | | | | TTGATAATATCTGGGCAGCACAGGCAGGGAAACATGAAGCCATTGTGAAGAA | | CFKASRTNASKKQREKLLE | |
| | | | | TGTACATGATCTCCTGGCAAAATTGGCATGGGATTTTTCTCCTGAACAACTTG | | LIRRLAEDDKDGVMAHRVL | |
| | | | | ATCATCCTTTTGATTGTTTTAAGGCCAGTCGGACAAATGCGAGTAAAAAGCAA | | NLLWNLAHSDDVPVDIMDL | |
| | | | | CGTGAAAAGCTACTTGAGCTGATACGTCGTCTTGCAGAAGATGATAAAGATG | | ALSAHIKILDYSCSQDRDTQ | |
| | | | | GTGTGATGGCACACAGAGTGTTGAACCTTCTGTGGAATCTGGCTCACAGTGA | | KIQWIDRFIEELRTNDKWVI | |
| | | | | TGATGTGCCTGTAGATATCATGGACCTGGCTCTCAGTGCCCACATAAAAATA | | PALKQIREICSLFGEAPQNL | |
| | | | | CTAGATTACAGTTGCTCCCAGGACCGTGATACACAAAAGATCCAATGGATAG | | SQTQRSPHVFYR | |
| | | | | ATCGCTTTATAGAAGAACTTCGCACAAATGACAAATGGGTTATTCCCGCACTG | | | |
| | | | | AAACAAATTAGAGAAATTTGTAGTTTGTTTGGTGAAGCGCCTCAAAATTTGAG | | | |
| | | | | TCAAACTCAGCGAAGTCCCCATGTGTTTTATCGCCA | | | |
| Shigella | 5 | prey5814 | 129 | CCATGCCAAACTTGGAGAAAGCAGCCTTAGTCCATCTCTTGACTCACTTTTCT | 330 | HAKLGESSLSPSLDSLFFG | |
| ipaC | | | | TTGGTCCTTCAGCCTCACAAGTGCTATATCTAACAGAGGTAGTCTATGCCTTG | | PSASQVLYLTEVVYALLMP | |
| | | | | TTAATGCCTGCTGGTGCACCTCTGGCTGATGATTCCTCTGATTTTCAGTTTCA | | AGAPLADDSSDFQFHFLKS | |
| | | | | CTTCTTGAAAAGTGGTGGCCTACCCCTTGTACTGAGTATGCTAACCAGAAAT | | GGLPLVLSMLTRNNFLPNA | |
| | | | | AACTTCCTACCGAATGCAGATATGGAAACTCGAAGGGGTGCCTACCTCAATG | | DMETRRGAYLNALKIAKLLL | |
| | | | | CTCTTAAAATAGCCAAGCTTTTGCTAACTGCCATTGGCTATGGTCATGTTCGA | | TAIGYGHVRAVAEACQPGV | |
| | | | | GCTGTGGCAGAAGCTTGTCAGCCAGGTGTAGAAGGTGTGAATCCCATGACA | | EGVNPMTQINQVTHDQAV | |
| | | | | CAGATCAACCAAGTTACCCATGATCAAGCAGTGGTGCTACAAAGTGCCCTTC | | VLQSALQSIPNPSSECMLR | |
| | | | | AGAGCATTCCTAATCCATCATCCGAGTGCATGCTTAGAAATGTGTCAGTTCGT | | NVSVRLAQQISDEASRYMP | |
| | | | | CTTGCTCAGCAGATATCTGATGAGGCTTCAAGATATATGCCTGATATTTGTGT | | DICVIRAIQKIIWASGCGSLQ | |
| | | | | AATTAGAGCTATACAAAAAATTATCTGGGCATCAGGATGTGGGTCGTTACAG | | LVFSPNEEITKIYEKTNAGN | |
| | | | | CTAGTATTTAGCCCAAATGAAGAAATCACTAAAATTTATGAGAAGACCAATGC | | EPDLEDEQVCCEALEVMTL | |
| | | | | AGGCAATGAGCCAGACTTGGAAGACGAACAGGTTTGCTGTGAAGCATTGGA | | CFALIPTALDALSKEKAWQT | |
| | | | | AGTGATGACCTTATGTTTTGCCTTGATTCCAACAGCCTTAGATGCTCTTAGTA | | FIIDLLLHCHSKTVRQVAQE | |
| | | | | AAGAAAAGGCTTGGCAGACATTCATCATTGACTTACTATTGCACTGTCACAGC | | QFFLMCTRCCMGHRPLLFF | |
| | | | | AAAACTGTTCGTCAGGTGGCACAGGAGCAGTTCTTTTTAATGTGCACCAGAT | | ITLLFTVLGSTARERAKHSG | |
| | | | | GTTGCATGGGACACCGGCCTCTACTTTTCTTCATTACTCTACTCTTTACTGTT | | DYFTLLRHLLNYAYNSNINV | |
| | | | | TTGGGGAGCACAGCAAGAGAGAGAGCTAAACACTCAGGCGACTACTTTACT | | PNAEVLFNNEIDWLKRIRD | |
| | | | | CTTTTAAGACACCTTCTTAATTACGCTTACAATAGTAATATTAATGTACCCAAT | | DVKRTGETGIEETILEGHLG | |
| | | | | GCTGAAGTTCTTTTCAATAATGAAATTGATTGGCTTAAAAGAATTAGGGATGA | | VTKELLAFQTSEKKFHIGCE | |
| | | | | TGTTAAAAGAACAGGAGAAACGGGTATTGAAGAGACGATCTTAGAGGGCCAC | | KGGANLIKELIDDFIFPASNV | |
| | | | | CTTGGAGTGACAAAGGAGTTACTGGCCTTTCAAACTTCTGAGAAAAAATTTCA | | YLQYMRNGELPAEQAIPVC | |
| | | | | TATTGGTTGTGAAAAAGGAGGTGCTAATCTCATTAAAGAATTAATTGATGATT | | GSPPTINAGFELLVALAVGC | |
| | | | | TCATATTTCCTGCATCCAATGTTTACCTACAGTATATGAGAAATGGAGAGCTT | | VRNLKQIVDSLTEMYYIGTA | |
| | | | | CCAGCTGAACAGGCTATTCCGGTCTGTGGTTCACCACCTACAATTAATGCTG | | ITTCEALTEWEYLPPVGPR | |
| | | | | GTTTTGAATTACTTGTAGCATTAGCTGTTGGCTGTGTGAGGAATCTCAAACAA | | PPKGFVGLKNAGATCYMN | |
| | | | | ATAGTAGATTCTTTGACTGAAATGTATTACATTGGCACAGCAATAACTACTTG | | SVIQQLYMIPSIRNGILAIEG | |
| | | | | TGAAGCACTTACTGAGTGGGAATATCTGCCACCTGTTGGACCCCGCCCACC | | TGSDVDDDMSGDEKQDNE | |
| | | | | CAAAGGATTCGTGGGGCTGAAAAATGCCGGTGCTACTTGTTACATGAATTCT | | SNVDPRDDVFGYPQQFED | |
| | | | | GTGATTCAGCAACTCTACATGATTCCTTCCATTAGGAACGGTATTCTTGCCAT | | KPALSKTEDRKEYNIGVLR | |
| | | | | TGAAGGCACAGGTAGTGATGTAGATGATGATATGTCTGGGGATGAGAAGCA | | HLQVIFGHLAASRLQYYVP | |
| | | | | GGACAATGAGAGCAATGTTGATCCCAGGGATGATGTATTTGGATATCCTCAA | | RGFWKQFRLWGEPVNLRE | |
| | | | | CAATTTGAAGATAAACCAGCATTAAGTAAAACTGAAGATAGAAAAGAGTACAA | | QHDALEFFNSLVDSLDEAL | |
| | | | | CATTGGTGTCCTAAGACACCTTCAGGTCATCTTTGGTCATTTAGCTGCTTCTC | | KALGHPAMLSKVLGGSFAD | |
| | | | | GACTGCAATACTATGTGCCCAGAGGATTTTGGAAACAGTTCAGGCTTTGGGG | | QKICQGCPHRYECEESFTT | |
| | | | | TGAGCCTGTTAATCTGCGTGAACAACACGATGCTTTAGAATTTTTTAATTCATT | | LNVDIRNHQNLLDSLEQYV | |
| | | | | GGTGGATAGTTTAGATGAAGCTTTAAAAGCTTTAGGACATCCAGCTATGCTAA | | KGDLLEGANAYHCEKCNK | |
| | | | | GTAAAGTCTTAGGAGGTTCCTTTGCTGATCAGAAGATCTGCCAAGGCTGCCC | | KVDTVKRLLIKKLPPVLAIQL | |
| | | | | ACATAGGTACGAATGTGAAGAATCTTTTACGACCCTAAACGTAGACATTAGAA | | KRFDYDWERECAIKFNDYF | |
| | | | | ATCACCAAAATCTTCTTGATTCTTTGGAACAGTATGTCAAAGGAGATTTACTA | | EFPRELDMEPYTVAGVAKL | |
| | | | | GAAGGTGCAAATGCATATCATTGTGAAAAATGCAATAAAAAGGTTGATACCGT | | EGDNVNPESQLIQQSEQSE | |
| | | | | AAAGCGCTTGCTGATTAAAAAATTACCTCCTGTTCTTGCTATACAACTAAAGC | | SETAGSTKYRLVGVLVHSG | |
| | | | | GATTTGACTATGACTGGGAAAGAGAATGTGCAATCAAGTTCAATGATTATTTT | | QASGGHYYSYIIQRNGGDG | |
| | | | | GAATTTCCTCGAGAGCTGGACATGGAACCTTACACAGTTGCAGGTGTCGCAA | | ERNRWYKFDDGDVTECKM | |
| | | | | AGCTGGAAGGGGATAATGTAAACCCAGAGAGTCAGTTGATACAACAGAGTGA | | DDDEEMKNQCFGGEYMG | |
| | | | | GCAGTCTGAAAGTGAGACAGCAGGAAGCACAAAATACAGACTTGTGGGTGT | | EVFDHMMKRMSYRRQKR | |
| | | | | GCTCGTACACAGTGGTCAAGCGAGTGGGGGGCATTATTATTCTTACATCATC | | WWNAYIPFYERMDTIDQDD | |
| | | | | CAAAGGAATGGTGGAGATGGTGAGAGAAATCGCTGGTATAAATTTGATGATG | | ELIRYISELAITTRPHQIIMPS | |
| | | | | GTGATGTAACAGAATGTAAAATGGATGATGACGAAGAAATGAAAAACCAGTG | | AIERSVRKQNVQFMHNRM | |
| | | | | TTTTGGTGGAGAGTACATGGGAGAAGTGTTTGATCACATGATGAAGCGTATG | | QYSMEYFQFMKKLLTCNG | |
| | | | | TCATACAGGCGCCAGAAAAGGTGGTGGAATGCTTATATACCTTTTTATGAAC | | VYLNPPPGQDHLLPEAEEIT | |
| | | | | GAATGGACACAATAGACCAAGATGATGAGTTGATAAGATATATATCAGAGCTT | | MISIQLAARFLFTTGFHTKK | |
| | | | | GCTATCACCACCAGACCTCATCAGATTATTATGCCATCAGCCATTGAGAGAA | | VVRGSASDWYDALCILLRH | |
| | | | | GTGTACGGAAACAGAACGTACAATTCATGCATAACCGAATGCAGTACAGTAT | | SKNVRFWFAHNVLFNVSN | |
| | | | | GGAGTATTTTCAGTTTATGAAAAAACTGCTTACATGTAATGGCGTTTACTTAAA | | RFSEYLLECPSAEVRGAFA | |
| | | | | CCCTCCTCCCGGGCAAGATCACCTGTTGCCTGAAGCAGAAGAAATCACTATG | | KLIVFIAHFSLQDGPCPSPF | |
| | | | | ATCAGTATTCAACTTGCTGCTAGGTTCCTCTTTACTACAGGATTTCACACAAA | | ASPGPSSQAYDNLSLSDHL | |
| | | | | GAAAGTAGTCCGTGGCTCTGCCAGTGATTGGTATGATGCATTGTGTATTCTC | | LRAVLNLLRREVSEHGRHL | |
| | | | | CTTCGTCACAGCAAGAATGTACGTTTTTGGTTTGCTCATAACGTCCTTTTTAA | | QQYFNLFVMYANLGVAEKT | |
| | | | | TGTTTCAAATCGCTTCTCCGAATACCTTCTGGAGTGCCCTAGTGCAGAAGTG | | QLLKLSVPATFMLVSLDEG | |
| | | | | AGGGGTGCGTTTGCAAAACTTATAGTCTTTATTGCACATTTTTCCTTGCAAGA | | PGPPIKYQYAELGKLYSVV | |
| | | | | TGGGCCATGTCCTTCACCTTTTGCCTCTCCTGGACCTTCTAGTCAGGCTTAT | | SQLIRCCNVSSRMQSSING | |
| | | | | GACAACTTAAGCTTGAGTGATCACTTACTAAGAGCAGTACTAAATCTCTTGAG | | NPPLPNPFGDPNLSQPIMPI | |
| | | | | AAGGGAAGTTTCAGAGCATGGGCGTCATTTACAGCAGTATTTCAACCTGTTT | | QQNVADILFVRTSYVKKIIE | |
| | | | | GTAATGTATGCCAATTTAGGTGTGGCAGAGAAGACACAGCTTCTGAAATTGA | | DCSNSEETVKLLRFCCWE | |
| | | | | GTGTACCTGCTACTTTTATGCTTGTGTCTTTAGATGAAGGTCCAGGTCCTCCA | | NPQFSSTVLSELLWQVAYS | |
| | | | | ATCAAATACCAGTATGCTGAATTAGGCAAATTATACTCAGTAGTGTCACAGCT | | YPYELRPYLDLLLQILLIEDS | |
| | | | | GATCCGCTGTTGCAATGTCTCTTCAAGAATGCAGTCTTCAATCAATGGTAATC | | WQTHRIHNALKGIPDDRDG | |
| | | | | CTCCTCTTCCCAATCCTTTTGGTGATCCTAATTTATCACAACCTATAATGCCAA | | LFDTIQRSKNHYQKRAYQC | |
| | | | | TTCAGCAGAATGTGGCAGACATTTTATTTGTGAGAACAAGTTATGTGAAGAAA | | IKCMVALFSNCPVAYQILQG | |
| | | | | ATCATTGAAGACTGCAGTAATTCAGAGGAAACCGTCAAATTGCTTCGTTTTTG | | NGDLKRKWTWAVEWLGD | |
| | | | | CTGCTGGGAGAATCCTCAGTTCTCATCTACTGTCCTCAGTGAACTTCTCTGG | | ELERRPYTGNPQYTYNNW | |
| | | | | CAGGTTGCATATTCCTATCCCTATGAACTGCGGCCCTATTTGGATCTGCTTTT | | SPPVQSNETSNGYFLERSH | |
| | | | | GCAAATCTTACTGATTGAGGACTCCTGGCAAACTCACAGAATTCATAATGCAC | | SARMTLAKACELCPEEVKK | |
| | | | | TGAAAGGAATTCCAGATGACCGAGATGGGCTGTTTGACACAATCCAGCGCTC | | ATSVQQIEMEESKEPDDQD | |
| | | | | TAAGAATCACTATCAAAAAAGAGCATACCAGTGTATAAAATGTATGGTAGCTC | | APDEHESPPPEDAPLYPHS | |
| | | | | TATTTAGTAACTGTCCTGTTGCTTACCAAATCCTGCAGGGCAATGGAGATCTT | | PGSQYQQNNHVHGQPYTG | |
| | | | | AAAAGAAAGTGGACCTGGGCAGTGGAATGGCTTGGAGATGAACTTGAAAGA | | PAAHHMNNPQRTGQRAQE | |
| | | | | AGACCATATACTGGCAATCCTCAGTACACTTACAACAATTGGTCTCCCCCAGT | | NYEGSEEVSPPQTKDQ* | |
| | | | | GCAAAGCAATGAAACGTCCAATGGTTATTTCTTGGAGAGATCACATAGTGCT | | | |
| | | | | AGGATGACACTTGCAAAAGCTTGTGAACTCTGTCCAGAGGAGGTAAAAAAAG | | | |
| | | | | CCACCAGTGTGCAGCAGATAGAAATGGAAGAGAGCAAAGAGCCAGATGACC | | | |
| | | | | AAGATGCTCCAGATGAACATGAGTCGCCTCCACCTGAAGATGCCCCATTGTA | | | |
| | | | | CCCCCATTCACCTGGATCTCAGTATCAACAGAATAACCATGTGCATGGACAG | | | |
| | | | | CCATATACAGGCCCAGCAGCACATCACATGAACAACCCTCAGAGAACTGGC | | | |
| | | | | CAACGAGCACAAGAAAATTATGAAGGCAGTGAAGAAGTATCCCCACCTCAAA | | | |
| | | | | CCAAGGATCAATGA | | | |
| Shigella | 5 | prey67479 | 130 | CGATGAGCTCATGAGACATCAGCCCACCCTTAAAACAGATGCAACGACTGCC | 331 | DELMRHQPTLKTDATTAIIK | |
| ipaC | | | | ATCATCAAGTTACTTGAAGAAATCTGTAATCTTGGAAGGGACCCCAAATACAT | | LLEEICNLGRDPKYICQKPS | |
| | | | | CTGTCAGAAGCCATCGATCCAGAAGGCAGATGGCACTGCCACTGCTCCTCC | | IQKADGTATAPPPRSNHAA | |
| | | | | CCCAAGGTCTAATCATGCCGCAGAAGAAGCCTCTAGTGAGGATGAGGAGGA | | EEASSEDEEEEEVQAMQS | |
| | | | | AGAGGAAGTACAGGCCATGCAGAGCTTTAATTCTACCCAGCAAAATGAAACT | | FNSTQQNETEPNQQVVGT | |
| | | | | GAGCCTAATCAGCAGGTTGTTGGTACAGAGGAACGTATTCCTATTCCCCTCA | | EERIPIPLMDYILNVMKFVE | |
| | | | | TGGATTACATCCTTAATGTGATGAAATTTGTGGAATCTATTCTGAGCAACAAT | | SILSNNTTDDHCQEFVNQK | |
| | | | | ACAACAGATGACCACTGCCAGGAATTTGTGAATCAGAAAGGACTGTTGCCTT | | GLLPLVTILGLPNLPIDFPTS | |
| | | | | TGGTTACCATTTTGGGTCTTCCCAATCTGCCCATTGACTTTCCCACATCTGCT | | AACQAVAGVCKSILTLSHE | |
| | | | | GCCTGTCAGGCTGTTGCAGGTGTCTGCAAATCCATATTGACACTGTCACATG | | PKVLQEGLLQLDSILSSLEP | |
| | | | | AACCCAAAGTCCTTCAAGAGGGTCTCCTTCAGTTGGACTCCATCCTCTCCTC | | LHR | |
| | | | | CCTGGAGCCCTTACACCGCCC | | | |
| Shigella | 5 | prey700 | 131 | ATGGGAATTGGTCTTTCTGCTCAAGGTGTGAACATGAATAGACTACCAGGTT | 332 | MGIGLSAQGVNMNRLPGW | |
| ipaC | | | | GGGATAAGCATTCATATGGTTACCATGGGGATGATGGACATTCGTTTTGTTCT | | DKHSYGYHGDDGHSFCSS | |
| | | | | TCTGGAACTGGACAACCTTATGGACCAACTTTCACTACTGGTGATGTCATTG | | GTGQPYGPTFTTGDVIGCC | |
| | | | | GCTGTTGTGTTAATCTTATCAACAATACCTGCTTTTACACCAAGAATGGACAT | | VNLINNTCFYTKNGHSLGIA | |
| | | | | AGTTTAGGTATTGCTTTCACTGACCTACCGCCAAATTTGTATCCTACTGTGGG | | FTDLPPNLYPTVGLQTPGE | |
| | | | | GCTTCAAACACCAGGAGAAGTGGTCGATGCCAATTTTGGGCAACATCCTTTC | | VVDANFGQHPFVFDIEDYM | |
| | | | | GTGTTTGATATAGAAGACTATATGCGGGAGTGGAGAACCAAAATCCAGGCAC | | REWRTKIQAQIDRFPIGDR | |
| | | | | AGATAGATCGATTTCCTATCGGAGATCGAGAAGGAGAATGGCAGACCATGAT | | EGEWQTMIQKMVSSYLVH | |
| | | | | ACAAAAAATGGTTTCATCTTATTTAGTCCACCATGGGTACTGTGCCACAGCAG | | HGYCATAEAFARSTDQTVL | |
| | | | | AGGCCTTTGCCAGATCTACAGACCAGACCGTTCTAGAAGAATTAGCTTCCAT | | EELASIKNRQRIQKLVLAGR | |
| | | | | TAAGAATAGACAAAGAATTCAGAAATTGGTATTAGCAGGAAGAATGGGAGAA | | MGEAIETTQQLYPSLLE | |
| | | | | GCCATTGAAACAACACAACAGTTATACCCAAGTTTACTTGAAAG | | | |
| Shigella | 5 | prey67481 | 132 | AAAACAAGACCAGAAAGCTCCAGATAAAGAGGCCATACTGCGGGCCACCGC | 333 | KQDQKAPDKEAILRATANL | |
| ipaC | | | | CAACCTGCCCTCCTACAACATGGACCGGGCCGCGGTCCAGACCAACATGAG | | PSYNMDRAAVQTNMRDFQ | |
| | | | | AGACTTCCAGACAGAACTCCGGAAGATACTGGTGTCTCTCATCGAGGTGGC | | TELRKILVSLIEVAQKLLALN | |
| | | | | GCAGAAGCTGTTAGCGCTGAACCCAGATGCGGTGGAATTGTTTAAGAAGGC | | PDAVELFKKANAMLDEDED | |
| | | | | GAATGCAATGCTGGACGAGGACGAGGATGAGCGTGTGGACGAGGCTGCCC | | ERVDEAALRQLTEMGFPEN | |
| | | | | TGCGGCAGCTCACGGAGATGGGCTTTCCGGAGAACAGAGCCACCAAGGCC | | RATKALQLNHMSVPQAME | |
| | | | | CTTCAGCTGAACCACATGTCGGTGCCTCAGGCCATGGAGTGGCTAATTGAAC | | WLIEHAEDP | |
| | | | | ACGCAGAAGACCCG | | | |
| Shigella | 5 | prey67488 | 133 | CTGTTCATGAAGAGTGAGCGACACGCAGCCGAGGCACAGCTGGCCACAGCA | 334 | LFMKSERHAAEAQLATAEQ | |
| ipaC | | | | GAGCAGCAGCTACGGGGGCTACGGACCGAGGCGGAAAGGGCTCGCCAGG | | QLRGLRTEAERARQAQSR | |
| | | | | CCCAGAGCCGGGCCCAGGAGGCTCTGGACAAGGCCAAGGAGAAGGACAAG | | AQEALDKAKEKDKKITELSK | |
| | | | | AAGATCACAGAACTCTCCAAAGAAGTCTTCAATCTTAAGGAAGCCTTGAAGG | | EVFNLKEALKEQPAALATP | |
| | | | | AGCAGCCGGCCGCCCTCGCCACCCCTGAGGTGGAGGCTCTCCGTGACCAG | | EVEALRDQVKDLQQQLQE | |
| | | | | GTGAAGGATTTACAGCAGCAGCTGCAGGAAGCTGCCAGGGACCACTCCAGC | | AARDHSSVVALYRSHLLYAI | |
| | | | | GTGGTGGCTTTGTACAGAAGCCACCTCCTATATGCCATTCAG | | Q | |
| Shigella | 5 | prey51967 | 134 | TGACCAACTTGTGTTGATATTTGCTGGAAAAATTTTGAAAGATCAAGATACCT | 335 | DQLVLIFAGKILKDQDTLSQ | |
| ipaC | | | | TGAGTCAGCATGGAATTCATGATGGACTTACTGTTCACCTTGTCATTAAAACA | | HGIHDGLTVHLVIKTQNRP | |
| | | | | CAAAACAGGCCTCAGGATCATTCAGCTCAGCAAACAAATACAGCTGGAAGCA | | QDHSAQQTNTAGSNVTTS | |
| | | | | ATGTTACTACATCATCAACTCCTAATAGTAACTCTACATCTGGTTCTGCTACTA | | STPNSNSTSGSATSNPFGL | |
| | | | | GCAACCCTTTTGGTTTAGGTGGCCTTGGGGGACTTGCAGGTCTGAGTAGCTT | | GGLGGLAGLSSLGLNTTNF | |
| | | | | GGGTTTGAATACTACCAACTTCTCTGAACTACAGAGTCAGATGCAGCGACAA | | SELQSQMQRQLLSNPEMM | |
| | | | | CTTTTGTCTAACCCTGAAATGATGGTCCAGATCATGGAAAATCCCTTTGTTCA | | VQIMENPFVQSMLSNPDLM | |
| | | | | GAGCATGCTCTCAAATCCTGACCTGATGAGACAGTTAATTATGGCCAATCCA | | RQLIMANPQMQQLIQRNPE | |
| | | | | CAAATGCAGCAGTTGATACAGAGAAATCCAGAAATTAGTCATATGTTGAATAA | | ISHMLNNPDIMRQTLELAR | |
| | | | | TCCAGATATAATGAGACAAACGTTGGAACTTGCCAGGAATCCAGCAATGATG | | NPAMMQEMMRNQDRALS | |
| | | | | CAGGAGATGATGAGGAACCAGGACCGAGCTTTGAGCAACCTAGAAAGCATC | | NLESIPGGYNALRRMYTDI | |
| | | | | CCAGGGGGATATAATGCTTTAAGGCGCATGTACACAGATATTCAGGAACCAA | | QEPMLSAAQEQFGGNPFA | |
| | | | | TGCTGAGTGCTGCACAAGAGCAGTTTGGTGGTAATCCATTTGCTTCCTTGGT | | SLVSNTSSGEGSQPSRTEN | |
| | | | | GAGCAATACATCCTCTGGTGAAGGTAGTCAACCTTCCCGTACAGAAAATAGA | | RDPLPNPWAPQTSQSSSA | |
| | | | | GATCCACTACCCAATCCATGGGCTCCACAGACTTCCCAGAGTTCATCAGCTT | | SSG | |
| | | | | CCAGCGGCAC | | | |
| Shigella | 5 | prey67491 | 135 | AAAGAAAGATGTCAAGCAGCCAGAAGAACTCCCTCCCATCACAACCACAACA | 336 | KKDVKQPEELPPITTTTTST | |
| ipaC | | | | ACTTCTACTACACCAGCTACCAACACCACTTGTACAGCCACGGTTCCACCAC | | TPATNTTCTATVPPQPQYS | |
| | | | | AGCCACAGTACAGCTACCACGACATCAATGTCTATTCCCTTGCGGGCTTGGC | | YHDINVYSLAGLAPHITLNP | |
| | | | | ACCACACATTACTCTAAATCCAACAATTCCCTTGTTTCAGGCCCATCCACAGT | | TIPLFQAHPQLKQCVRQAIE | |
| | | | | TGAAGCAGTGTGTGCGTCAGGCAATTGAACGGGCTGTCCAGGAGCTGGTCC | | RAVQELVHPVVDRSIKIAMT | |
| | | | | ATCCTGTGGTGGATCGATCAATTAAGATTGCCATGACTACTTGTGAGCAAATA | | TCEQIVRKDFALDSEESRM | |
| | | | | GTCAGGAAGGATTTTGCCCTGGATTCGGAGGAATCTCGAATGCGAATAGCA | | RIAAHHMMRNLTAGMAMIT | |
| | | | | GCTCATCACATGATGCGTAACTTGACAGCTGGAATGGCTATGATTACATGCA | | CREPLLMSISTNLKNSFASA | |
| | | | | GGGAACCTTTGCTCATGAGCATATCTACCAACTTAAAAAACAGTTTTGCCTCA | | LRTASPQQREMMDQAAAQ | |
| | | | | GCCCTTCGTACTGCTTCCCCACAACAAAGAGAAATGATGGATCAGGCAGCTG | | LAQDNCELACCFIQKTAVE | |
| | | | | CTCAATTAGCTCAGGACAATTGTGAGTTGGCTTGCTGTTTTATTCAGAAGACT | | KAGPEMDKRLATEFELRKH | |
| | | | | GCAGTAGAAAAAGCAGGCCCTGAGATGGACAAGAGATTAGCAACTGAATTTG | | ARQEGRRYCDPVVLTYQA | |
| | | | | AGCTGAGAAAACATGCTAGGCAAGAAGGACGCAGATACTGTGATCCTGTTGT | | ERMPEQIRLKVGGVDPKQL | |
| | | | | TTTAACATATCAAGCTGAACGGATGCCAGAGCAAATCAGGCTGAAAGTTGGT | | AVYEEFARNVPGFLPTNDL | |
| | | | | GGTGTGGACCCAAAGCAGTTGGCTGTTTACGAAGAGTTTGCACGCAATGTTC | | SQPTGFLAQPMKQAWATD | |
| | | | | CTGGCTTCTTACCTACAAATGACTTAAGTCAGCCCACGGGATTTTTAGCCCA | | DVAQIYDKCITELEQHLHAI | |
| | | | | GCCCATGAAGCAAGCTTGGGCAACAGATGATGTAGCTCAGATTTATGATAAG | | PPTLAMNPQAQALRSLLEV | |
| | | | | TGTATTACAGAACTGGAGCAACATCTACATGCCATCCCACCAACTTTGGCCA | | VVLSRNSRDAIAALGLLQKA | |
| | | | | TGAACCCTCAAGCTCAGGCTCTTCGAAGTCTCTTGGAGGTTGTAGTTTTATCT | | VEGLLDATSGADADLLLRY | |
| | | | | CGAAACTCTCGGGATGCCATAGCTGCTCTTGGATTGCTCCAAAAGGCTGTAG | | | |
| | | | | AGGGCTTACTAGATGCCACAAGTGGTGCTGATGCTGACCTTCTGCTGCGCTA | | | |
| | | | | C | | | |
| Shigella | 5 | prey323 | 136 | AGACTCTATTCCGACACCCTCCAACATGGAGGAAACGCAACAGAAATCCAAT | 337 | DSIPTPSNMEETQQKSNLE | |
| ipaC | | | | CTAGAGCTGCTCCGCATCTCCCTGCTGCTCATCGAGTCGTGGCTGGAGCCC | | LLRISLLLIESWLEPVRFLRS | |
| | | | | GTGCGGTTCCTCAGGAGTATGTTCGCCAACAACCTGGTGTATGACACCTCG | | MFANNLVYDTSDSDDYHLL | |
| | | | | GACAGCGATGACTATCACCTCCTAAAGGACCTAGAGGAAGGCATCCAAACG | | KDLEEGIQTLMGRLEDGSR | |
| | | | | CTGATGGGGAGGCTGGAAGACGGCAGCCGCCGGACTGGGCAGATCCTCAA | | RTGQILKQTYSKFDTNSHN | |
| | | | | GCAGACCTACAGCAAGTTTGACACAAACTCGCACAACCATGACGCACTGCTC | | HDALLKNYGLLYCFRKDMD | |
| | | | | AAGAACTACGGGCTGCTCTACTGCTTCAGGAAGGACATGGACAAGGTCGAG | | KVETFLRMVQCRSVEGSC | |
| | | | | ACATTCCTGCGCATGGTGCAGTGCCGCTCTGTGGAGGGCAGCTGTGGCTTC | | GF* | |
| | | | | TAG | | | |
| Shigella | 5 | prey67495 | 137 | GCAGCAGTCTCTGTGCTGAAACCCTTCTCCAAGGGCGCGCCTTCTACCTCCA | 338 | AAVSVLKPFSKGAPSTSSP | |
| ipaC | | | | GCCCTGCAAAAGCCCTACCACAGGTGAGAGACAGATGGAAAGACTTAACCC | | AKALPQVRDRWKDLTHAISI | |
| | | | | ACGCTATTTCCATTTTAGAAAGTGCAAAGGCTAGAGTTACAAATACGAAGACG | | LESAKARVTNTKTSKPIVHA | |
| | | | | TCTAAACCAATCGTACATGCCAGAAAAAAATACCGCTTTCACAAAACTCGCTC | | RKKYRFHKTRSHVTHRTPK | |
| | | | | CCACGTGACCCACAGAACACCCAAAGTCAAAAAGAGTCCAAAGGTCAGAAA | | VKKSPKVRKKSYLS | |
| | | | | GAAAAGTTATCTGAGTA | | | |
| Shigella | 5 | prey67506 | 138 | GAGAGCCATCCCCAATCAGGGGGAGATCCTGGTGATCCGCAGGGGCTGGC | 339 | RAIPNQGEILVIRRGWLTIN | |
| ipaC | | | | TGACCATCAACAACATCAGCCTGATGAAAGGCGGCTCCAAGGAGTACTGGTT | | NISLMKGGSKEYWFVLTAE | |
| | | | | TGTGCTGACTGCCGAGTCACTGTCCTGGTACAAGGATGAGGAGGAGAAAGA | | SLSWYKDEEEKEKKYMLPL | |
| | | | | GAAGAAGTACATGCTGCCTCTGGACAACCTCAAGATCCGTGATGTGGAGAA | | DNLKIRDVEKGFMSNKHVF | |
| | | | | GGGCTTCATGTCCAACAAGCACGTCTTCGCCATCTTCAACACGGAGCAGAGA | | AIFNTEQRNVYKDLRQIELA | |
| | | | | AACGTCTACAAGGACCTGCGGCAGATCGAGCTGGCCTGTGACTCCCAGGAA | | CDSQEDVDSWKASFLRAG | |
| | | | | GACGTGGACAGCTGGAAGGCCTCGTTCCTCCGAGCTGGCGTCTACCCCGAG | | VYPEKDQAENEDGAQENT | |
| | | | | AAGGACCAGGCAGAAAACGAGGATGGGGCCCAGGAGAACACCTTCTCCATG | | FSMDPQLERQVETIRNLVD | |
| | | | | GACCCCCAACTGGAGCGGCAGGTGGAGACCATTCGCAACCTGGTGGACTCA | | SYVAIINKSIRDLMPKTIMHL | |
| | | | | TACGTGGCCATCATCAACAAGTCCATCCGCGACCTCATGCCAAAGACCATCA | | MINNTKAFIHHELLAYLYSS | |
| | | | | TGCACCTCATGATCAACAATACGAAGGCCTTCATCCACCACGAGCTGCTGGC | | ADQSSLMEESADQAQRRD | |
| | | | | CTACCTATACTCCTCGGCAGACCAGAGCAGCCTCATGGAGGAGTCGGCTGA | | DMLRMYHALKEALNIIGDIS | |
| | | | | CCAGGCACAGCGGCGGGACGACATGCTGCGCATGTACCATGCCCTCAAGG | | TSTVSTPVPP | |
| | | | | AGGCGCTCAACATCATCGGTGACATCAGCACCAGCACTGTGTCCACGCCTG | | | |
| | | | | TACCCCCGCC | | | |
| Shigella | 5 | prey4578 | 139 | CCAGAAGCAGCTGGAGTCCAATAAGATCCCAGAGCTGGACATGACTGAGGT | 340 | QKQLESNKIPELDMTEVVA | |
| ipaC | | | | GGTGGCCCCCTTCATGGCCAACATCCCTCTCCTCCTCTACCCTCAGGACGG | | PFMANIPLLLYPQDGPRSK | |
| | | | | CCCCCGCAGCAAGCCCCAGCCAAAGGATAATGGGGACGTTTGCCAGGACTG | | PQPKDNGDVCQDCIQMVT | |
| | | | | CATTCAGATGGTGACTGACATCCAGACTGCTGTACGGACCAACTCCACCTTT | | DIQTAVRTNSTFVQALVEH | |
| | | | | GTCCAGGCCTTGGTGGAACATGTCAAGGAGGAGTGTGACCGCCTGGGCCCT | | VKEECDRLGPGMADICKNY | |
| | | | | GGCATGGCCGACATATGCAAGAACTATATCAGCCAGTATTCTGAAATTGCTA | | ISQYSEIAIQMMMHMQPKEI | |
| | | | | TCCAGATGATGATGCACATGCAACCCAAGGAGATCTGTGCGCTGGTTGGGTT | | CALVGFCDEVKEMPMQTL | |
| | | | | CTGTGATGAGGTGAAAGAGATGCCCATGCAGACTCTGGTCCCCGCCAAAGT | | VPAKVASKNVIPALELVEPI | |
| | | | | GGCCTCCAAGAATGTCATCCCTGCCCTGGAACTGGTGGAGCCCATTAAGAA | | KKHEVPAKSDVYCEVCEFL | |
| | | | | GCACGAGGTCCCAGCAAAGTCTGATGTTTACTGTGAGGTGTGTGAATTCCTG | | VKEVTKLIDNNKTEKEILDA | |
| | | | | GTGAAGGAGGTGACCAAGCTGATTGACAACAACAAGACTGAGAAAGAAATAC | | FDKMCSKLPKSLSEECQE | |
| | | | | TCGACGCTTTTGACAAAATGTGCTCGAAGCTGCCGAAGTCCCTGTCGGAAGA | | | |
| | | | | GTGCCAGGAGG | | | |
| Shigella | 5 | prey1135 | 140 | TGCAGCCTTAGTGGCATCTAAAGTATTTTATCACCTGGGGGCTTTTGAGGAG | 341 | AALVASKVFYHLGAFEESL | |
| ipaC | | | | TCTCTGAATTATGCTCTTGGAGCAAGGGACCTCTTCAATGTCAATGATAACTC | | NYALGARDLFNVNDNSEYV | |
| | | | | TGAATATGTGGAAACTATTATAGCAAAATGCATTGATCACTACACCAAACAAT | | ETIIAKCIDHYTKQCVENAD | |
| | | | | GTGTGGAAAATGCAGATTTGCCTGAAGGAGAAAAAAAACCAATTGACCAGAG | | LPEGEKKPIDQRLEGIVNK | |
| | | | | ATTGGAAGGCATCGTAAATAAAATGTTCCAGCGATGTCTAGATGATCACAAGT | | MFQRCLDDHKYKQAIGIAL | |
| | | | | ATAAACAGGCTATTGGCATTGCTCTGGAGACACGAAGACTGGACGTCTTTGA | | ETRRLDVFEKTILESNDVPG | |
| | | | | AAAGACCATACTGGAGTCGAATGATGTCCCAGGAATGTTAGCTTATAGCCTT | | MLAYSLKLCMSLMQNKQF | |
| | | | | AAGCTCTGCATGTCTTTAATGCAGAATAAACAGTTTCGGAATAAAGTACTAAG | | RNKVLRVLVKIYMNLEKPD | |
| | | | | AGTTCTAGTTAAAATCTACATGAACTTGGAGAAACCTGATTTCATCAATGTTT | | FINVCQCLIFLDDPQAVSDIL | |
| | | | | GTCAGTGCTTAATTTTCTTAGATGATCCTCAGGCTGTGAGTGATATCTTAGAG | | EKLVKEDNLLMAYQICFDLY | |
| | | | | AAACTGGTAAAGGAAGACAACCTCCTGATGGCATATCAGATTTGTTTTGATTT | | ESASQQFLSSVIQNLRTVG | |
| | | | | GTATGAAAGTGCTAGCCAGCAGTTTTTGTCATCTGTAATCCAGAATCTTCGAA | | TPIASVPGSTNTGTVPGSE | |
| | | | | CTGTTGGCACCCCTATTGCTTCTGTGCCTGGATCCACTAATACGGGTACTGT | | KDSDSMETEEKTSSAFVGK | |
| | | | | TCCGGGATCAGAGAAAGACAGTGACTCGATGGAAACAGAAGAAAAGACAAG | | T | |
| | | | | CAGTGCATTTGTAGGAAAGACAC | | | |
| Shigella | 5 | prey67465 | 141 | CACTGCGCCGCTGCCCATGATGCCCGTGGCCGAGGACGAGATCAAGCCCTA | 342 | TAPLPMMPVAEDEIKPYISR | |
| ipaC | | | | CATCAGCCGCTGTTCTGTGTGTGAGGCCCCGGCCATCGCCATCGCGGTCCA | | CSVCEAPAIAIAVHSQDVSI | |
| | | | | CAGTCAGGATGTCTCCATCCCACACTGCCCAGCTGGGTGGCGGAGTTTGTG | | PHCPAGWRSLWIGYSFLM | |
| | | | | GATCGGATATTCCTTCCTCATGCACACGGCGGCGGGAGACGAAGGCGGTGG | | HTAAGDEGGGQSLVSPGS | |
| | | | | CCAATCACTGGTGTCACCGGGCAGCTGTCTAGAGGACTTCCGCGCCACACC | | CLEDFRATPFIECNGGRGT | |
| | | | | ATTCATCGAATGCAATGGAGGCCGCGGCACCTGCCACTACTACGCCAACAA | | CHYYANKYSFWLTTIPEQS | |
| | | | | GTACAGCTTCTGGCTGACCACCATTCCCGAGCAGAGCTTCCAGGGCTCGCC | | FQGSPSADTLKAGLIRTHIS | |
| | | | | CTCCGCCGACACGCTCAAGGCCGGCCTCATCCGCACACACATCAGCCGCTG | | RCQVCMKNL* | |
| | | | | CCAGGTGTGCATGAAGAACCTGTGA | | | |
| Shigella | 5 | prey28880 | 142 | AAGATCAAGTGGCTTACCTTATCCAACAAAATGTTATCCCACCTTTTTGCAAC | 343 | DQVAYLIQQNVIPPFCNLLT | |
| ipaC | | | | TTGCTGACTGTAAAAGATGCACAAGTTGTGCAAGTAGTACTCGATGGACTAA | | VKDAQVVQVVLDGLSNILK | |
| | | | | GTAATATATTAAAAATGGCTGAAGATGAGGCAGAAACCATAGGCAATCTTATA | | MAEDEAETIGNLIEECGGLE | |
| | | | | GAAGAATGTGGAGGGCTGGAGAAAATTGAACAACTTCAAAATCATGAAAATG | | KIEQLQNHENEDIYKLAYEII | |
| | | | | AAGACATCTACAAATTGGCCTATGAGATCATTGATCAGTTCTTCTCTTCAGAT | | DQFFSSDDIDEDPSLVPEAI | |
| | | | | GATATTGATGAAGACCCTAGCCTTGTTCCAGAGGCAATTCAAGGCGGAACAT | | QGGTFGFNSSANVPTEGF | |
| | | | | TTGGTTTCAATTCATCTGCCAATGTACCAACAGAAGGGTTCCAGTTTTAG | | QF* | |
| Shigella | 5 | prey3599 | 143 | GGCAGTTATTGAGATGTGTCAGTTACTGGTCATGGGAAATGAGGAGACACTG | 344 | AVIEMCQLLVMGNEETLGG | |
| ipaC | | | | GGAGGGTTTCCTGTCAAGAGTGTTGTTCCAGCTTTGATTACGTTACTTCAGAT | | FPVKSVVPALITLLQMEHNF | |
| | | | | GGAGCACAATTTTGATATTATGAACCATGCTTGTCGAGCCTTAACATACATGA | | DIMNHACRALTYMMEALPR | |
| | | | | TGGAAGCACTTCCTCGATCTTCTGCTGTTGTAGTAGATGCTATTCCTGTCTTT | | SSAVVVDAIPVFLEKLQVIQ | |
| | | | | TTAGAAAAGCTGCAAGTTATTCAGTGTATTGATGTGGCAGAGCAGGCCTTGA | | CIDVAEQALTALEMLSRRH | |
| | | | | CTGCCTTGGAGATGTTGTCACGGAGACATAGTAAAGCCATTCTACAGGCGG | | SKAILQAGGLADCLLYLEFF | |
| | | | | GTGGTTTGGCAGACTGCTTGCTGTACCTAGAATTCTTCAGCATAAATGCCCA | | SINAQRNALAIAANCCQSIT | |
| | | | | AAGAAATGCATTAGCAATTGCAGCTAATTGCTGCCAGAGTATCACGCCAGAT | | PDEFHFVADSLPLLTQRLT | |
| | | | | GAATTTCATTTTGTGGCAGATTCACTCCCATTGCTAACCCAAAGGCTAACACA | | HQDKKSVESTCLCFARLVD | |
| | | | | TCAGGATAAAAAGTCAGTAGAAAGCACTTGCCTTTGTTTTGCACGCCTAGTG | | NFQHEENLLQQVASKDLLT | |
| | | | | GACAACTTCCAGCATGAGGAGAATTTACTCCAGCAGGTTGCTTCCAAAGATC | | NVQQLLVVTPPILSSGMFIM | |
| | | | | TGCTTACAAATGTTCAACAGCTGTTGGTAGTGACTCCACCCATTTTAAGTTCT | | VVRMFSLMCSNCPTLAVQL | |
| | | | | GGGATGTTTATAATGGTGGTTCGCATGTTTTCTCTGATGTGTTCCAACTGTCC | | MKQNIAETLHFLLCGASNG | |
| | | | | AACTTTAGCTGTTCAACTTATGAAACAAAACATTGCAGAAACGCTTCACTTTC | | SCQEQIDLVPRSPQELYEL | |
| | | | | TCCTGTGTGGTGCCTCCAATGGAAGTTGTCAGGAACAGATTGATCTTGTTCC | | TSLICELMPCLPKEGIFAVD | |
| | | | | ACGAAGCCCTCAAGAGTTGTATGAACTGACATCTCTGATTTGTGAACTTATGC | | TMLKKGNAQNTDGAIWQW | |
| | | | | CATGTTTACCAAAAGAAGGCATTTTTGCAGTTGATACCATGTTGAAGAAGGGA | | RDDRGLWHPYNRIDSRIIE | |
| | | | | AATGCACAGAACACAGATGGTGGGATATGGCAGTGGCGTGATGATCGGGGC | | QINEDTGTARAIQRKPNPLA | |
| | | | | CTCTGGCATCCATATAACAGGATTGACAGCCGGATCATTGAGCAAATCAATG | | NSNTSGYSESKKDDARAQ | |
| | | | | AGGACACGGGAACAGCACGTGCCATTCAGAGAAAACCTAACCCGTTAGCCA | | LMKEDPELAKSFIKTLFGVL | |
| | | | | ATAGTAACACTAGTGGATATTCAGAGTCAAAGAAGGATGATGCTCGAGCACA | | YEVYSSSAGPAVRHKCLRA | |
| | | | | GCTTATGAAAGAGGATCCGGAACTGGCTAAGTCTTTTATTAAGACATTATTTG | | ILRIIYFADAELLKDVLKNHA | |
| | | | | GTGTTCTTTATGAAGTGTATAGTTCCTCAGCAGGACCTGCGGTCAGACATAA | | VSSHIASMLSSQDLKIVVGA | |
| | | | | GTGCCTTAGAGCAATTCTTAGGATAATTTATTTTGCGGATGCTGAACTTCTGA | | LQMAEILMQKLPDIFSVYFR | |
| | | | | AGGATGTTCTGAAAAATCATGCTGTTTCAAGTCACATTGCTTCCATGCTGTCA | | REGVMHQVKHLAESESLLT | |
| | | | | AGCCAAGACCTGAAGATAGTAGTGGGAGCACTTCAGATGGCAGAAATTTTAA | | SPPKACTNGSGSMGSTTS | |
| | | | | TGCAGAAGTTACCTGATATTTTTAGTGTTTACTTCAGAAGAGAAGGTGTAATG | | VSSGTATAATHAAADLGSP | |
| | | | | CATCAAGTAAAACACTTAGCAGAATCAGAGTCTTTGTTGACAAGTCCACCAAA | | SLQHSRDDSLDLSPQGRLS | |
| | | | | GGCATGTACGAATGGATCGGGATCCATGGGATCCACAACTTCAGTCAGCAG | | DVLKRKRLPKRGPRRPKYS | |
| | | | | TGGGACAGCCACAGCTGCCACTCATGCTGCAGCTGACTTGGGATCACCCAG | | PPRDDDKVDNQAKSPTTT | |
| | | | | CTTGCAGCACAGCAGGGATGATTCTTTAGATCTCAGCCCTCAAGGTCGATTA | | QSPKSSFLASLNPKTWGRL | |
| | | | | AGTGATGTTCTAAAGAGAAAACGACTGCCAAAACGAGGGCCAAGAAGGCCA | | STQSNSNNIEPARTAGGSG | |
| | | | | AAGTACTCACCTCCAAGAGATGATGACAAAGTAGACAATCAAGCTAAAAGCC | | LARAASKDTISNNREKIKG | |
| | | | | CCACCACTACTCAGTCACCTAAATCTTCTTTCCTGGCAAGCTTGAATCCAAAA | | WIKEQAHKFVERYFSSENM | |
| | | | | ACATGGGGAAGGTTAAGTACACAGTCCAACAGCAACAACATTGAGCCAGCAC | | DGSNPALNVLQRLCAATEQ | |
| | | | | GGACTGCGGGAGGTAGTGGCCTTGCCAGGGCTGCCTCAAAGGATACCATCT | | LNLQVDGGAECLVEIRSIVS | |
| | | | | CCAATAATAGAGAAAAAATTAAAGGTTGGATTAAGGAGCAGGCACATAAATTT | | ESDVSSFEIQHSGFVKQLLL | |
| | | | | GTAGAACGTTATTTCAGTTCTGAGAATATGGATGGAAGCAACCCTGCATTGA | | YLTSKSEKDAVSREIRLKRF | |
| | | | | ATGTCCTTCAGAGACTTTGTGCTGCAACCGAACAACTCAACCTCCAGGTGGA | | LHVFFSSPLPGEEPIGRVEP | |
| | | | | TGGTGGAGCTGAGTGCCTTGTAGAAATCCGTAGCATAGTCTCAGAGTCAGAT | | VGNAPLLALVHKMNNCLSQ | |
| | | | | GTTTCATCATTTGAAATCCAACATAGTGGATTTGTGAAGCAGCTGTTGCTTTA | | MEQFPVKVHDFPSGNGTG | |
| | | | | TTTGACATCTAAAAGTGAAAAGGATGCTGTGAGCAGAGAGATCAGATTAAAG | | GSFSLNRGSQALKFFNTHQ | |
| | | | | CGATTTCTTCATGTATTTTTTTCTTCTCCACTTCCTGGAGAAGAGCCCATTGG | | LKCQLQRHPDCANVKQWK | |
| | | | | AAGAGTGGAACCAGTGGGTAATGCACCTTTGTTGGCATTAGTTCACAAGATG | | GGPVKIDPLALVQAIERYLV | |
| | | | | AACAACTGCCTCAGCCAGATGGAACAATTTCCAGTCAAAGTACATGATTTCC | | VRGYGRVREDDEDSDDDG | |
| | | | | CTAGTGGAAATGGGACAGGAGGCAGCTTTTCTCTCAACAGAGGATCACAGG | | SDEEIDESLAAQFLNSGNV | |
| | | | | CTTTAAAATTTTTCAACACACATCAATTAAAATGCCAGTTACAAAGGCATCCA | | RHRLQFYIGEHLLPYNMTV | |
| | | | | GACTGTGCAAATGTGAAGCAGTGGAAGGGTGGACCTGTCAAGATTGACCCT | | YQAVRQFSIQAEDERESTD | |
| | | | | CTGGCTTTGGTACAAGCCATCGAGAGATACCTTGTAGTTAGAGGGTATGGAA | | DESNPLGRAGIWTKTHTIW | |
| | | | | GAGTAAGAGAAGATGATGAAGACAGCGATGACGATGGATCAGATGAGGAAA | | YKPVREDEESNKDCVGGK | |
| | | | | TAGATGAGTCTCTGGCTGCTCAGTTCCTAAATTCAGGAAATGTAAGACACAG | | RGRAQTAPTKTSPRNAKK | |
| | | | | GCTGCAGTTTTATATTGGAGAACATTTGCTGCCGTATAACATGACTGTGTATC | | HDELWHDGVCPSVSNPLE | |
| | | | | AGGCAGTACGGCAGTTTAGTATACAGGCTGAAGATGAAAGAGAATCCACAGA | | VYLIPTPPENITFEDPSLDVI | |
| | | | | TGATGAGAGCAATCCTCTAGGCAGAGCTGGTATTTGGACAAAGACTCATACA | | LLLRVLHAISRYWYYLYDNA | |
| | | | | ATATGGTATAAACCTGTGAGAGAGGATGAAGAAAGTAATAAAGATTGTGTTG | | MCKEIIPTSEFINSKLTAKAN | |
| | | | | GTGGTAAAAGAGGAAGAGCCCAAACAGCTCCAACGAAAACTTCCCCTAGAAA | | RQLQDPLVIMTGNIPTWLT | |
| | | | | TGCAAAAAAGCATGATGAGTTATGGCACGATGGAGTGTGCCCATCAGTATCA | | ELGKTCPFFFPFDTRQMLF | |
| | | | | AATCCTTTAGAAGTTTACCTCATTCCCACACCACCTGAAAATATAACATTTGAA | | YVTAFDRDRAMQRLLDTNP | |
| | | | | GACCCGTCATTAGATGTGATCCTTCTTTTAAGAGTTTTACATGCTATCAGTCG | | EINQSDSQDSRVAPRLDRK | |
| | | | | ATACTGGTATTACTTGTATGATAATGCAATGTGCAAGGAAATTATTCCAACTA | | KRTVNREELLKQAESVMQ | |
| | | | | GTGAATTTATTAACAGTAAGTTAACAGCAAAAGCAAATAGGCAACTTCAAGAT | | DLGSSRAMLEIQYENEVGT | |
| | | | | CCTTTAGTAATCATGACAGGAAACATCCCAACATGGCTTACTGAGCTAGGAA | | GLGPTLEFYALVSQELQRA | |
| | | | | AAACCTGCCCATTTTTCTTTCCTTTTGATACCCGGCAAATGCTTTTTTATGTAA | | DLGLWRGEEVTLSNPKGS | |
| | | | | CTGCATTTGATCGGGACCGAGCAATGCAAAGATTACTTGATACCAACCCAGA | | QEGTKYIQNLQGLFALPFG | |
| | | | | AATCAACCAGTCTGATTCTCAAGATAGCAGAGTTGCACCTAGATTGGATAGA | | RTAKPAHIAKVKMKFRFLG | |
| | | | | AAAAAACGTACTGTGAACCGAGAGGAGCTGCTGAAACAGGCGGAGTCTGTG | | KLMAKAIMDFRLVDLPLGLP | |
| | | | | ATGCAGGACCTCGGCAGCTCACGGGCCATGTTAGAAATCCAGTATGAAAATG | | FYKWMLRQETSLTSHDLFD | |
| | | | | AGGTTGGTACAGGTCTTGGGCCTACACTGGAGTTTTATGCGCTTGTATCTCA | | IDPVVARSVYHLEDIVRQKK | |
| | | | | GGAACTACAGAGAGCTGACTTGGGTCTTTGGAGAGGTGAAGAAGTAACTCTT | | RLEQDKSQTKESLQYALET | |
| | | | | AGCAATCCAAAAGGGAGCCAAGAAGGGACCAAGTATATTCAAAACCTCCAGG | | LTMNGCSVEDLGLDFTLPG | |
| | | | | GCCTGTTTGCGCTTCCCTTTGGTAGGACAGCAAAGCCAGCTCATATCGCAAA | | FPNIELKKGGKDIPVTIHNLE | |
| | | | | GGTTAAGATGAAGTTTCGCTTCTTAGGAAAATTAATGGCCAAGGCTATCATG | | EYLRLVIFWALNEGVSRQF | |
| | | | | GATTTCAGATTGGTGGACCTTCCCCTTGGCTTACCCTTTTATAAATGGATGCT | | DSFRDGFESVFPLSHLQYF | |
| | | | | ACGGCAAGAAACTTCACTGACATCACACGATTTGTTTGACATCGACCCAGTT | | YPEELDQLLCGSKADTWD | |
| | | | | GTAGCCAGATCAGTTTATCACCTAGAAGACATTGTCAGACAGAAGAAAAGAC | | AKTLMECCRPDHGYTHDS | |
| | | | | TTGAACAAGATAAATCCCAGACCAAAGAGAGTCTACAGTATGCATTAGAAAC | | RAVKFLFEILSSFDNEQQRL | |
| | | | | CTTGACTATGAATGGCTGCTCAGTTGAAGATCTAGGACTGGATTTCACTCTG | | FLQFVTGSPRLPVGGFRSL | |
| | | | | CCAGGGTTTCCCAATATCGAACTGAAGAAAGGAGGGAAGGATATACCAGTCA | | NPPLTIVRKTFESTENPDDF | |
| | | | | CTATCCACAATTTAGAGGAGTATCTAAGACTGGTTATATTCTGGGCACTAAAT | | LPSVMTCVNYLKLPDYSSIE | |
| | | | | GAAGGCGTTTCTAGGCAATTTGATTCGTTCAGAGATGGATTTGAATCAGTCTT | | IMREKLLIAAREGQQSFHLS | |
| | | | | CCCACTCAGTCATCTTCAGTACTTCTACCCGGAGGAACTGGATCAGCTCCTT | | * | |
| | | | | TGTGGCAGTAAAGCAGACACTTGGGATGCAAAGACACTGATGGAATGCTGTA | | | |
| | | | | GGCCTGATCATGGTTATACTCATGACAGTCGGGCTGTGAAGTTTTTGTTTGA | | | |
| | | | | GATTCTCAGTAGTTTTGATAATGAGCAGCAGAGGTTATTTCTCCAGTTTGTGA | | | |
| | | | | CTGGTAGCCCAAGATTGCCTGTTGGAGGATTCCGGAGTTTGAATCCACCTTT | | | |
| | | | | GACAATTGTCCGAAAGACGTTTGAATCAACAGAAAACCCAGATGACTTCTTG | | | |
| | | | | CCCTCTGTAATGACTTGTGTGAACTATCTTAAGTTGCCGGACTATTCAAGCAT | | | |
| | | | | TGAGATAATGCGTGAAAAACTGTTGATAGCAGCAAGAGAAGGGCAGCAGTC | | | |
| | | | | GTTCCATCTTTCCTGA | | | |
| Shigella | 6 | prey67717 | 144 | GCGGGACATCCAGTATTGGGCTCACGTGCATAAGACTGTCCCAGACAGCAG | 345 | AGHPVLGSRA*DCPRQQH | |
| ipaH9.8 | | | | CACAATCACGTTCAGCCAAGTGGAGTTTCCGACGCACTTGTGTGGCAGCCG | | NHVQPSGVSDALVWQPRE | |
| | | | | CGTGAATGTGAGCCGATATGCAGCTGGGAGGGGTTGTGGGCCTCCTGCGGT | | CEPICSWEGLWASCGEGL | |
| | | | | GAGGGGCTCCTGCCAGGAGCTCTGAGAAGCCTCCACAGAATCAGCCGTCG | | LPGALRSLHRISRRAPSAA | |
| | | | | GGCTCCTTCAGCAGCAGCTCCCCTTATCTGTGCCAACGACTGGGGGCCTAA | | APLICANDWGPNSRVPARL | |
| | | | | CTCAAGGGTGCCAGCCCGTCTTCCGCCAATACAGACTGTGGGATTCTGAGA | | PPIQTVGF*ELGAWGPLGW | |
| | | | | GTTAGGAGCCTGGGGTCCCCTGGGGTGGGGTGGTCAGGGTGAGCAGGTGG | | GGQGEQVGSVSLFPHALT | |
| | | | | GCTCTGTGAGCCTGTTTCCCCATGCCCTGACTCACCCCAATCCCTGGGTGA | | HPNPWVRTELLKATEGGA | |
| | | | | GGACAGAGCTCCTGAAGGCCACTGAAGGAGGTGCAGCACACTCCACCTGG | | AHSTWVAFRSSALFLPAGS | |
| | | | | GTGGCCTTCCGCAGCTCAGCCCTCTTCCTGCCAGCAGGAAGCCTCTGCCTG | | LCLRSLS*PSSPPPGSSETE | |
| | | | | CGCTCCTTAAGTTAGCCATCCTCACCCCCTCCGGGCAGCTCTGAGACTGAG | | PGPLAAPRPRPFSDRGATT | |
| | | | | CCAGGGCCACTAGCAGCACCCAGACCTCGACCCTTCTCAGACCGAGGCGCC | | PGRGGKEGRPKSRGLSW | |
| | | | | ACCACCCCAGGCCGAGGAGGCAAGGAGGGAAGACCAAAGTCTAGAGGACT | | WPWASLELWCHHLQKGG | |
| | | | | GTCTTGGTGGCCCTGGGCGAGTCTTGAACTTTGGTGCCATCATCTGCAAAAG | | KNACVVQLRGYAVKTRMV | |
| | | | | GGAGGAAAGAATGCCTGCGTGGTGCAGCTACGTGGATACGCAGTGAAGACC | | GRLALNNGSIWPGAVAHAC | |
| | | | | CGCATGGTGGGACGCCTGGCACTTAACAATGGTAGCATTTGGCCGGGCGCG | | NPSTLGGRGGRITRSGDQ | |
| | | | | GTGGCTCACGCCTGTAATCCCAGCACTTTGGGAGGCCGAGGCGGGCGGAT | | DHPG*NGETPSLLKIQKISR | |
| | | | | CACGAGGTCAGGAGATCAAGACCATCCCGGCTAAAACGGTGAAACCCCGTC | | A*WRAPVVPATWEAEAGE | |
| | | | | TCTACTAAAAATACAAAAAATTAGCCGGGCGTAGTGGCGGGCGCCTGTAGTC | | WCEPGRRSLQ*AEIPPLHS | |
| | | | | CCAGCTACTTGGGAGGCTGAGGCAGGAGAATGGTGTGAACCCGGGAGGCG | | SLGDRARLRLKKKKKNNGS | |
| | | | | GAGCTTGCAGTGAGCCGAGATCCCGCCACTGCACTCCAGCCTGGGCGACA | | IVFSAQEEGSWDRERATTP | |
| | | | | GAGCGAGACTCCGTCTCAAAAAAAAAAAAAAAAACAATGGTAGCATCGTTTTC | | HPSLYNRRATFSSSEQDRL | |
| | | | | AGTGCCCAGGAAGAAGGCAGCTGGGACAGGGAAAGGGCCACCACACCACA | | VAKSRK*GLVPARWLIPVIP | |
| | | | | CCCAAGCCTATACAACAGGAGAGCCACTTTCAGCAGCTCTGAGCAGGACAG | | VLWEAEAGAGWIT*GQGFE | |
| | | | | ACTTGTGGCCAAGTCAAGAAAGTAAGGTCTGGTCCCAGCGAGGTGGCTCAT | | TSPTNMVKPRLY*EYKN*P | |
| | | | | CCCTGTAATCCCAGTGCTTTGGGAGGCCGAAGCGGGGGCGGGGTGGATCA | | GVVARACNLSCLGG*GRRI | |
| | | | | CTTGAGGTCAGGGGTTTGAGACCAGCCCGACCAACATGGTGAAACCCCGTC | | A*TREAEVAVSRDRATTVQ | |
| | | | | TCTACTAAGAATATAAAAATTAGCCGGGCGTGGTGGCGCGTGCCTGTAATCT | | PGGSVRLGL | |
| | | | | CAGCTGCTTGGGAGGCTGAGGCAGGAGAATCGCTTGAACCCGGGAGGCAG | | | |
| | | | | AGGTTGCAGTGAGCCGAGATCGAGCCACTACTGTCCAGCCCGGCGGCAGT | | | |
| | | | | GTGAGGCTCGGTCTC | | | |
| Shigella | 6 | prey700 | 145 | ATGGGAATTGGTCTTTCTGCTCAAGGTGTGAACATGAATAGACTACCAGGTT | 346 | MGIGLSAQGVNMNRLPGW | |
| ipaH9.8 | | | | GGGATAAGCATTCATATGGTTACCATGGGGATGATGGACATTCGTTTTGTTCT | | DKHSYGYHGDDGHSFCSS | |
| | | | | TCTGGAACTGGACAACCTTATGGACCAACTTTCACTACTGGTGATGTCATTG | | GTGQPYGPTFTTGDVIGCC | |
| | | | | GCTGTTGTGTTAATCTTATCAACAATACCTGCTTTTACACCAAGAATGGACAT | | VNLINNTCFYTKNGHSLGIA | |
| | | | | AGTTTAGGTATTGCTTTCACTGACCTACCGCCAAATTTGTATCCTACTGTGGG | | FTDLPPNLYPTVGLQTPGE | |
| | | | | GCTTCAAACACCAGGAGAAGTGGTCGATGCCAATTTTGGGCAACATCCTTTC | | VVDANFGQHPFVFDIEDYM | |
| | | | | GTGTTTGATATAGAAGACTATATGCGGGAGTGGAGAACCAAAATCCAGGCAC | | REWRTKIQAQIDRFPIGDR | |
| | | | | AGATAGATCGATTTCCTATCGGAGATCGAGAAGGAGAATGGCAGACCATGAT | | EGEWQTMIQKMVSSYLVH | |
| | | | | ACAAAAAATGGTTTCATCTTATTTAGTCCACCATGGGTACTGTGCCACAGCAG | | HGYCATAE | |
| | | | | AGGC | | | |
| Shigella | 6 | prey67718 | 146 | ATGGGTGGATTATTTTCTCGATGGAGGACAAAACCTTCAACTGTAGAAGTTCT | 347 | MGGLFSRWRTKPSTVEVL | |
| ipaH9.8 | | | | AGAAAGTATAGATAAGGAAATTCAAGCATTGGAAGAATTTAGGGAAAAAAATC | | ESIDKEIQALEEFREKNQRL | |
| | | | | AGAGATTACAAAAATTATGGGTTGGAAGATTAATTCTGTATTCCTCAGTTCTC | | QKLWVGRLILYSSVLYLFTC | |
| | | | | TATCTGTTTACATGCTTAATTGTATATTTGTGGTATCTTCCTGATGAATTTACA | | LIVYLWYLPDEFTARLAMTL | |
| | | | | GCAAGACTTGCCATGACACTCCCATTTTTTGCTTTTCCATTGATCATCTGGAG | | PFFAFPLIIWSIRTVIIFFFSK | |
| | | | | CATAAGAACAGTAATTATTTTCTTCTTTTCCAAGAGAACAGAAAGAAATAATGA | | RTERNNEALDDLKSQRKKI | |
| | | | | AGCATTGGATGATTTAAAATCCCAGAGGAAAAAAATACTTGAAGAAGTCATGG | | LEEVMEKETYKT | |
| | | | | AAAAAGAAACTTACAAGACG | | | |
| Shigella | 6 | prey2530 | 147 | ATGGGCGACAAAGGGACCCGAGTGTTCAAGAAGGCCAGTCCAAATGGAAAG | 348 | MGDKGTRVFKKASPNGKL | |
| ipaH9.8 | | | | CTCACCGTCTACCTGGGAAAGCGGGACTTTGTGGACCACATCGACCTCGTG | | TVYLGKRDFVDHIDLVDPV | |
| | | | | GACCCTGTGGATGGTGTGGTCCTGGTGGATCCTGAGTATCTCAAAGAGCGG | | DGVVLVDPEYLKERRVYVT | |
| | | | | AGAGTCTATGTGACGCTGACCTGCGCCTTCCGCTATGGCCGGGAGGACCTG | | LTCAFRYGREDLDVLGLTF | |
| | | | | GATGTCCTGGGCCTGACCTTTCGCAAGGACCTGTTTGTGGCCAACGTACAGT | | RKDLFVANVQSFPPAPEDK | |
| | | | | CGTTCCCACCGGCCCCCGAGGACAAGAAGCCCCTGACGCGGCTGCAGGAA | | KPLTRLQERLIKKLGEHAYP | |
| | | | | CGCCTCATCAAGAAGCTGGGCGAGCACGCTTACCCTTTCACCTTTGAGATCC | | FTFEIPPNLPCSVTLQPGPE | |
| | | | | CTCCAAACCTTCCATGTTCTGTGACACTGCAGCCGGGGCCCGAAGACACGG | | DTGKACGVDYEVKAFCAE | |
| | | | | GGAAGGCTTGCGGTGTGGACTATGAAGTCAAAGCCTTCTGCGCGGAGAATT | | NLEEKIHKRNSVRLVIRKVQ | |
| | | | | TGGAGGAGAAGATCCACAAGCGGAATTCTGTGCGTCTGGTCATCCGGAAGG | | YAPERPGPQPTAETTRQFL | |
| | | | | TTCAGTATGCCCCAGAGAGGCCTGGCCCCCAGCCCACAGCCGAGACCACCA | | MSDKPLHLEASLDKEIYYH | |
| | | | | GGCAGTTCCTCATGTCGGACAAGCCCTTGCACCTAGAAGCCTCTCTGGATAA | | GEPISVNVHVTNNTNKTVK | |
| | | | | GGAGATCTATTACCATGGAGAACCCATCAGCGTCAACGTCCACGTCACCAAC | | KIKISVRQYADICLFNTAQY | |
| | | | | AACACCAACAAGACGGTGAAGAAGATCAAGATCTCAGTGCGCCAGTATGCA | | KCPVAMEEADDTVAPSSTF | |
| | | | | GACATCTGCCTTTTCAACACAGCTCAGTACAAGTGCCCTGTTGCCATGGAAG | | CKVYTLTPFLANNREKRGL | |
| | | | | AGGCTGATGACACTGTGGCACCCAGCTCGACGTTCTGCAAGGTCTACACAC | | ALDGKLKHEDTNLASSTLL | |
| | | | | TGACCCCCTTCCTAGCCAATAACCGAGAGAAGCGGGGCCTCGCCTTGGACG | | REGANREILGIIVSYKVKVK | |
| | | | | GGAAGCTCAAGCACGAAGACACGAACTTGGCCTCTAGCACCCTGTTGAGGG | | LVVSRGGLLGDLASSDVAV | |
| | | | | AAGGTGCCAACCGTGAGATCCTGGGGATCATTGTTTCCTACAAAGTGAAAGT | | ELPFTLMHPKPKEEPPHRE | |
| | | | | GAAGCTGGTGGTGTCTCGGGGCGGCCTGTTGGGAGATCTTGCATCCAGCGA | | VPENETPVDTNLIELDTNDD | |
| | | | | CGTGGCCGTGGAACTGCCCTTCACCCTAATGCACCCCAAGCCCAAAGAGGA | | DIVFEDFARQRLKGMKDDK | |
| | | | | ACCCCCGCATCGGGAAGTTCCAGAGAACGAGACGCCAGTAGATACCAATCT | | EEEEDGTGSPQLNNR* | |
| | | | | CATAGAACTTGACACAAATGATGACGACATTGTATTTGAGGACTTTGCTCGCC | | | |
| | | | | AGAGACTGAAAGGCATGAAGGATGACAAGGAGGAAGAGGAGGATGGTACCG | | | |
| | | | | GCTCTCCACAGCTCAACAACAGATAG | | | |
| Shigella | 6 | prey67731 | 148 | ATGTCAATAGCAGGAGTTGCTGCTCAGGAGATCAGAGTCCCATTAAAAACTG | 349 | MSIAGVAAQEIRVPLKTGFL | |
| ipaH9.8 | | | | GATTTCTACATAATGGCCGAGCCATGGGGAATATGAGGAAGACCTACTGGAG | | HNGRAMGNMRKTYWSSR | |
| | | | | CAGTCGCAGTGAGTTTAAAAACAACTTTTTAAATATTGACCCGATAACCATGG | | SEFKNNFLNIDPITMAYSLN | |
| | | | | CCTACAGTCTGAACTCTTCTGCTCAGGAGCGCCTAATACCACTTGGGCATGC | | SSAQERLIPLGHASKSAPM | |
| | | | | TTCCAAATCTGCTCCGATGAATGGCCACTGCTTTGCAGAAAATGGTCCATCT | | NGHCFAENGPSQKSSLPPL | |
| | | | | CAAAAGTCCAGCTTGCCCCCTCTTCTTATTCCCCCAAGTGAAAACTTGGGAC | | LIPPSENLGPHEEDQVVCG | |
| | | | | CACATGAAGAGGATCAAGTTGTATGTGGTTTTAAGAAACTCACAGTGAATGG | | FKKLTVNGVCASTPPLTPIK | |
| | | | | GGTTTGTGCTTCCACCCCTCCACTGACACCCATAAAAAACTCCCCTTCCCTTT | | NSPSLFPCAPLCERGSRPL | |
| | | | | TCCCCTGTGCCCCTCTTTGTGAACGGGGTTCTAGGCCTCTTCCACCGTTGCC | | PPLPISEALSLDDTDCEVEF | |
| | | | | AATCTCTGAAGCCCTCTCTCTGGATGACACAGACTGTGAGGTGGAATTCCTA | | LTSSDTDFLLEDSTLSDFKY | |
| | | | | ACTAGCTCAGATACAGACTTCCTTTTAGAAGACTCTACACTTTCTGATTTCAA | | DVPGRRSFRGCGQINYAYF | |
| | | | | ATATGATGTTCCTGGCAGGCGAAGCTTCCGTGGGTGTGGACAAATCAACTAT | | DTPAVSAADLSYVSDQNG | |
| | | | | GCATATTTTGATACCCCAGCTGTTTCTGCAGCAGATCTCAGCTATGTGTCTGA | | GVPDPNPPPPQTHRRLRR | |
| | | | | CCAAAATGGAGGTGTCCCAGATCCAAATCCTCCTCCACCTCAGACCCACCGA | | SHSGPAGSFNKPAIRISNC | |
| | | | | AGATTAAGAAGGTCTCATTCGGGACCAGCTGGCTCCTTTAACAAGCCAGCCA | | CIHRASPNSDEDKPEVPPR | |
| | | | | TAAGGATATCCAACTGTTGTATACACAGAGCTTCTCCTAACTCCGATGAAGAC | | VPIPPRPVKPDYRRWSAEV | |
| | | | | AAACCTGAGGTTCCCCCCAGAGTTCCCATACCTCCTAGACCAGTAAAGCCAG | | TSSTYSDEDRPPKVPPREP | |
| | | | | ATTATAGAAGATGGTCAGCAGAAGTTACTTCGAGCACCTATAGTGATGAAGA | | LSPSNSRTPSPKSLPSYLN | |
| | | | | CAGGCCTCCCAAAGTACCGCCAAGAGAACCTTTGTCACCGAGTAACTCGCG | | GVMPPTQSFAPDPKYVSS | |
| | | | | CACACCGAGTCCCAAAAGCCTTCCGTCTTACCTCAATGGGGTCATGCCCCC | | KALQRQNSEGSASKVPCIL | |
| | | | | GACACAGAGCTTTGCCCCTGATCCCAAGTATGTCAGCAGCAAAGCACTGCAA | | PIIENGKKVSSTHYYLLPER | |
| | | | | AGACAGAACAGCGAAGGATCTGCCAGTAAGGTTCCTTGCATTCTGCCCATTA | | PPYLDKYEKFFREAEETNG | |
| | | | | TTGAAAATGGGAAGAAGGTTAGTTCAACACATTATTACCTACTACCTGAACGA | | GAQIQPLPADCGISSATEKP | |
| | | | | CCACCATACCTGGACAAATATGAAAAATTTTTTAGGGAAGCAGAAGAAACAAA | | DSKTKMDLGGHVKRKHLS | |
| | | | | TGGAGGCGCCCAAATCCAGCCATTACCTGCTGACTGCGGTATATCTTCAGCC | | YVVSP* | |
| | | | | ACAGAAAAGCCAGACTCAAAAACAAAAATGGATCTGGGTGGCCACGTGAAG | | | |
| | | | | CGTAAACATTTATCCTATGTGGTTTCTCCTTAG | | | |
| Shigella | 6 | prey7155 | 149 | GCTCCCGGACGTCCCTGCTCCTGGCTTTTGCCCTGCTCTGCCTGCCCTGGC | 350 | SRTSLLLAFALLCLPWLQEA | |
| ipaH9.8 | | | | TTCAAGAGGCTGGTGCCGTCCAAACCGTTCCGTTATCCAGGCTTTTTGACCA | | GAVQTVPLSRLFDHAMLQA | |
| | | | | CGCTATGCTCCAAGCCCATCGCGCGCACCAGCTGGCCATTGACACCTACCA | | HRAHQLAIDTYQEFEETYIP | |
| | | | | GGAGTTTGAAGAAACCTATATCCCAAAGGACCAGAAGTATTCATTCCTGCAT | | KDQKYSFLHDSQTSFCFSD | |
| | | | | GACTCCCAGACCTCCTTCTGCTTCTCAGACTCTATTCCGACACCCTCCAACA | | SIPTPSNMEETQQKSNLEL | |
| | | | | TGGAGGAAACGCAACAGAAATCCAATCTAGAGCTGCTCCGCATCTCCCTGCT | | LRISLLLIESWLEPVRFLRS | |
| | | | | GCTCATCGAGTCGTGGCTGGAGCCCGTGCGGTTCCTCAGGAGTATGTTCGC | | MFANNLVYDTSDSDDYHLL | |
| | | | | CAACAACCTGGTGTATGACACCTCGGACAGCGATGACTATCACCTCCTAAAG | | KDLEEGIQTLMGVRVAPGV | |
| | | | | GACCTAGAGGAAGGCATCCAAACGCTGATGGGGGTGAGGGTGGCGCCAGG | | ANPGTPLA* | |
| | | | | GGTCGCCAATCCTGGAACCCCACTGGCTTAG | | | |
| Shigella | 6 | prey1687 | 150 | GGAGTATGATGCAGAGCGGCCCCCCAGCAAGCCTCCACCGGTTGAACTGCG | 351 | EYDAERPPSKPPPVELRAA | |
| ipaH9.8 | | | | GGCTGCTGCCCTTCGTGCAGAGATCACAGATGCTGAAGGCCTGGGTTTGAA | | ALRAEITDAEGLGLKLEDRE | |
| | | | | GCTCGAAGATCGAGAGACAGTTATTAAGGAGTTGAAGAAGTCACTCAAGATT | | TVIKELKKSLKIKGEELSEA | |
| | | | | AAGGGAGAGGAGCTAAGTGAGGCCAATGTGCGGCTGAGCCTCCTGGAGAA | | NVRLSLLEKKLDSAAKDAD | |
| | | | | GAAGTTGGACAGTGCTGCCAAGGATGCAGATGAGCGCATCGAGAAAGTCCA | | ERIEKVQTRLEETQALLRKK | |
| | | | | GACTCGGCTGGAGGAGACCCAGGCACTGCTGCGAAAGAAGGAGAAAGAGTT | | EKEFEETMDALQADIDQLE | |
| | | | | TGAGGAGACAATGGATGCACTCCAGGCTGACATCGACCAGCTGGAGGCAGA | | AEKAELKQRLNSQSKRTIE | |
| | | | | GAAGGCAGAACTAAAGCAGCGTCTGAACAGCCAGTCCAAACGCACGATTGA | | GLRGPPPSGIATLVSGIAGE | |
| | | | | GGGACTCCGGGGCCCTCCTCCTTCAGGCATTGCTACTCTGGTCTCTGGCAT | | EQQRGAIPGQAPGSVPGP | |
| | | | | TGCTGGTGAAGAACAGCAGCGAGGAGCCATCCCTGGGCAGGCTCCAGGGT | | GLVKDSPLLLQQISAMRLHI | |
| | | | | CTGTGCCAGGCCCAGGGCTGGTGAAGGACTCACCACTGCTGCTTCAGCAGA | | SQLQHENSILKGAQMKASL | |
| | | | | TCTCTGCCATGAGGCTGCACATCTCCCAGCTCCAGCATGAGAACAGCATCCT | | ASL | |
| | | | | CAAGGGAGCCCAGATGAAGGCATCCTTGGCATCCCTGC | | | |
| Shigella | 6 | prey67734 | 151 | ATGAGCCAGAGGGACACGCTGGTGCATCTGTTTGCCGGAGGATGTGGTGGT | 352 | MSQRDTLVHLFAGGCGGT | |
| ipaH9.8 | | | | ACAGTGGGAGCTATTCTGACATGTCCACTGGAAGTTGTAAAAACACGACTGC | | VGAILTCPLEVVKTRLQSSS | |
| | | | | AGTCATCTTCTGTGACGCTTTATATTTCTGAAGTTCAGCTGAACACCATGGCT | | VTLISEVQLNTMAGASVN | |
| | | | | GGAGCCAGTGTCAACCGAGTAGTGTCTCCCGGACCTCTTCATTGCCTAAAG | | RVVSPGPLHCLKVILEKEG | |
| | | | | GTGATCTTGGAAAAAGAAGGGCCTCGTTCCTTGTTTAGAGGACTAGGCCCCA | | PRSLFRGLGPNLVGVAPSR | |
| | | | | ATTTAGTGGGGGTAGCCCCTTCCAGAGCAATATACTTTGCTGCTTATTCAAAC | | AIYFAAYSNCKEKLNDVFD | |
| | | | | TGCAAGGAAAAGTTGAATGATGTATTTGATCCTGATTCTACCCAAGTACATAT | | PDSTQVHMISAAMAGFTAI | |
| | | | | GATTTCAGCTGCAATGGCAGGTTTTACTGCAATCACAGCAACCAACCCCATT | | TATNPIWLIKTRLQLDARNR | |
| | | | | TGGCTTATAAAGACTCGGTTACAGCTTGATGCAAGGAACCGCGGGGAAAGG | | GERRMGAFECVRKVYQTD | |
| | | | | CGAATGGGTGCTTTTGAATGTGTTCGTAAAGTGTATCAGACAGATGGACTAA | | GLKGFYRGMSASYAGISET | |
| | | | | AAGGATTTTATAGGGGCATGTCTGCTTCATATGCTGGTATATCAGAGACTGTT | | VIHFVIYESIKQKLLEYKTAS | |
| | | | | ATCCATTTTGTTATTTATGAAAGTATAAAACAAAAACTACTGGAATATAAGACT | | TMENGEESVKEASDFVGM | |
| | | | | GCTTCTACAATGGAAAATGGTGAAGAGTCTGTGAAAGAAGCATCAGATTTTG | | MLAAATSKTCATTIAYPHVV | |
| | | | | TGGGAATGATGCTAGCTGCTGCCACCTCAAAAACTTGTGCCACAACTATAGC | | RTRLREEGTKYRSFFQTLS | |
| | | | | ATATCCACATGTTGTAAGAACAAGACTACGTGAAGAGGGAACAAAATACAGA | | LLVQEEGYGSLYRGLTTHL | |
| | | | | TCTTTTTTTCAGACTCTATCTTTGCTTGTTCAAGAAGAAGGTTATGGGTCTCTT | | VRQIPNTAIMMATYELVVYL | |
| | | | | TATCGTGGTCTGACAACTCATCTAGTGAGACAGATTCCAAACACAGCCATTAT | | LNG* | |
| | | | | GATGGCCACCTATGAATTGGTGGTTTACCTACTCAATGGATAG | | | |
| Shigella | 6 | prey2694 | 152 | ATGGCACACGCTATGGAAAACTCCTGGACAATCAGTAAAGAGTACCATATTG | 353 | MAHAMENSWTISKEYHIDE | |
| ipaH9.8 | | | | ATGAAGAAGTGGGCTTTGCTCTGCCAAATCCACAGGAAAATCTACCTGATTTT | | EVGFALPNPQENLPDFYND | |
| | | | | TATAATGACTGGATGTTCATTGCTAAACATCTGCCTGATCTCATAGAGTCTGG | | WMFIAKHLPDLIESGQLRE | |
| | | | | CCAGCTTCGAGAAAGAGTTGAGAAGTTAAACATGCTCAGCATTGATCATCTC | | RVEKLNMLSIDHLTDHKSQ | |
| | | | | ACAGACCACAAGTCACAGCGCCTTGCACGTCTAGTTCTGGGATGCATCACCA | | RLARLVLGCITMAYVWGKG | |
| | | | | TGGCATATGTGTGGGGCAAAGGTCATGGAGATGTCCGTAAGGTCTTGCCAA | | HGDVRKVLPRNIAVPYCQL | |
| | | | | GAAATATTGCTGTTCCTTACTGCCAACTCTCCAAGAAACTGGAACTGCCTCCT | | SKKLELPPILVYADCVLANW | |
| | | | | ATTTTGGTTTATGCAGACTGTGTCTTGGCAAACTGGAAGAAAAAGGATCCTAA | | KKKDPNKPLTYENMDVLFS | |
| | | | | TAAGCCCCTGACTTATGAGAACATGGACGTTTTGTTCTCATTTCGTGATGGAG | | FRDGDCSKGFFLVSLLVEIA | |
| | | | | ACTGCAGTAAAGGATTCTTCCTGGTCTCTCTATTGGTGGAAATAGCAGCTGC | | AASAIKVIPTVFKAMQMQE | |
| | | | | TTCTGCAATCAAAGTAATTCCTACTGTATTCAAGGCAATGCAAATGCAAGAAC | | RDTLLKALLEIASCLEKALQ | |
| | | | | GGGACACTTTGCTAAAGGCGCTGTTGGAAATAGCTTCTTGCTTGGAGAAAGC | | VFHQIHDHVN | |
| | | | | CCTTCAAGTGTTTCACCAAATCCACGATCATGTGAAC | | | |
| Shigella | 6 | prey67740 | 153 | GNATGNATTACNTGCNATANTGTAGAAATTGGGCATGNGGACAAGGGGATG | 354 | XXITCXXVEIGHXDKGMVH | |
| ipaH9.8 | | | | GTTCATGTATCTCTTAACTGTCTGACATGGNAACATNGTCTATACCNAGTTNG | | VSLNCLTWXHXLYXVXVHF | |
| | | | | NGTGCACTTTTAAAATGAATCCGATTTGTCTGCACTNNNNTNCCNCNTCTNCC | | *NESDLSALXXXXXLXXCXC | |
| | | | | TCNTTNTATGTGNGTGCAGCGTTTACNCTACTNCANTCTGANTGTACTTANTG | | SVYXTXX*XYLXVIXXAXXX | |
| | | | | GTNATCTTNCNTGCNNTTGNGGNTGGNGANGGTGNTCGCNTTTTTNTTCTGT | | GXGXRXFXLCTXXGG | |
| | | | | GTACCNGNNNGGGGGGGN | | | |
| Shigella | 6 | prey67703 | 154 | GGCCATTGAGAAACTACTCGCTCTTCTCAACACGCTGGACAGGTGGATTGAT | 355 | AIEKLLALLNTLDRWIDETP | |
| ipaH9.8 | | | | GAGACTCCTCCAGTGGACCAGCCCTCTCGGTTTGGGAATAAGGCATACAGG | | PVDQPSRFGNKAYRTWYA | |
| | | | | ACCTGGTATGCCAAACTTGATGAGGAAGCAGAAAACTTGGTGGCCACAGTG | | KLDEEAENLVATVVPTHLA | |
| | | | | GTCCCTACCCATCTGGCAGCTGCTGTGCCTGAGGTGGCTGTTTACCTAAAG | | AAVPEVAVYLKESVGNSTR | |
| | | | | GAGTCAGTGGGGAACTCCACGCGCATTGACTACGGCACAGGGCATGAGGCA | | IDYGTGHEAAFAAFLCCLC | |
| | | | | GCCTTCGCTGCTTTCCTCTGCTGTCTCTGCAAGATTGGGGTGCTCCGGGTG | | KIGVLRVDDQIAIVFKVFNR | |
| | | | | GATGACCAAATAGCTATTGTCTTCAAGGTGTTCAATCGGTACCTTGAGGTTAT | | YLEVMRKLQKTYRMEPAG | |
| | | | | GCGGAAACTCCAGAAAACATACAGGATGGAGCCAGCCGGCAGCCAGGGAG | | SQGVWGLDDFQFLPFIWG | |
| | | | | TGTGGGGTCTGGATGACTTCCAGTTTCTGCCCTTCATCTGGGGCAGTTCGCA | | SSQLIDH | |
| | | | | GCTGATAGACCAC | | | |
| Shigella | 6 | prey67741 | 155 | GACAAGTTGAGCCAAGCAAAAGCCTACTGCAACTTGGGCCTAGCATTCAAGG | 356 | DKLSQAKAYCNLGLAFKAL | |
| ipaH9.8 | | | | CTCTGCTGAATTTCAGTAAAGCTGAAGAGTGTCANGAAGTACCTACTGTCCC | | LNFSKAEECXEVPTVPSPV | |
| | | | | TAGCCCAGTCTCTGAATAATTCCCAGGCTAAATTTCGAGCCCTAGGAAACCT | | SE*FPG*ISSPRKPGRYIHL* | |
| | | | | GGGCGATATATTCATCTGTAAAAAAGATATAAATGGTGCAATAAAATTCTATG | | KRYKWCNKIL*AATGLSSP | |
| | | | | AGCAGCAACTGGGCTTAGCTCACCAGGTAAAGGACAGAAGATTAGAAGCCA | | GKGQKIRSQCICSP | |
| | | | | GTGCATATGCAGCCCT | | | |
| Shigella | 6 | prey67742 | 156 | AGGTAATGGAGCTGGTGGTGGCAGCAGCCAGAAAACTCCACTCTTTGAAACT | 357 | GNGAGGGSSQKTPLFETY | |
| ipaH9.8 | | | | TACTCGGATTGGGACAGAGAAATCAAGAGGACAGGTGCTTCCGGGTGGAGA | | SDWDREIKRTGASGWRVC | |
| | | | | GTTTGTTCTATTAACGAGGGTTACATGATATCCACTTGCCTTCCAGAATACAT | | SINEGYMISTCLPEYIVVPS | |
| | | | | TGTAGTGCCAAGTTCTTTAGCAGACCAAGATCTAAAGATCTTTTCCCATTCTT | | SLADQDLKIFSHSFVGRRM | |
| | | | | TTGTTGGGAGAAGGATGCCACTCTGGTGCTGGAGCCACTCTAACGGCAGTG | | PLWCWSHSNGSALVRMAL | |
| | | | | CTCTTGTGCGAATGGCCCTCATCAAAGACGTGCTGCAGCAGAGGAAGATTG | | IKDVLQQRKIDQRICNAITKS | |
| | | | | ACCAGAGGATTTGTAATGCAATAACTAAAAGTCACCCACAGAGAAGTGATGT | | HPQRSDVYKSDLDKTLPNI | |
| | | | | TTACAAATCAGATTTGGATAAGACCTTGCCTAATATTCAAGAAGTACAAGCAG | | QEVQAAFVKLKQLCVNEPF | |
| | | | | CATTTGTAAAACTGAAGCAGCTATGCGTTAATGAGCCTTTTGAAGAAACTGAA | | EETEEKWLSSLENTRWLEY | |
| | | | | GAGAAATGGTTATCTTCACTGGAAAATACTCGATGGTTAGAATATGTAAGGG | | VRAFLKHSAELVYMLESKH | |
| | | | | CATTCCTTAAGCATTCAGCAGAACTTGTATACATGCTAGAAAGCAAACATCTC | | LSVVLQEEEGRDLSCCVAS | |
| | | | | TCTGTAGTCCTACAAGAGGAGGAAGGAAGAGACTTGAGCTGTTGTGTAGCTT | | LVQVMLDPYFRTITGFQSLI | |
| | | | | CTCTTGTTCAAGTGATGCTGGATCCCTATTTTAGGACAATTACTGGATTTCAG | | QKEWVMAGYQFLDRCNHL | |
| | | | | AGTCTGATACAGAAGGAGTGGGTCATGGCAGGATATCAGTTTCTAGACAGAT | | KRSEKESPLFLLFLDATWQ | |
| | | | | GCAACCATCTAAAGAGATCAGAGAAAGAGTCTCCTTTATTTTTGCTATTCTTG | | LLEQYPAAFEFSETYLAVLY | |
| | | | | GATGCCACCTGGCAGCTGTTAGAACAATATCCTGCAGCTTTTGAGTTCTCCG | | DSTRISLFGTFLFNSPHQRV | |
| | | | | AAACCTACCTGGCAGTGTTGTATGACAGCACCCGGATCTCACTGTTTGGCAC | | KQSTVSRIKSCTKQDYFPS | |
| | | | | CTTCCTGTTCAACTCCCCTCACCAGCGAGTGAAGCAAAGCACGGTCAGTAG | | RV* | |
| | | | | GATAAAAAGTTGTACAAAACAAGATTATTTTCCTTCACGAGTTTGA | | | |
| Shigella | 6 | prey67339 | 157 | GGAAGAAGAAGAGACAGAGCTGCCCACTGTGCCCCCAGTGCCCACAGAACC | 358 | EEEETELPTVPPVPTEPSP | |
| ipaH9.8 | | | | CAGTCCCATGCCAGACCCTTGCAGTAGTGAACTGGATGCCATGATGCTGGG | | MPDPCSSELDAMMLGPRG | |
| | | | | GCCCCGTGGGAAGACCTATGCTTTCAAGGGGGACTATGTGTGGACTGTATC | | KTYAFKGDYVWTVSDSGP | |
| | | | | AGATTCAGGACCGGGCCCCTTGTTCCGAGTGTCTGCCCTTTGGGAGGGGCT | | GPLFRVSALWEGLPGNLDA | |
| | | | | CCCCGGAAACCTGGATGCTGCTGTCTACTCGCCTCGAACACAATGGATTCAC | | AVYSPRTQWIHFFKGDKV | |
| | | | | TTCTTTAAGGGAGACAAGGTGTGGCGCTACATTAATTTCAAGATGTCTCCTG | | WRYINFKMSPGFPKKLNRV | |
| | | | | GCTTCCCCAAGAAGCTGAATAGGGTAGAACCTAACCTGGATGCAGCTCTCTA | | EPNLDAALYWPLNQKVFLF | |
| | | | | TTGGCCTCTCAACCAAAAGGTGTTCCTCTTTAAGGGCTCCGGGTACTGGCAG | | KGSGYWQWDELARTDFSS | |
| | | | | TGGGACGAGCTAGCCCGAACTGACTTCAGCAGCTACCCCAAACCAATCAAG | | YPKPIKGLFTGVPNQP | |
| | | | | GGTTTGTTTACGGGAGTGCCAAACCAGCCC | | | |
| Shigella | 6 | prey67337 | 158 | GGCTCCCTTGACCTTCCAAGAGGTGCAGGCTGGTGCGGCTGACATCCGCCT | 359 | APLTFQEVQAGAADIRLSF | |
| ipaH9.8 | | | | CTCCTTCCATGGCCGCCAAAGCTCGTACTGTTCCAATACTTTTGATGGGCCT | | HGRQSSYCSNTFDGPGRV | |
| | | | | GGGAGAGTCCTGGCCCATGCCGACATCCCAGAGCTGGGCAGTGTGCACTTC | | LAHADIPELGSVHFDEDEF | |
| | | | | GACGAAGACGAGTTCTGGACTGAGGGGACCTACCGTGGGGTGAACCTGCG | | WTEGTYRGVNLRIIAAHEV | |
| | | | | CATCATTGCAGCCCATGAAGTGGGCCATGCTCTGGGGCTTGGGCACTCCCG | | GHALGLGHSRYSQALMAP | |
| | | | | ATATTCCCAGGCCCTCATGGCCCCAGTCTACGAGGGCTACCGGCCCCACTT | | VYEGYRPHFKLHPDDVAGI | |
| | | | | TAAGCTGCACCCAGATGATGTGGCAGGGATCCAGGCTCTCTATGGCAAGAA | | QALYGKKSPVIRDEEEEET | |
| | | | | GAGTCCAGTGATAAGGGATGAGGAAGAAGAAGAGACAGAGCTGCCCACTGT | | ELPTVPPVPTEPSPMPDPC | |
| | | | | GCCCCCAGTGCCCACAGAACCCAGTCCCATGCCAGACCCTTGCAGTAGTGA | | SSELDAMMLGEAPPLQAV | |
| | | | | ACTGGATGCCATGATGCTGGGTGAGGCCCCTCCCCTCCAGGCTGTTGGCAG | | GRRWGQPADPEAWTNGS | |
| | | | | GCGGTGGGGGCAGCCTGCTGATCCTGAGGCCTGGACAAATGGGAGTGACA | | DMGLQHEQWRAPWEDLC | |
| | | | | TGGGACTTCAGCATGAGCAATGGAGGGCCCCGTGGGAAGACCTATGCTTTC | | FQGGLCVDCIRFRTGPLVP | |
| | | | | AAGGGGGACTATGTGTGGACTGTATCAGATTCAGGACCGGGCCCCTTGTTC | | SVCPLGGAPRKPGCCCLLA | |
| | | | | CGAGTGTCTGCCCTTTGGGAGGGGCTCCCCGGAAACCTGGATGCTGCTGTC | | SNTMDSLL* | |
| | | | | TACTCGCCTCGAACACAATGGATTCACTTCTTTAA | | | |
| Shigella | 6 | prey67746 | 159 | ATGGAGAAATATTCAATAATGAAGAGCATGAATATGCATCGAAAAAAAGGAAA | 360 | MEKYSIMKSMNMHRKKGK | |
| ipaH9.8 | | | | AAGGACCATTTTAGAAATGACACAAATACTCAAAAGGCATGGCTATTGCACCT | | RTILEMTQILKRHGYCTLGE | |
| | | | | TGGGAGAAGCCTTTAATCGGTTAGACTTCTCAAGTGCAATTCAAGATATCCG | | AFNRLDFSSAIQDIRTFNYV | |
| | | | | AACGTTCAATTATGTGGTCAAACTGTTGCAGCTAATTGCAAAATCCCAGTTAA | | VKLLQLIAKSQLTSLSGVAQ | |
| | | | | CTTCATTGAGTGGCGTGGCACAGAAGAATTACTTCAACATTTTGGATAAAATC | | KNYFNILDKIVQKVLDDHHN | |
| | | | | GTTCAAAAGGTTCTTGATGACCACCACAATCCTCGCTTAATCAAAGATCTTCT | | PRLIKDLLQDLSSTLCILIRG | |
| | | | | GCAAGACCTAAGCTCTACCCTCTGCATTCTTATTAGAGGAGTAGGGAAGTCT | | VGKSVLVGNINIWICRLETIL | |
| | | | | GTATTAGTGGGAAACATCAATATTTGGATTTGCCGATTAGAAACTATTCTCGC | | AWQQQLQDLQMTKQVNN | |
| | | | | CTGGCAACAACAGCTACAGGATCTTCAGATGACTAAGCAAGTGAACAATGGC | | GLTLSDLPLHMLNNILYRFS | |
| | | | | CTCACCCTCAGTGACCTTCCTCTGCACATGCTGAACAACATCCTATACCGGT | | DGWDIITLGQVTPTLYMLSE | |
| | | | | TCTCAGACGGATGGGACATCATCACCTTAGGCCAGGTGACCCCCACGTTGT | | DRQLWKKLCQYHFAEKQF | |
| | | | | ATATGCTTAGTGAAGACAGACAGCTGTGGAAGAAGCTTTGTCAGTACCATTTT | | CRHLILSEKGHIEWKLMYFA | |
| | | | | GCTGAAAAGCAGTTTTGTAGACATTTGATCCTTTCAGAAAAAGGTCATATTGA | | LQKHYPAKEQYGDTLHFCR | |
| | | | | ATGGAAGTTGATGTACTTTGCACTTCAGAAACATTACCCAGCGAAGGAGCAG | | HCSILFWKDSGHPCTAADP | |
| | | | | TACGGAGACACACTGCATTTCTGTCGGCACTGCAGCATTCTCTTTTGGAAGG | | DSCFTPVSPQHFIDLFKF* | |
| | | | | ACTCAGGACACCCCTGCACGGCGGCCGACCCTGACAGCTGCTTCACGCCTG | | | |
| | | | | TGTCTCCGCAGCACTTCATCGACCTCTTCAAGTTTTAA | | | |
| Shigella | 6 | prey54430 | 160 | GCTGTCCAAAACCAACAGGACCCTCTTTATATTTGGTGTCACAAAGTATATTG | 361 | LSKTNRTLFIFGVTKYIAGP | |
| ipaH9.8 | | | | CAGGACCCTATGAATGTGAAATACGGAACCCAGTGAGTGCCAGCCGCAGTG | | YECEIRNPVSASRSDPVTL | |
| | | | | ACCCAGTCACCCTGAATCTCCTCCATGGTCCAGACCTCCCCAGCATTTACCC | | NLLHGPDLPSIYPSFTYYRS | |
| | | | | TTCATTCACCTATTACCGTTCAGGAGAAAACCTCTACTTGTCCTGCTTCGCCG | | GENLYLSCFAESNPRAQYS | |
| | | | | AGTCTAACCCACGGGCACAATATTCTTGGACAATTAATGGGAAGTTTCAGCT | | WTINGKFQLSGQKLSIPQIT | |
| | | | | ATCAGGACAAAAGCTCTCTATCCCCCAAATAACTACAAAGCATAGTGGGCTC | | TKHSGLYACSVRNSATGKE | |
| | | | | TATGCTTGCTCTGTTCGTAACTCAGCCACTGGCAAGGAAAGCTCCAAATCCA | | SSKSITVKVSDWILP* | |
| | | | | TCACAGTCAAAGTCTCTGACTGGATATTACCCTGA | | | |
| Shigella | 6 | prey67749 | 161 | AAGAAATTTAAGTATATTGAGAATTTGGAAAAATGTGTTAAACTTGAAGTACTG | 362 | KKFKYIENLEKCVKLEVLNL | |
| ipaH9.8 | | | | AATCTCAGCTATAATCTAATAGGGAAGATTGAAAAGTTGGACAAGCTGTTAAA | | SYNLIGKIEKLDKLLKLRELN | |
| | | | | ATTACGTGAACTCAACTTATCATATAACAAAATCAGCAAAATTGAAGGCATAG | | LSYNKISKIEGIENMCNLQK | |
| | | | | AAAATATGTGTAATCTGCAAAAGCTTAACCTTGCAGGAAATGAAATTGAGCAT | | LNLAGNEIEHIPVWLGKKLK | |
| | | | | ATTCCAGTATGGTTAGGGAAGAAGTTAAAATCTTTGCGAGTCCTCA | | | |
| Shigella | 6 | prey67751 | 162 | GGAGGCAGAGCAAGACACTGTCTCTTAAAAAAAGGAAAGAAAACTCGACAAG | 363 | GGRARHCLLKKGKKTRQE | |
| ipaH9.8 | | | | AATCCTAGTGGGAGAGGCAGGACCATCCTGTGATGGGTCAATAATGACCCA | | S*WERQDHPVMGQ**PSHG | |
| | | | | GTCATGGAGCACAGTGATGCAGGAAAAGGGGTTGTGAGTGCCAGGAAGGCC | | AQ*CRKRGCECQEGQFRT | |
| | | | | AGTTTCGAACAACGTGGCAAGGGAAGCAGGCCTGTGAGAACGGGCCCTCTG | | TWQGKQACENGPSEPELR | |
| | | | | AGCCGGAACTGAGGGAGGAGTTGAGCCTGGGGCTCTCTGGGGGTGCAGTG | | EELSLGLSGGAVFXVG | |
| | | | | TTCCANGTGGGGGA | | | |
| Shigella | 6 | prey8739 | 163 | GGCTGAGCCACCCGTCCCCTCACCTCTGCCACTGGCCTCATCCCCTGAATC | 364 | AEPPVPSPLPLASSPESAR | |
| ipaH9.8 | | | | AGCCCGACCCAAGCCCCGTGCCCGGCCCCCTGAAGAAGGTGAAGATACCC | | PKPRARPPEEGEDTRPPRL | |
| | | | | GTCCTCCTCGCCTCAAGAAATGGAAAGGAGTGCGCTGGAAGCGGCTTCGGC | | KKWKGVRWKRLRLLLTIQK | |
| | | | | TGCTGCTGACCATCCAGAAGGGCAGTGGACGGCAGGAGGATGAGCGGGAA | | GSGRQEDEREVAEFMEQL | |
| | | | | GTGGCAGAGTTTATGGAGCAGCTTGGCACAGCCTTGCGACCTGACAAGGTA | | GTALRPDKVPRDMRRCCF | |
| | | | | CCGCGAGACATGCGTCGCTGCTGTTTCTGTCATGAGGAGGGTGACGGGGCC | | CHEEGDGATDGPARLLNL | |
| | | | | ACTGATGGGCCTGCCCGTCTGCTGAACCTGGACCTGGACCTGTGGGTGCAC | | DLDLWVHLNCALWSTEVY | |
| | | | | CTCAACTGTGCCCTTTGGTCCACGGAGGTGTATGAGACCCAGGGCGGAGCA | | ETQGGALMNVEVALHRGLL | |
| | | | | CTGATGAATGTGGAGGTTGCCCTGCACCGAGGACTGCTAACCAAGTGCTCC | | TKCSLCQRTGATSSCNRM | |
| | | | | CTGTGCCAGCGAACTGGTGCCACCAGCAGCTGCAATCGCATGCGTTGCCCC | | RCPNVYHFGCAIRAKCMFF | |
| | | | | AATGTCTACCATTTTGGTTGTGCCATCCGCGCCAAGTGCATGTTCTTCAAGG | | KDKTMLCPMHKIKGPCEQE | |
| | | | | ACAAGACCATGCTGTGTCCAATGCATAAGATCAAGGGGCCCTGTGAGCAAG | | LSSFAVFRR | |
| | | | | AGCTGAGCTCTTTTGCTGTCTTCCGGCGGG | | | |
| Shigella | 6 | prey18232 | 164 | CAGTGATATGATGCTGAACATCATCAACAGCTCTATTACTACCAAAGCCATCA | 365 | SDMMLNIINSSITTKAISRW | |
| ipaH9.8 | | | | GCCGGTGGTCATCTTTGGCTTGCAACATTGCCCTGGATGCTGTCAAGATGGT | | SSLACNIALDAVKMVQFEE | |
| | | | | ACAGTTTGAGGAGAATGGTCGGAAAGAGATTGACATAAAAAAATATGCAAGA | | NGRKEIDIKKYARVEKIPGG | |
| | | | | GTGGAAAAGATACCTGGAGGCATCATTGAAGACTCCTGTGTCTTGCGTGGAG | | IIEDSCVLRGVMINKDVTHP | |
| | | | | TCATGATTAACAAGGATGTGACCCATCCACGTATGCGGCGCTATATCAAGAA | | RMRRYIKNPRIVLLDSSLEY | |
| | | | | CCCTCGCATTGTGCTGCTGGATTCTTCTCTGGAATACAAGAAAGGAGGAAGC | | KKGGSQTDIEITREEDFTRI | |
| | | | | CAGACTGACATTGAGATTACACGAGAGGAGGACTTCACCCGAATTCTCCAGA | | LQMEEEYIQQLCEDIIQLKP | |
| | | | | TGGAGGAAGAGTACATCCAGCAGCTCTGTGAGGACATTATCCAACTGAAGCC | | DVVITEKGISDLAQHYLMRA | |
| | | | | CGATGTGGTCATCACTGAAAAGGGCATCTCAGATTTAGCTCAGCACTACCTT | | NITAIRRVRKTDNNRIARAC | |
| | | | | ATGCGGGCCAATATCACAGCCATCCGCAGAGTCCGGAAGACAGACAATAAT | | GARIVSRPEELREDDVGTG | |
| | | | | CGCATTGCTAGAGCCTGTGGGGCCCGGATAGTCAGCCGACCAGAGGAACTG | | AGLLEIKKIGDEYFTFITDCK | |
| | | | | AGAGAAGATGATGTTGGAACAGGAGCAGGCCTGTTGGAAATCAAGAAAATTG | | DPK | |
| | | | | GAGATGAATACTTTACTTTCATCACTGACTGCAAAGACCCCAAGGC | | | |
| Shigella | 6 | prey66739 | 165 | ATGGACGACAAGGAGTTAATTGAATACTTTAAGTCTCAGATGAAAGAAGATCC | 366 | MDDKELIEYFKSQMKEDPD | |
| ipaH9.8 | | | | TGACATGGCCTCAGCAGTGGCTGCCATCCGGACGTTGCTGGAGTTCTTGAA | | MASAVAAIRTLLEFLKRDKG | |
| | | | | GAGAGATAAAGGGGAGACAATCCAGGGTCTGAGGGCGAATCTCACCAGTGC | | ETIQGLRANLTSAIETLCGV | |
| | | | | CATAGAAACCCTGTGTGGTGTGGACTCCTCTGTGGCAGTGTCCTCTGGCGG | | DSSVAVSSGGELFLRFISLA | |
| | | | | GGAGCTCTTCCTCCGCTTCATCAGTCTTGCCTCCCTGGAATACTCCGATTAC | | SLEYSDYSKCKKIMIERGEL | |
| | | | | TCCAAATGTAAAAAGATCATGATTGAGCGGGGAGAACTTTTTCTCAGGAGAA | | FLRRISLSRNKIADLCHTFIK | |
| | | | | TATCACTGTCAAGAAACAAAATTGCAGATCTGTGCCATACTTTCATCAAAGAT | | DGATILTHAYSRVVLRVLEA | |
| | | | | GGAGCGACAATATTGACTCACGCCTACTCCAGAGTGGTCCTGAGAGTCCTG | | AVAAKKRFSVYVTESQPDL | |
| | | | | GAAGCAGCCGTGGCGGCCAAGAAGCGATTTAGTGTATACGTCACAGAGTCA | | SGKKMAKALCHLNVPVTVV | |
| | | | | CAGCCTGATTTGTCAGGTAAGAAAATGGCCAAAGCCCTCTGCCACCTCAACG | | LDAAVGYIMEKADLVIVGAE | |
| | | | | TCCCTGTCACTGTGGTGCTAGATGCTGCTGTCGGCTACATCATGGAGAAAGC | | GVVENGGIINKIGTNQMAV | |
| | | | | AGATCTTGTCATAGTTGGTGCTGAAGGAGTTGTTGAAAACGGAGGAATTATT | | CAKAQNKPFYVVAESFKFV | |
| | | | | AACAAGATTGGAACCAACCAGATGGCTGTGTGTGCCAAAGCACAGAACAAAC | | RLFPLNQQDVPDKFKYKAD | |
| | | | | CTTTCTATGTGGTTGCAGAAAGTTTCAAGTTTGTCCGGCTCTTTCCACTAAAC | | TLKVAQTGQDLKEEHPWV | |
| | | | | CAGCAAGACGTCCCAGATAAGTTTAAGTATAAGGCAGACACTCTCAAGGTCG | | DYTAPSLITLLFTDL | |
| | | | | CGCAGACTGGACAAGACCTCAAAGAGGAGCATCCGTGGGTCGACTACACTG | | | |
| | | | | CCCCTTCCTTAATCACTCTGCTGTTTACAGACCTGGG | | | |
| Shigella | 6 | prey67769 | 166 | GCAGCCTTCAAGGTCGCCACGCCGTATTCCCTGTATGTCTGTCCCGAGGGG | 367 | AAFKVATPYSLYVCPEGQN | |
| ipaH9.8 | | | | CAGAACGTCACCCTCACCTGCAGGCTCTTGGGCCCTGTGGACAAAGGGCAC | | VTLTCRLLGPVDKGHDVTF | |
| | | | | GATGTGACCTTCTACAAGACGTGGTACCGCAGCTCGAGGGGCGAGGTGCAG | | YKTWYRSSRGEVQTCSER | |
| | | | | ACCTGCTCAGAGCGCCGGCCCATCCGCAACCTCACGTTCCAGGACCTTCAC | | RPIRNLTFQDLHLHHGGHQ | |
| | | | | CTGCACCATGGAGGCCACCAGGCTGCCAACACCAGCCACGACCTGGCTCAG | | AANTSHDLAQRHGLESASD | |
| | | | | CGCCACGGGCTGGAGTCGGCCTCCGACCACCATGGCAACTTCTCCATCACC | | HHGNFSITMRNLTLLDSGL | |
| | | | | ATGCGCAACCTGACCCTGCTGGATAGCGGCCTCTACTGCTGCCTGGTGGTG | | YCCLVVEIRHHHSEHRVHG | |
| | | | | GAGATCAGGCACCACCACTCGGAGCACAGGGTCCATGGTGCCATGGAGCTG | | AMELQVQTGKDAPSNCVV | |
| | | | | CAGGTGCAGACAGGCAAAGATGCACCATCCAACTGTGTGGTGTACCCATCC | | YPSSSQDSENITAAALATG | |
| | | | | TCCTCCCAGGATAGTGAAAACATCACGGCTGCAGCCCTGGCTACGGGTGCC | | ACIVGILCLPLILLLVYKQRQ | |
| | | | | TGCATCGTAGGAATCCTCTGCCTCCCCCTCATCCTGCTCCTGGTCTACAAGC | | AAS | |
| | | | | AAAGGCAGGCAGCCTCCAA | | | |
| Shigella | 6 | prey13613 | 167 | CCTTGGAGCTGGTCCTTTCAGCCATATGATAAAATTAAAAACTAAGCCTCTCC | 368 | LGAGPFSHMIKLKTKPLPP | |
| ipaH9.8 | | | | CTCCTGATCCACCTCGTCTGGAATGTGTTGCCTTTAGCCACCAGAACCTTAA | | DPPRLECVAFSHQNLKLKW | |
| | | | | GCTGAAATGGGGAGAAGGAACTCCAAAGACATTGTCAACCGATTCTATTCAG | | GEGTPKTLSTDSIQYHLQM | |
| | | | | TACCACCTTCAGATGGAGGATAAGAATGGACGGTTTGTATCCCTATACAGAG | | EDKNGRFVSLYRGPCHTY | |
| | | | | GACCATGTCATACATACAAAGTACAAAGACTTAATGAGTCAACATCCTATAAA | | KVQRLNESTSYKFCIQACN | |
| | | | | TTCTGTATTCAAGCTTGTAATGAAGCTGGGGAAGGTCCCCTCTCCCAAGAAT | | EAGEGPLSQEYIFTTPKSV | |
| | | | | ATATTTTCACTACTCCAAAATCTGTCCCAGCTGCCTTGAAAGCCCCCAAAATA | | PAALKAPKIEKVNDHICEIT | |
| | | | | GAGAAAGTAAATGATCACATTTGTGAAATTACATGGGAGTGTTTACAGCCAAT | | WECLQPMKGDPVIYSLQV | |
| | | | | GAAAGGTGATCCAGTTATTTACAGTCTTCAAGTTATGTTGGGAAAAGATTCAG | | MLGKDSEFKQIYKGPDSSF | |
| | | | | AATTCAAACAGATTTACAAGGGTCCCGACTCTTCCTTCCGGTATTCCAGCCTT | | RYSSLQLNCEYRFRVCAIR | |
| | | | | CAGCTGAACTGTGAATATCGCTTCCGTGTATGTGCCATTCGCC | | | |
| Shigella | 6 | prey3337 | 168 | GGCTCGGCTGAAGGACCTGGAGGCTCTGCTGAACTCCAAGGAGGCCGCAC | 369 | ARLKDLEALLNSKEAALSTA | |
| ipaH9.8 | | | | TGAGCACTGCTCTCAGTGAGAAGCGCACGCTGGAGGGCGAGCTGCATGATC | | LSEKRTLEGELHDLRGQVA | |
| | | | | TGCGGGGCCAGGTGGCCAAGCTTGAGGCAGCCCTAGGTGAGGCCAAGAAG | | KLEAALGEAKKQLQDEMLR | |
| | | | | CAACTTCAGGATGAGATGCTGCGGCGGGTGGATGCTGAGAACAGGCTGCAG | | RVDAENRLQTMKEELDFQ | |
| | | | | ACCATGAAGGAGGAACTGGACTTCCAGAAGAACATCTACAGTGAGGAGCTG | | KNIYSEELRETKRRHETRLV | |
| | | | | CGTGAGACCAAGCGCCGTCATGAGACCCGACTGGTGGAGATTGACAATGGG | | EIDNGKQREFESRLADALQ | |
| | | | | AAGCAGCGTGAGTTTGAGAGCCGGCTGGCGGATGCGCTGCAGGAACTGCG | | ELRAQHEDQVEQYKKELEK | |
| | | | | GGCCCAGCATGAGGACCAGGTGGAGCAGTATAAGAAGGAGCTGGAGAAGA | | TYSAKLDNARQSAERNSNL | |
| | | | | CTTATTCTGCCAAGCTGGACAATGCCAGGCAGTCTGCTGAGAGGAACAGCA | | VGAAHEELQQSRIRIDSLSA | |
| | | | | ACCTGGTGGGGGCTGCCCACGAGGAGCTGCAGCAGTCGCGCATCCGCATC | | QLSQLQKQLAAKEAKLRDL | |
| | | | | GACAGCCTCTCTGCCCAGCTCAGCCAGCTCCAGAAGCAGCTGGCAGCCAAG | | EDSLARERDTSRRLLAEKE | |
| | | | | GAGGCGAAGCTTCGAGACCTGGAGGACTCACTGGCCCGTGAGCGGGACAC | | REMAEMRARMQQQLDEY | |
| | | | | CAGCCGGCGGCTGCTGGCGGAAAAGGAGCGGGAGATGGCCGAGATGCGG | | QELLDIKLALDMEIHAYRKL | |
| | | | | GCAAGGATGCAGCAGCAGCTGGACGAGTACCAGGAGCTTCTGGACATCAAG | | LEGEEERLRLSPSPTSQRS | |
| | | | | CTGGCCCTGGACATGGAGATCCACGCCTACCGCAAGCTCTTGGAGGGCGAG | | RGRASSHSSQTQGGGSVT | |
| | | | | GAGGAGAGGCTACGCCTGTCCCCCAGCCCTACCTCGCAGCGCAGCCGTGG | | KKRKLESTESRSSFSQHAR | |
| | | | | CCGTGCTTCCTCTCACTCATCCCAGACACAGGGTGGGGGCAGCGTCACCAA | | TSGRVAVEEVDEEGKFVRL | |
| | | | | AAAGCGCAAACTGGAGTCCACTGAGAGCCGCAGCAGCTTCTCACAGCACGC | | RNKSNEDQSMGNWQIKRQ | |
| | | | | ACGCACTAGCGGGCGCGTGGCCGTGGAGGAGGTGGATGAGGAGGGCAAGT | | NGDDPLLTYRFPPKFTLKA | |
| | | | | TTGTCCGGCTGCGCAACAAGTCCAATGAGGACCAGTCCATGGGCAATTGGC | | GQVVTIWAAGAGATHSPPT | |
| | | | | AGATCAAGCGCCAGAATGGAGATGATCCCTTGCTGACTTACCGGTTCCCACC | | DLVWKAQNTWGCGNSLRT | |
| | | | | AAAGTTCACCCTGAAGGCTGGGCAGGTGGTGACGATCTGGGCTGCAGGAGC | | ALINSTGEEVAMRKLVRSV | |
| | | | | TGGGGCCACCCACAGCCCCCCTACCGACCTGGTGTGGAAGGCACAGAACA | | TVVEDDEDEDGDDLLHHH | |
| | | | | CCTGGGGCTGCGGGAACAGCCTGCGTACGGCTCTCATCAACTCCACTGGGG | | HVSGSRR* | |
| | | | | AAGAAGTGGCCATGCGCAAGCTGGTGCGCTCAGTGACTGTGGTTGAGGACG | | | |
| | | | | ACGAGGATGAGGATGGAGATGACCTGCTCCATCACCACCATGTGAGTGGTA | | | |
| | | | | GCCGCCGCTGA | | | |
| Shigella | 6 | prey67774 | 169 | CCCACCTCCTGGCCGGTCCTTGAAGTTTTCTGGGGTCTATGGGCCAATAATC | 370 | PPPGRSLKFSGVYGPIICQ | |
| ipaH9.8 | | | | TGCCAGAGACCAAGTACCAATGAGCTTCCCCTATTTGACTTTCCTGTCAAAG | | RPSTNELPLFDFPVKEVFEL | |
| | | | | AGGTTTTTGAACTGCTCGGGGTGGAGAATGTGTTTCAGCTTTTTACTTGTGC | | LGVENVFQLFTCALLEFQIL | |
| | | | | CCTTCTGGAGTTTCAAATCCTGCTCTACTCACAGCATTACCAGAGACTGATGA | | LYSQHYQRLMTVAETITAL | |
| | | | | CTGTGGCGGAGACGATTACAGCTCTCATGTTTCCTTTCCAGTGGCAGCATGT | | MFPFQWQHVYVPILPASLL | |
| | | | | CTATGTCCCTATTCTCCCAGCTTCTCTCCTGCATTTCTTAGATGCTCCTGTTC | | HFLDAPVPYLMGLHSNGLD | |
| | | | | CATACCTGATGGGTTTGCATTCCAATGGCCTGGATGACCGGTCAAAGCTGGA | | DRSKLELPQEANLCFVDID | |
| | | | | GCTGCCTCAAGAGGCTAACCTCTGCTTTGTGGACATTGACAACCACTTCATT | | NHFIELPEDLPQFPNKLEFV | |
| | | | | GAGTTGCCAGAGGACTTGCCACAGTTCCCCAACAAATTGGAGTTTGTCCAGG | | QEVSEILMAFGIPPEGNLHC | |
| | | | | AAGTCTCTGAGATTCTCATGGCATTTGGAATTCCCCCTGAAGGGAATCTTCAT | | SESASKLKRLRASELVSDK | |
| | | | | TGCAGTGAGAGTGCCTCCAAGCTGAAGAGGCTGCGGGCCTCTGAGCTTGTC | | RNGNIAGSPLHSYELLKEN | |
| | | | | TCGGACAAGAGGAATGGGAACATTGCTGGCTCCCCTTTGCATTCCTACGAGC | | ETIARLQALVKRTGVSLEKL | |
| | | | | TTCTTAAGGAGAATGAAACTATTGCCCGGCTGCAAGCCTTGGTCAAGAGAAC | | EVREDPSSNKDLKVQCDE | |
| | | | | TGGGGTGAGCCTGGAAAAGTTGGAAGTGCGTGAAGACCCCAGCAGCAATAA | | EELRIYQLNIQIREVFANRFT | |
| | | | | GGATCTCAAAGTTCAGTGTGATGAAGAAGAACTCAGGATTTACCAGCTAAAC | | QMFADYEVFVIQPSQDKES | |
| | | | | ATTCAGATCCGGGAAGTTTTTGCAAATCGTTTCACTCAGATGTTTGCAGATTA | | WFTNREQMQNFDKASFLS | |
| | | | | TGAGGTGTTTGTCATCCAACCCAGCCAGGATAAGGAATCCTGGTTTACCAAC | | DQPEPYLPFLSRFLETQMF | |
| | | | | AGGGAGCAAATGCAAAACTTTGATAAAGCATCTTTTCTGTCAGATCAGCCTGA | | ASFIDNKIMCHDDDDKDPV | |
| | | | | GCCCTACCTGCCCTTCCTCTCAAGATTCCTGGAGACCCAGATGTTTGCATCT | | LRVFDSRVDKIRLLNVRTPT | |
| | | | | TTCATTGACAACAAAATAATGTGTCATGATGATGATGATAAAGACCCTGTACT | | LRTSMYQKCTTVDEAEKAI | |
| | | | | CCGGGTATTTGATTCCCGAGTTGACAAGATCAGGCTGTTGAATGTTCGGACA | | ELRLAKIDHTAIHPHLLDMKI | |
| | | | | CCTACTCTCCGTACATCCATGTACCAGAAGTGTACCACTGTGGATGAAGCAG | | GQGKYEPGFFPKLQSDVLS | |
| | | | | AGAAAGCAATTGAGCTGCGTCTGGCAAAAATTGACCATACTGCAATTCACCC | | TGPASNKWTKRNAPAQWR | |
| | | | | ACATTTACTTGACATGAAGATTGGACAAGGGAAATATGAGCCGGGCTTCTTC | | RKDRQKQHTEHLRLDNDQ | |
| | | | | CCTAAGCTGCAGTCTGATGTACTTTCCACTGGGCCAGCCAGCAACAAGTGGA | | REKYIQEARTMGSTIRQ | |
| | | | | CGAAAAGGAATGCCCCTGCCCAGTGGAGGCGGAAAGATCGGCAGAAGCAG | | | |
| | | | | CACACAGAACACCTGCGTTTAGATAATGACCAGAGGGAGAAGTACATCCAGG | | | |
| | | | | AAGCCAGGACTATGGGCAGCACTATCCGCCAG | | | |
| Shigella | 6 | prey67776 | 170 | TGGGATTCAACTAAAATTAGCAAAGCATACTACAAAGCAATGGTAATTAGCAC | 371 | WDSTKISKAYYKAMVISTW | |
| ipaH9.8 | | | | TTGGTGTTACTGGCTAAGAAAGAGGCACTTGATGCATGAAACAGACTCACGT | | CYWLRKRHLMHETDSRVP | |
| | | | | GTACCTGTGAGTTTATTATTTGATACAAGTGCCATTTCAAATCAGCAAGGGAA | | VSLLFDTSAISNQQGNWAN | |
| | | | | TTGGGCCAATTTGTTATCCATTTTGAAAACATATNAAGTTTGATNCCTACNTG | | LLSILKTYXV*XLXDNVLXN | |
| | | | | ACAACGTNCTNTNAAATGGGTGGGAGGTGGATNGGNCATGTGGGTGTNANG | | GWEVDXXCGCXAVXA | |
| | | | | CGGTGNNGGCGG | | | |
| Shigella | 6 | prey4758 | 171 | GCTCAGTGCTCTGGAGTCCACGGTGCCTCCCAGCCAGCCTCCACCTGTGGG | 372 | LSALESTVPPSQPPPVGTS | |
| ipaH9.8 | | | | CACCTCAGCCATCCACATGAGCCTGCTTGAGATGAGGCGGAGCGTGGCGGA | | AIHMSLLEMRRSVAELRLQ | |
| | | | | ACTCAGGCTCCAGCTCCAGCAGATGCGGCAGCTCCAGCTGCAGAACCAGGA | | LQQMRQLQLQNQELLRAM | |
| | | | | GTTGCTGAGGGCAATGATGAAGAAGGCCGAGCTGGAAATCAGTGGCAAAGT | | MKKAELEISGKVMETMKRL | |
| | | | | GATGGAAACAATGAAGAGACTGGAGGATCCCGTGCAGCGACAGCGCGTCCT | | EDPVQRQRVLVEQERQKY | |
| | | | | AGTGGAGCAAGAGAGACAAAAATATCTTCATGAGGAAGAGAAGATCGTCAAG | | LHEEEKIVKKLCELEDFVED | |
| | | | | AAGTTGTGCGAGTTGGAAGACTTTGTTGAAGACTTGAAGAAGGACTCCACGG | | LKKDSTAASRLVTLKDVED | |
| | | | | CAGCCAGCCGATTGGTTACTCTGAAAGACGTGGAAGACGGGGCTTTCCTCC | | GAFLLRQVGEAVATLKGEF | |
| | | | | TGCGTCAAGTGGGAGAGGCTGTAGCTACCCTGAAAGGAGAATTTCCAACCTT | | PTLQNKMRAILRIEVEAVRF | |
| | | | | ACAAAACAAGATGCGAGCCATCCTGCGCATAGAAGTGGAGGCCGTGCGGTT | | LKEEPHKLDSLLKRVRSMT | |
| | | | | TCTGAAGGAGGAGCCACACAAGCTGGACAGTCTCCTGAAGCGTGTGCGCAG | | DVLTMLRRHVTDGLLKGTD | |
| | | | | CATGACAGACGTCCTGACCATGCTGCGGAGACATGTCACTGATGGGCTCCT | | AAQAAQYMAMEKATAAEV | |
| | | | | GAAAGGCACGGACGCAGCCCAAGCCGCACAGTACATGGCTATGGAAAAGGC | | LKSQEEAAHTSGQPFHSTG | |
| | | | | CACAGCCGCAGAAGTCCTGAAGAGTCAGGAGGAGGCAGCCCACACCTCCG | | APGDAKSEVVPLSGMMVR | |
| | | | | GCCAGCCCTTCCACAGCACAGGTGCCCCTGGCGATGCGAAGTCGGAAGTG | | HAQSSPVVIQPSQHSVALL | |
| | | | | GTGCCTTTGTCCGGCATGATGGTTCGCCACGCGCAGAGCTCCCCTGTGGTC | | NPAQNLPHVASSPAV | |
| | | | | ATCCAGCCCTCCCAGCACTCCGTGGCCCTGCTGAACCCTGCTCAGAACTTG | | | |
| | | | | CCTCACGTGGCCAGCTCCCCAGCCGTC | | | |
| Shigella | 6 | prey67781 | 172 | CCTGAGGACCAACCACATTGGGTGGGTGCAGGAGTTCCTCAATGAAGAGAA | 373 | LRTNHIGWVQEFLNEENRG | |
| ipaH9.8 | | | | CCGTGGCCTGGATGTGCTGCTCGAGTACCTGGCCTTTGCCCAGTGCTCTGT | | LDVLLEYLAFAQCSVTYDM | |
| | | | | CACGTATGACATGGAGAGCACAGACAACGGGGCTTCCAACTCAGAGAAAAA | | ESTDNGASNSEKNKPLEQS | |
| | | | | CAAGCCCCTGGAGCAGTCTGTGGAAGACCTCAGCAAGGGTCCACCCTCCTC | | VEDLSKGPPSSVPKSRHLTI | |
| | | | | CGTGCCCAAAAGCCGCCACCTGACCATCAAGCTGACCCCAGCCCACAGCAG | | KLTPAHSRKALR | |
| | | | | GAAGGCCCTGCGG | | | |
| Shigella | 6 | prey2109 | 173 | GACTAAGGATCACCATTACTTTAAGTACTGCAAAATCTCAGCATTGGCTCTTC | 374 | TKDHHYFKYCKISALALLKM | |
| ipaH9.8 | | | | TGAAGATGGTGATGCATGCCAGATCGGGAGGCAATTTGGAAGTGATGGGTC | | VMHARSGGNLEVMGLMLG | |
| | | | | TGATGCTAGGAAAGGTGGATGGTGAAACCATGATCATTATGGACAGTTTTGC | | KVDGETMIIMDSFALPVEGT | |
| | | | | TTTGCCTGTGGAGGGCACTGAAACCCGAGTAAATGCTCAGGCTGCTGCATAT | | ETRVNAQAAAYEYMAAYIE | |
| | | | | GAATACATGGCTGCATACATAGAAAATGCAAAACAGGTTGGCCGCCTTGAAA | | NAKQVGRLENAIGWYHSH | |
| | | | | ATGCAATCGGGTGGTATCATAGCCACCCTGGCTATGGCTGCTGGCTTTCTGG | | PGYGCWLSGIDVSTQMLN | |
| | | | | GATTGATGTTAGTACTCAGATGCTCAATCAGCAGTTCCAGGAACCATTTGTAG | | QQFQEPFVAVVIDPTRTISA | |
| | | | | CAGTGGTGATTGATCCAACAAGAACAATATCCGCAGGGAAAGTGAATCTTGG | | GKVNLGAFRTYPKGYKPPD | |
| | | | | CGCCTTTAGGACATACCCAAAGGGCTACAAACCTCCTGATGAAGGACCTTCT | | EGPSEYQTIPLNKIEDFGVH | |
| | | | | GAGTACCAGACTATTCCACTTAATAAAATAGAAGATTTTGGTGTACACTGCAA | | CKQYYALEVSYFKSSLDRK | |
| | | | | ACAATATTATGCCTTAGAAGTCTCATATTTCAAATCCTCTTTGGATCGCAAATT | | LLELLWNKYWVNTLSSSSL | |
| | | | | GCTTGAGCTGTTGTGGAATAAATACTGGGTGAATACGTTGAGTTCTTCTAGCT | | LTN | |
| | | | | TGCTTACTAATGC | | | |
| Shigella | 6 | prey4060 | 174 | GGCAAATCACTTTTTCTTCAAAAAGGATTATAGTAAAGTCCAGCATCTGGCCC | 375 | ANHFFFKKDYSKVQHLALH | |
| ipaH9.8 | | | | TCCATGCATTCCATAATACAGAAGTGGAAGCTATGCAAGCAGAGAGCTGCTA | | AFHNTEVEAMQAESCYQL | |
| | | | | TCAGCTAGCTAGATCATTCCATGTTCAGGAAGATTATGACCAAGCTTTTCAGT | | ARSFHVQEDYDQAFQYYY | |
| | | | | ACTATTATCAAGCCACACAGTTTGCCTCATCCTCTTTTGTGCTCCCATTTTTTG | | QATQFASSSFVLPFFGLGQ | |
| | | | | GTTTGGGACAAATGTATATTTATCGAGGTGACAAAGAAAATGCATCTCAGTGC | | MYIYRGDKENASQCFEKVL | |
| | | | | TTTGAGAAGGTTTTGAAAGCTTATCCTAATAATTACGAAACTATGAAAATTCTC | | KAYPNNYETMKILGSLYAA | |
| | | | | GGCTCTCTCTATGCTGCCTCAGAAGATCAAGAAAAACGAGATATTGCCAAGG | | SEDQEKRDIAKGHLKKVTE | |
| | | | | GCCATTTGAAGAAGGTCACAGAACAGTATCCCGATGATGTTGAAGCTTGGAT | | QYPDDVEAWIELAQILEQT | |
| | | | | TGAATTGGCACAAATCTTAGAACAGACTGATATACAGGGTGCCCTTTCAGCC | | DIQGALSAYGTATRILQEKV | |
| | | | | TATGGAACAGCAACACGAATCCTTCAGGAGAAAGTGCAGGCCGATGTTCCTC | | QADVPPEILNNVGALHFRL | |
| | | | | CAGAGATTCTCAATAATGTGGGTGCCCTCCATTTTAGACTTGGAAACCTAGG | | GNLGEAKKYFLASLDRAKA | |
| | | | | GGAGGCTAAGAAATATTTTTTGGCGTCATTGGACCGTGCAAAAGCAGAAGCG | | EAEHDEHYYNAISVTTSYN | |
| | | | | GAACACGATGAGCATTACTATAACGCCATTTCCGTTACCACGTCATATAATCT | | LARLYEAMCEFHEAEKLYK | |
| | | | | CGCCAGGCTATATGAGGCGATGTGTGAATTCCATGAAGCAGAAAAACTGTAT | | NILREHPNYVDCYLRLGAM | |
| | | | | AAAAACATCTTACGCGAACATCCTAATTATGTTGACTGCTATTTGCGCCTAGG | | ARDKGNFYEASDWFKEAL | |
| | | | | AGCCATGGCTAGAGATAAGGGAAACTTTTATGAGGCTTCAGATTGGTTTAAG | | QINQDHPDAWSLIGNLHLA | |
| | | | | GAAGCTCTTCAGATTAATCAGGATCATCCAGATGCTTGGTCTTTGATTGGCAA | | KQEWGPGQKKFERILKQP | |
| | | | | TCTTCATTTGGCAAAACAAGAATGGGGTCCTGGGCAGAAGAAGTTTGAGAGG | | STQSDTYSMLALGNVWLQ | |
| | | | | ATATTAAAACAGCCATCCACACAGAGTGATACCTATTCTATGCTAGCCCTTGG | | TLHQPTRDREKEKRHQDR | |
| | | | | CAACGTGTGGCTCCAAACTTTACATCAGCCCACCCGAGATCGAGAAAAGGAA | | ALAIYKQVLRNDAKNLYAA | |
| | | | | AAGCGTCATCAAGATCGTGCTCTGGCCATCTACAAACAAGTACTCAGAAATG | | NGIGAVLAHKGYFREARDV | |
| | | | | ATGCAAAGAATCTGTATGCTGCCAATGGCATAGGAGCTGTTTTGGCCCACAA | | FAQVREATADISDVWLNLA | |
| | | | | AGGATATTTTCGTGAAGCTCGTGATGTATTTGCCCAAGTAAGAGAAGCAACA | | HIYVEQKQYISAVQMYENC | |
| | | | | GCAGATATTAGTGATGTGTGGCTGAACTTAGCACACATCTATGTGGAGCAAA | | LRKFYK | |
| | | | | AGCAGTACATCAGCGCCGTTCAGATGTATGAAAACTGCCTCCGAAAGTTCTA | | | |
| | | | | TAAGCA | | | |
| Shigella | 6 | prey49284 | 175 | CTCATCAACTACGTGGGCTTCATCAACTACCTCTTCTATGGGGGCACGGTTG | 376 | LINYVGFINYLFYGGTVAGQ | |
| ipaH9.8 | | | | CTGGACAGATAGTCCTTCGCTGGAAGAAGCCTGATATCCCCCGCCCCATCAA | | IVLRWKKPDIPRPIKINLLFPI | |
| | | | | GATCAACCTGCTGTTCCCCATCATCTACTTGCTGTTCTGGGCCTTCCTGCTG | | IYLLFWAFLLVFSLWSEPVV | |
| | | | | GTCTTCAGCCTGTGGTCAGAGCCGGTGGTGTGTGGCATTGGCCTGGCCATC | | CGIGLAIMLTGVPVYFLGVY | |
| | | | | ATGCTGACAGGAGTGCCTGTCTATTTCCTGGGTGTTTACTGGCAACACAAGC | | WQHKPKCFSDFIELLTLVS | |
| | | | | CCAAGTGTTTCAGTGACTTCATTGAGCTGCTAACCCTGGTGAGCCAGAAGAT | | QKMCVVVYPEVERGSGTE | |
| | | | | GTGTGTGGTCGTGTACCCCGAGGTGGAGCGGGGCTCAGGGACAGAGGAGG | | EANEDMEEQQQPMYQPTP | |
| | | | | CTAATGAGGACATGGAGGAGCAGCAGCAGCCCATGTACCAACCCACTCCCA | | TKDKDVAGQPQP* | |
| | | | | CGAAGGACAAGGACGTGGCGGGGCAGCCCCAGCCCTGA | | | |
| Shigella | 6 | prey67686 | 176 | CTGGGATTACAGGCATGAGCCACAGCACCTGGCTGAGTTTTCTCAGCACCAT | 377 | LGLQA*ATAPG*VFSAPFIE* | |
| ipaH9.8 | | | | TTATTGAATAGACTGTCCTTTCCCTGGTGTATGTTATTGCATTTGTTGAAAATG | | TVLSLVYVIAFVENEFTIDV*I | |
| | | | | AGTTCACCATAGATGTGTAGATTTATTTCTGGGTTCTCTATCCTGTTCTGTTG | | YFWVLYPVLLVYMSVFMLV | |
| | | | | GTCTATATGTCTGTTTTCATGCTGGTACCATGCTGTTTTGGTTACTACGGCTC | | PCCFGYYGSVV*SEVR*CD | |
| | | | | TGTAGTATAATCTGAAGTCAGGTAATGTGATTCCTCCANTTTTGTTCTTTCTG | | SSXFVLSAX | |
| | | | | CTNANG | | | |
| Shigella | 6 | prey66872 | 177 | TTTCACTCAAGAAGATATTGACAGAGCTATTGCTTACCTTTTCCCAAGTGGTT | 378 | FTQEDIDRAIAYLFPSGLFE | |
| ipaH9.8 | | | | TGTTTGAGAAACGAGCCAGGCCAGTAATGAAGCATCCTGAACAGATTTTTCC | | KRARPVMKHPEQIFPRQRA | |
| | | | | AAGACAAAGAGCAATCCAGTGGGGAGAAGATGGCCGTCCATTTCACTATCTC | | IQWGEDGRPFHYLFYTGK | |
| | | | | TTCTATACTGGCAAACAGTCATACTATTCATTAATGATTACCAGCTTTACTTCC | | QSYYSLMITSFTSRSHRTE | |
| | | | | CGATCACACAGGACAGAGAACAGCTGA | | NS* | |
| Shigella | 6 | prey67690 | 178 | ATGGAGATGAGGCTTCCAGTGGCTCGCAAGCCTCTTAGCGAGAGACTGGGC | 379 | MEMRLPVARKPLSERLGR | |
| ipaH9.8 | | | | CGCGACACTAAGAAACATCTAGTGGTGCCGGGGGATACAATCACTACGGAC | | DTKKHLVVPGDTITTDTGF | |
| | | | | ACAGGATTCATGCGGGGCCATGGAACGTATATGGGAGAAGAGAAGCTCATT | | MRGHGTYMGEEKLIASVA | |
| | | | | GCATCTGTTGCTGGCTCTGTGGAGAGAGTAAACAAGTTGATCTGTGTGAAAG | | GSVERVNKLICVKALKTRYI | |
| | | | | CTTTGAAAACCAGATACATTGGTGAAGTAGGAGACATCGTAGTGGGACGAAT | | GEVGDIVVGRITERRRSAE | |
| | | | | CACAGAGAGGAGAAGATCTGCAGAAGATGAGCTTGCAATGAGAGGTTTCTTA | | DELAMRGFLQEGDLISAEV | |
| | | | | CAGGAAGGGGACCTTATCAGTGCTGAGGTCCAGGCAGTGTTCTCTGACGGA | | QAVFSDGAVSLHTRSLKYG | |
| | | | | GCTGTCTCTTTGCACACGAGGAGCCTGAAATATGGAAAACTAGGTCAGGGG | | KLGQGVLVQVSPSLVKRQK | |
| | | | | GTTTTGGTCCAGGTTTCCCCCTCCCTGGTGAAACGGCAGAAGACCCACTTTC | | THFHDLPCGASVILGNNGFI | |
| | | | | ATGATTTGCCATGTGGTGCCTCAGTGATTCTCGGTAACAACGGCTTCATCTG | | WIYPTPEHKEEEAGGFIANL | |
| | | | | GATTTACCCAACACCTGAGCACAAAGAAGAGGAAGCAGGGGGCTTCATTGC | | EPVSLADREVISRLRNCIISL | |
| | | | | AAACCTGGAGCCTGTCTCTCTTGCTGATCGAGAGGTGATATCCCGGCTTCGG | | VTQRMMLYDTSILYCYEAS | |
| | | | | AACTGCATCATCTCGCTGGTAACTCAGAGGATGATGCTGTATGATACCAGCA | | LPHQIKDILKPEIMEEIVMET | |
| | | | | TCCTGTACTGCTATGAAGCATCCCTTCCACATCAGATCAAAGACATCTTAAAG | | RQRLLEQEG* | |
| | | | | CCAGAAATAATGGAGGAGATTGTGATGGAAACACGCCAGAGGCTTTTGGAAC | | | |
| | | | | AGGAGGGATAA | | | |
| Shigella | 6 | prey67695 | 179 | CAAAGATTTAAATATGAATGTGAACAGCTTTCAAAGGAAATTTGTGAATGAAG | 380 | KDLNMNVNSFQRKFVNEV | |
| ipaH9.8 | | | | TCAGAAGGTGTGAATCACTGGAGAGAATCCTCCGTTTTCTGGAAGACGAGAT | | RRCESLERILRFLEDEMQN | |
| | | | | GCAAAATGAGATTGTAGTTCAGTTGCTCGAGAAAAGCCCACTGACCCCGCTC | | EIVVQLLEKSPLTPLPREMI | |
| | | | | CCACGGGAAATGATTACCCTGGAGACTGTTCTAGAAAAACTGGAAGGAGAGT | | TLETVLEKLEGELQEANQN | |
| | | | | TACAGGAAGCCAACCAGAACCAGCAGGCCTTGAAACAAAGCTTCCTAGAACT | | QQALKQSFLELTELKYLLKK | |
| | | | | GACAGAACTGAAATACCTCCTGAAGAAAACCCAAGACTTCTTTGAGACGGAA | | TQDFFETETNLADDFFTED | |
| | | | | ACCAATTTAGCTGATGATTTCTTTACTGAGGACACTTCTGGCCTCCTGGAGTT | | TSGLLELKAVPAYMTGKLG | |
| | | | | GAAAGCAGTGCCTGCATATATGACCGGAAAGTTGGGGTTCATAGCCGGTGT | | FIAGVINRERMASFERLLW | |
| | | | | GATCAACAGGGAGAGGATGGCTTCCTTTGAGCGGTTACTGTGGCGAATCTG | | RICRGNVYLKFSEMDAPLE | |
| | | | | CCGAGGAAACGTGTACTTGAAGTTCAGTGAGATGGACGCCCCTCTGGAGGA | | DPVTKEEIQKNIFIIFYQGEQ | |
| | | | | TCCTGTGACGAAAGAAGAAATTCAGAAGAACATATTCATCATATTTTACCAAG | | LRQKIKKICDGFRATVYPCP | |
| | | | | GAGAGCAGCTCAGGCAGAAAATCAAGAAGATCTGTGATGGGTTTCGAGCCA | | EPAVERREMLESVNVRLED | |
| | | | | CTGTCTACCCTTGCCCAGAGCCTGCGGTGGAGCGCAGAGAGATGTTGGAGA | | LITVITQTESHRQRLLQEAA | |
| | | | | GCGTCAATGTGAGGCTGGAAGATTTAATCACCGTCATAACACAAACAGAGTC | | ANWHSWLIKVQKMKAVYHI | |
| | | | | TCACCGCCAGCGCCTGCTGCAGGAAGCCGCTGCCAACTGGCACTCCTGGCT | | LNMCNIDVTQQCVIAEIWFP | |
| | | | | CATCAAGGTGCAGAAGATGAAAGCTGTCTACCACATCCTGAACATGTGCAAC | | VADATRIKRALEQGMELSG | |
| | | | | ATCGACGTCACCCAGCAGTGTGTCATCGCCGAGATCTGGTTCCCGGTGGCA | | SSMAPIMTTVQSKTAPPTF | |
| | | | | GATGCCACACGTATCAAGAGGGCACTGGAGCAAGGCATGGAACTAAGTGGC | | NR | |
| | | | | TCCTCCATGGCCCCCATCATGACCACAGTGCAATCTAAAACAGCCCCTCCCA | | | |
| | | | | CATTTAACAGGAC | | | |
| Shigella | 6 | prey67336 | 180 | ATGGGAGTGACATGGGACTTCAGCATGAGCAATGGAGGGCCCCGTGGGAA | 381 | MGVTWDFSMSNGGPRGK | |
| ipaH9.8 | | | | GACCTATGCTTTCAAGGGGGACTATGTGTGGACTGTATCAGATTCAGGACCG | | TYAFKGDYVWTVSDSGPG | |
| | | | | GGCCCCTTGTTCCGAGTGTCTGCCCTTTGGGAGGGGCTCCCCGGAAACCTG | | PLFRVSALWEGLPGNLDAA | |
| | | | | GATGCTGCTGTCTACTCGCCTCGAACACAATGGATTCACTTCTTTAAGGGAG | | VYSPRTQWIHFFKGDKVW | |
| | | | | ACAAGGTGTGGCGCTACATTAATTTCAAGATGTCTCCTGGCTTCCCCAAGAA | | RYINFKMSPGFPKKLNRVE | |
| | | | | GCTGAATAGGGTAGAACCTAACCTGGATGCAGCTCTCTATTGGCCTCTCAAC | | PNLDAALYWPLNQKVFLFK | |
| | | | | CAAAAGGTGTTCCTCTTTAAGGGCTCCGGGTACTGGCAGTGGGACGAGCTA | | GSGYWQWDELARTDFSSY | |
| | | | | GCCCGAACTGACTTCAGCAGCTACCCCAAACCAATCAAGGGTTTGTTTACGG | | PKPIKGLFTGVPNQPSAAM | |
| | | | | GAGTGCCAAACCAGCCCTCGGCTGCTATGAGTTGGCAAGATGGCCGAGTCT | | SWQDGRVYFFKGKVYWRL | |
| | | | | ACTTCTTCAAGGGCAAAGTCTACTGGCGCCTCAACCAGCAGCTTCGAGTAGA | | NQQLRVEKGYPRNISHNW | |
| | | | | GAAAGGCTATCCCAGAAATATTTCCCACAACTGGATGCACTGTCGTCCCCGG | | MHCRPRTIDTTPSGGNTTP | |
| | | | | ACTATAGACACTACCCCATCAGGTGGGAATACCACTCCCTCAGGTACGGGCA | | SGTGITLDTTLSATETTFEY* | |
| | | | | TAACCTTGGATACCACTCTCTCAGCCACAGAAACCACGTTTGAATACTGA | | | |
| Shigella | 6 | prey6299 | 181 | AGACCAGAGCCATGTTGTTCAAGAGCATTTAAGTGAAGAAAAGGATGAAAGA | 382 | DQSHVVQEHLSEEKDERL | |
| ipaH9.8 | | | | CTACACTGTGAGAATAATGATAAAGCCCCTGAATCAGAGTCAGAGAAGCCAA | | HCENNDKAPESESEKPTPL | |
| | | | | CTCCTCTGTCCACTGGGCAAGGTAATAGAGCTGAAGAGGGACCAAACGCTA | | STGQGNRAEEGPNASSGF | |
| | | | | GTTCAGGTTTCATGAAGACTGCTGTACTAGGACCTACACTGAAAAATGTAATG | | MKTAVLGPTLKNVMMKNN | |
| | | | | ATGAAAAATAATAAACTAGCAGTTTCCCCTAACTATAATGCTACGTTTATGGG | | KLAVSPNYNATFMGFKMM | |
| | | | | CTTCAAGATGATGGATGGAAAACAGCATATTGTATTAAAATTGGTGCCTATCA | | DGKQHIVLKLVPIKQNVCSP | |
| | | | | AACAAAATGTATGTTCACCAGGCTCACAGTCAGGTGCTGCAAAGGACGGTAC | | GSQSGAAKDGTANLQPQT | |
| | | | | TGCTAATTTGCAGCCCCAGACTTTGGACACTAATGGATTTTTAACAGGAGTAA | | LDTNGFLTGVTTELNDTVY | |
| | | | | CAACTGAGTTAAATGACACAGTTTATATGAAAGCAGCTACTCCATTTTCATGT | | MKAATPFSCSSSILSGKAS | |
| | | | | TCATCTTCTATACTTTCAGGGAAAGCAAGTTCAGAAAAAGAAATGACTTTGAT | | SEKEMTLISQRNNMLQTMD | |
| | | | | ATCTCAAAGGAATAATATGCTTCAAACAATGGATTATGAGAAAAGTGTATCTT | | YEKSVSSLSATSELVTASV | |
| | | | | CTTTGTCAGCAACATCAGAATTGGTTACAGCATCAGTGAATTTGACCACAAAA | | NLTTKFETRDNVDFWGNHL | |
| | | | | TTTGAAACAAGAGATAATGTTGACTTCTGGGGAAATCATCTCACTCAGAGTCA | | TQSHPEVLGTTIKSPDKVN | |
| | | | | CCCCGAGGTATTAGGTACCACCATTAAAAGTCCAGATAAAGTCAACTGTGTT | | CVAKPNAYNSGDMHNYCI | |
| | | | | GCCAAACCAAATGCATACAACAGTGGAGATATGCATAATTATTGCATTAATTA | | NYGNCELPVESSNQGSLPF | |
| | | | | TGGCAACTGTGAGTTACCTGTTGAATCCTCCAACCAAGGATCATTACCTTTTC | | HNYSKVNNSNKRRRFSGT | |
| | | | | ATAATTACTCAAAAGTGAATAATTCTAATAAACGTCGTAGGTTTTCAGGAACA | | AVYENPQRESSSSKTVVQ | |
| | | | | GCAGTGTATGAAAACCCTCAAAGAGAATCTTCATCCAGCAAAACAGTTGTCC | | QPISESFLSLVRQESSKPD | |
| | | | | AACAACCAATTAGTGAATCATTTTTATCACTAGTGAGGCAGGAGAGCTCAAAA | | SLLASISLLNDKDGTLKAKS | |
| | | | | CCAGATAGCCTATTAGCATCTATTAGCCTTTTAAATGATAAAGATGGAACTTT | | EIEEQYVLEKGQNIDGQNL | |
| | | | | AAAAGCAAAATCTGAAATTGAAGAACAGTATGTTTTAGAAAAAGGACAAAACA | | YSNENQNLECATEKSKWE | |
| | | | | TTGATGGACAAAACCTGTACAGTAATGAAAATCAAAATTTAGAGTGTGCGACT | | DFSNVDSPMMPRITSVFSL | |
| | | | | GAAAAATCTAAATGGGAAGACTTTTCTAATGTCGATTCACCTATGATGCCTAG | | QSQQASEFLPPEVNQLLQD | |
| | | | | AATCACATCTGTTTTCTCTCTCCAGAGCCAACAGGCATCAGAATTTCTGCCAC | | VLKIKPDVKQDSSNTPNKG | |
| | | | | CTGAAGTAAACCAATTGCTTCAGGATGTATTGAAAATAAAACCTGATGTAAAA | | LPLHCDQSFQKHEREGKIV | |
| | | | | CAAGACTCTAGTAACACTCCAAATAAAGGCTTGCCACTTCATTGTGACCAGTC | | ESSKDFKVQGIFPVPPGSV | |
| | | | | ATTTCAAAAACACGAGAGAGAAGGCAAAATTGTTGAATCTTCGAAAGATTTCA | | GINVPTNDLNLKFGKEKQV | |
| | | | | AAGTGCAAGGCATCTTCCCAGTTCCACCTGGCAGTGTGGGTATTAATGTGCC | | SSIPQDVRDSEKMPRISGF | |
| | | | | TACAAATGATTTGAATTTGAAATTTGGAAAAGAAAAACAAGTGTCATCAATAC | | GTLLKTQSDAIITQQLVKDK | |
| | | | | CACAAGATGTGAGAGATTCAGAGAAGATGCCTAGAATTTCAGGTTTTGGCAC | | LRATTQNLGSFYMQSPLLN | |
| | | | | ATTACTTAAGACTCAGTCAGATGCGATAATAACACAGCAGCTTGTAAAAGACA | | SEQKKTIIVQTSKGFLIPLNI | |
| | | | | AACTACGAGCCACCACACAAAATTTAGGTTCTTTTTATATGCAGAGTCCACTT | | TNKPGLPVIPGNALPLVNS | |
| | | | | TTAAATTCAGAACAAAAAAAAACTATAATTGTTCAGACTTCAAAAGGATTCTTA | | QGIPASLFVNKKPGMVLTL | |
| | | | | ATACCATTGAACATTACTAACAAGCCTGGGCTACCAGTTATTCCTGGAAATGC | | NNGKLEGVSAVKTEGAPA | |
| | | | | ACTTCCATTGGTTAATTCACAAGGTATCCCTGCTTCTCTTTTTGTAAACAAGAA | | RGTVTKEPCKTPILKVEPN | |
| | | | | ACCTGGGATGGTTTTAACACTTAATAATGGGAAACTTGAAGGTGTTTCCGCT | | NNCLTPGLCSSIGSCLSMK | |
| | | | | GTCAAAACCGAGGGTGCCCCAGCTCGTGGAACTGTGACTAAGGAGCCTTGC | | SSSENTLPLKGPYILKPTSS | |
| | | | | AAAACACCTATTTTGAAGGTAGAACCAAACAATAATTGTCTTACACCTGGACT | | VKAVLIPNMLSEQQSTKLNI | |
| | | | | TTGTTCCAGCATTGGCAGTTGTTTGAGCATGAAAAGTAGCTCAGAAAATACTT | | SDSVKQQNEIFPKPPLYTFL | |
| | | | | TGCCATTAAAAGGCCCTTACATTTTGAAACCAACGAGTTCTGTGAAAGCTGTT | | PDGKQAVFLKCVMPNKTEL | |
| | | | | CTTATTCCTAACATGCTATCTGAGCAACAGAGCACTAAGTTGAATATCTCCGA | | LKPKLVQNSTYQNIQPKKP | |
| | | | | TTCAGTAAAACAGCAGAATGAGATTTTTCCAAAACCACCTCTTTATACCTTCTT | | EGTPQRILLKIFNPVLNVTA | |
| | | | | GCCTGATGGCAAACAAGCTGTTTTTTTAAAGTGTGTGATGCCAAATAAAACTG | | ANNLSVSNSASSLQKDNVP | |
| | | | | AGCTGCTTAAGCCCAAATTAGTCCAAAATAGTACTTATCAAAATATACAGCCA | | SNQIIGGEQKEPESRDALP | |
| | | | | AAGAAACCTGAAGGAACACCACAAAGAATATTGCTGAAAATTTTTAACCCTGT | | FLLDDLMPANEIVITSTATC | |
| | | | | TTTAAATGTGACTGCTGCTAATAATCTGTCAGTAAGCAACTCTGCATCCTCAT | | PESSEEPICVSDCSESRVL | |
| | | | | TGCAAAAAGACAACGTACCATCTAATCAGATTATAGGAGGAGAGCAGAAAGA | | RCKTNCRIERNFNRKKTSK | |
| | | | | GCCAGAATCTAGAGATGCCTTACCCTTCTTACTAGATGACTTAATGCCAGCAA | | KNFFKNKNSWK* | |
| | | | | ATGAAATTGTGATAACTTCTACTGCAACATGCCCAGAATCTTCTGAGGAACCA | | | |
| | | | | ATATGTGTCAGTGACTGTTCAGAGTCCAGGGTATTAAGGTGTAAAACAAATTG | | | |
| | | | | TAGAATTGAGAGGAACTTCAATAGAAAAAAGACTTCCAAAAAAAATTTTTTCAA | | | |
| | | | | AAACAAAAACTCATGGAAGTAA | | | |
| Shigella | 6 | prey6586 | 182 | CGCGCCGTGGAAGAAGATCCAGCAGAACACTTTCACGCGCTGGTGCAACGA | 383 | APWKKIQQNTFTRWCNEH | |
| ipaH9.8 | | | | GCACCTGAAGTGCGTGAGCAAGCGCATCGCCAACCTGCAGACGGACCTGAG | | LKCVSKRIANLQTDLSDGL | |
| | | | | CGACGGGCTGCGGCTTATCGCGCTGTTGGAGGTGCTCAGCCAGAAGAAGAT | | RLIALLEVLSQKKMHRKHN | |
| | | | | GCACCGCAAGCACAACCAGCGGCCCACTTTCCGCCAAATGCAGCTTGAGAA | | QRPTFRQMQLENVSVALEF | |
| | | | | CGTGTCGGTGGCGCTCGAGTTCCTGGACCGCGAGAGCATCAAACTGGTGTC | | LDRESIKLVSIDSKAIVDGNL | |
| | | | | CATCGACAGCAAGGCCATCGTGGACGGGAACCTGAAGCTGATCCTGGGCCT | | KLILGLIWTLILHYSISMPMW | |
| | | | | CATCTGGACCCTGATCCTGCACTACTCCATCTCCATGCCCATGTGGGACGAG | | DEEEDEEAKKQTPKQRLLG | |
| | | | | GAGGAGGATGAGGAGGCCAAGAAGCAGACCCCCAAGCAGAGGCTCCTGGG | | WIQNKLPQLPITNFSRDWQ | |
| | | | | CTGGATCCAGAACAAGCTGCCGCAGCTGCCCATCACCAACTTCAGCCGGGA | | SGRALGALVDSCAPGLCPD | |
| | | | | CTGGCAGAGCGGCCGGGCCCTGGGCGCCCTGGTGGACAGCTGTGCCCCG | | WDSWDASKPVTNAREAM | |
| | | | | GGCCTGTGTCCTGACTGGGACTCTTGGGACGCCAGCAAGCCCGTTACCAAT | | QQADDWLGIPQVITPEEIVD | |
| | | | | GCGCGAGAGGCCATGCAGCAGGCGGATGACTGGCTGGGCATCCCCCAGGT | | PNVDEHSVMTYLSQFPKAK | |
| | | | | GATCACCCCCGAGGAGATTGTGGACCCCAACGTGGACGAGCACTCTGTCAT | | LKPGAPLRPKLNPKKARAY | |
| | | | | GACCTACCTGTCCCAGTTCCCCAAGGCCAAGCTGAAGCCAGGGGCTCCCTT | | GPGIEPTGNMVKKRAEFTV | |
| | | | | GCGCCCCAAACTGAACCCGAAGAAAGCCCGTGCCTACGGGCCAGGCATCG | | ETRSAGQGEVLVYVEDPA | |
| | | | | AGCCCACAGGCAACATGGTGAAGAAGCGGGCAGAGTTCACTGTGGAGACCA | | GHQEEAKVTANNDKNRTF | |
| | | | | GAAGTGCTGGCCAGGGAGAGGTGCTGGTGTACGTGGAGGACCCGGCCGGA | | SVWYVPEVTGTHKVTVLFA | |
| | | | | CACCAGGAGGAGGCAAAAGTGACCGCCAATAACGACAAGAACCGCACCTTC | | GQHIAKSPFEVYVDKSQGD | |
| | | | | TCCGTCTGGTACGTCCCCGAGGTGACGGGGACTCATAAGGTTACTGTGCTC | | ASKVTAQGPGLEPSGNIAN | |
| | | | | TTTGCTGGCCAGCACATCGCCAAGAGCCCCTTCGAGGTGTACGTGGATAAG | | KTTYFEIFTAGAGTGEVEVV | |
| | | | | TCACAGGGTGACGCCAGCAAAGTGACAGCCCAAGGTCCCGGCCTGGAGCC | | IQDPMGQKGTVEPQLEAR | |
| | | | | CAGTGGCAACATCGCCAACAAGACCACCTACTTTGAGATCTTTACGGCAGGA | | GDSTYRCSYQPTMEGVHT | |
| | | | | GCTGGCACGGGCGAGGTCGAGGTTGTGATCCAGGACCCCATGGGACAGAA | | VHVTFAGVPIPRSPYTVTV | |
| | | | | GGGCACGGTAGAGCCTCAGCTGGAGGCCCGGGGCGACAGCACATACCGCT | | GQACNPSACRAVGRGLQP | |
| | | | | GCAGCTACCAGCCCACCATGGAGGGCGTCCACACCGTGCACGTCACGTTTG | | KGVRVKETADFKVYTKGAG | |
| | | | | CCGGCGTGCCCATCCCTCGCAGCCCCTACACTGTCACTGTTGGCCAAGCCT | | SGELKVTVKGPKGEERVK | |
| | | | | GTAACCCGAGTGCCTGCCGGGCGGTTGGCCGGGGCCTCCAGCCCAAGGGT | | QKDLGDGVYGFEYYPMVP | |
| | | | | GTGCGGGTGAAGGAGACAGCTGACTTCAAGGTGTACACAAAGGGCGCTGGC | | GTYIVTITWGGQNIGRSPFE | |
| | | | | AGTGGGGAGCTGAAGGTCACCGTGAAGGGCCCCAAGGGAGAGGAGCGCGT | | VKVGTECGNQKVRAWGPG | |
| | | | | GAAGCAGAAGGACCTGGGGGATGGCGTGTATGGCTTCGAGTATTACCCCAT | | LEGGVVGKSADFVVEAIGD | |
| | | | | GGTCCCTGGAACCTATATCGTCACCATCACGTGGGGTGGTCAGAACATCGG | | DVGTLGFSVEGPSQAKIEC | |
| | | | | GCGCAGTCCCTTCGAAGTGAAGGTGGGCACCGAGTGTGGCAATCAGAAGGT | | DDKGDGSCDVRYWPQEA | |
| | | | | ACGGGCCTGGGGCCCTGGGCTGGAGGGCGGCGTCGTTGGCAAGTCAGCAG | | GEYAVHVLCNSEDIRLSPF | |
| | | | | ACTTTGTGGTGGAGGCTATCGGGGACGACGTGGGCACGCTGGGCTTCTCG | | MADIRDAPQDFHPDRVKAR | |
| | | | | GTGGAAGGGCCATCGCAGGCTAAGATCGAATGTGACGACAAGGGCGACGG | | GPGLEKTGVAVNKPAEFTV | |
| | | | | CTCCTGTGATGTGCGCTACTGGCCGCAGGAGGCTGGCGAGTATGCCGTTCA | | DAKHGGKAPLRVQVQDNE | |
| | | | | CGTGCTGTGCAACAGCGAAGACATCCGCCTCAGCCCCTTCATGGCTGACAT | | GCPVEALVKDNGNGTYSC | |
| | | | | CCGTGACGCGCCCCAGGACTTCCACCCAGACAGGGTGAAGGCACGTGGGC | | SYVPRKPVKHTAMVSWGG | |
| | | | | CTGGATTGGAGAAGACAGGTGTGGCCGTCAACAAGCCAGCAGAGTTCACAG | | VSIPNSPFRVNVGAGSHPN | |
| | | | | TGGATGCCAAGCACGGTGGCAAGGCCCCACTTCGGGTCCAAGTCCAGGACA | | KVKVYGPGVAKTGLKAHEP | |
| | | | | ATGAAGGCTGCCCTGTGGAGGCGTTGGTCAAGGACAACGGCAATGGCACTT | | TYFTVDCAEAGQGDVSIGI | |
| | | | | ACAGCTGCTCCTACGTGCCCAGGAAGCCGGTGAAGCACACAGCCATGGTGT | | KCAPGVVGPAEADIDFDIIR | |
| | | | | CCTGGGGAGGCGTCAGCATCCCCAACAGCCCCTTCAGGGTGAATGTGGGA | | NDNDTFTVKYTPRGAGSYT | |
| | | | | GCTGGCAGCCACCCCAACAAGGTCAAAGTATACGGCCCCGGAGTAGCCAAG | | IMVLFADQATPTSPIRVKVE | |
| | | | | ACAGGGCTCAAGGCCCACGAGCCCACCTACTTCACTGTGGACTGCGCCGAG | | PSHDASKVKAEGPGLSRT | |
| | | | | GCTGGCCAGGGGGACGTCAGCATCGGCATCAAGTGTGCCCCTGGAGTGGT | | GVELGKPTHFTVNAKAAGK | |
| | | | | AGGCCCCGCCGAAGCTGACATCGACTTCGACATCATCCGCAATGACAATGA | | GKLDVQFSGLTKGDAVRD | |
| | | | | CACCTTCACGGTCAAGTACACGCCCCGGGGGGCTGGCAGCTACACCATTAT | | VDIIDHHDNTYTVKYTPVQQ | |
| | | | | GGTCCTCTTTGCTGACCAGGCCACGCCCACCAGCCCCATCCGAGTCAAGGT | | GPVGVNVTYGGDPIPKSPF | |
| | | | | GGAGCCCTCTCATGACGCCAGTAAGGTGAAGGCCGAGGGCCCTGGCCTCA | | SVAVSPSLDLSKIKVSGLGE | |
| | | | | GTCGCACTGGTGTCGAGCTTGGCAAGCCCACCCACTTCACAGTAAATGCCA | | KVDVGKDQEFTVKSKGAG | |
| | | | | AAGCTGCTGGCAAAGGCAAGCTGGACGTCCAGTTCTCAGGACTCACCAAGG | | GQGKVASKIVGPSGAAVPC | |
| | | | | GGGATGCAGTGCGAGATGTGGACATCATCGACCACCATGACAACACCTACA | | KVEPGLGADNSVVRFLPRE | |
| | | | | CAGTCAAGTACACGCCTGTCCAGCAGGGTCCAGTAGGCGTCAATGTCACTTA | | EGPYEVEVTYDGVPVPGS | |
| | | | | TGGAGGGGATCCCATCCCTAAGAGCCCTTTCTCAGTGGCAGTATCTCCAAGC | | PFPLEAVAPTKPSKVKAFG | |
| | | | | CTGGACCTCAGCAAGATCAAGGTGTCTGGCCTGGGAGAGAAGGTGGACGTT | | PGLQGGSAGSPARFTIDTK | |
| | | | | GGCAAAGACCAGGAGTTCACAGTCAAATCAAAGGGTGCTGGTGGTCAAGGC | | GAGTGGLGLTVEGPCEAQ | |
| | | | | AAAGTGGCATCCAAGATTGTGGGCCCCTCGGGTGCAGCGGTGCCCTGCAAG | | LECLDNGDGTCSVSYVPTE | |
| | | | | GTGGAGCCAGGCCTGGGGGCTGACAACAGTGTGGTGCGCTTCCTGCCCCG | | PGDYNINILFADTHIPGSPF | |
| | | | | TGAGGAAGGGCCCTATGAGGTGGAGGTGACCTATGACGGCGTGCCCGTGC | | KAHVVPCFDASKVKCSGP | |
| | | | | CTGGCAGCCCCTTTCCTCTGGAAGCTGTGGCCCCCACCAAGCCTAGCAAGG | | GLERATAGEVGQFQVDCS | |
| | | | | TGAAGGCGTTTGGGCCGGGGCTGCAGGGAGGCAGTGCGGGCTCCCCCGCC | | SAGSAELTIEICSEAGLPAE | |
| | | | | CGCTTCACCATCGACACCAAGGGCGCCGGCACAGGTGGCCTGGGCCTGAC | | VYIQDHGDGTHTITYIPLCP | |
| | | | | GGTGGAGGGCCCCTGTGAGGCGCAGCTCGAGTGCTTGGACAATGGGGATG | | GAYTVTIKYGGQPVPNFPS | |
| | | | | GCACATGTTCCGTGTCCTACGTGCCCACCGAGCCCGGGGACTACAACATCA | | KLQVEPAVDTSGVQCYGP | |
| | | | | ACATCCTCTTCGCTGACACCCACATCCCTGGCTCCCCATTCAAGGCCCACGT | | GIEGQGVFREATTEFSVDA | |
| | | | | GGTTCCCTGCTTTGACGCATCCAAAGTCAAGTGCTCAGGCCCCGGGCTGGA | | RALTQTGGPHVKARVANP | |
| | | | | GCGGGCCACCGCTGGGGAGGTGGGCCAATTCCAAGTGGACTGCTCGAGCG | | SGNLTETYVQDRGDGMYK | |
| | | | | CGGGCAGCGCGGAGCTGACCATTGAGATCTGCTCGGAGGCGGGGCTTCCG | | VEYTPYEEGLHSVDVTYDG | |
| | | | | GCCGAGGTGTACATCCAGGACCACGGTGATGGCACGCACACCATTACCTAC | | SPVPSSPFQVPVTEGCDPS | |
| | | | | ATTCCCCTCTGCCCCGGGGCCTACACCGTCACCATCAAGTACGGCGGCCAG | | RVRVHGPGIQSGTTNKPNK | |
| | | | | CCCGTGCCCAACTTCCCCAGCAAGCTGCAGGTGGAACCTGCGGTGGACACT | | FTVETRGAGTGGLGLAVEG | |
| | | | | TCCGGTGTCCAGTGCTATGGGCCTGGTATTGAGGGCCAGGGTGTCTTCCGT | | PSEAKMSCMDNKDGSCSV | |
| | | | | GAGGCCACCACTGAGTTCAGTGTGGACGCCCGGGCTCTGACACAGACCGG | | EYIPYEAGTYSLNVTYGGH | |
| | | | | AGGGCCGCACGTCAAGGCCCGTGTGGCCAACCCCTCAGGCAACCTGACGG | | QVPGSPFKVPVHDVTDASK | |
| | | | | AGACCTACGTTCAGGACCGTGGCGATGGCATGTACAAAGTGGAGTACACGC | | VKCSGPGLSPGMVRANLP | |
| | | | | CTTACGAGGAGGGACTGCACTCCGTGGACGTGACCTATGACGGCAGTCCCG | | QSFQVDTSKAGVAPLQVKV | |
| | | | | TGCCCAGCAGCCCCTTCCAGGTGCCCGTGACCGAGGGCTGCGACCCCTCC | | QGPKGLVEPVDVVDNADG | |
| | | | | CGGGTGCGTGTCCACGGGCCAGGCATCCAAAGTGGCACCACCAACAAGCC | | TQTVNYVPSREGPYSISVL | |
| | | | | CAACAAGTTCACTGTGGAGACCAGGGGAGCTGGCACGGGCGGCCTGGGCC | | YGDEEVPRSPFKVKVLPTH | |
| | | | | TGGCTGTAGAGGGCCCCTCCGAGGCCAAGATGTCCTGCATGGATAACAAGG | | DASKVKASGPGLNTTGVPA | |
| | | | | ACGGCAGCTGCTCGGTCGAGTACATCCCTTATGAGGCTGGCACCTACAGCC | | SLPVEFTIDAKDAGEGLLAV | |
| | | | | TCAACGTCACCTATGGTGGCCATCAAGTGCCAGGCAGTCCTTTCAAGGTCCC | | QITDPEGKPKKTHIQDNHD | |
| | | | | TGTGCATGATGTGACAGATGCGTCCAAGGTCAAGTGCTCTGGGCCCGGCCT | | GTYTVAYVPDVTGRYTILIK | |
| | | | | GAGCCCAGGCATGGTTCGTGCCAACCTCCCTCAGTCCTTCCAGGTGGACAC | | YGGDEIPFSPYRVRAVPTG | |
| | | | | AAGCAAGGCTGGTGTGGCCCCATTGCAGGTCAAAGTGCAAGGGCCCAAAGG | | DASKCTVTVSIGGHGLGAG | |
| | | | | CCTGGTGGAGCCAGTGGACGTGGTAGACAACGCTGATGGCACCCAGACCGT | | IGPTIQIGEETVITVDTKAAG | |
| | | | | CAATTATGTGCCCAGCCGAGAAGGGCCCTACAGCATCTCAGTACTGTATGGA | | KGKVTCTVCTPDGSEVDV | |
| | | | | GATGAAGAGGTACCCCGGAGCCCCTTCAAGGTCAAGGTGCTGCCTACTCAT | | DVVENEDGTFDIFYTAPQP | |
| | | | | GATGCCAGCAAGGTGAAGGCCAGTGGCCCCGGGCTCAACACCACTGGCGT | | GKYVICVRFGGEHVPNSPF | |
| | | | | GCCTGCCAGCCTGCCCGTGGAGTTCACCATCGATGCAAAGGACGCCGGGG | | QVTALAGDQPSVQPPLRS | |
| | | | | AGGGCCTGCTGGCTGTCCAGATCACGGATCCCGAAGGCAAGCCGAAGAAGA | | QQLAPQYTYAQGGQQTWA | |
| | | | | CACACATCCAAGACAACCATGACGGCACGTATACAGTGGCCTACGTGCCAG | | PERPLVGVNGLDVTSLRPF | |
| | | | | ACGTGACAGGTCGCTACACCATCCTCATCAAGTACGGTGGTGACGAGATCC | | DLVIPFTIKKGEITGEVRMP | |
| | | | | CCTTCTCCCCGTACCGCGTGCGTGCCGTGCCCACCGGGGACGCCAGCAAG | | SGKVAQPTITDNKDGTVTV | |
| | | | | TGCACTGTCACAGTGTCAATCGGAGGTCACGGGCTAGGTGCTGGCATCGGC | | RYAPSEAGLHEMDIRYDNM | |
| | | | | CCCACCATTCAGATTGGGGAGGAGACGGTGATCACTGTGGACACTAAGGCG | | HIPGSPLQFYVDYVNCGHV | |
| | | | | GCAGGCAAAGGCAAAGTGACGTGCACCGTGTGCACGCCTGATGGCTCAGAG | | TAYGPGLTHGVVNKPATFT | |
| | | | | GTGGATGTGGACGTGGTGGAGAATGAGGACGGCACTTTCGACATCTTCTAC | | VNTKDAGEGGLSLAIEGPS | |
| | | | | ACGGCCCCCCAGCCGGGCAAATACGTCATCTGTGTGCGCTTTGGTGGCGAG | | KAEISCTDNQDGTCSVSYL | |
| | | | | CACGTGCCCAACAGCCCCTTCCAAGTGACGGCTCTGGCTGGGGACCAGCCC | | PVLPGDYSILVKYNEQHVP | |
| | | | | TCGGTGCAGCCCCCTCTACGGTCTCAGCAGCTGGCCCCACAGTACACCTAC | | GSPFTARVTGDDSMRMSH | |
| | | | | GCCCAGGGCGGCCAGCAGACTTGGGCCCCGGAGAGGCCCCTGGTGGGTGT | | LKVGSAADIPINISETDLSLL | |
| | | | | CAATGGGCTGGATGTGACCAGCCTGAGGCCCTTTGACCTTGTCATCCCCTTC | | TATVVPPSGREEPCLLKRL | |
| | | | | ACCATCAAGAAGGGCGAGATCACAGGGGAGGTTCGGATGCCCTCAGGCAAG | | RNGHVGISFVPKETGEHLV | |
| | | | | GTGGCGCAGCCCACCATCACTGACAACAAAGACGGCACCGTGACCGTGCG | | HVKKNGQHVASSPIPVVIS | |
| | | | | GTATGCACCCAGCGAGGCTGGCCTGCACGAGATGGACATCCGCTATGACAA | | QSEIGDASRVRVSGQGLHE | |
| | | | | CATGCACATCCCAGGAAGCCCCTTGCAGTTCTATGTGGATTACGTCAACTGT | | GHTFEPAEFIIDTRDAGYG | |
| | | | | GGCCATGTCACTGCCTATGGGCCTGGCCTCACCCATGGAGTAGTGAACAAG | | GLSLSIEGPSKVDINTEDLE | |
| | | | | CCTGCCACCTTCACCGTCAACACCAAGGATGCAGGAGAGGGGGGCCTGTCT | | DGTCRVTYCPTEPGNYIINI | |
| | | | | CTGGCCATTGAGGGCCCGTCCAAAGCAGAAATCAGCTGCACTGACAACCAG | | KFADQHVPGSPFSVKVTGE | |
| | | | | GATGGGACATGCAGCGTGTCCTACCTGCCTGTGCTGCCGGGGGACTACAGC | | GRVKESITRRRRAPSVANV | |
| | | | | ATTCTAGTCAAGTACAATGAACAGCACGTCCCAGGCAGCCCCTTCACTGCTC | | GSHCDLSLKIPEISIQDMTA | |
| | | | | GGGTCACAGGTGACGACTCCATGCGTATGTCCCACCTAAAGGTCGGCTCTG | | QVTSPSGKTHEAEIVEGEN | |
| | | | | CTGCCGACATCCCCATCAACATCTCAGAGACGGATCTCAGCCTGCTGACGG | | HTYCIRFVPAEMGTHTVSV | |
| | | | | CCACTGTGGTCCCGCCCTCGGGCCGGGAGGAGCCCTGTTTGCTGAAGCGG | | KYKGQHVPGSPFQFTVGP | |
| | | | | CTGCGTAATGGCCACGTGGGGATTTCATTCGTGCCCAAGGAGACGGGGGAG | | LGEGGAHKVRAGGPGLER | |
| | | | | CACCTGGTGCATGTGAAGAAAAATGGCCAGCACGTGGCCAGCAGCCCCATC | | AEAGVPAEFSIWTREAGAG | |
| | | | | CCGGTGGTGATCAGCCAGTCGGAAATTGGGGATGCCAGTCGTGTTCGGGTC | | GLAIAVEGPSKAEISFEDRK | |
| | | | | TCTGGTCAGGGCCTTCACGAAGGCCACACCTTTGAGCCTGCAGAGTTTATCA | | DGSCGVAYVVQEPGDYEV | |
| | | | | TTGATACCCGCGATGCAGGCTATGGTGGGCTCAGCCTGTCCATTGAGGGCC | | SVKFNEEHIPDSPFVVPVA | |
| | | | | CCAGCAAGGTGGACATCAACACAGAGGACCTGGAGGACGGGACGTGCAGG | | SPSGDARRLTVSSLQESGL | |
| | | | | GTCACCTACTGCCCCACAGAGCCAGGCAACTACATCATCAACATCAAGTTTG | | KVNQPASFAVSLNGAKGAI | |
| | | | | CCGACCAGCACGTGCCTGGCAGCCCCTTCTCTGTGAAGGTGACAGGCGAG | | DAKVHSPSGALEECYVTEI | |
| | | | | GGCCGGGTGAAAGAGAGCATCACCCGCAGGCGTCGGGCTCCTTCAGTGGC | | DQDKYAVRFIPRENGVYLID | |
| | | | | CAACGTTGGTAGTCATTGTGACCTCAGCCTGAAAATCCCTGAAATTAGCATC | | VKFNGTHIPGSPFKIRVGEP | |
| | | | | CAGGATATGACAGCCCAGGTGACCAGCCCATCGGGCAAGACCCATGAGGCC | | GHGGDPGLVSAYGAGLEG | |
| | | | | GAGATCGTGGAAGGGGAGAACCACACCTACTGCATCCGCTTTGTTCCCGCT | | GVTGNPAEFVVNYSNAGA | |
| | | | | GAGATGGGCACACACACAGTCAGCGTCAAGTACAAGGGCCAGCACGTGCCT | | GALSVTIDGPSKVKMDCQE | |
| | | | | GGGAGCCCCTTCCAGTTCACCGTGGGGCCCCTAGGGGAAGGGGGAGCCCA | | CPEGYRVTYTPMAPGSYLI | |
| | | | | CAAGGTCCGAGCTGGGGGCCCTGGCCTGGAGAGAGCTGAAGCTGGAGTGC | | SIKYGGPYHIGGSPFKAKVT | |
| | | | | CAGCCGAATTCAGTATCTGGACCCGGGAAGCTGGTGCTGGAGGCCTGGCCA | | GPRLVSNHSLHETSSVFVD | |
| | | | | TTGCTGTCGAGGGCCCCAGCAAGGCTGAGATCTCTTTTGAGGACCGCAAGG | | SLTKATCAPQHGAPGPGP | |
| | | | | ACGGCTCCTGTGGTGTGGCTTATGTGGTCCAGGAGCCAGGTGACTACGAAG | | ADASKVVAKGLGLSKAYVG | |
| | | | | TCTCAGTCAAGTTCAACGAGGAACACATTCCCGACAGCCCCTTCGTGGTGCC | | QKSSFTVDCSKAGNNMLLV | |
| | | | | TGTGGCTTCTCCGTCTGGCGACGCCCGCCGCCTCACTGTTTCTAGCCTTCA | | GVHGPRTPCEEILVKHVGS | |
| | | | | GGAGTCAGGGCTAAAGGTCAACCAGCCAGCCTCTTTTGCAGTCAGCCTGAA | | RLYSVSYLLKDKGEYTLVV | |
| | | | | CGGGGCCAAGGGGGCGATCGATGCCAAGGTGCACAGCCCCTCAGGAGCCC | | KWGHEHIPGSPYRVVVP* | |
| | | | | TGGAGGAGTGCTATGTCACAGAAATTGACCAAGATAAGTATGCTGTGCGCTT | | | |
| | | | | CATCCCTCGGGAGAATGGCGTTTACCTGATTGACGTCAAGTTCAACGGTACC | | | |
| | | | | CACATCCCTGGAAGCCCCTTCAAGATCCGAGTTGGGGAGCCTGGGCATGGA | | | |
| | | | | GGGGACCCAGGCTTGGTGTCTGCTTACGGAGCAGGTCTGGAAGGCGGTGT | | | |
| | | | | CACAGGGAACCCAGCTGAGTTCGTCGTGAACACGAGCAATGCGGGAGCTGG | | | |
| | | | | TGCCCTGTCGGTGACCATTGACGGCCCCTCCAAGGTGAAGATGGATTGCCA | | | |
| | | | | GGAGTGCCCTGAGGGCTACCGCGTCACCTATACCCCCATGGCACCTGGCAG | | | |
| | | | | CTACCTCATCTCCATCAAGTACGGCGGCCCCTACCACATTGGGGGCAGCCC | | | |
| | | | | CTTCAAGGCCAAAGTCACAGGCCCCCGTCTCGTCAGCAACCACAGCCTCCA | | | |
| | | | | CGAGACATCATCAGTGTTTGTAGACTCTCTGACCAAGGCCACCTGTGCCCCC | | | |
| | | | | CAGCATGGGGCCCCGGGTCCTGGGCCTGCTGACGCCAGCAAGGTGGTGGC | | | |
| | | | | CAAGGGCCTGGGGCTGAGCAAGGCCTACGTAGGCCAGAAGAGCAGCTTCA | | | |
| | | | | CAGTAGACTGCAGCAAAGCAGGCAACAACATGCTGCTGGTGGGGGTTCATG | | | |
| | | | | GCCCAAGGACCCCCTGCGAGGAGATCCTGGTGAAGCACGTGGGCAGCCGG | | | |
| | | | | CTCTACAGCGTGTCCTACCTGCTCAAGGACAAGGGGGAGTACACACTGGTG | | | |
| | | | | GTCAAATGGGGGCACGAGCACATCCCAGGCAGCCCCTACCGCGTTGTGGTG | | | |
| | | | | CCCTGA | | | |
| Shigella | 6 | prey56789 | 183 | CCCCAACATCATCCAGTTTGTGCCAGCTGATGGGCCCCTATTTGGGGACACT | 384 | PNIIQFVPADGPLFGDTVTS | |
| ipaH9.8 | | | | GTCACCAGCTCAGAGCACCTCTGTGGCATCAACTTCACAGGCAGTGTGCCC | | SEHLCGINFTGSVPTFKHL | |
| | | | | ACCTTCAAACACCTGTGGAAGCAGGTGGCCCAGAACCTGGACCGGTTCCAC | | WKQVAQNLDRFHTFPRLA | |
| | | | | ACCTTCCCACGCCTGGCTGGAGAGTGCGGCGGAAAGAACTTCCACTTCGTG | | GECGGKNFHFVHRSADVE | |
| | | | | CACCGCTCGGCCGACGTGGAGAGCGTGGTGAGCGGGACCCTCCGCTCAGC | | SVVSGTLRSAFEYGGQKC | |
| | | | | CTTCGAGTACGGTGGCCAGAAGTGTTCCGCCTGCTCGCGTCTCTACGTGCC | | SACSRLYVPHSLWPQIKGR | |
| | | | | GCACTCGCTGTGGCCGCAGATCAAAGGGCGGCTGCTGGAGGAGCACAGTC | | LLEEHSRIKVGDPAEDFGT | |
| | | | | GGATCAAAGTGGGCGACCCTGCAGAGGATTTTGGGACCTTCTTCTCTGCAGT | | FFSAVIDAKSFARIKKWLEH | |
| | | | | GATTGATGCCAAGTCCTTTGCCCGTATCAAGAAGTGGCTGGAGCACGCGCG | | ARSSPSLTILAGGKCDDSV | |
| | | | | CTCCTCGCCCAGCCTCACCATCCTGGCTGGGGGCAAGTGTGATGACTCCGT | | GYFVEPCIVESKDPQEPIM | |
| | | | | GGGCTACTTTGTGGAGCCCTGCATCGTGGAGAGCAAGGACCCTCAGGAGCC | | KEEIFGPVLSVYVYPDDKY | |
| | | | | CATCATGAAGGAGGAGATCTTCGGGCCTGTACTGTCTGTGTACGTCTACCCG | | KETLQLVDSTTSYGLTGAV | |
| | | | | GACGACAAGTACAAGGAGACGCTGCAGCTGGTTGACAGCACCACCAGCTAT | | FSQDKDVVQEATKVLRNAA | |
| | | | | GGCCTCACGGGGGCAGTGTTCTCCCAGGATAAGGACGTCGTGCAGGAGGC | | GNFYINDKSTGSIVGQQPF | |
| | | | | CACAAAGGTGCTGAGGAATGCTGCCGGCAACTTCTACATCAACGACAAGTCC | | GGARASGTNDKPGGPHYIL | |
| | | | | ACTGGCTCGATAGTGGGCCAGCAGCCCTTTGGGGGGGCCCGAGCCTCTGG | | RWTSPQVIKETHKPLGDW | |
| | | | | AACCAATGACAAGCCAGGGGGCCCACACTACATCCTGCGCTGGACGTCGCC | | SYAYMQ* | |
| | | | | GCAGGTCATCAAGGAGACACATAAGCCCCTGGGGGACTGGAGCTACGCGTA | | | |
| | | | | CATGCAGTGA | | | |
| Shigella | 6 | prey67711 | 184 | AACAGAGCTGCCTCCTGGCTCTTTGGGAGCCTGGGAGGAGAAGGAGCCGG | 385 | NRAASWLFGSLGGEGAGR | |
| ipaH9.8 | | | | GAGGGGCGCTGCGGGGAAGCCACCTGCGGATTCACTGGCTGCTGCTCCGC | | GAAGKPPADSLAAAPPRTA | |
| | | | | CCAGGACTGCTAGCAAGCACGGAGGGCTGCCAGACCTGGGGCTCCCTGCT | | SKHGGLPDLGLPAPCVRLG | |
| | | | | CCGTGCGTCAGGTTGGGGAAACCACCGTCTGCCCCAGACCCTGACCCAGGA | | KPPSAPDPDPGPAWRKL | |
| | | | | CCCGCCTGGAGGAAGCTGGG | | | |
| Shigella | 6 | prey2118 | 185 | ATGTCTCAGGCTGTGCAGACAAACGGAACTCAACCATTAAGCAAAACATGGG | 386 | MSQAVQTNGTQPLSKTWE | |
| ipaH9.8 | | | | AACTCAGTTTATATGAGTTACAACGAACACCTCAGGAGGCAATAACAGATGG | | LSLYELQRTPQEAITDGLEI | |
| | | | | CTTAGAAATTGTGGTTTCACCTCGAAGTCTACACAGTGAATTAATGTGCCCAA | | VVSPRSLHSELMCPICLDM | |
| | | | | TTTGTTTGGATATGTTGAAGAACACCATGACTACAAAGGAGTGTTTACATCGT | | LKNTMTTKECLHRFCADCII | |
| | | | | TTTTGTGCAGACTGCATCATCACAGCCCTTAGAAGTGGCAACAAAGAATGTC | | TALRSGNKECPTCRKKLVS | |
| | | | | CTACCTGTCGGAAAAAACTAGTTTCCAAAAGATCACTAAGGCCAGACCCAAA | | KRSLRPDPNFDALISKIYPS | |
| | | | | CTTTGATGCACTCATCAGCAAAATTTATCCAAGTCGTGATGAGTATGAAGCTC | | RDEYEAHQERVLARINKHN | |
| | | | | ATCAAGAGAGAGTATTAGCCAGGATCAACAAGCACAATAATCAGCAAGCACT | | NQQALSHSIEEGLKIQAMN | |
| | | | | CAGTCACAGCATTGAGGAAGGACTGAAGATACAGGCCATGAACAGACTGCA | | RLQRGKKQQIENGSGAED | |
| | | | | GCGAGGCAAGAAACAACAGATTGAAAATGGTAGTGGAGCAGAAGATAATGG | | NGDSSHCSNASTHSNQEA | |
| | | | | TGACAGTTCACACTGCAGTAATGCATCCACACATAGCAATCAGGAAGCAGGC | | GPSNKRTKTSDDSGLELDN | |
| | | | | CCTAGTAACAAACGGACCAAAACATCTGATGATTCTGGGCTAGAGCTTGATA | | NNAAMAIDPVMDGASEIEL | |
| | | | | ATAACAATGCAGCAATGGCAATTGATCCAGTAATGGATGGTGCTAGTGAAAT | | VFRPHPTLMEKDDSAQTRY | |
| | | | | TGAATTAGTATTCAGGCCTCATCCCACACTTATGGAAAAAGATGACAGTGCA | | IKTSGNATVDHLSKYLAVRL | |
| | | | | CAGACGAGATACATAAAGACTTCTGGTAACGCCACTGTTGATCACTTATCCAA | | ALEELRSKGESNQMNLDTA | |
| | | | | GTATCTGGCTGTGAGGTTAGCTTTAGAAGAACTTCGAAGCAAAGGTGAATCA | | SEKQYTIYIATASGQFTVLN | |
| | | | | AACCAGATGAACCTTGATACAGCCAGTGAGAAGCAGTATACCATTTATATAG | | GSFSLELVSEKYWKVNKP | |
| | | | | CAACAGCCAGTGGCCAGTTCACTGTATTAAATGGCTCTTTTTCTTTGGAATTG | | MELYYAPTKEHK* | |
| | | | | GTCAGTGAGAAATACTGGAAAGTGAACAAACCCATGGAACTTTATTACGCAC | | | |
| | | | | CTACAAAGGAGCACAAATGA | | | |
| Shigella | 6 | prey3596 | 186 | ATGTCCAAGCGGCACCGGTTGGACCTAGGGGAGGATTACCCCTCTGGCAAG | 387 | MSKRHRLDLGEDYPSGKK | |
| ipaH9.8 | | | | AAGCGTGCGGGGACCGATGGGAAGGATCGAGATCGAGACCGGGATCGTGA | | RAGTDGKDRDRDRDREDR | |
| | | | | AGATCGGTCTAAAGATCGAGACCGAGAACGTGATAGAGGAGATAGAGAGCG | | SKDRDRERDRGDRERERE | |
| | | | | AGAGAGGGAGAAAGAAAAGGAGAAGGAGTTGCGAGCTTCAACAAATGCTAT | | KEKEKELRASTNAMLISAGL | |
| | | | | GCTTATCAGTGCTGGATTACCACCCCTGAAAGCTTCCCATTCAGCTCACTCA | | PPLKASHSAHSTHSAHSTH | |
| | | | | ACCCACTCAGCACATTCAACGCATTCTACACATTCTGCTCATTCAACGCATGC | | STHSAHSTHAGHAGHTSLP | |
| | | | | CGGACATGCAGGTCACACGTCACTTCCACAGTGCATTAATCCGTTCACCAAC | | QCINPFTNLPHTPRYYDILK | |
| | | | | TTACCCCATACTCCTCGATACTATGATATTCTAAAGAAACGTCTTCAGCTCCC | | KRLQLPVWEYKDRFTDILG | |
| | | | | TGTTTGGGAATACAAGGATAGGTTTACAGATATTCTGGGTAGACATCAGTCCT | | RHQSFVLVGETGSGKTTQI | |
| | | | | TTGTACTGGTTGGTGAGACTGGGTCTGGTAAAACAACACAAATTCCACACCG | | PHRCVEYMRSLPGPKRGV | |
| | | | | GTGTGTGGAGTACATGCGATCATTACCAGGACCCAAGAGAGGAGTTGCCTG | | ACTQPRRVAAMSVAQRVA | |
| | | | | TACCCAACCCAGGAGAGTGGCTGCAATGAGTGTGGCTCAGAGAGTTGCTGA | | DEMDVMLGQEVGYSIRFE | |
| | | | | TGAGATGGATGTGATGTTGGGCCAGGAAGTTGGTTACTCCATTCGATTTGAA | | DCSSAKTFFMYMTDGMLL | |
| | | | | GACTGCAGTAGTGCAAAAACATTTTTTATGTATATGACTGATGGGATGTTACT | | REAMNDPLLERYGVIILDEA | |
| | | | | TCGTGAAGCTATGAATGATCCCCTCCTGGAGCGTTATGGTGTAATAATTCTTG | | HERTLATDILMGVLKEVVR | |
| | | | | ATGAGGCTCATGAGAGGACACTGGCTACAGATATTCTAATGGGTGTTCTGAA | | QRSDLKVIVMSATLDA | |
| | | | | GGAAGTTGTAAGACAGAGATCAGATTTAAAGGTTATAGTTATGAGCGCTACT | | | |
| | | | | CTAGATGCAGG | | | |
| Shigella | 6 | prey666 | 187 | CATCACATCCCGGTTGGAATCTGTGCACATCATACTGAGAGATGGCCTGGAA | 388 | ITSRLESVHIILRDGLEDPLE | |
| ipaH9.8 | | | | GATCCCCTGGAGGATACGGGGCTGGTCCAGCAGCAGTTGGACCAGCTGTCC | | DTGLVQQQLDQLSTIGRCE | |
| | | | | ACCATTGGGCGTTGTGAATATGAGAAGACGTGTGCACTCCTCGTGCAGTTGT | | YEKTCALLVQLFDQSAQSY | |
| | | | | TTGACCAGTCGGCCCAGTCGTACCAGGAGCTGCTACAGAGCGCCAGCGCAA | | QELLQSASASPMDIAVQEG | |
| | | | | GCCCAATGGACATTGCAGTGCAGGAGGGAAGGCTGACATGGCTGGTTTACA | | RLTWLVYIIGAVIGGRVSFA | |
| | | | | TTATTGGAGCAGTGATCGGTGGCCGGGTTTCTTTTGCCAGCACTGATGAGCA | | STDEQDAMDGELVCRVLQ | |
| | | | | AGACGCCATGGATGGTGAGCTTGTCTGTCGGGTGCTCCAGCTGATGAACCT | | LMNLTDSRLAQAGNEKLEL | |
| | | | | AACAGATTCTCGTTTGGCCCAGGCGGGTAATGAGAAGCTAGAGTTGGCCAT | | AMLSFFEQFRKIYIGDQVQ | |
| | | | | GCTGAGCTTTTTTGAACAGTTTCGTAAGATCTACATTGGGGACCAAGTGCAG | | KSSKLYRR | |
| | | | | AAATCCTCTAAGCTGTACCGCCGAC | | | |
| Shigella | 7 | prey3917 | 188 | GATGACCACGCTATACACCGCCAAGAAGTACGCGGTGCCAGCGCTCGAGGC | 389 | MTTLYTAKKYAVPALEAHC | |
| ospG | | | | CCATTGCGTGGAGTTCCTGAAGAAGAACCTGCGAGCCGACAACGCCTTCAT | | VEFLKKNLRADNAFMLLTQ | |
| | | | | GCTGCTCACGCAGGCGCGACTCTTCGATGAACCGCAGCTGGCCAGCCTGTG | | ARLFDEPQLASLCLENIDKN | |
| | | | | CCTGGAGAACATCGACAAAAACACTGCAGACGCCATCACCGCGGAGGGCTT | | TADAITAEGFTDIDLDTLVA | |
| | | | | CACCGACATTGACCTGGACACGCTGGTGGCTGTCCTGGAGCGCGACACACT | | VLERDTLGIREVRLFNAVVR | |
| | | | | GGGCATCCGTGAGGTGCGGCTGTTCAATGCCGTTGTCCGCTGGTCCGAGGC | | WSEAECQRQQLQVTPENR | |
| | | | | CGAGTGTCAGCGGCAGCAGCTGCAGGTGACGCCAGAGAACAGGCGGAAGG | | RKVLGKALGLIRFPLMTIEE | |
| | | | | TTCTGGGCAAGGCCCTGGGCCTCATTCGCTTCCCGCTCATGACCATCGAGG | | FAAGPAQSGILVDREVVSL | |
| | | | | AGTTCGCTGCAGGTCCCGCACAGTCGGGCATCCTGGTGGACCGCGAGGTG | | FLHFTVNPKPRVEFIDRPR | |
| | | | | GTCAGCCTCTTCCTGCACTTCACCGTCAACCCCAAGCCACGAGTGGAGTTCA | | CCLRGKECSINRFQQVESR | |
| | | | | TTGACCGGCCCCGCTGCTGCCTGCGTGGGAAGGAGTGCAGCATCAACCGCT | | WGYSGTSDRIRFSVNKRIF | |
| | | | | TCCAGCAGGTGGAGAGTCGCTGGGGCTACAGCGGGACCAGTGACCGCATC | | VVGFGLYGSIHGPTDYQVN | |
| | | | | AGGTTCTCAGTCAACAAGCGCATCTTCGTGGTGGGATTTGGGCTGTATGGAT | | IQIIHTDSNTVLGQNDTGFS | |
| | | | | CCATCCACGGGCCCACCGACTACCAAGTGAACATCCAGATTATTCACACCGA | | CDGSASTFRVMFKEPVEVL | |
| | | | | TAGCAACACCGTCTTGGGCCAGAACGACACGGGCTTCAGCTGCGACGGCTC | | PNVNYTACATLKGPDSHYG | |
| | | | | AGCCAGCACCTTCCGCGTCATGTTCAAGGAGCCGGTGGAGGTGCTGCCCAA | | TKGLRKVTHESPTTGAKTC | |
| | | | | CGTCAACTACACGGCCTGTGCCACGCTCAAGGGCCCAGACTCCCACTACGG | | FTFCYAAGNNNGTSVEDG | |
| | | | | CACCAAAGGCCTGCGCAAGGTGACACACGAGTCGCCCACCACGGGCGCCA | | QIPEVIFYT* | |
| | | | | AGACCTGCTTCACCTTTTGCTACGCGGCCGGGAACAACAATGGCACATCCGT | | | |
| | | | | GGAGGACGGCCAGATCCCCGAGGTCATCTTCTACACCTAG | | | |
| Shigella | 7 | prey63632 | 189 | CTGTGGGAAAGCCTTCAGTTGGAAATCACACCTTATTGAGCATCAAAGAACT | 390 | CGKAFSWKSHLIEHQRTHT | |
| ospG | | | | CACACTGGTGAGAAACCTTATCACTGTACCAAATGTAAGAAGAGCTTTAGTC | | GEKPYHCTKCKKSFSRNSL | |
| | | | | GAAATTCATTGCTTGTTGAGCATCAAAGAATTCACACTGGGGAAAGACCCCA | | LVEHQRIHTGERPHKCGEC | |
| | | | | TAAATGTGGTGAATGTGGGAAAGCCTTTCGATTAAGCACATACCTTATACAAC | | GKAFRLSTYLIQHQKIHTGE | |
| | | | | ACCAAAAAATTCACACTGGCGAGAAGCCTTTTCTTTGTATTGAGTGTGGAAAA | | KPFLCIECGKSFSRSSFLIE | |
| | | | | AGTTTCAGTCGGAGCTCATTCCTTATTGAACATCAGAGGATCCATACTGGTG | | HQRIHTGERPYQCKECGK | |
| | | | | AAAGACCTTATCAGTGCAAAGAGTGTGGGAAAAGTTTCAGTCAGCTTTGCAA | | SFSQLCNLTRHQRIHTGDK | |
| | | | | CCTTACTCGTCATCAGAGAATTCACACAGGAGACAAGCCCCATAAATGTGAG | | PHKCEECGKAFSRSSGLIQ | |
| | | | | GAATGTGGAAAAGCCTTTAGTAGAAGCTCAGGTCTTATTCAGCATCAGAGAA | | HQRIHTREKTYPYNETKES | |
| | | | | TTCACACCAGGGAGAAGACTTATCCATACAATGAAACTAAGGAAAGTTTTGAT | | FDPNCSLVIQQEVYPKEKS | |
| | | | | CCAAATTGCAGTCTTGTTATACAGCAGGAAGTCTACCCTAAGGAGAAATCTTA | | YKCDECGKTFSVSAHLVQH | |
| | | | | TAAATGTGATGAATGTGGGAAAACTTTTAGTGTTAGTGCTCATCTTGTACAAC | | QRIHTGEKPYLCTVCGKSF | |
| | | | | ATCAAAGAATCCACACTGGTGAAAAGCCCTATCTATGTACTGTCTGTGGGAA | | SRSSFLIEHQRIHTGERPYL | |
| | | | | GAGCTTCAGCCGGAGCTCATTTCTTATTGAACATCAGAGAATCCACACTGGA | | CRQCGKSFSQLCNLIRHQG | |
| | | | | GAGAGACCCTATCTGTGCAGACAGTGTGGAAAAAGCTTTAGTCAGCTTTGTA | | VHTGNKPHKCDECGKAFS | |
| | | | | ATCTTATTCGACATCAGGGTGTTCACACAGGTAATAAACCCCATAAATGTGAT | | RNSGLIQHQRIHTGEKPYK | |
| | | | | GAATGTGGAAAGGCCTTTAGCCGGAACTCGGGTCTTATTCAGCATCAGAGAA | | CEKCDKSFSQQRSLVNHQ | |
| | | | | TACACACAGGAGAGAAACCTTATAAGTGTGAGAAGTGCGACAAAAGTTTCAG | | MIHAEVKTQETHECDACGE | |
| | | | | TCAACAGCGCAGTCTTGTCAACCATCAGATGATCCATGCAGAGGTGAAAACC | | AFNCRISLIQHQKLHTAWM | |
| | | | | CAAGAAACCCATGAATGTGATGCTTGTGGTGAAGCCTTTAATTGCCGTATTTC | | Q* | |
| | | | | TCTTATTCAGCATCAGAAATTGCACACAGCATGGATGCAATAA | GAATACATGGCTGCATACATAGAAAATGCAAAACAGGTTGGCCGCCTTGAAA | | NAKQVGRLENAIGWYHSH |
| | | | | ATGCAATCGGGTGGTATCATAGCCACCCTGGCTATGGCTGCTGGCTTTCTGG | | PGYGCWLSGIDVSTQMLN | |
| | | | | GATTGATGTTAGTACTCAGATGCTCAATCAGCAGTTCCAGGAACCATTTGTAG | | QQFQEPFVAVVIDPTRTISA | |
| | | | | CAGTGGTGATTGATCCAACAAGAACAATATCCGCAGGGAAAGTGAATCTTGG | | GKVNLGAFRTYPKGYKPPD | |
| | | | | CGCCTTTAGGACATACCCAAAGGGCTACAAACCTCCTGATGAAGGACCTTCT | | EGPSEYQTIPLNKIEDFGVH | |
| | | | | GAGTACCAGACTATTCCACTTAATAAAATAGAAGATTTTGGTGTACACTGCAA | | CKQYYALEVSYFKSSLDRK | |
| | | | | ACAATATTATGCCTTAGAAGTCTCATATTTCAAATCCTCTTTGGATCGCAAATT | | LLE | |
| | | | | GCTTGAGCT | | | |
| Shigella | 7 | prey54201 | 191 | ACGGATTAATAAGGAACTTAGTGATTTGGCCCGTGACCCTCCAGCACAATGT | 392 | RINKELSDLARDPPAQCSA | |
| ospG | | | | TCTGCAGGTCCAGTTGGGGATGATATGTTTCATTGGCAAGCCACAATTATGG | | GPVGDDMFHWQATIMGPN | |
| | | | | GACCTAATGACAGCCCATATCAAGGCGGTGTATTCTTTTTGACAATTCATTT | | DSPYQGGVFFLTIHFPTDY | |
| | | | | CCTACAGACTACCCCTTCAAACCACCTAAGGTTGCATTTACAACAAGAATTTA | | PFKPPKVAFTTRIYHPNINS | |
| | | | | TCATCCAAATATTAACAGTAATGGCAGCATTTGTCTCGATATTCTAAGATCAC | | NGSICLDILRSQWSPALTIS | |
| | | | | AGTGGTCGCCTGCTTTAACAATTTCTAAAGTTCTTTTATCCATTTGTTCACTGC | | KVLLSICSLLCDPNPDDPLV | |
| | | | | TATGTGATCCAAACCCAGATGACCCCCTAGTGCCAGAGATTGCACGGATCTA | | PEIARIYKTDRDKYNRISRE | |
| | | | | TAAAACAGACAGAGATAAGTACAACAGAATATCTCGGGAATGGACTCAGAAG | | WTQKYAM* | |
| | | | | TATGCCATGTGA | | | |
| Shigella | 7 | prey1922 | 192 | AACTGGTGCTGCTCCTGCTAAGGCCAAGCCGGCTGAAGCTCCTGCTGCTGC | 393 | TGAAPAKAKPAEAPAAAAP | |
| ospG | | | | AGCCCCAAAAGCAGAACCTACAGCAGCGGCAGTTCCTCCCCCTGCAGCACC | | KAEPTAAAVPPPAAPIPTQ | |
| | | | | CATACCCACTCAGATGCCACCGGTGCCCTCGCCCTCACAGCCTCCTTCTGG | | MPPVPSPSQPPSGKPVSA | |
| | | | | CAAACCTGTGTCTGCAGTAAAACCCACTGTTGCCCCACCACTAGCTGAGCCA | | VKPTVAPPLAEPGAGKGLR | |
| | | | | GGAGCTGGCAAAGGTCTGCGTTCAGAACATCGGGAGAAAATGAACAGGATG | | SEHREKMNRMRQRIAQRL | |
| | | | | CGGCAGCGCATTGCTCAGCGTCTGAAGGAGGCCCAGAATACATGTGCAATG | | KEAQNTCAMLTTFNEIDMS | |
| | | | | CTGACAACTTTTAATGAGATTGACATGAGTAACATCCAGGAGATGAGGGCTC | | NIQEMRARHKEAFLKKHNL | |
| | | | | GGCACAAAGAGGCTTTTTTGAAGAAACATAACCTCAAACTAGGCTTCATGTC | | KLGFMSAFVKASAFALQEQ | |
| | | | | GGCATTTGTGAAGGCCTCAGCCTTTGCCTTGCAGGAACAGCCTGTTGTAAAT | | PVVNAVIDDTTKEVVYRDYI | |
| | | | | GCAGTGATTGACGACACAACCAAAGAGGTGGTGTATAGGGATTATATTGACA | | DISVAVATPRGLVVPVIRNV | |
| | | | | TCAGTGTTGCAGTGGCCACCCCACGGGGTCTGGTGGTTCCAGTCATCAGGA | | EAMNFADIERTITELGEKAR | |
| | | | | ATGTGGAAGCTATGAATTTTGCAGATATTGAACGGACCATCACTGAACTGGG | | KNELAIEDMDGGTFTISNG | |
| | | | | AGAGAAGGCCCGAAAGAATGAACTTGCCATTGAAGATATGGATGGCGGTAC | | GVFGSLFGTPIINPPQSAIL | |
| | | | | CTTCACCATTAGCAATGGAGGCGTTTTTGGCTCGCTCTTTGGAACACCCATT | | GMHGIFDRPVAIGGKVEVR | |
| | | | | ATCAACCCCCCTCAGTCTGCCATCCTGGGGATGCATGGCATCTTTGACAGGC | | PMMYVALTYDHRLIDGREA | |
| | | | | CAGTGGCTATAGGAGGCAAGGTAGAGGTGCGGCCCATGATGTACGTGGCAC | | VTFLRKIKAAVEDPRVLLLD | |
| | | | | TGACCTATGATCACCGGCTGATTGATGGCAGAGAGGCTGTGACTTTCCTCCG | | L* | |
| | | | | CAAAATCAAGGCAGCGGTAGAGGATCCCAGAGTCCTCCTCCTGGATCTTTAG | | | |
| Shigella | 7 | prey67418 | 193 | GGCGGCCAGCAGGAGGCTGATGAAGGAGCTTGAAGAAATCCGCAAATGTGG | 394 | AASRRLMKELEEIRKCGMK | |
| ospG | | | | GATGAAAAACTTCCGTAACATCCAGGTTGATGAAGCTAATTTATTGACTTGGC | | NFRNIQVDEANLLTWQGLI | |
| | | | | AAGGGCTTATTGTTCCTGACAACCCTCCATATGATAAGGGAGCCTTCAGAAT | | VPDNPPYDKGAFRIEINFPA | |
| | | | | CGAAATCAACTTTCCAGCAGAGTACCCATTCAAACCACCGAAGATCACATTTA | | EYPFKPPKITFKTKIYHPNID | |
| | | | | AAACAAAGATCTATCACCCAAACATCGACGAAAAGGGGCAGGTCTGTCTGCC | | EKGQVCLPVISAENWKPAT | |
| | | | | AGTAATTAGTGCCGAAAACTGGAAGCCAGCAACCAAAACCGACCAAGTAATC | | KTDQVIQSLIALVNDPQPEH | |
| | | | | CAGTCCCTCATAGCACTGGTGAATGACCCCCAGCCTGAGCACCCGCTTCGG | | PLRADLAEEYSKDRKKFCK | |
| | | | | GCTGACCTAGCTGAAGAATACTCTAAGGACCGTAAAAAATTCTGTAAGAATG | | NAEEFTKKYGEKRPVD* | |
| | | | | CTGAAGAGTTTACAAAGAAATATGGGGAAAAGCGACCTGTGGACTAA | | | |
| Shigella | 7 | prey67314 | 194 | ATGATGGCGAGCATGCGAGTGGTGAAGGAGCTGGAGGATCTTCAGAAGAAG | 395 | MMASMRVVKELEDLQKKP | |
| ospG | | | | CCTCCCCCATACCTGCGGAACCTGTCCAGCGATGATGCCAATGTCCTGGTG | | PPYLRNLSSDDANVLVWHA | |
| | | | | TGGCACGCTCTCCTCCTACCCGACCAACCTCCCTACCACCTGAAAGCCTTCA | | LLLPDQPPYHLKAFNLRISF | |
| | | | | ACCTGCGCATCAGCTTCCCGCCGGAGTATCCGTTCAAGCCTCCCATGATCAA | | PPEYPFKPPMIKFTTKIYHP | |
| | | | | ATTCACAACCAAGATCTACCACCCCAACGTGGACGAGAACGGACAGATTTGC | | NVDENGQICLPIISSENWKP | |
| | | | | CTGCCCATCATCAGCAGTGAGAACTGGAAGCCTTGCACCAAGACTTGCCAA | | CTKTCQVLEALNVLVNRPNI | |
| | | | | GTCCTGGAGGCCCTCAATGTGCTGGTGAATAGACCGAATATCAGGGAGCCC | | REPLRMDLADLLTQNPELF | |
| | | | | CTGCGGATGGACCTCGCTGACCTGCTGACACAGAATCCGGAGCTGTTCAGA | | RKNAEEFTLRFGVDRPS* | |
| | | | | AAGAATGCCGAAGAGTTCACCCTCCGATTCGGAGTGGACCGGCCCTCCTAA | | | |
| Shigella | 7 | prey67435 | 195 | ATGTCAGTTGGGCACAAGGCCCAGGAGAGCAAGATTCGATACAAAACCAAT | 396 | MSVGHKAQESKIRYKTNEP | |
| ospG | | | | GAACCTGTGTGGGAGGAAAACTTCACTTTCTTCATTCACAATCCCAAGCGCC | | VWEENFTFFIHNPKRQDLE | |
| | | | | AGGACCTTGAAGTTGAGGTCAGAGACGAGCAGCACCAGTGTTCCCTGGGGA | | VEVRDEQHQCSLGNLKVPL | |
| | | | | ACCTGAAGGTCCCCCTCAGCCAGCTGCTCACCAGTGAGGACATGACTGTGA | | SQLLTSEDMTVSQRFQLSN | |
| | | | | GCCAGCGCTTCCAGCTCAGTAACTCGGGTCCAAACAGCACCATCAAGATGA | | SGPNSTIKMKIALRVLHLEK | |
| | | | | AGATTGCCCTGCGGGTGCTCCATCTCGAAAAGCGAGAAAGGCCTCCAGACC | | RERPPD | |
| Shigella | 7 | prey67443 | 196 | CTGGGATGCCCTCAAGGCTGCCGCCTATGCTGCTGAAGCCAACGACCACGA | 397 | WDALKAAAYAAEANDHELA | |
| ospG | | | | GCTGGCCCAGGCCATCCTGGATGGAGCCAGCATCACCCTGCCTCATGGCAC | | QAILDGASITLPHGTLCECY | |
| | | | | CCTCTGTGAATGCTACGATGAGCTGGGCAATCGCTACCAGCTGCCCATCTAC | | DELGNRYQLPIYCLSPPVN | |
| | | | | TGCCTGTCACCGCCGGTGAACCTGCTGCTGGAGCACACGGAGGAGGAGAG | | LLLEHTEEESLEPPEPPPSV | |
| | | | | CCTGGAGCCCCCCGAGCCTCCACCCAGCGTGCGCCGTGAGTTCCCGCTGA | | RREFPLKVRLSTGKDVRLS | |
| | | | | AGGTGCGCCTGTCCACGGGCAAGGACGTGAGGCTCAGCGCCAGCCTGCCC | | ASLPDTVGQLKRQLHAQE | |
| | | | | GACACAGTGGGGCAGCTCAAGAGGCAGCTGCACGCCCAGGAGGGCATCGA | | GIEPSWQRWFFSGKLLTDR | |
| | | | | GCCATCGTGGCAGCGGTGGTTCTTCTCCGGGAAGCTGCTCACAGACCGCAC | | TRLQETKIQKDFVIQVIIN | |
| | | | | ACGGCTCCAGGAGACCAAGATCCAGAAAGATTTTGTCATCCAGGTCATCATC | | | |
| | | | | AAC | | | |
| Shigella | 7 | prey67317 | 197 | CGTCTGTGCCGTCTGCCGCAAGAAGTTCGTCAGCTCCATCAGGCTGCGCAC | 398 | SVPSAARSSSAPSGCAPTS | |
| ospG | | | | CCACATCAAAGAGGTGCACGGGGCTGCCCAGGAGGCCTTGGTCTTCACCAG | | KRCTGLPRRPWSSPVPST | |
| | | | | TTCCATCAACCAGAGCTTCTGCCTCCTGGAACCTGGTGGGGACATCCAGCAA | | RASASWNLVGTSSKKLWG | |
| | | | | GAAGCTCTGGGGGACCAGCTACAGCTGGTGGAAGAGGAGTTTGCCCTCCAG | | TSYSWWKRSLPSRA* | |
| | | | | GGCGTGA | | | |
| Shigella | 7 | prey67393 | 198 | GAGAATCCACAAGGAATTGAATGATCTGGCACGGGACCCTCCAGCACAGTG | 399 | RIHKELNDLARDPPAQCSA | |
| ospG | | | | TTCAGCAGGTCCTGTTGGAGATGATATGTTCCATTGGCAAGCTACAATAATG | | GPVGDDMFHWQATIMGPN | |
| | | | | GGGCCAAATGACAGTCCCTATCAGGGTGGAGTATTTTTCTTGACAATTCATTT | | DSPYQGGVFFLTIHFPTDY | |
| | | | | CCCAACAGATTACCCCTTCAAACCACCTAAGGTTGCATTTACAACAAGAATTT | | PFKPPKVAFTTRIYHPNINS | |
| | | | | ATCATCCAAATATTAACAGTAATGGCAGCATTTGTCTTGATATTCTACGATCA | | NGSICLDILRSQWSPALTIS | |
| | | | | CAGTGGTCTCCAGCACTAACTATTTCAAAAGTACTCTTGTCCATCTGTTCTCT | | KVLLSICSLLCDPNPDDPLV | |
| | | | | GTTGTGTGATCCCAATCCAGATGATCCTTTAGTGCCTGAGATTGCTCGGATC | | PEIARIYKTDREKYNRIARE | |
| | | | | TACAAAACAGATAGAGAAAAGTACAACAGAATAGCTCGGGAATGGACTCAGA | | WTQKYAM* | |
| | | | | AGTATGCGATGTAA | | | |
| Shigella | 7 | prey700 | 199 | ATGGGAATTGGTCTTTCTGCTCAAGGTGTGAACATGAATAGACTACCAGGTT | 400 | MGIGLSAQGVNMNRLPGW | |
| ospG | | | | GGGATAAGCATTCATATGGTTACCATGGGGATGATGGACATTCGTTTTGTTCT | | DKHSYGYHGDDGHSFCSS | |
| | | | | TCTGGAACTGGACAACCTTATGGACCAACTTTCACTACTGGTGATGTCATTG | | GTGQPYGPTFTTGDVIGCC | |
| | | | | GCTGTTGTGTTAATCTTATCAACAATACCTGCTTTTACACCAAGAATGGACAT | | VNLINNTCFYTKNGHSLGIA | |
| | | | | AGTTTAGGTATTGCTTTCACTGACCTACCGCCAAATTTGTATCCTACTGTGGG | | FTDLPPNLYPTVGLQTPGE | |
| | | | | GCTTCAAACACCAGGAGAAGTGGTCGATGCCAATTTTGGGCAACATCCTTTC | | VVDANFGQHPFVFDIEDYM | |
| | | | | GTGTTTGATATAGAAGACTATATGCGGGAGTGGAGAACCAAAATCCAGGCAC | | REWRTKIQAQIDRFPIGDR | |
| | | | | AGATAGATCGATTTCCTATCGGAGATCGAGAAGGAGAATGGCAGACCATGAT | | HGYCATAEAFARSTDQTVL | |
| | | | | ACAAAAAATGGTTTCATCTTATTTAGTCCACCATGGGTACTGTGCCACAGCAG | | EELASIKNRQRIQKLVLAGR | |
| | | | | AGGCCTTTGCCAGATCTACAGACCAGACCGTTCTAGAAGAATTAGCTTCCAT | | MGEAIETTQ | |
| | | | | TAAGAATAGACAAAGAATTCAGAAATTGGTATTAGCAGGAAGAATGGGAGAA | | | |
| | | | | GCCATTGAAACAACACAAC | | | |
| Shigella | 7 | prey67411 | 200 | GCCTGAAGAACAAGAGGAAAGAAAACCTTCTGCCACCCAGCAGAAGAAAAA | 401 | PEEQEERKPSATQQKKNT | |
| ospG | | | | CACCAAACTCTCTAGCAAAACCACTGCTAAGTTATCCACTAGTGCTAAAAGAA | | KLSSKTTAKLSTSAKRIQKE | |
| | | | | TTCAGAAGGAGCTAGCTGAAATAACCCTTGATCCTCCTCCTAATTGCAGTGC | | LAEITLDPPPNCSAGPKGD | |
| | | | | TGGGCCTAAAGGAGATAACATTTATGAATGGAGATCAACTATACTTGGTCCA | | NIYEWRSTILGPPGSVYEG | |
| | | | | CCGGGTTCTGTATATGAAGGTGGTGTGTTTTTTCTGGATATCACATTTTCATC | | GVFFLDITFSSDYPFKPPKV | |
| | | | | AGATTATCCATTTAAGCCACCAAAGGTTACTTTCCGCACCAGAATCTATCACT | | TFRTRIYHCNINSQGVICLDI | |
| | | | | GCAACATCAACAGTCAGGGAGTCATCTGTCTGGACATCCTTAAAGACAACTG | | LKDNWSPALTISKVLLSICS | |
| | | | | GAGTCCCGCTTTGACTATTTCAAAGGTTTTGCTGTCTATTTGTTCCCTTTTGA | | LLTDCNPADPLVGSIATQYL | |
| | | | | CAGACTGCAACCCTGCGGATCCTCTGGTTGGAAGCATAGCCACTCAGTATTT | | TNRAEHDRIARQWTKRYAT | |
| | | | | GACCAACAGAGCAGAACACGACAGGATAGCCAGACAGTGGACCAAGAGATA | | * | |
| | | | | CGCAACATAA | | | |
| Shigella | 7 | prey67423 | 201 | ATGAGTTCTCAACAGTTTCCTCGGTTAGGAGCCCCTTCTACCGGGCTGAGCC | 402 | MSSQQFPRLGAPSTGLSQ | |
| ospG | | | | AGGCCCCTTCTCAGATTGCAAACAGTGGTTCTGCTGGATTGATAAACCCAGC | | APSQIANSGSAGLINPAATV | |
| | | | | TGCTACAGTCAATGATGAATCTGGTCGAGATTCTGAAGTCAGTGCCAGGGAG | | NDESGRDSEVSAREHMSS | |
| | | | | CACATGAGTTCCAGCAGCTCCCTCCAGTCCCGGGAGGAGAAGCAAGAGCCT | | SSSLQSREEKQEPVVVRPY | |
| | | | | GTTGTGGTAAGGCCCTATCCACAGGTGCAGATGTTGTCGACACACCATGCTG | | PQVQMLSTHHAVASATPVA | |
| | | | | TCGCATCAGCCACACCTGTTGCAGTGACAGCCCCGCCAGCACACCTGACGC | | VTAPPAHLTPAVPLSFSEG | |
| | | | | CAGCAGTGCCACTTTCATTTTCGGAGGGACTTATGAAGCCGCCCCCGAAGC | | LMKPPPKPTMPSRPIAPAP | |
| | | | | CCACCATGCCTAGCCGTCCCATTGCTCCTGCTCCACCTTCTACCCTGTCACT | | PSTLSLPPKVPGQVTVTME | |
| | | | | TCCCCCCAAGGTTCCAGGGCAGGTTACCGTTACCATGGAGAGTAGCATCCC | | SSIPQASAIPVATISGQQGH | |
| | | | | TCAAGCTTCAGCCATTCCTGTGGCAACAATCAGTGGACAACAGGGCCATCCC | | PSNLHHIMTTNVQMSIIRSN | |
| | | | | AGTAACCTGCATCACATCATGACTACAAATGTGCAAATGTCTATCATCCGCAG | | APGPPLHIGASHLPRGAAA | |
| | | | | CAATGCTCCTGGGCCCCCTCTTCACATTGGAGCTTCTCATTTACCTCGAGGT | | AAVMSSSKVTTVLRPTSQL | |
| | | | | GCAGCTGCTGCTGCTGTGATGTCCAGTTCTAAAGTAACCACAGTCCTGAGGC | | PNAATAQPAVQHIIH | |
| | | | | CGACCTCACAGCTGCCAAATGCTGCTACTGCTCAGCCAGCAGTACAGCACAT | | | |
| | | | | CATTCACC | | | |
| Shigella | 7 | prey67298 | 202 | GATATTCTAGGTGTTAGGGTGCTGCAATCCCCTGGAACTGTATTAGTTGATTT | 403 | DILGVRVLQSPGTVLVDFIS | |
| ospG | | | | TATTTCATGAGTGTGCATAAAACACCTTCTATCTATGGGACTGGCATGGGGC | | *VCIKHLLSMGLAWGLVLXT | |
| | | | | TTGGTGCTTANAACATATAGATGAACAAGATCTTTGCTAGCAAGGAGCTGAG | | YR*TRSLLARS*ELSEERVK | |
| | | | | AGCTTAGTGAAGAAAGAGTGAAAAGTCCACAGTGAGAACATGGAGGNGCAC | | SPQ*EHGGAHTWAAGTLP | |
| | | | | ATACCTGGGCTGCAGGCACACTGCCTNTGCCTGATCCAGTCCTGACACTGA | | XPDPVLTLKNVXMIXRXG | |
| | | | | AAAATGTGNNCATGATANGAAGANGGGGG | | | |
| Shigella | 7 | prey67464 | 203 | NTTGNTGGGTGNGNTNGGGGTGATAAGGAAAGAGTGTGAGAAAATGGCATC | 404 | XXGXXXGDKERV*ENGIKQ | |
| ospG | | | | AAACAGGGAACAAGTAAGAGGTCTGGTGGCAAGCGGACAAGAGATGAGTCC | | GTSKRSGGKRTRDESVNP | |
| | | | | GTCAACCCCCACAACTGAGACTTGAGAGGGATGAGTGGGTCCTGAGAACTC | | HN*DLRGMSGS*ELRQS*V | |
| | | | | AGGCAAAGCTGAGTAGGTGGCCCCACTATCAATTAAAAAAGAGATCAGCTTA | | GGPTIN*KRDQLTCYXXSY | |
| | | | | CCTGCTACTANTANAGTTACCCTGGGCTCCGATGCANTGATGGCAGTGGGG | | PGLRCXDGSGGRXPXPXG | |
| | | | | GCCGGNAGCCGGNGCCCANGGGCCCTGGCCTNATNANTNTTGAG | | PGLXXXE | |
| Shigella | 7 | prey67320 | 204 | TCAGTGCCTGCTAGATACTTTGACAAGTTGGCTAGAACAGCGTTGTTCAGAT | 405 | SVPARYFDKLARTALFRWS | |
| ospG | | | | GGAGCATAGAACATCGAGATTACTTTTCTTCACCATGGCAATTGAGTACTGAT | | IEHRDYFSSPWQLSTDLCL | |
| | | | | CTTTGTCTTCCATCTCTTAAGTACATTTACTTCTGAACTATGTATGCTATATAA | | PSLKYIYF*TMYAI*FISVIVV | |
| | | | | TTCATATCTGTGATAGTAGTGGGTGACTTGATAGATATTATCTGGCTATGTGT | | GDLIDIIWLCVLPC*QVIYVS | |
| | | | | ACTTCCATGTTAGCAAGTGATTTATGTGTCAAAGTTTCTACCCAGTGGGAATT | | KFLPSGN*VSLIL | |
| | | | | AGGTCAGTTTAATTTTG | | | |
| Shigella | 7 | prey67321 | 205 | GTGTTGAGTATNCTCAGANNTNACGTTGCAATTGAAGNNCTGGNTCAGGAAC | 406 | VLSXLRXXVAIEXLXQEP*K | |
| ospG | | | | CCTGAAAAGATGTTNCCAGCTANNGATNAAGCAAGNCCGCTGGTGGGNGTC | | DVXSXXXSKXAGGXPXYH | |
| | | | | CCTTNTACCATNTNGGGGCTTTTGNNNNNTTNCTATCAANGCGTGCTTTTCTT | | XGAFXXXLSXRAFLFQLXX | |
| | | | | TTCCAACTACANANGCACATGGAAGTGGTCACTATCCGCTCTCTCCAGTATT | | HMEVVTIRSLQYYXHQNXF | |
| | | | | ATANCCATCAGAATNNCTTCTTGCAGGANNNACTGGTTGTGNNGANGCNTNT | | LQXXLVVXXXXWXLDXAEX | |
| | | | | GTGGGANTTAGACANNGCNGAGNNGGTNTNCGGGGGTTNNT | | VXGGX | |
| Shigella | 7 | prey35777 | 206 | ATGGGGCCCCTCTCAGCCCCTCCCTGCACAGAGCACATCAAATGGAAGGGG | 407 | MGPLSAPPCTEHIKWKGLL | |
| ospG | | | | CTCCTGGTCACAGCATCACTTTTAAACTTCTGGAACCTGCCCACCACTGCCC | | VTASLLNFWNLPTTAQVTIE | |
| | | | | AAGTCACGATTGAAGCCCAGCCACCAAAAGTTTCCGAGGGGAAGGATGTTCT | | AQPPKVSEGKDVLLLVHNL | |
| | | | | TCTACTTGTCCACAATTTGCCCCAGAATCTTACTGGCTACATCTGGTACAAAG | | PQNLTGYIWYKGQIRDLYH | |
| | | | | GGCAAATCAGGGACCTCTACCATTACATTACATCATATGTAGTAGACGGTCA | | YITSYVVDGQIIIYGPAYSGR | |
| | | | | AATAATTATATATGGGCCTGCATATAGTGGACGAGAAACAGCATATTCCAATG | | ETAYSNASLLIQNVTREDA | |
| | | | | CATCCCTGCTGATCCAGAATGTCACCCGGGAGGACGCAGGATCCTACACCT | | GSYTLHIIKRGDGTRGVTG | |
| | | | | TACACATCATAAAGCGAGGTGATGGGACTAGAGGAGTAACTGGATATTTCAC | | YFTFTLYLETPKPSISSSNL | |
| | | | | CTTCACCTTATACCTGGAGACTCCCAAGCCCTCCATCTCCAGCAGCAACTTA | | NPREAMETVILTCDPETPD | |
| | | | | AACCCCAGGGAGGCCATGGAAACTGTGATCTTAACCTGTGATCCTGAGACTC | | TSYQWWMNGQSLPMTHR | |
| | | | | CGGACACAAGCTACCAGTGGTGGATGAATGGTCAGAGCCTCCCTATGACTC | | FQLSETNRTLFLFGVTKYTA | |
| | | | | ATAGGTTTCAGCTGTCCGAAACCAACAGGACCCTCTTTCTATTTGGTGTCACA | | GPYECEIRNSGSASRSDPV | |
| | | | | AAGTATACTGCAGGACCCTATGAATGTGAAATACGGAACTCAGGGAGTGCCA | | TLNLLHGPDLPRIHPSYTNY | |
| | | | | GCCGCAGTGACCCAGTCACCCTGAATCTCCTCCATGGTCCAGACCTCCCCA | | RSGDNLYLSCFANSNPPAQ | |
| | | | | GAATTCACCCTTCATACACCAATTACCGTTCAGGAGATAACCTCTACTTGTCT | | YSWTINGKFQQSGQNLFIP | |
| | | | | TGCTTCGCGAACTCTAACCCACCGGCACAGTATTCTTGGACAATTAATGGGA | | QITTKHSGLYVCSVRNSAT | |
| | | | | AGTTTCAGCAATCAGGACAAAATCTGTTTATCCCCCAAATTACTACAAAGCAT | | GQESSTSLTVKVSASTRIGL | |
| | | | | AGCGGGCTCTATGTTTGCTCTGTTCGTAACTCAGCCACTGGGCAGGAAAGCT | | LPLLNPT* | |
| | | | | CCACATCGTTGACAGTCAAAGTCTCTGCTTCTACAAGAATAGGACTTCTTCCT | | | |
| | | | | CTCCTTAATCCAACATAG | | | |
| Shigella | 7 | prey67327 | 207 | GCAGGCTTTGAACTTTACCCGTTTTCTTGACCAGTCAGGACCCCCATCTGGG | 408 | QALNFTRFLDQSGPPSGDV | |
| ospG | | | | GATGTGAATTCCCTTGATAAGAAGTTGGTGCTGGCATTCAGGCACCTGAAGC | | NSLDKKLVLAFRHLKLPTE | |
| | | | | TGCCCACGGAGTGGAATGTATTGGGGACAGATCAGAGTTTGCATGATGCTG | | WNVLGTDQSLHDAGPRET | |
| | | | | GCCCGCGAGAGACATTGATGCATTTTGCTGTGCGGCTGGGACTGCTGAGGT | | LMHFAVRLGLLRLTWFLLQ | |
| | | | | TGACGTGGTTCCTGTTGCAGAAGCCAGGTGGCCGCAGAGCTCTCAGTATCC | | KPGGRRALSIHNQEGATPV | |
| | | | | ACAACCAGGAAGGGGCGACGCCTGTGAGCTTGGCCTTGGAGCGAGGCTAT | | SLALERGYHKLHQLLTEEN | |
| | | | | CACAAGCTGCACCAGCTTCTAACCGAGGAGAATGCTGGAGAACCAGACTCC | | AGEPDSWSSLSYEIPYGDC | |
| | | | | TGGAGCAGTTTATCCTATGAAATACCGTATGGAGACTGTTCTGTGAGGCATC | | SVRHHRELDIYTLTSESDS | |
| | | | | ATCGAGAGTTGGACATCTATACATTAACCTCTGAGTCTGATTCACATCATGAA | | HHEHPFPGDGCTGPIFKLM | |
| | | | | CACCCATTTCCTGGAGACGGTTGCACTGGACCAATTTTTAAACTTATGAACAT | | NIQQQLMKTNLKQMDSLM | |
| | | | | CCAACAGCAACTAATGAAAACAAACCTCAAGCAGATGGACAGTCTTATGCCC | | PLMMTAQDPSSAPETDGQ | |
| | | | | TTAATGATGACAGCACAGGATCCTTCCAGTGCCCCAGAGACAGATGGCCAGT | | FLPCAPEPTDPQRLSSSEE | |
| | | | | TTCTTCCCTGTGCACCGGAGCCCACGGACCCTCAGCGACTTTCTTCTTCTGA | | TESTQCCPGS | |
| | | | | AGAGACTGAGAGCACTCAGTGCTGCCCAGGGAGCCC | | | |
| Shigella | 7 | prey412 | 208 | GAGCATTGCACCCAAAACTACCCGGGTGACATACCCAGCCAAAGCCAAGGG | 409 | SIAPKTTRVTYPAKAKGTFI | |
| ospG | | | | CACATTCATCGCAGACAGCCACCAGAACTTCGCCTTGTTCTTCCAGCTGGTA | | ADSHQNFALFFQLVDMNT | |
| | | | | GATATGAACACTGGTGCTGAACTCACTCCTCACCAGACATTTGTCCGACTCC | | GAELTPHQTFVRLHNQKTG | |
| | | | | ATAACCAGAAGACTGGCCAGGAAGTGGTGTTTGTTGCCGAGCCAGACAACA | | QEVVFVAEPDNKNVYKFEL | |
| | | | | AGAACGTGTACAAGTTTGAACTGGATACCTCTGAAAGAAAGATTGAATTTGAC | | DTSERKIEFDSASGTYTLYL | |
| | | | | TCTGCCTCTGGCACCTACACTCTCTACTTAATCATTGGAGATGCCACTTTGAA | | IIGDATLKNPILWNVADVVIK | |
| | | | | GAACCCAATCCTCTGGAATGTGGCTGATGTGGTCATCAAGTTCCCTGAGGAA | | FPEEEAPSTVLSQNLFTPK | |
| | | | | GAAGCTCCCTCGACTGTCTTGTCCCAGAACCTTTTCACTCCAAAACAGGAAA | | QEIQHLFREPEKRPPT | |
| | | | | TTCAGCACCTGTTCCGCGAGCCTGAGAAGAGGCCCCCCACCG | | | |
| Shigella | 7 | prey50598 | 209 | CCTCCGTGTCCGCAGCCTGCCCGGAGAGGACCTGAGGGCCCGTGTTAGCT | 410 | LRVRSLPGEDLRARVSYRL | |
| ospG | | | | ACAGGCTGCTGGGGGTCATCTCACTGCTGCACCTGGTGCTGTCCATGGGGC | | LGVISLLHLVLSMGLQLYGF | |
| | | | | TGCAGCTGTACGGTTTCAGGCAGCGGCAGCGAGCCAGGAAGGAGTGGAGG | | RQRQRARKEWRLHRGLSH | |
| | | | | CTGCACCGCGGCCTGTCTCACCGCAGGGCCTCCTTGGAGGAGAGAGCCGT | | RRASLEERAVSRNPLCTLC | |
| | | | | TTCCAGAAACCCCCTGTGCACCCTGTGCCTGGAGGAGCGCAGGCACCCAAC | | LEERRHPTATPCGHLFCW | |
| | | | | AGCCACGCCCTGCGGCCACCTGTTCTGCTGGGAGTGCATCACCGCGTGGTG | | ECITAWCSSKAECPLCREK | |
| | | | | CAGCAGCAAGGCGGAGTGTCCCCTCTGCCGGGAGAAGTTCCCTCCCCAGAA | | FPPQKLIYLRHYR* | |
| | | | | GCTCATCTACCTTCGGCACTACCGCTGA | | | |
| Shigella | 7 | prey67364 | 210 | TTATTAAATGAAACAACAGTGGAAATATAGCCAGACCTGACTAACCTTGCCTG | 411 | LLNETTVEI*PDLTNLACIFL* | |
| ospG | | | | TATTTTCTTGTAGGCAGGAGAAAATCAGAGGCATCAAGATCTGGTAGAAGGG | | AGENQRHQDLVEGPVCCL | |
| | | | | CCGGTCTGCTGTTTAACACATACCAGCAGACAGGTCCCACGTGGGAGGCAC | | THTSRQVPRGRHHRPLR*G | |
| | | | | CACAGACCTTTAAGATAGGGTGAAGCCTTGATAGAAGGAGAAACAGAAGCTG | | EALIEGETEAAHCLYLEVEN | |
| | | | | CCCACTGTCTTTACTTAGAAGTGGAGAACATGGNATTCTGTATTTATTTATGT | | MXFCIYLC*LRXFTFXN | |
| | | | | TGACTGCGCANCTTTACNTTTNTAAACC | | | |
| Shigella | 7 | prey67367 | 211 | ATCCAGCAAAACCGCTGCTAAATTGTCAACTAGTGCTAAAAGAATTCAGAAG | 412 | SSKTAAKLSTSAKRIQKELA | |
| ospG | | | | GAACTTGCAGAAATCACATTGGACCCTCCTCCCAACTGTAGTGCTGGACCCA | | EITLDPPPNCSAGPKGDNIY | |
| | | | | AAGGAGACAACATTTATGAATGGAGGTCAACTATATTGGGACCCCCAGGATC | | EWRSTILGPPGSVYEGGVF | |
| | | | | TGTCTATGAAGGAGGGGTGTTCTTTCTTGACATTACCTTTTCACCAGACTATC | | FLDITFSPDYPFKPPKVTFR | |
| | | | | CGTTTAAACCCCCTAAGGTTACCTTCCGAACAAGAATCTATCACTGTAATATT | | TRIYHCNINSQGVICLDILKD | |
| | | | | AACAGCCAAGGTGTGATCTGTCTGGACATCTTAAAGGACAACTGGAGTCCGG | | NWSPALTISKVLLSICSLLT | |
| | | | | CTTTAACTATTTCTAAAGTTCTCCTCTCCATCTGCTCACTTCTTACAGATTGCA | | DCNPADPLVGSIATQYMTN | |
| | | | | ACCCTGCTGACCCTCTGGTGGGCAGCATCGCCACACAGTACATGACCAACA | | RAEHDRMARQWTKRYAT* | |
| | | | | GAGCAGAGCATGACCGGATGGCCAGACAGTGGACCAAGCGGTACGCCACA | | | |
| | | | | TAG | | | |
| Shigella | 7 | prey67369 | 212 | GTTGCAATGAGCCGAGATGGTGCCACTCATGTATATGAAACTCATCCATGGT | 413 | VAMSRDGATHVYETHPWW | |
| ospG | | | | GGAACTTTTTTCAGATGTGTGAGCTCTGTAACCTTTTAAGGTCCTGGAAACAT | | NFFQMCELCNLLRSWKHSI | |
| | | | | AGTATTTTTAAAAGTACACTGTATATCTCTATCAGGAAATTAAAATTGTTAGCT | | FKSTLYISIRKLKLLAYIYISIK | |
| | | | | TATATCTACATTTCAATAAAATGTAAGCCTGTTGCTATGTTGATAGCAAATCTG | | CKPVAMLIANLFNLLVIRLLR | |
| | | | | TTTAACTTACTGGTCATTAGGCTGTTACGTACGTCAATGAACTGGTGAAAGGA | | TSMNW*KEKIYETXLN | |
| | | | | GAAAATTTATGAAACATANCTCAAC | | | |
| Shigella | 7 | prey67372 | 213 | GAGATAAGGTGATGTCAGAGTTTAATAACAACTTCCGGCAGCAGATGGAGAA | 414 | DKVMSEFNNNFRQQMENY | |
| ospG | | | | TTACCCGAAAAACAACCACACTGCTTCGATCCTGGACAGGATGCAGGCAGAT | | PKNNHTASILDRMQADFKC | |
| | | | | TTTAAGTGCTGTGGGGCTGCTAACTACACAGATTGGGAGAAAATCCCTTCCA | | CGAANYTDWEKIPSMSKN | |
| | | | | TGTCGAAGAACCGAGTCCCCGACTCCTGCTGCATTAATGTTACTGTGGGCTG | | RVPDSCCINVTVGCGINFN | |
| | | | | TGGGATTAATTTCAACGAGAAGGCGATCCATAAGGAGGGCTGTGTGGAGAA | | EKAIHKEGCVEKIGGWLRK | |
| | | | | GATTGGGGGCTGGCTGAGGAAAAATGTGCTGGTGGTAGCTGCAGCAGCCCT | | NVLVVAAAALGIAFVEVLGI | |
| | | | | TGGAATTGCTTTTGTCGAGGTTTTGGGAATTGTCTTTGCCTGCTGCCTCGTG | | VFACCLVKSIRSGYEVM* | |
| | | | | AAGAGTATCAGAAGTGGCTACGAGGTGATGTAG | | | |
| Shigella | 7 | prey67379 | 214 | NAAANCNGTCTTAATCGCCACNTACTTCTCCNNNNCACATGTAAAACATANTT | 415 | XXXLNRHXLLXXTCKTXLX | |
| ospG | | | | GNTGTTNNGGGCCACNGNNGGCTGTNANTACTGNATTTNANATNNNTATTGG | | XXATXGCXYXIXXXYWXLA | |
| | | | | NNNCTNGCACATGTTAAAGGNNNCACAGTTTCTGNACTCTAGGAGANATTCT | | HVKGXTVSXL*EXFLXC*XX | |
| | | | | TGNCCTGTTAGNGTNAAAGTACTTTTCACTNGATAAGCTATGNTGACGTTNCT | | STFHXISYXDVXYXNXXXX* | |
| | | | | TATNAGAACNGNNNTTANTGNTGANTGCATGATNTCCATTCATNATGTATTTG | | XHDXHSXCICHEXLIXXTCR | |
| | | | | CCATGAGNNGCTAATTNNCAANACGTGTCGTAATGAGAATAA | | NEN | |
| Shigella | 7 | prey67381 | 215 | ATGACAGTCCAAGCACTAGTGGAGGAAGTTCCGATGGAGATCAACGTGAAA | 416 | MTVQALVEEVPMEINVKVF | |
| ospG | | | | GTGTTCAGCAAGAACCAGAAAGAGAACAAGTTCAGCCCAAGAAAAAGGAGG | | SKNQKENKFSPRKRREKY | |
| | | | | GAAAAATATCCAGCAAAACCGCTGCTAAATTGTCAACTAGTGCTAAAAGAATT | | PAKPLLNCQLVLKEFRRNL | |
| | | | | CAGAAGGAACTTGCAGAAATCACATTGGACCCTCCTCCCAACTGTAGTGCTG | | QKSHWTLLPTVVLDPKETT | |
| | | | | GACCCAAAGGAGACAACATTTATGAATGGAGGTCAACTATATTGGGACCCCC | | FMNGGQLYWDPQDLSMKE | |
| | | | | AGGATCTGTCTATGAAGGAGGGGTGTTCTTTCTTGACATTACCTTTTCACCAG | | GCSFLTLPFHQTIRLNPLRL | |
| | | | | ACTATCCGTTTAAACCCCCTAAGGTTACCTTCCGAACAAGAATCTATCACTGT | | PSEQESITVILTAKV* | |
| | | | | AATATTAACAGCCAAGGTGTGA | | | |

What is claimed is:

1. A complex between a Shigella flexneri polypeptide and a mammalian polypeptide as defined in columns 1 and 3 respectively of Table II.

2. A complex between a Shigella flexneri polynucleotide encoding a polypeptide as defined in column 1 of Table II, and a mammalian polynucleotide encoding a polypeptide as defined in column 3 of Table II.

3. A recombinant host cell expressing a polynucleotide encoding a Shigella flexneri polypeptide as defined in column 1 of Table II and a polynucleotide encoding a mammalian polypeptide as defined in column 3 of Table II.

4. The complex of claim 1 or claim 2 wherein said mammalian polypeptide is a human placenta polypeptide.

5. A method for selecting a modulating compound that inhibits or activates the protein-protein interactions between a Shigella flexneri polypeptide and a human placenta polypeptide in Table II comprising:
(a) cultivating a recombinant host cell on a selective medium containing a modulating compound and a reporter gene the expression of which is toxic for said recombinant host cell wherein said recombinant host cell is transformed with two vectors:
(i) wherein said first vector comprises a polynucleotide encoding a first hybrid polypeptide and a DNA bonding domain; and
(ii) wherein said second vector comprises a polynucleotide encoding a second hybrid polypeptide and an activating domain that activates said toxic reporter gene when the first and second hybrid polypeptides interact; and
(b) selecting said modulating compound which inhibits the growth of said recombinant host cell.

6. A modulating compound obtained from the method of claim 5.

7. A SID® polypeptide comprising one of SEQ ID Nos. 216 to 416.

8. A SID® polynucleotide comprising one of SEQ ID Nos. 15 to 215.

9. A vector comprising the SID® polynucleotide of claim 8.

10. A fragment of the SID® polypeptide of claim 7.

11. A variant of the SID® polypeptide of claim 7.

12. A fragment of the SID® polynucleotide of claim 8.

13. A variant of said SID® polynucleotide of claim 8.

14. A vector comprising the fragment of the SID® polynucleotide of claim 12.

15. A recombinant host cell containing the vector of claim 9.

16. A pharmaceutical composition comprising a modulating compound of claim 6 and a pharmaceutically acceptable carrier.

17. A pharmaceutical composition comprising a SID® polypeptide of SEQ ID Nos. 216 to 416 and a pharmaceutically acceptable carrier.

18. A pharmaceutical composition comprising the recombinant host cell of claim 15 and a pharmaceutically acceptable carrier.

19. A protein chip comprising a Shigella flexneri polypeptide of SEQ ID NOS. 1 to 7 or a mammalian polypeptide of Column 3, Table II.

20. A record comprising all or part of the data set forth in Tables I and II.
